SEQUENCE LISTING (188 sequences)

SEQ ID NO: 1 - protein, 124 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, synthetic construct [sequence omitted]

SEQ ID NO: 3 - protein, 8 aa, synthetic construct
GFTFRDYA

SEQ ID NO: 4 - protein, 8 aa, synthetic construct
ISGSGGNT

SEQ ID NO: 5 - protein, 16 aa, synthetic construct
AKDRLSITIRPRYYGL

SEQ ID NO: 6 - protein, 11 aa, synthetic construct
QSLLYSIGYNY

SEQ ID NO: 7 - skipped

SEQ ID NO: 8 - protein, 9 aa, synthetic construct
MQALQTPYT

SEQ ID NO: 9 - protein, 451 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 219 aa, synthetic construct
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYGSSPPWTFGQGTKVEIK

SEQ ID NO: 12 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPWTFGQGTKVEIK

SEQ ID NO: 13 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYGSSPPWTFGQGTKVEIK

SEQ ID NO: 14 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPPWTFGQGTKVEIK

SEQ ID NO: 15 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYGSSPPWTFGQGTKVEIK

SEQ ID NO: 16 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYGSSPPWTFGQGTKVEIK

SEQ ID NO: 17 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSPPWTFGQGTKVEIK

SEQ ID NO: 18 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSAGWTFGQGTKVEIK

SEQ ID NO: 19 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSAGWTFGQGTKVEIK

SEQ ID NO: 20 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSPPWTFGQGTKVEIK

SEQ ID NO: 21 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSPPWTFGQGTKVEIK

SEQ ID NO: 22 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSPPWTFGQGTKVEIK

SEQ ID NO: 23 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYDHSAGWTFGQGTKVEIK

SEQ ID NO: 24 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYDHSAGWTFGQGTKVEIK

SEQ ID NO: 25 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYDHSAGWTFGQGTKVEIK

SEQ ID NO: 26 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSAGWTFGQGTKVEIK

SEQ ID NO: 27 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSAGWTFGQGTKVEIK

SEQ ID NO: 28 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSAGWTFGQGTKVEIK

SEQ ID NO: 29 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSPPWTFGQGTKVEIK

SEQ ID NO: 30 - protein, 109 aa, synthetic construct
EIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIK

SEQ ID NO: 31 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 32 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 33 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 34 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFDYWGQGTLVTVSS

SEQ ID NO: 35 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFDYWGQGTLVTVSS

SEQ ID NO: 36 - protein, 115 aa, synthetic construct
EVQLVESGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 37 - protein, 115 aa, synthetic construct
EVQLVESGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 38 - protein, 115 aa, synthetic construct
EVQLVQSGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 39 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 40 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 41 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 42 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 43 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 44 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 45 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 46 - protein, 115 aa, synthetic construct
EVQLVQSGGGLVHPGRSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 47 - protein, 115 aa, synthetic construct
EVQLVQSGGGLVHPGGSLRLTCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 48 - protein, 115 aa, synthetic construct
EVQLVQSGGGLVHPGGSLRLSCAGSGFTFSRNAMHWVRQAPGKGLEWVSGIGTGGATNYADSVKGRFTISRDNAKNSLYLQMNSLRAEDMAVYYCARGRYYFDYWGQGTLVTVSS

SEQ ID NO: 49 - protein, 115 aa, synthetic construct
EVQLVQSGGGLVHPGGSLRLSCAGSGFTFSRNAMFWVRQAPGEGLEWVSGIGTGGATNYADSVKGRFTISRDNAKNSLYLQMNSLRAEDMAVYYCARGRYYFDYWGQGTLVTVSS

SEQ ID NO: 50 - protein, 115 aa, synthetic construct
EVQLVQSGGGLVHPGGSLRLSCAGSGFTFSRNAMFWVRQAPGKGLEWVSGIGTGGATNYADSVKGRFTISRDEAKNSLYLQMNSLRAEDMAVYYCARGRYYFDYWGQGTLVTVSS

SEQ ID NO: 51 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAGDMAVYYCARGRYYFDYWGQGTLVTVSS

SEQ ID NO: 52 - protein, 115 aa, synthetic construct
EVQLVQSGGGLVHPGGSLRLSCAGSGFTFDDYAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 53 - protein, 115 aa, synthetic construct
EVQLVQSGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 54 - protein, 115 aa, synthetic construct
EVQLVESGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 55 - protein, 115 aa, synthetic construct
EVQLVESGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 56 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 57 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 58 - protein, 115 aa, synthetic construct
EVQLVQSGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 59 - protein, 115 aa, synthetic construct
EVQLVQSGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 60 - protein, 115 aa, synthetic construct
EVQLVESGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 61 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 62 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 63 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 64 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 65 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 66 - protein, 5 aa, synthetic construct
RNAMF

SEQ ID NO: 67 - protein, 16 aa, synthetic construct
GIGTGGATSYADSVKG

SEQ ID NO: 68 - protein, 7 aa, synthetic construct
GRYYFDY

SEQ ID NO: 69 - protein, 12 aa, synthetic construct
RASQSVSSSYLA

SEQ ID NO: 70 - protein, 7 aa, synthetic construct
GASSRAT

SEQ ID NO: 71 - protein, 10 aa, synthetic construct
QQYDHSAGWT

SEQ ID NO: 72 - protein, 7 aa, synthetic construct
GASSRAP

SEQ ID NO: 73 - protein, 10 aa, synthetic construct
QQYGSSPPWT

SEQ ID NO: 74 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLDYWGKGTLVTVSS

SEQ ID NO: 75 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSLSANYVFGTGTKLTVL

SEQ ID NO: 76 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSQPPNPLFGTGTKLTVL

SEQ ID NO: 77 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKLLKNPWGKGTLVTVSS

SEQ ID NO: 78 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWFGTPASNYVFGTGTKLTVL

SEQ ID NO: 79 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSSPPQPIFGTGTKLTVL

SEQ ID NO: 80 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYDWGKGTLVTVSS

SEQ ID NO: 81 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 82 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTYHPIFGTGTKLTVL

SEQ ID NO: 83 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQYWGKGTLVTVSS

SEQ ID NO: 84 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTMYPLFGTGTKLTVL

SEQ ID NO: 85 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVLTPIFGTGTKLTVL

SEQ ID NO: 86 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWFYDWGKGTLVTVSS

SEQ ID NO: 87 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPSMIPLFGTGTKLTVL

SEQ ID NO: 88 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTLQPLFGTGTKLTVL

SEQ ID NO: 89 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPPTKPLFGTGTKLTVL

SEQ ID NO: 90 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTHRHPLFGTGTKLTVL

SEQ ID NO: 91 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQHWGKGTLVTVSS

SEQ ID NO: 92 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVDRPIFGTGTKLTVL

SEQ ID NO: 93 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTPMPVFGTGTKLTVL

SEQ ID NO: 94 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVWEWPFGTGTKLTVL

SEQ ID NO: 95 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEAVYFCGTWDTSTVWEWPFGTGTKLTVL

SEQ ID NO: 96 - protein, 111 aa, synthetic construct
QPVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 97 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 98 - protein, 111 aa, synthetic construct
QSVLTQPPSVSAAPGQKVTISCSGGSSNIGNNYVSWYQQLPGTAPKLLIYDNNKRPPGIPDRFSGSKSGTSATLAITGLQTGDEADYYCGTWDTSTVWEWPFGTGTKLTVL

SEQ ID NO: 99 - protein, 116 aa, synthetic construct
QVQLVQSGAEVKKPGASVKVSCKASGYAFTSYYMHWARQAPGQGLEWMGIINPRGGSTSYAQKFQGRVTMTRDTSTSTVYMELSSLRSEDTAVYYCARGKYWMYDWGKGTLVTVSS

SEQ ID NO: 100 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 101 - protein, 116 aa, synthetic construct
QVQLVQSGAEVKKPGASVKVSCKASGYAFTSYYMHWARQAPGQGLEWMGIINPRGGSASYAQKFQGRVSMTRDTSTSTVYMELSSLRSEDTAVYYCARGKYWMYDWGKGTLVTVSS

SEQ ID NO: 102 - protein, 111 aa, synthetic construct
QSVLTQPPSVSAAPGQKVTISCSGGSSNIGNSYVSWYQQLPGTAPKLLIYDNNKRPSGIPDRFSGSKSGTTATLAITGLQTGDEADYYCGTWVTSTVWEWPFGTGTKLTVL

SEQ ID NO: 103 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTQVTVSS

SEQ ID NO: 104 - protein, 111 aa, synthetic construct
QSVLTQPPLVSAAPGQKVTISCSGGSSNIGNSYVSWYQRLPGTAPKLLIYDNNKRPSGIPDRFSGSKSGTSATLAITGLQTGDEADYYCGTWDTSTVWEWPFGTGTKLTVL

SEQ ID NO: 105 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGNGTLVTVSS

SEQ ID NO: 106 - protein, 111 aa, synthetic construct
LPVLTQPPSVSAAPGQKVTISCSGGSSSIGNSYVSWYQQLPGAAPKLLIYDNNKRPSGIPDRFSGFRSGTSATLAITGLQTGDEADYYCGTWDTSPVWEWPFGTGTKLTVL

SEQ ID NO: 107 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTRVTVSS

SEQ ID NO: 108 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVWEWPFGTGTKLTVL

SEQ ID NO: 109 - protein, 111 aa, synthetic construct
QSVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTVWEWPFGTGTKLTVL

SEQ ID NO: 110 - protein, 111 aa, synthetic construct
QSVLTQPPSVSAAPGQKVTISCSGGSSSIGNSYVSWYQQLPGTAPKLLIYDNNKRPSGIPDRFSGSKSGTSATLAITGLQTGDEADYYCGTWATSPVWEWPFGTGTKLTVL

SEQ ID NO: 111 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTAWEWPFGTGTKLTVL

SEQ ID NO: 112 - protein, 116 aa, synthetic construct
QVQLVQSGAEEKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 113 - protein, 111 aa, synthetic construct
QSALTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 114 - protein, 116 aa, synthetic construct
QVQLVQSGAEVKKPGASVKVSCKASGYAFTSYYMHWARQAPGQGLEWMGIINPSGGSTSYAQKFQGRVSMTRDTSTSTVYMELSSLRSEDTAVYYCARGKYWMYDWGKGTLVTVSS

SEQ ID NO: 115 - protein, 116 aa, synthetic construct
QVQLVQSGAEVKKPGASVKVSCKASGYAFTSYYMHWARQAPGQGLEWMGIINPRGGSTSYAQKFQGRVAMTRDTSTSTVYMELSSLRPEDTAVYYCARGKYWMYDWGKGTLVTVSS

SEQ ID NO: 116 - protein, 111 aa, synthetic construct
QSVLTQPPSVSAAPGQKVTISCSGGGSSIGNSYVSWYQQLPGTAPKLLIYDNNKRPSGVPDRFSGSKSGTSATLAITGLQTGDEADYYCGTWDTSPVWEWPFGTGTKLTVL

SEQ ID NO: 117 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTAWEWPFGTGTKLTVL

SEQ ID NO: 118 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTLVTVSG

SEQ ID NO: 119 - protein, 111 aa, synthetic construct
QSVLTQPPSVSAAPGQKVTISCSGGSTNIGNSYVSWYQRLPGTAPKLLIYDNNKRPPGIPDRFSGSKSGTSATLAITGLQTGDEADYYCGTWDTSTVWEWPFGTGTKLTVL

SEQ ID NO: 120 - protein, 116 aa, synthetic construct
QVQLVQSGAEVKKPGASVKVSCKASGYAFTSYYMHWARQAPGQGLEWVGIINPSGGSTSYAQKFQGRVTMTRDTSTSTVYMELSSLRSGDTAVYYCARGKYWMYDWGKGTLVTVSS

SEQ ID NO: 121 - protein, 111 aa, synthetic construct
QAVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTGWEWPFGTGTKLTVL

SEQ ID NO: 122 - protein, 116 aa, synthetic construct
QVQLVQSGAEVKKPGASVKVSCKASGYAFTSYYMHWVRQAPGQGLEWMGIINPRGGSTSYAQKFQGRVTMTRDTSTSTVYMELSSLRSEDTAVYYCARGKYWMYDWGKGTLVTVSS

SEQ ID NO: 123 - protein, 111 aa, synthetic construct
QSVLTQPPSVSAAPGQKVTISCSGGGSSIGNSYVSWYQQLPGTAPKLLIYDNNKRPSGIPDRFSGSKSGTSATLGITGLQTGDEADYYCGTWDTSPVWEWPFGTGTKLTVL

SEQ ID NO: 124 - protein, 5 aa, synthetic construct
SYYMH

SEQ ID NO: 125 - protein, 17 aa, synthetic construct
IINPRGGSTSYAQKFQG

SEQ ID NO: 126 - protein, 7 aa, synthetic construct
GKYWMYD

SEQ ID NO: 127 - protein, 13 aa, synthetic construct
SGGGSSIGNSYVS

SEQ ID NO: 128 - protein, 7 aa, synthetic construct
DNNKRPS

SEQ ID NO: 129 - protein, 12 aa, synthetic construct
GTWDTSPVWEWP

SEQ ID NO: 130 - protein, 116 aa, synthetic construct
EVQLLESGGGLVQPGGSLRLSCAVSGFTFSNYAMSWVRQAPGKGLEWVSAISSGGGNIYYADSVKGRFTISRDNSKNTLYLQMNSLRAEDTAVYYCAKLRRYFDYWGQGTLVTVSS

SEQ ID NO: 131 - protein, 121 aa, synthetic construct
EVQLLESGGGLVQPGGSLRLSCAASGFTFSDYAMSWVRQAPGKGLEWVSAISSGGSSIYYADSVKGRFTISRDNSKNTLHLQMNSLRAEDTAVYYCARGPQRSATAVFDYWGQGTLVTVSS

SEQ ID NO: 132 - protein, 127 aa, synthetic construct
EVQLLESGGGLVQPGGSLRLSCAASGFTFSNYAMSWVRQAPGKGLEWVSWISPNSGNIYYADSVKGRFTISRDNSKNTLYLQMNSLRAEDTAVYYCARRPLSAAWSHSSYYNAMDVWGQGTLVTVSS

SEQ ID NO: 133 - protein, 116 aa, synthetic construct
EVQLLESGGGLVQPGGSLRLSCAASGFTFSGYAMSWVRQAPGKGLEWVSLISHSGSNTYYADSVKGRFTISRDNSKNTLYLQMNSLRAEDTAVYYCARPHRAFDYWGQGTLVTVSS

SEQ ID NO: 134 - protein, 116 aa, synthetic construct
EVQLLESGGGLVQPGGSLRLSCAASGFTFSNYAMSWVRQAPGKGLEWVSGISHGSGSIYYADSVKGRFTISRDNSKNTLYLQMNSLRAEDTAVYYCARPHRAFDYWGQGTLVTVSS

SEQ ID NO: 135 - protein, 116 aa, synthetic construct
EVQLLESGGGLVQPGGSLRLSCAASGFTFSNYAMSWVRQAPGKGLEWVSGISHGNGSIYYADSVKGRFTISRDNSKNTLYLQMNSLRAEDTAVYYCAKTGRHFDYWGQGTLVTVSS

SEQ ID NO: 136 - protein, 116 aa, synthetic construct
EVQLLESGGGLVQPGGSLRLSCAASGFTFSNYAMSWVRQAPGKGLEWVSSISPSGSSIYYADSVKGRFTISRDNSKNTLYLQMNSLRAEDTAVYYCARSYRAFDYWGQGTLVTVSS

SEQ ID NO: 137 - protein, 116 aa, synthetic construct
EVQLLESGGGLVQPGGSLRLSCAASGFTFSNYAMSWVRQAPGKGLEWVSAISPSGGSIYYADSVKGRFTISRDNSKNTLYLQMNSLRAEDTAVYYCARAKRAFDYWGQGTLVTVSS

SEQ ID NO: 138 - protein, 116 aa, synthetic construct
EVQLLESGGGLVQPGGSLRLSCAASGFTFSNYAMSWVRQAPGKGLEWVSAISPGSGSTYYADSVKGRFTISRDNSKNTLYLQMNSLRAEDTAVYYCAKFRRHFDYWGQGTLVTVSS

SEQ ID NO: 139 - protein, 116 aa, synthetic construct
EVQLLESGGGLVQPGGSLRLSCAASGFTFSNYAMSWVRQAPGKGLEWVSAISSGGGNIYYADSVKGRFTISRDNSKNTLYLQMNSLRAEDTAVYYCARVHRAFDYWGQGTLVTVSS

SEQ ID NO: 140 - protein, 116 aa, synthetic construct
EVQLLESGGGLVQPGGSLRLSCAASGFTFSNYAMSWVRQAPGKGLEWVSAITSSGRSIYYADSVKGRFTISRDNSKNTLYLQMNSLRAEDTAVYYCARVHRAFDYWGQGTLVTVSS

SEQ ID NO: 141 - protein, 116 aa, synthetic construct
EVQLLESGGGLVQPGGSLRLSCAASGFTFSNYAMSWVRQAPGKGLEWVSAITSSGANIYYADSVKGRFTISRDNSKNTLYLQMNSLRAEDTAVYYCARVHRAFDYWGQGTLVTVSS

SEQ ID NO: 142 - protein, 116 aa, synthetic construct
EVQLLESGGGLVQPGGSLRLSCAASGFTFSNYAMSWVRQAPGKGLEWVSAITSSGGNIYYADSVKGRFTISRDNSKNTLYLQMNSLRAEDTAVYYCARVHRAFDYWGQGTLVTVSS

SEQ ID NO: 143 - protein, 116 aa, synthetic construct
EVQLLESGGGLVQPGGSLRLSCAASGFTFSNYAMSWVRQAPGKGLEWVSAITAGGGSIYYADSVKGRFTISRDNSKNTLYLQMNSLRAEDTAVYYCARVHRAFDYWGQGTLVTVSS

SEQ ID NO: 144 - protein, 116 aa, synthetic construct
EVQLLESGGGLVQPGGSLRLSCAASGFTFSRHAMAWVRQAPGKGLEWVSAITSSGRSIYYADSVKGRFTISRDNSKNTLYLQMNSLRAEDTAVYYCARVHRAFDYWGQGTLVTVSS

SEQ ID NO: 145 - protein, 110 aa, synthetic construct
QSVLTQPPSASGTPGQRVTISCSGSSSNIGNNYVNWYQQLPGTAPKLLIYDNSHRPSGVPDRFSGSKSGTSASLAISGLRSEDEADYYCGTWDASLSAYVFGGGTKLTVL

SEQ ID NO: 146 - protein, 110 aa, synthetic construct
QSVLTQPPSASGTPGQRVTISCSGSSSNIGNNNVSWYQQLPGTAPKLLIYANSKRPSGVPDRFSGSKSGTSASLAISGLRSEDEADYYCGSWDDSLSAYVFGGGTKLTVL

SEQ ID NO: 147 - protein, 110 aa, synthetic construct
QSVLTQPPSAPGTPGQRVTISCTGSSSNIGSNSVNWYQQLPGTAPKLLIYDDSHRPSGVPDRFSGSKSGTSASLAISGLRSEDEADYYCDAWDSSLSAYVFGGGTKLTVL

SEQ ID NO: 148 - protein, 110 aa, synthetic construct
QSVLTQPPSASGTPGQRVTLSCTGSSSNIGSNYVSWYQQLPGTAPKLLIYADSQRPSGVPDRFSGSKSGTSASLAISGLRSEDEADYYCGTWDDSLSGYVFGGGTKLTVL

SEQ ID NO: 149 - protein, 110 aa, synthetic construct
QSVLTQPPSASGTPGQRVTISCSSSSSNIGSNYVSWYQQLPGTAPKLLIYSDSHRPSGVPDRFSGSKSGTSASLAISGLRSEDEADYYCGSWDYSLSAYVFGGGTKLTVL

SEQ ID NO: 150 - protein, 110 aa, synthetic construct
QSVLTQPPSASGTPGQRVTISCTGSSSNIGNNTVSWYQQLPGTAPKLLIYDNSHRPSGVPDRFSGSKSGTSASLAISGLQSEDEADYYCGSWDYSLSAYVFGGGTKLTVL

SEQ ID NO: 151 - protein, 110 aa, synthetic construct
QSVLTQPPSASGTPGQRVTISCTGSSSNIGNNDVNWYQQLPGTAPKLLIYYDSQRPSGVPDRFSGSKSGTSASLAISGLRSEDEADYYCATWDASLSAYVFGGGTKLTVL

SEQ ID NO: 152 - protein, 110 aa, synthetic construct
QSVLTQPPSASGTPGQRVTISCSGSSSNIGSNAVNWYQQLPGTAPKLLIYYDNQRPSGVPDRFSGSKSGTSASLAISGLRSEDEADYYCGTWDDSLNGYVFGGGTKLTVL

SEQ ID NO: 153 - protein, 110 aa, synthetic construct
QSVLTQPPSASGTPGQRVTISCSGSSSNIGNNAVTWYQQLPGTAPKLLIYDDSHRPSGVPDRFSGSKSGTSASLAISGLRSEDEADYYCGSWDYSLSAYVFGGGTKLTVL

SEQ ID NO: 154 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLSGYVLGGGTKLTVL

SEQ ID NO: 155 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLRGYVLGGGTKLTVL

SEQ ID NO: 156 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGYWDYSLSGYVLGGGTKLTVL

SEQ ID NO: 157 - protein, 111 aa, synthetic construct
QSVLTQPPSASGTPGQRVTISCSGSSANSRTDGFNWYQQLPGTAPKLLIYADSHRPSGVPDRFSGSKSGTSASLAISGLRSEDEADYYCGTWDYSLSGYVLGGGTKLTVLG

SEQ ID NO: 158 - protein, 111 aa, synthetic construct
QSVLTQPPSASGTPGQRVTISCSGSAQFGSRDNFNWYQQLPGTAPKLLIYADSHRPSGVPDRFSGSKSGTSASLAISGLRSEDEADYYCGTWDYSLSGYVLGGGTKLTVLG

SEQ ID NO: 159 - protein, 111 aa, synthetic construct
QSVLTQPPSASGTPGQRVTISCSGSTKQMHNYQFNWYQQLPGTAPKLLIYADSHRPSGVPDRFSGSKSGTSASLAISGLRSEDEADYYCGTWDYSLSGYVLGGGTKLTVLG

SEQ ID NO: 160 - protein, 111 aa, synthetic construct
QSVLTQPPSASGTPGQRVTISCSGSLLRGENLQFNWYQQLPGTAPKLLIYADSHRPSGVPDRFSGSKSGTSASLAISGLRSEDEADYYCGTWDYSLSGYVLGGGTKLTVLG

SEQ ID NO: 161 - protein, 111 aa, synthetic construct
QSVLTQPPSASGTPGQRVTISCSGSPLFPDSGSFNWYQQLPGTAPKLLIYADSHRPSGVPDRFSGSKSGTSASLAISGLRSEDEADYYCGTWDYSLSGYVLGGGTKLTVLG

SEQ ID NO: 162 - protein, 111 aa, synthetic construct
QSVLTQPPSASGTPGQRVTISCSGSAALDLSPSFNWYQQLPGTAPKLLIYADSHRPSGVPDRFSGSKSGTSASLAISGLRSEDEADYYCGTWDYSLSGYVLGGGTKLTVLG

SEQ ID NO: 163 - protein, 5 aa, synthetic construct
RHAMA

SEQ ID NO: 164 - protein, 17 aa, synthetic construct
AITSSGRSIYYADSVKG

SEQ ID NO: 165 - protein, 7 aa, synthetic construct
VHRAFDY

SEQ ID NO: 166 - protein, 13 aa, synthetic construct
SGSPLFPDSGSFN

SEQ ID NO: 167 - protein, 7 aa, synthetic construct
ADSHRPS

SEQ ID NO: 168 - protein, 11 aa, synthetic construct
GTWDYSLSGYV

SEQ ID NO: 169 - protein, 117 aa, synthetic construct
QVQLVQSGAEVKKPGASVKVSCKASGYTFTNYGISWVRQAPGQGLEWMGWISVYNGKTNYAQKLQGRVTMTTDTSTTTAYMEMRSLRSDDTAVYYCARGSGYDLDYWGQGTLVSVSS

SEQ ID NO: 170 - protein, 128 aa, synthetic construct
EVQLVESGGGLVQPGGSLRLSCAASGFTFSSFWMTWVRQAPGKGLEWVANIKQDGSEKYYVDSVKGRFTISRDNAKNSLYLQMNSLRAEDTAVYYCARDPGRTMVRGGIRYYYGMDVWGQGTTVTVSS

SEQ ID NO: 171 - protein, 122 aa, synthetic construct
EVKLAESGGGLVQPGGSLRLSCAASGFTFSSHWMNWVRQAPGKGLEWVANIKQDGSDKYYVDSVKGRFTISRDNAKNSLYLQLNSLIAEDTAVYYCARDRGVRPPRGAFDIWGQGTMVTVSS

SEQ ID NO: 172 - protein, 128 aa, synthetic construct
QVQLVQSGAEVKKPGASVKVSCKASGYTFNSYGISWVRQAPGQGLEWMGWIRTYNGNTNYAQKLQGRVTMTTDTSTSTAYMELRSLRSDDTAVYYCARDEARIVVAGTTPYYYGMDVWGQGTTVTVSS

SEQ ID NO: 173 - protein, 117 aa, synthetic construct
QVQLVESGGGLVQPGGSLRLSCAVSGFTISDHYMSWIRQAPGKGLEWISYISSSGSKIYYADSVKGRFTISRDNAKNSLFLQMNSLRAEDTAVYYCARTRQLVGDYWGQGTLVTVSS

SEQ ID NO: 174 - protein, 124 aa, synthetic construct
EVQLVESGGGLVQPGRSLRLSCAASGFTFDNYAMHWVRQAPGKGLEWVSGIRWNSGSIGYADSVKGRFTISRDNAKNSLYLQMNSLRAEDTALYYCAKEGGYSGYRPGPFFDYWGQGTLVTVSS

SEQ ID NO: 175 - protein, 117 aa, synthetic construct
QVQLVQSGAEVKKPGASVKVSCKASGYTFTNYGISWVRQAPGQGLEWMGWISVYNGHTNYAQKLQGRVTMTTDTSTSTAYMELRSLRSDDTAVYYCARGSGYDFDSWGQGTLVTVSS

SEQ ID NO: 176 - protein, 116 aa, synthetic construct
QVQLVQSGAEVKKPGASVKVSCKASRYTFTSYDINWVRQATGQGLEWMGWMNPNSGNTGYAQKFQGRVTMTRNTSTSTAYMELSSLRSEDTAVYYCARVRRFFDYWGQGTLVTVSS

SEQ ID NO: 177 - protein, 117 aa, synthetic construct
QVQLVQSGPEVKKPGASVKVSCKASGYTFTNYGISWVRQAPGQGLEWMGWISVYNGNINYAQKLQGRVTMTTDTSTSTAYMDLRSLRSDDTAVYYCARGSGYDFDYWGQGTLVTVSS

SEQ ID NO: 178 - protein, 122 aa, synthetic construct
QVQLVQSGAEVKKPGASVKVSCKDSAYTFNRYGISWVRQAPGQGLEWMGWISAYTGNTVYAQKLQGRVTMTTDNSTSTAYMELRSLRSDDTAVYYCARDKSIFGVVRGFDYWGQGTLVTVSS

SEQ ID NO: 179 - protein, 107 aa, synthetic construct
AIQMTQSPSSLSASVGDRVTITCRASQGIRNALGWYQQKPGKAPKLLIYAASSLQSGVPSRFSGSGSGTDFTLTFSSLQPEDFATYYCLQDFNYPYTFGQGTKLEIK

SEQ ID NO: 180 - protein, 107 aa, synthetic construct
DIQMTQSPSSVSASVGDRVTISCRASQGVSSWLAWYQQKPGNAPKLLISAASSIQSGVPSRFSGSGSGTDFTLTISSLQPEDFATYYCQQANSFPLTFGGGTKVEIK

SEQ ID NO: 181 - protein, 107 aa, synthetic construct
DIQMTQSPSSVSASVGDRVTITCRASQGISSWLAWYQQKPGKAPKLLIYAASSFQSGVPSRFSGSGSGTDFTLTISSLQPEDFATYFCQQANSFPLTFGGGTTVEIK

SEQ ID NO: 182 - protein, 107 aa, synthetic construct
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLATK

SEQ ID NO: 183 - protein, 107 aa, synthetic construct
DIQLTQSPSFLSASVGDRVTITCWASQGISSYLAWYQQKPGKAPKLLIFAASTLQSGVPSRFSGSGSGTEFTLTISSLQPEDFATYYCQQLNSYPLTFGGGTKVEIR

SEQ ID NO: 184 - protein, 107 aa, synthetic construct
EIVMTQSPATLSVSPGERATLSCRASQSVNYNLAWYQHKPGQAPRLLIYGASTRATGIPARFSGSGSGTEFTLTISSLQSEDFAVYYCQQYNNWPLTFGGGTKVEIK

SEQ ID NO: 185 - protein, 107 aa, synthetic construct
AIQMTQSSSSLSASVGDRVTITCRASQAIRNALGWYQQKPGKAPKVLIYAASSLQSGIPSRFSGSGSGTDFTLTISSLQPEDFATYYCLQDYDYPYTFGQGTKLEIK

SEQ ID NO: 186 - protein, 107 aa, synthetic construct
DIQLTQSPSFLSASVGDRVTITCWASQGIISYLAWYQQKPGKAPKLLIYAASTLHSGVPSRFSGSGSGTEFTLTISSLQPEDFATYYCHQLKSYPITFGQGTRLEIK

SEQ ID NO: 187 - protein, 107 aa, synthetic construct
AIQMTQSPSSLSASVGDRVTITCRASQDIRNALGWYQQKPGKAPKLLIYAASSLQSGVPSRFSGSASGTDFTLTISSLQPEDFAAYYCLQDYNYPYTFGQGTKLEIK

SEQ ID NO: 188 - protein, 107 aa, synthetic construct
EIVMTQSPVTLSLSPGERATLPCRASQSVSSSLAWYQQKAGQSPRLLIYGASTRATGIPARFSGSGSGTEFTLTISNLQSEDFAVYYCQQYNNWPLTFGGGTKVEIK

(19) United States
(12) Patent Application Publication
(10) Pub. No.: US 2023/0332201 A1
(43) Pub. Date: Oct. 19, 2023

(54) CELL CULTURE METHODS FOR ANTIBODY PRODUCTION

(71) Applicant: Regeneron Pharmaceuticals, Inc., Tarrytown, NY (US)

(72) Inventors: John Mattila, Nyack, NY (US); Xiaolin Tang, Tarrytown, NY (US); Hanne Bak, Tarrytown, NY (US); Shawn M. Lawrence, Nyack, NY (US); Amy S. Johnson, Briarcliff Manor, NY (US); Meghan Casey, Ringwood, NJ (US); Michelle LaFond, Tarrytown, NY (US); Andrew Tustian, Millwood, NY (US); Philip Mellors, Limerick (IE); John Hourihan, Limerick (IE); John Crowley, Limerick (IE); Laura Callinan, Monkstown (IE); Shadia Abike Oshodi, Tarrytown, NY (US); Ashley Witmer, Tarrytown, NY (US); Daniel Corbett, Tarrytown, NY (US); James Reilly, Tarrytown, NY (US); Ankit Vartak, Greenwich, CT (US); Mark Chiboroski, Tarrytown, NY (US); Alessandra Starling, Tarrytown, NY (US); Robert Stairs, Wellborn, FL (US); Hai-Yuan Goh, Limerick (IE); Liam Nicholl, Dublin (IE); Aishling Conlon, Castlebank (IE)

(21) Appl. No.: 18/115,321

(22) Filed: Feb. 28, 2023

Related U.S. Application Data
(60) Provisional application No. 63/315,897, filed on Mar. 2, 2022.
(60) Provisional application No. 63/411,899, filed on Sep. 30, 2022.
(60) Provisional application No. 63/417,873, filed on Oct. 20, 2022.
(60) Provisional application No. 63/436,854, filed on Jan. 3, 2023.
(60) Provisional application No. 63/448,655, filed on Feb. 27, 2023.

(51) Int. Cl. C12P 21/02; C12N 5/00; C12M 1/34; C07K 16/28

(52) U.S. Cl. CPC C12P 21/02; C12N 5/00; C12M 41/46; C12M 41/32; C07K 16/2866; C12N 2500/60; C12N 2500/02; C12N 2501/33; C12N 2500/32; C12N 2500/42; C07K 2317/21

(57) ABSTRACT

The present invention pertains to methods for manufacturing high titer antibody products. In particular, the invention pertains, in part, to improved serum-free animal cell culture medium, which can used for the production of a protein of interest. Additionally, the present invention further pertains to chromatographic procedures employed to successfully isolate the antibody product subject of the present disclosure.

### CROSS-REFERENCE TO RELATED APPLICATIONS

This application incorporates by reference and claims priority to and the benefit of Provisional Patent Application No. 63/315,897, filed on Mar. 2, 2022; Provisional Patent Application No. 63/411,899 filed on Sep. 30, 2022; Provisional Patent Application No. 63/417,873 filed on Oct. 20, 2022, Provisional Patent Application No. 63/436,854 filed on Jan. 3, 2023 and Provisional Patent Application No. 63/448,655 filed on Feb. 27, 2023.

### SEQUENCE LISTING

The instant application contains a Sequence Listing which has been filed electronically in .xml format and is hereby incorporated by reference in its entirety. Said .xml copy, created on Jan. 31, 2023, is Sequence Listing.xml and is 180,965 bytes in size.

### FIELD

This application relates to producing recombinant protein in high-productivity cells using serum-free media cultured for large-scale production, with improved systems and methods for measuring and maintaining cell culture conditions, harvesting, and purifying protein having reduced heterogeneity and improved quality with higher yield.

### BACKGROUND

Manufacturing processes for a therapeutic protein product should be optimized according to factors including, but not limited to, protein production conditions, protein structure and functional characteristics, and the desired final drug product. Even when using the same therapeutic protein for the same final drug product, changing protein production conditions may be required to optimize titer, batch size, yield, or quality of the product. In turn, the increased and optimized titers, batch sizes and yields require optimizing harvesting and purification processes to process the increased titers and loads while maintaining acceptable quality attributes.

To support increased demand for recombinantly produced protein drug products, cell production processes have been developed that are capable of generating higher titers and improved quality, including reduced heterogeneity and reduced sequence variants, while improving overall yield. Therefore, there is a need for improved cell culture media to meet these demands and specialized cell conditions.

Additionally, measuring and maintaining optimized cell culture conditions are critical and require specially designed equipment, including probes and sensors for measuring dissolved gasses such as oxygen. The use of large-scale vessels and bioreactors, however, can impact the equipment used to measure cell culture conditions, including their accuracy and maintenance requirements. Because the biopharmaceutical industry is highly regulated, irregularities in measurements may require investigation, potentially requiring additional resources and adding delays in manufacturing process. Therefore, there is a need for improved systems and methods for measuring dissolved gasses in large-scale bioreactors that provide improved accuracy and reduce maintenance requirements.

After production, isolating highly concentrated protein presents additional challenges, including limited process volume, filtration capacity, chromatographic column load capacity, and potentially high drug substance viscosity, each of which may affect outcomes such as material use, time spent, product quality, batch purity and batch yield. Thus, novel approaches for harvesting, purifying and formulating high titers of therapeutic protein have also been developed to meet these challenges, at each unit operation of an exemplary harvesting and purification process.

In order to accommodate processing higher titer drug product, improved methods and systems have been developed for manufacturing, harvesting and purifying high titer antibody drug products. For example, the increased titer of drug product and drug loading can exceed the capacity of certain chromatographic purification processes and alter the viscosities which negatively impacts the transfer of drug product along the different chromatography steps. Accordingly, there is a need for improved processes that not only address individual purification steps, but also the entire manufacturing process.

### SUMMARY

The present disclosure in part provides an improved process for large scale production of anti-IL4Rα antibody in high-productivity cells. Improved cell culture media and processes for maintaining optimal cell culture conditions have been developed to increase titer and improve quality. Improved processes have also been developed to harvest and purify the increased protein loads while increasing overall yields, reducing manufacturing and operation complexities, and improving overall quality in the drug substance and formulated drug product.

In some embodiments, the anti-IL-4Rα antibody is Dupilumab. In one aspect, the anti-IL-4Rα antibody comprises a heavy chain variable region (HCVR) comprising the amino acid sequence of SEQ ID NO:1 and a light chain variable region (LCVR) comprising the amino acid sequence of SEQ ID NO:2. In one aspect, the anti-IL-4Rα antibody comprises three heavy chain complementarity determining region (HCDR) sequences comprising SEQ ID NOs: 3, 4, and 5, and three light chain complementarity determining region (LCDR) sequences comprising SEQ ID NOs: 6, 7, and 8.

In some embodiments the protein of interest is an antigen-binding protein, blocking antibody, or receptor antagonist (e.g., interleukin-4 receptor α (IL-4Rα)). In some aspects, the protein of interest is a protein that has an Fc domain. Thus, in some aspects, the protein of interest is an antibody, such as a human antibody, humanized antibody, chimeric antibody, an antibody fragment, such as a Fab or F(ab′)₂, an ScFv molecule, or the like.

In some embodiments, the manufacturing process includes culturing cells capable of expressing an anti-IL4Rα antibody, or receptor antagonist as described above, in a cell culture media that comprises a base medium that is chemically defined, such as a custom formulation or a commercially available base medium. In another embodiment, the complete media is free of sera (“serum free” media) and/or free of hydrolysate. In another embodiment, the method includes the step of adding one or more point-of-use additions to the cell culture media described below. In some aspects, the point-of-use additions described below can also be included in the cell culture media at the outset.

It has been found that polyamine-supplemented media (“PS media”) increases cell viability and density, reduces cell doubling time, and permits high titer protein production by cells that are grown in such supplemented media. It has been found that PS media provides restored cell viability and density, cell doubling time, and high titer protein production, with or without sera and/or supplemental hydrolysate.

In one embodiment, cells cultured in PS media have an average doubling time that is no more than 30 hours. In one aspect, the cell doubling time is no more than 24 hours. Likewise, the inclusion of polyamines in serum-free media allows cultured cells to reach a higher viable cell density (VCD) than without the inclusion of polyamines.

In one embodiment, the media comprises one or more polyamines selected from the group consisting of ornithine, putrescine, spermine, spermidine or their combinations, in cell culture media that is free of sera and/or free of hydrolysate.

In one embodiment, the PS media is serum-free and comprises ornithine at a concentration of between 30 μM and 900 μM. In one aspect, the ornithine is present in the medium at a concentration (expressed in micromoles per liter) of at least about 30, 40, 50, 60, 70, 80, 90, 100, 150, 200, 250, 300, 350, 400, 450, 500, 540, 545, 550, 555, 560, 565, 568, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 620, 625, 630, 635, 640, 645, 650, 700, 750, 800, 850, or 900 μM. Ornithine may be present at between about 0.09 mM and about 0.9 mM. In some embodiments, the PS media comprises ≤7.5 g/L hydrolysate. In some embodiments, the PS media is free of any hydrolysate.

In one embodiment, PS media is serum-free and comprises putrescine at a concentration of between 30 μM and 900 μM. In one aspect the putrescine is present in the medium at a concentration (expressed in micromoles per liter) of at least about 30, 40, 50, 60, 70, 80, 90, 100, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 260, 365, 370, 375, 380, 385, 390, 395, 400, 405, or 410 μM. Putrescine may be present at between about 0.20 mM and about 0.714 mM. In one embodiment, the PS media contains 57 mg/L±8.55 mg/L putrescine·2HCl in addition to ≥15 mg/L±2.25 mg/L ornithine·HCl. In some embodiments, the medium comprises ≤7.5 g/L hydrolysate. In some embodiments, the PS media is free of any hydrolysate.

In one embodiment, the PS media is serum-free and comprises spermine at a concentration of between 10 μM and 900 μM. In one aspect the spermine is present in the medium at a concentration (expressed in micromoles per liter) of at least about 10, 20, 30, 40, 50, 60, 70, 80, 90, 100, 150, 200, 250, 300, 350, 400, 450, 500, 540, 545, 550, 555, 560, 565, 568, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 620, 625, 630, 635, 640, 645, 650, 700, 750, 800, 850, or 900 μM. In some embodiments, the PS media comprises ≤7.5 g/L hydrolysate. In some embodiments, the PS media is free of any hydrolysate.

In one embodiment, PS media is serum-free and comprises spermidine at a concentration of between 10 μM and 900 μM. In one aspect the spermidine is present in the medium at a concentration (expressed in micromoles per liter) of at least about 10, 20, 30, 40, 50, 60, 70, 80, 90, 100, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 260, 365, 370, 375, 380, 385, 390, 395, 400, 405, or 410 μM. In one embodiment, the PS media contains 57 mg/L±8.55 mg/L spermine·4HCl in addition to ≥15 mg/L±2.25 mg/L spermidine·HCl. In some embodiments, the medium comprises ≤7.5 g/L hydrolysate. In some embodiments, the medium contains ≤16 g/L hydrolysate. In some embodiments, the PS media is free of any hydrolysate.

In one embodiment, the media comprises a mixture of amino acids (with the notable exception of glutamine, which may be added back to the medium as a point of use addition) selected from the group of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and a combination thereof.

In some embodiments, the media comprises at least 40±6 mM or at least 70±10.5 mM of a mixture of amino acids or amino acid salts. In one embodiment, the media comprises at least 40 mM of a mixture of amino acids. In this or another aspect, the media comprises at least 70 mM of a mixture of amino acids. In some embodiments, the total process, including the base medium and feeds, contains a total of at least 115 mM of a mixture of amino acids or amino acid salts.

In some embodiments, the media comprises one or more fatty acids. In one aspect, the medium comprises a mixture of fatty acids (or fatty acid derivatives) and a tocopherol. In one aspect, fatty acids or fatty acid derivatives are selected from the group consisting of linoleic acid, linolenic acid, oleic acid, palmitic acid, stearic acid, arachidic acid, lauric acid, behenic acid, decanoic acid, dodecanoic acid, hexanoic acid, lignoceric acid, myristic acid, and octanoic acid and may can also include thioctic acid derived from fatty acids and a combination thereof.

In some embodiments, the media comprises one or more nucleosides. In one aspect, nucleosides are selected from the group consisting of adenosine, guanosine, cytidine, uridine, thymidine, hypoxanthine, and a combination thereof.

In some embodiments, the cell culture media comprises one or more salts. In some aspects, salts are selected from the group of divalent cations, such as calcium, magnesium, and a combination thereof. In one embodiment, the medium comprises calcium chloride, magnesium sulfate and a combination thereof. In another aspect, salts may also include those of phosphate.

In some embodiments, the cell culture media comprises any one or more of NaHCO₃, glutamine, insulin, glucose, CuSO₄, ZnSO₄, FeCl₃, NiSO₄, Na₄ EDTA, Na₃ Citrate, and a combination thereof. In one aspect, the method employs the step of adding one or more of the point-of-use chemicals to the cell culture media selected from the group consisting of NaHCO₃, glutamine, insulin, glucose, CuSO₄, ZnSO₄, FeCl₃, NiSO₄, Na₄ EDTA, Na₃ Citrate, and a combination thereof.

In some embodiments, the point-of-use addition comprises taurine, phosphate, poloxamer 188, and a combination thereof. In some aspects, the point-of-use additions can be included in the medium at the outset.

It has been found that the inclusion of taurine in a cell culture medium increases cellular specific productivity and allows for lower ammonia byproduct by those cells. In one aspect, the cell culture media is serum-free and comprises about 0.1 mM to about 10 mM taurine, about 1 mM to about 9 mM taurine, about 1 mM to about 8 mM taurine, about 1 mM to about 7 mM taurine, about 1 mM to about 6 mM taurine, about 1 mM to about 5 mM taurine, about 1 mM to about 4 mM taurine, about 1 mM to about 3 mM taurine, about 1 mM to about 2 mM taurine, about 0.1 mM to about 1 mM taurine, about 0.2 mM to about 1 mM taurine, about 0.3 mM to about 1 mM taurine, about 0.4 mM to about 1 mM taurine, or about 0.5 mM to about 1 mM taurine.

In some embodiments, the disclosure provides a method for producing an anti-IL-4Rα antibody or antigen binding fragment thereof by employing the steps of (1) introducing into a cell a nucleic acid sequence that encodes a protein of interest; (2) selecting a cell carrying that nucleic acid sequence; (3) culturing the selected cell in embodiments of the serum-free cell culture medium described in this disclosure; and (4) expressing the protein of interest in the cell, wherein the protein of interest is secreted into the medium. The biotherapeutic protein may be a blocking antibody or receptor antagonist (e.g., blocking IL-4Rα and IL-13), and/or an antigen-binding protein, which may comprise an Fc domain. In some embodiments, the antibody is a human, humanized, chimeric or non-human monoclonal antibody or an antibody fragment.

In some embodiments, the cell or cells are mammalian cells, avian cells, insect cells, yeast cells, or bacteria cells. In one aspect, the cells are mammalian cells useful in the production of recombinant proteins, such as CHO cells or the derivative CHO-K1. In some aspects, the cells express a protein of interest, such as a biotherapeutic protein. In some aspects, the cell used in the production of the protein is a mammalian cell capable of producing a biotherapeutic, such as CHO, HEK293, and BHK cell, or any derivatives of them. In one embodiment, the cell is a CHO cell, such as a CHO-K1 cell.

In some embodiments, the cell media (1) is serum-free; (2) comprises hydrolysate; (3) comprises one or more polyamines, including, at least, ornithine or spermine; (4) comprises at least about 40 mM or at least about 70 mM of a mixture of amino acids, including at least one of the following: alanine, arginine, asparagine, aspartic acid, cysteine, glutamic acid, glycine, histidine, isoleucine, leucine, lysine, methionine, phenylalanine, proline, serine, threonine, tryptophan, tyrosine, valine, and a combination thereof; (5) comprises tocopherol and a mixture of fatty acids; (6) comprises a mixture of nucleosides including at least one of the following: adenosine, guanosine, cytidine, uridine, thymidine, hypoxanthine, and a combination thereof; (7) comprises salts of at least one of the following: calcium, magnesium, and phosphate; and (8) comprises at least one of the following point-of-use additions: NaHCO₃, glutamine, insulin, glucose, CuSO₄, ZnSO₄, FeCl₃, NiSO₄, Na₄ EDTA, Na₃ citrate, and a combination thereof.

In some embodiments, the cell media (1) is serum-free; (2) comprises one or more polyamines, including, at least, ornithine or spermine; (3) comprises at least about 40 mM or at least about 70 mM of a mixture of amino acids, including at least one of the following: alanine, arginine, asparagine, aspartic acid, cysteine, glutamic acid, glycine, histidine, isoleucine, leucine, lysine, methionine, phenylalanine, proline, serine, threonine, tryptophan, tyrosine, valine, and a combination thereof; (4) comprises tocopherol and a mixture of fatty acids; (5) comprises a mixture of nucleosides including at least one of the following: adenosine, guanosine, cytidine, uridine, thymidine, hypoxanthine, and a combination thereof; (6) comprises salts, including at least one of the following: calcium, magnesium, phosphate, and a combination thereof; and (7) comprises at least one of the following point-of-use additions: NaHCO₃, glutamine, insulin, glucose, CuSO₄, ZnSO₄, FeCl₃, NiSO₄, Na₄ EDTA, Na₃ citrate, and a combination thereof.

In some embodiments, the cell media (1) is serum-free; (2) comprises ≤7.5 g/L of a hydrolysate; (3) comprises 0.09±0.014 mM, 0.3±0.05 mM, 0.6±0.09 mM, or 0.9±0.14 mM ornithine; (4) optionally additionally comprises 0.10±0.03 mM, 0.20±0.03 mM, 0.35±0.06 mM, or 0.714±0.11 mM putrescine, spermidine or spermine; (5) comprises at least about 40 mM or at least about 70 mM of a mixture of amino acids including at least one of alanine, arginine, asparagine, aspartic acid, cysteine, glutamic acid, glycine, histidine, isoleucine, leucine, lysine, methionine, phenylalanine, proline, serine, threonine, tryptophan, tyrosine, valine, and a combination thereof; (6) comprises tocopherol and optionally a mixture of fatty acids; (7) comprises a mixture of nucleosides including adenosine, guanosine, cytidine, uridine, thymidine, hypoxanthine, and a combination thereof; and (8) comprises salts of calcium, magnesium, phosphate, and a combination thereof.

In some embodiments, the cell media (1) comprises no hydrolysate; (2) comprises ornithine at a concentration of at least 90 μM±14 μM; (3) and optionally comprises putrescine, such as at least 150 μM±14 μM. In other embodiments, other polyamines such as spermine, spermidine, and the like are envisaged to be within the scope of the present invention.

In some embodiments, this disclosure provides a method for cultivating cells in a serum-free medium which comprises (1) ornithine at either 0.09±0.014 mM, 0.3±0.05 mM, 0.6±0.09 mM, or 0.9±0.14 mM; (2) optionally additionally putrescine at either 0.20±0.03 mM, 0.35±0.06, or 0.714±0.11 mM; (3) at least about 40 mM or at least about 70 mM of a mixture of amino acids including alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine; (4) tocopherol and a mixture of fatty acids; (5) a mixture of nucleosides including adenosine, guanosine, cytidine, uridine, thymidine, and hypoxanthine; and (6) salts of calcium, magnesium, and phosphate, wherein the cells cultured according to this method have an average doubling time that is no more than 24 hours or no more than one third the doubling time of cells cultured using similar cell culture medium that comprises less than 0.09±0.015 mM polyamines. In another embodiment, the cell culture is capable of attaining a viable cell density that is at least 3-fold greater than a similar cell culture in a similar cell culture medium that comprises less than 0.09±0.014 mM ornithine (or less than 0.09±0.014 mM ornithine and less than 0.2±0.03 mM putrescine). In one embodiment, the medium comprises ≤7.5 g/L hydrolysate; and in another embodiment, the medium is free of hydrolysates.

In some embodiments, the protein of interest is produced by (1) introducing into a CHO cell a nucleic acid sequence that encodes a protein of interest, such as an antibody or other antigen-binding protein; (2) selecting a cell carrying that nucleic acid sequence; (3) culturing the selected cell in a serum-free cell culture medium which comprises (a) ornithine at either 0.09±0.014, 0.3±0.05 mM, 0.6±0.09 mM, or 0.9±0.14 mM; (b) optionally additionally putrescine at either 0.20±0.03 mM, 0.35±0.06, or 0.714±0.11 mM; (c) at least 40 mM or at least 70 mM of a mixture of amino acids including one or more of: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine; (d) tocopherol and optionally a mixture of fatty acids; (e) a mixture of nucleosides including one or more of: adenosine, guanosine, cytidine, uridine, thymidine, hypoxanthine, and a combination thereof; and (f) salts including one or more of: calcium, magnesium, phosphate and a combination thereof; and (4) expressing the protein of interest in the CHO cell, wherein the protein of interest is secreted into the medium. In some embodiments, the serum-free cell culture medium may include ≤7.5 g/L hydrolysates; or in other embodiments no hydrolysates at all.

In some embodiments, the protein of interest is produced by (1) introducing into a CHO cell a nucleic acid sequence that encodes a protein of interest, such as an antibody or other antigen-binding protein; (2) selecting a cell carrying that nucleic acid sequence; (3) culturing the selected cell in a serum-free cell culture medium which comprises (a) ornithine at either 0.09±0.014, 0.3±0.05 mM, 0.6±0.09 mM, or 0.9±0.14 mM; (b) optionally additionally putrescine at either 0.20±0.03 mM, 0.35±0.06, or 0.714±0.11 mM; (c) at least 40 mM or at least 70 mM of a mixture of amino acids including one or more of: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and a combination thereof; (d) tocopherol and a mixture of fatty acids; (e) a mixture of nucleosides including one or more of: adenosine, guanosine, cytidine, uridine, thymidine, hypoxanthine, and a combination thereof; and (f) salts including one or more of: calcium, magnesium, and phosphate; and (4) expressing the protein of interest in the CHO cell, wherein the protein of interest is secreted into the medium. In some embodiments, the serum-free cell culture medium may include ≤7.5 g/L hydrolysates; or in other embodiments no hydrolysates at all.

The present disclosure in part provides an improved seed train process for increasing titer. In one embodiment, the initial viable cell density (VCD) at each step (from N-5 to N-1) is increased to from about 3.5×10⁵ to about 5.43×10⁵ cells/mL compared to standard initial VCD ranging from 1.7×10⁵ to 2.3×10⁵ cells/mL. In some aspects, the initial VCD (in N-5 to N-1) is about 1.3×, 1.4×, 1.5×, 1.6., 1.7×, 1.8×, 1.9×, 2.0×, 2.1×, 2.2×, 2.3×, 2.4×, 2.5×, 2.6×, 2.7×, 2.8×, 2.9×, or 3.0× greater than an alternative initial VCD in a standard seed train. In one aspect, the optimized seed train resulted in a 2%, 3%, 4%, 5%, 6%, 7%, 8%, 9%, 10%, 11%, 12%, 13%, 14%, 15%, 1%, 17%, 18%, 19%, or 20% increase in final titer (g/L). In one aspect, there is no substantial difference in peak lactate observed in the final production vessel compared to a standard seed train.

The present disclosure in part provides improved vessels and bioreactors for measuring and controlling cell culturing conditions, including dissolved gasses such as oxygen and carbon dioxide, which are important process parameters for maintenance of a healthy cell culture production process within the biopharmaceutical industry.

The present disclosure in part provides improved methods and systems for air sparging, agitation and measurement of dissolved gasses. More specifically, the present disclosure provides optimized dissolved gas concentrations at various intervals as well as improved bioreactors with optimized agitation rates and working volumes to provide necessary agitation while minimizing shear stress.

In certain exemplary embodiments, the starting volume, air sparge, and agitation setpoints in vessels and bioreactors, including various sensors, along the seed train during the growth phase and production phases are optimized to improve process robustness through minimization of shear stress on the cell culture and optimization of viable cell densities (VCD), dissolved oxygen, carbon dioxide, and pH at various times.

In some embodiments, the sparging rates may be used to vary pCO₂ levels within the cell culture to modulate acidic and basic charge variants for cultured cells expressing Dupilumab.

The present disclosure also provides improved methods and systems for measuring dissolved gasses. Electrochemical probes and optical probes may also be used to measure other dissolved gases such as carbon dioxide. Electrochemical probes have been conventionally used in vessels and bioreactors over optical probes because they have a long history of demonstrating a wide linear range and faster response time compared to optical probes or sensors. Electrochemical dissolved oxygen probes, however, require significant upkeep in terms of frequent replacement of membranes and electrolyte solution, and lengthy polarization times. In addition, electrochemical probes are known to provide noisy readings due to bubble interference, with evidence of this occurrence in a large-scale bioreactor provided in the examples below. Such noise can cause excursions outside of normal operating ranges that must be investigated and documented through the quality compliance system of a biopharmaceutical company, due to high regulations within the industry. In the event of short excursions on the high side, generally attributed to bubble interference, an investigation is performed, resulting in strains on manufacturing resources.

Although optical probes are less influenced by bubble interference from agitation, stirring and sparging, and do not have the same electrolyte changing and polarization requirements, optical probes can be less accurate and less responsive compared to electrochemical probes. Further, prior studies assessing the use of optical probes have been limited to water monitoring applications and small-scale cell culture applications. The largest vessel used in the reviewed literature during testing of optical probes was a 50 L single-use bioreactor (Marvell et al., 2009), leaving a gap in the reported scientific literature relating to the use of optical probes in large-scale stainless-steel production (>1,000 L) that have not addressed the performance issues with optical probes.

Accordingly, the present disclosure provides an improved system and method for reducing signal noise in electrochemical probes, which reduces or eliminates false positives attributable to bubble interference. Additionally, the present disclosure provides a system and method for improving accuracy of optical probes used for measuring dissolved gasses in large-scale vessels and bioreactors, which require lower maintenance and are not susceptible to bubble interference compared to conventional electrochemical probes. As discussed in more detail below, vessels and bioreactors with electrochemical probes and optical probes were developed with improved data processing methods to optimize the accuracy of the measurements while minimizing maintenance by reducing or eliminating unwanted excursion events outside of normal operating ranges, which require lengthy quality investigations in the highly regulated biopharmaceutical industry.

In one embodiment, the present disclosure provides methods for measuring VCD of cells cultured in a bioreactor by applying an electric field to cells cultured in a bioreactor, measuring the capacitance, and correlating the measured capacitance to viable cell density.

In one embodiment, the protein of interest is capable of being produced at an average seven-day titer that is at least 7% greater, at least 14% greater, at least 80% greater, at least two-fold greater, or at least three-fold greater than the average seven-day titer produced by a similar cell in a serum-free cell culture medium that comprises less than 0.09±0.014 mM ornithine (or less than 0.09±0.014 mM ornithine and less than 0.2±0.03 mM putrescine).

To harvest and purify the increased protein production, new and improved methods were developed that not only can accommodate the increased protein production but improve on the overall yield percentage. The methods include, for example, optimizations of harvest pre-treatment, protein A chromatography, viral inactivation, anion exchange chromatography, cation exchange chromatography, hydrophobic interaction chromatography, virus retentive filtration, concentration and diafiltration, and drug substance adjustment, which are further described below.

This disclosure provides a method for purifying an anti-IL4Rα antibody. In some exemplary embodiments, the method comprises (a) subjecting said antibody to pre-treatment; (b) harvesting said antibody; (c) subjecting said antibody to affinity chromatography; (d) subjecting antibody pooled from eluate in step (c) to viral inactivation at a pH from about 3 to about 4.5 and then adjusting the pH to from about 5 to about 8; (e) subjecting said antibody pooled from step (d) to anion exchange chromatography in flowthrough mode; (f) subjecting said antibody pooled from flowthrough fractions of step (e) to cation exchange chromatography in bind and elute mode; (g) subjecting said antibody pooled from eluate of step (f) to hydrophobic interaction chromatography in flowthrough mode; and (h) subjecting said antibody pooled from flowthrough fractions of step (g) to virus retentive filtration.

In one aspect, the antibody of step (h) is further subjected to concentration and diafiltration using a diafiltration buffer having a pH between 4.0 and 4.5.

In one aspect, the diafiltration buffer comprises from about 4 mM acetate to about 6 mM acetate.

In one aspect, harvest pre-treatment includes subjecting said antibody to transient pH levels from about 4.5 to 5.0 or about 4.0 to about 5.5 and then increasing the pH levels to from about 5.5 to 6.5.

In one aspect, the affinity chromatography is Protein A chromatography.

In one aspect, the harvesting of step (b) includes centrifugation and depth filtration.

In one aspect, the antibody is harvested in a 3 kL to 25 kL bioreactor.

In one aspect, the Protein A column load pH is between 7 and 8. In one aspect, the Protein A column load pH is between 6 and 8. In one aspect, the Protein A column load pH is about 6.

In alternate exemplary embodiments, the method comprises (a) culturing cells expressing the anti-IL-4Rα antibody or an antigen-binding fragment thereof; (b) subjecting said cells to transient pH levels from about 4.5 to 5.0 or about 4.0 to about 5.5, then adjusting the pH to from about 5.5 to 6.5; (c) harvesting said cells by centrifugation to separate cell debris from clarified media comprising the anti-IL-4Rα antibody or antigen-binding fragment thereof; (d) subjecting said clarified media to affinity chromatography; (e), subjecting the anti-IL-4Rα antibody or antigen-binding fragment thereof pooled from eluate in step (d) to viral inactivation at a pH from about 3 to about 4.5 and then adjusting the pH to from about 5 to about 8; (f) subjecting the anti-IL-4Rα antibody or antigen-binding fragment thereof pooled from step (e) to anion exchange chromatography in flowthrough mode; (g) subjecting the anti-IL-4Rα antibody or antigen-binding fragment thereof pooled from flowthrough fractions of step (f) to cation exchange chromatography in bind and elute mode; (h) subjecting the anti-IL-4Rα antibody or antigen-binding fragment thereof pooled from eluate of step (g) to hydrophobic interaction chromatography in flowthrough mode; and (i) subjecting the anti-IL-4Rα antibody or antigen-binding fragment thereof pooled from flowthrough fractions of step (h) to virus retentive filtration, thereby producing the anti-IL-4Rα antibody or antigen-binding fragment thereof.

In one aspect, the method further comprises subjecting said anti-IL-4Rα antibody or antigen-binding fragment to ultrafiltration and diafiltration (UF/DF) after step (i).

In one aspect, the affinity chromatography is Protein A chromatography.

In one aspect, the anti-IL-4Rα antibody or antigen-binding fragment thereof comprises three heavy chain complementarity determining region (HCDR) sequences comprising SEQ ID NOs: 3, 4, and 5, and three light chain complementarity determining region (LCDR) sequences comprising SEQ ID NOs: 6, 7, and 8.

In one aspect, the anti-IL-4Rα antibody or antigen-binding fragment thereof comprises a heavy chain variable region (HCVR) comprising the amino acid sequence of SEQ ID NO: 1 and a light chain variable region (LCVR) comprising the amino acid sequence of SEQ ID NO: 2.

In one aspect, the anti-IL-4Rα antibody is Dupilumab.

These and other aspects of the present disclosure will be better appreciated and understood when considered in conjunction with the following description and accompanying drawings. The following description, while indicating various embodiments and numerous specific details thereof, is given by way of illustration and not of limitation. Various substitutions, modifications, additions, or rearrangements may be made within the scope of the invention, and additional Enumerated Examples are provided below describing additional aspects of the present disclosure.

### BRIEF DESCRIPTION OF THE DRAWINGS

FIG. 1 illustrates the structure of an antibody product, Dupilumab, according to an exemplary embodiment.

FIG. 2 represents the effect of insulin supplementation on titer profile of Dupilumab, according to an exemplary embodiment.

FIG. 3 represents the effect of insulin supplementation on viability of cells expressing Dupilumab, according to an exemplary embodiment.

FIG. 4 shows the effect of insulin supplementation on the reduction of ammonia levels in the culture comprising cells expressing Dupilumab, according to an exemplary embodiment.

FIG. 5 shows the effect of insulin concentration of 15 mg/L and feed schedule on growth rate of the titer curve, upper asymptote (highest achievable titer), inflection point, and titer, according to an exemplary embodiment.

FIG. 6 shows the effect of insulin supplementation using the profiler for day 13 on high molecular weight species, according to an exemplary embodiment.

FIG. 7 shows the effect of insulin supplementation using the profiler for day 13 on levels of non-reduced purity, according to an exemplary embodiment.

FIG. 8 shows the effect of insulin supplementation schedule using the profiler for day 13 on non-glycosylated heavy chain (NGHC) levels, according to an exemplary embodiment.

FIG. 9 shows the effect of tyrosine supplementation on titer and ammonia levels in a cell culture expressing Dupilumab, according to an exemplary embodiment.

FIG. 10 shows the effect of phosphate redistribution strategy 6 and 8 on Dupilumab titer (g/L), according to an exemplary embodiment.

FIG. 11 shows phosphate redistribution profiles on days 0, 2, 4, 6, and 8, according to an exemplary embodiment.

FIG. 12A shows a contour plot of central composite design (CCD) model predicting host cell protein clearance factor (CF), according to an exemplary embodiment.

FIG. 12B shows a contour plot of CCD model predicting normalized supernatant turbidity (NTU), according to an exemplary embodiment.

FIG. 13A shows a contour plot of CCD model predicting host cell protein clearance factor, according to an exemplary embodiment.

FIG. 13B shows a contour plot of CCD model predicting normalized supernatant turbidity (NTU), according to an exemplary embodiment.

FIG. 14 shows the initial and final viable cell density (VCD) for an average standard seed train and an optimized seed train with an increased initial VCD in comparison to a standard seed train, according to an exemplary embodiment.

FIG. 15 shows the biomass decline in a 10 kL production bioreactor for cells cultured using an optimized seed train with an increased initial VCD in comparison to a standard seed train, according to an exemplary embodiment.

FIG. 16A and FIG. 16B show the final titer (g/L) of an optimized seed train (Sample 1) with an increased initial VCD in comparison to standard seed trains (Samples 2 to 9), according to an exemplary embodiment.

FIG. 17 shows a bivariate linear regression of the on-line capacitance measurements and off-line VCD measurements of cell line A (CLA) during seed train, according to an exemplary embodiment of the present invention.

FIG. 18 shows bivariate linear regressions of the on-line capacitance measurements and off-line VCD measurements of CLA during seed train N-3, N-2 and N-1 phases, according to an exemplary embodiment of the present invention.

FIG. 19 shows predictions of the on-line VCD measurements of CLA during seed train N-3, N-2 and N-1 phases which were determined using the on-line capacitance measurements and the correlation equation presented in FIG. 21, according to an exemplary embodiment of the present invention. The dots (circles) indicate VCD values determined off-line using a bioanalyzer.

FIG. 20 shows a bivariate linear regression of the on-line capacitance measurements and off-line VCD measurements of cell line B (CLB) during seed train, according to an exemplary embodiment of the present invention.

FIG. 21 shows bivariate linear regressions of the on-line capacitance measurements and off-line VCD measurements of CLB during seed train N-3, N-2 and N-1 phases, according to an exemplary embodiment of the present invention.

FIG. 22 shows predictions of the on-line VCD measurements of CLB during seed train N-3, N-2 and N-1 phases, which were determined using the on-line capacitance measurements and the correlation equation presented in FIG. 24, according to an exemplary embodiment of the present invention. The dots (circles) indicate VCD values determined off-line using a bioanalyzer.

FIG. 23 shows a bivariate linear regression of the on-line capacitance measurements and off-line VCD measurements of CLA and CLB during seed train, according to an exemplary embodiment of the present invention.

FIG. 24A, FIG. 24B, and FIG. 24C show predictions of the on-line VCD measurements of CLA during seed train N-3, N-2 and N-1 phases, which were determined using the on-line capacitance measurements presented in FIG. 17 and the correlation equations presented in FIG. 17 (FIG. 24A), FIG. 20 (FIG. 24B) and FIG. 23 (FIG. 24C), according to an exemplary embodiment of the present invention. The dots (circles) indicate VCD values determined off-line using a bioanalyzer.

FIG. 25A, FIG. 25B, and FIG. 25C show predictions of the on-line VCD measurements of CLB during seed train N-3, N-2 and N-1 phases (black trace), which were determined using the on-line capacitance measurements presented in FIG. 21 and the correlation equations presented in FIG. 20 (FIG. 25A), FIG. 17 (FIG. 25B) and FIG. 23 (FIG. 25C), according to an exemplary embodiment of the present invention. The dots (circles) indicate VCD values determined off-line using a bioanalyzer.

FIG. 26A and FIG. 26B show predictions of the on-line VCD measurements of CLB during seed train and production phases, which were determined using on-line capacitance measurements and the correlation equation presented in FIG. 20, according to an exemplary embodiment of the present invention. Panels A and B show the data for two separate cultures, each of which used a single probe to perform on-line capacitance measurements. In FIG. 26A, the line depicts the predictions of the on-line VCD measurements and the dots (circles) indicate VCD values determined off-line using a bioanalyzer. In FIG. 26B, the line depicts the predictions of the on-line VCD measurements and the dots (circles) indicate VCD values determined off-line using a bioanalyzer.

FIG. 27A shows the host cell protein values and Dupilumab yield post-acid precipitation of Dupilumab cell culture fluid using 1M phosphoric acid, according to an exemplary embodiment.

FIG. 27B shows the host cell protein values and Dupilumab yield post-acid precipitation of Dupilumab cell culture fluid using 1.75 M acetic acid, according to an exemplary embodiment.

FIG. 28 shows a schematic of an exemplary outline regarding the use of depth filters, polish filter and guard filters, according to an exemplary embodiment.

FIG. 29 shows a prediction profiler modeling the factors and response studied in the centrifuge characterization study, according to an exemplary embodiment.

FIG. 30 shows a schematic of exemplary purification steps of the optimized process, according to an exemplary embodiment.

FIG. 31 shows a schematic of exemplary purification steps of the optimized process, according to an exemplary embodiment.

FIG. 32 shows a visualization of models generated for a multivariate study of affinity capture and viral inactivation risk factors and responses, according to an exemplary embodiment.

FIG. 33 shows HCP levels in affinity capture pool when using various affinity wash buffers, according to an exemplary embodiment.

FIG. 34 shows HCP levels following AEX or HIC processing downstream of affinity capture chromatography using various affinity wash buffers, according to an exemplary embodiment.

FIG. 35 shows protein yield and HMW species in affinity capture pool when using various affinity wash buffers, according to an exemplary embodiment.

FIG. 36 shows a visualization of models generated for a multivariate study of AEX risk factors and responses, according to an exemplary embodiment.

FIG. 37 shows a visualization of models generated for a multivariate study of CEX risk factors and responses, according to an exemplary embodiment.

FIG. 38 shows a visualization of models generated for a multivariate study of HIC risk factors and responses, according to an exemplary embodiment.

FIG. 39 shows a visualization of models generated for a multivariate study of VRF risk factors and responses, according to an exemplary embodiment.

FIG. 40 shows a visualization of models generated for a multivariate study of UF/DF risk factors and responses, according to an exemplary embodiment.

FIG. 41 shows bioreactor titer for Dupilumab 500 L confirmation batches compared to 10,000 L process performance qualification batches and 2 L mirror productions, according to an exemplary embodiment.

FIG. 42 shows step yield by unit operation for 500 L confirmation batches compared to 10,000 L process performance qualification batches, according to an exemplary embodiment.

FIG. 43 shows HMW content by unit operation for 500 L confirmation batches compared to 10,000 L process performance qualification batches, according to an exemplary embodiment.

FIG. 44 shows HCP content by unit operation for 500 L confirmation batches compared to 10,000 L process performance qualification batches, according to an exemplary embodiment.

FIG. 45 illustrates an example of a stepped agitation and air sparging rate over time, according to an exemplary embodiment.

FIG. 46 illustrates an example of signal filtering for cell culture with a 59 second sampling rate and ˜10-minute sample window applying an Agile filter, according to an exemplary embodiment. The label “A” illustrates that excursions on the low side remain for the smoothed signal. The label “B” illustrates good agreement between smoothed and unfiltered signals for expected process disturbances.

FIG. 47 illustrates an example of signal filtering for cell culture with a 59 second sampling rate and ˜33-minute sample window, applying an Agile filter, according to an exemplary embodiment. The label “A” illustrates that excursions on the low side are removed for the smoothed signal. The label “B” illustrates good agreement between smoothed and unfiltered signals for expected process disturbances.

FIG. 48 illustrates an example of signal filtering for cell culture with a 59 second sampling rate and ˜33-minute sample window, applying a Savitzky-Golay filter, according to an exemplary embodiment. The label “A” illustrates that noise remains for the filtered signal. The label “B” illustrates good agreement between smoothed and unfiltered signals for expected process disturbances.

FIG. 49 illustrates a stacked comparison of the filtered signal from FIG. 46-FIG. 48, according to an exemplary embodiment.

FIG. 50 illustrates Run 1 dissolved oxygen trends (where the light shaded line is Electrochemical, medium shaded line is Optical Probe B, darkest shaded line is Optical Probe A, Dashed lines: setpoint and normal operating range upper and lower limits), according to an exemplary embodiment.

FIG. 51 illustrates Run 2 dissolved oxygen trends (where the light shaded line is Electrochemical, medium shaded line is Optical Probe B, darkest shaded line is Optical Probe A, Dashed lines: setpoint and normal operating range upper and lower limits), according to an exemplary embodiment.

FIG. 52 illustrates Run 3 dissolved oxygen trends (where the light shaded line is Electrochemical, medium shaded line is Optical Probe B, darkest shaded line is Optical Probe A, Dashed lines: setpoint and normal operating range upper and lower limits), according to an exemplary embodiment.

FIG. 53 illustrates Run 4 dissolved oxygen trends (where the light shaded line is Electrochemical, medium shaded line is Optical Probe B, darkest shaded line is Optical Probe A, Dashed lines: setpoint and normal operating range upper and lower limits), according to an exemplary embodiment.

FIG. 54 illustrates dissolved oxygen data generated using Optical Probe B with an anti-bubble cap for Runs 1-3 and a standard cap for Run 4, according to an exemplary embodiment.

FIG. 55 illustrates dissolved oxygen data as determined at time segments at ‘Start’, ‘Middle’ and ‘End’ of Run 2 examined as part of the offset assessment (where the light shaded line is Electrochemical, medium shaded line is Optical Probe B, darkest shaded line is Optical Probe A, the bottom most line is Electrochemical with Agile filter (59 s sampling rate and default sample window)), according to an exemplary embodiment.

FIG. 56 illustrates Run 1 Probe B Offset correction (where the medium shaded line is Optical Probe B, the darkest shaded line is Optical Probe B adjusted upwards by 3.0%, the light shaded line is Electrochemical with Agile filter (59 s sampling rate and default sample window), Dashed lines: setpoint and normal operating range upper/lower limits), according to an exemplary embodiment.

FIG. 57 illustrates Run 3 Probe B Offset Correction (where the medium shaded line is Optical Probe B, the darkest shaded line is Optical Probe B adjusted upwards by 3.0%, the light shaded line is Electrochemical with Agile filter (59 s sampling rate and default sample window), Dashed lines: setpoint and normal operating range upper/lower limits), according to an exemplary embodiment.

FIG. 58A and FIG. 58B illustrate examples of varying dextrose concentrations at various times.

FIG. 59 illustrates pCO₂ profiles of culture media with varying sparging conditions in mmHg (y-axis) over process time (days), according to an exemplary embodiment. Medium pCO2 was selected as a mid-point control.

FIG. 60 shows a visualization of models generated for a multivariate study of mixed-mode chromatography (MMC) risk factors and responses using Capto Adhere resin, according to an exemplary embodiment.

FIG. 61 shows a visualization of models generated for a multivariate study of MMC risk factors and responses using PPA HyperCel resin, according to an exemplary embodiment.

### DETAILED DESCRIPTION

Manufacturing processes for a therapeutic protein product, for example a monoclonal antibody drug, should be optimized according to factors including, but not limited to, recombinant host cell characteristics, protein production conditions, protein structure and functional characteristics, and the desired final drug product. To support increased demand for recombinantly produced protein drug products, cell lines and cell culture media have been developed that are capable of generating higher titers and larger batch sizes, with improved quality and yield.

Even when using the same therapeutic protein for the same final drug product, changing protein production conditions may be required to optimize titer, batch size, yield, or quality of the product. The applicants have made the discovery that the addition of polyamines, such as one or a combination of ornithine, putrescine, spermine and spermidine, to a medium (“PS media”) improves viable cell density, cell doubling time, and protein production of Dupilumab by a cell in a cell culture relative to a serum-free medium that comprises very little or no polyamines such as ornithine, putrescine, spermine and spermidine (“non-PS media”).

Conventional methods for harvesting and purifying protein drug products rely on a series of filtering and chromatography steps that may be limited in terms of their capacity to process increased titers and corresponding batch size, protein concentrations and viscosity of drug product using similarly sized equipment, while maintaining purity and quality standards. For example, pumps previously used to move drug product may fail when moving drug product with higher protein concentrations, due to increased viscosity levels. Techniques have been developed to reduce sample viscosity, for example using the addition of arginine, a hydrophobic salt. However, adding arginine during harvesting and purification, particularly during concentration and diafiltration, leads to the loss of unknown quantities of arginine in later processing steps, which must then be measured and compensated for further downstream, and may add variability to the final drug product. Therefore, a need exists for systems and methods for harvesting a high titer of antibody drug product while maintaining product consistency, high yield and quality.

The disclosure herein provides improvements to manufacturing, harvesting and purification processes for improving the production of a high titer antibody product. In some exemplary embodiments, a high titer antibody product of interest is Dupilumab.

Dupilumab is a human monoclonal immunoglobulin G4 (IgG4) antibody expressed and prepared from Chinese hamster ovary cells (CHO-K1) targeting interleukin-4 receptor subunit a (IL-4R-α). After cell cultivation, harvesting, isolation and purification, it is prepared for administration with, for example, histidine, histidine hydrochloride, arginine hydrochloride, polysorbate 80, sodium acetate, glacial acetic acid, sucrose and water.

Dupilumab inhibits interleukin-4 (IL-4) and interleukin-13 (IL-13) signaling by specifically binding to the IL-4 receptor α (IL-4Rα) subunit shared by the IL-4 and IL-13 receptor complexes. Dupilumab inhibits IL-4 signaling via the Type I receptor and both IL-4 and IL-13 signaling through the Type II receptor. Blocking IL4Rα with Dupilumab inhibits IL-4 and IL-13 cytokine-induced inflammatory responses, including the release of proinflammatory cytokines, chemokines, nitric oxide, and IgE. By blocking the obligate shared component of the IL-4/IL-13 receptor complex, Dupilumab inhibits IL-4 and IL-13 signaling, key disease drivers of Type II inflammation, and provides clinical benefit and long-term disease control without the side effects commonly observed with existing non-selective systemic immunosuppressants.

Dupilumab received its first worldwide marketing approval for the treatment of moderate-to-severe atopic dermatitis (AD) in adults in the United States on 28 Mar. 2017, then in the European Union on 28 Sep. 2017, in Japan on 19 Jan. 2018, and subsequently in multiple other countries. Since then, Dupilumab has been approved for treatment of other diseases associated with Type II inflammation, including moderate-to-severe asthma, chronic rhinosinusitis with nasal polyposis (CRSwNP), eosinophilic esophagitis (EoE), chronic spontaneous urticaria (CSU), and prurigo nodularis (PN). In addition, a subcutaneous administration of Dupilumab is currently being developed for the treatment of patients with type 2 inflammatory diseases, including AD (pediatric patients (6 months to <6 years)), asthma (pediatric patients (6 months to <6 years)), chronic obstructive pulmonary disease (COPD), allergic fungal rhino-sinusitis (AFRS), chronic rhinosinusitis without nasal polyps (CRSsNP), allergies (e.g. grass allergy, peanut allergy, and dairy allergy), bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, alopecia areata, and allergic rhinitis.

In order to meet a high demand for Dupilumab, a novel manufacturing process was developed, as described further below. The novel manufacturing process allows for, for example, the production and purification of Dupilumab 150 mg/mL and 175 mg/mL formulated drug substance (FDS) at 10,000 L scale and 25,000 L scale.

### DESCRIPTION OF TERMS

Unless described otherwise, all technical and scientific terms used herein have the same meaning as commonly understood by one of ordinary skill in the art to which this invention belongs. Methods and materials similar or equivalent to those described herein known to the skilled artisan can be used in the practice of particular embodiments described herein. All publications mentioned are hereby incorporated by reference in their entirety.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “upstream process technology,” in the context of protein preparation, refers to activities involving the production of proteins from cells in a cell culture. As used herein, the term “cell culture” refers to methods for generating and maintaining a population of host cells capable of producing a recombinant protein of interest, as well as the methods and techniques for optimizing the production and collection of the protein of interest. For example, once an expression vector has been incorporated into an appropriate host cell, the host cell can be maintained under conditions suitable for expression of the relevant nucleotide coding sequences, and the collection and production of the desired recombinant protein.

The present disclosure provides a serum-free medium that is useful in culturing cells and producing a biopharmaceutical drug substance. “Serum-free” applies to a cell culture medium that does not comprise animal sera, such as fetal bovine serum. The serum-free media may comprise ≤7.5 g/L of hydrolysates, such as soy hydrolysate. The present disclosure also provides chemically defined media (CDM), which is not only serum-free, but also hydrolysate-free.

“Hydrolysate-free” applies to cell culture media that comprises no exogenous protein hydrolysates, such as animal or plant protein hydrolysates, including, for example, peptones, tryptones and the like.

“Cell culture” or “culture” means the growth and propagation of cells outside of a multicellular organism or tissue. Suitable culture conditions for mammalian cells are known in the art. See e.g., Animal cell culture: A Practical Approach, D. Rickwood, ed., Oxford University Press, New York (1992). Mammalian cells may be cultured in suspension or while attached to a solid substrate. Fluidized bed bioreactors, hollow fiber bioreactors, roller bottles, flasks, or stirred tank bioreactors, with or without microcarriers, and operated in a batch, fed batch, continuous, semi-continuous, or perfusion mode are available for mammalian cell culture. Cell culture media or concentrated feed media may be added to the culture continuously or at intervals during the culture. For example, a culture may be fed once per day, every other day, every three days, or may be fed when the concentration of a specific medium component, which is being monitored, falls outside a desired range.

As used herein, the term “chemically defined medium” or “chemically defined media” (both abbreviated “CDM”) refers to a synthetic growth medium in which the identity and concentration of all the ingredients are defined. Chemically defined media do not comprise bacterial, yeast, animal, or plant extracts, animal serum, or plasma, although individual plant or animal-derived components (e.g., proteins, polypeptides, etc.) may be added. Chemically defined media may comprise inorganic salts such as phosphates, sulfates, and the like needed to support growth. The carbon source is defined, and is usually a sugar such as glucose, lactose, galactose, and the like, or other compounds such as glycerol, lactate, acetate, and the like. While certain chemically defined culture media also use phosphate salts as a buffer, other buffers may be employed such as sodium bicarbonate, N-2-hydroxyethylpiperazine-N′-2-ethanesulfonic acid (HEPES), citrate, triethanolamine, and the like. Examples of commercially available chemically defined media include, but are not limited to, various Dulbecco's Modified Eagle's (DME) media (Sigma-Aldrich Co; SAFC Biosciences, Inc.), Ham's Nutrient Mixture (Sigma-Aldrich Co; SAFC Biosciences, Inc.), various EX-CELLs mediums (Sigma-Aldrich Co; SAFC Biosciences, Inc.), various IS CHO-CD mediums (FUJIFILM Irvine Scientific), combinations thereof, and the like. Methods of preparing chemically defined culture media are known in the art, for example, in U.S. Pat. Nos. 6,171,825 and 6,936,441, WO 2007/077217, and U.S. Patent Application Publication Nos. 2008/0009040 and 2007/0212770, the entire teachings of which are herein incorporated by reference.

As used herein, the term “recombinant host cell” (or simply “host cell”) includes a cell into which a recombinant expression vector coding for a protein of interest has been introduced. It should be understood that such a term is intended to refer not only to a particular subject cell but to a progeny of such a cell. Because certain modifications may occur in succeeding generations due to either mutation or environmental influences, such progeny may not, in fact, be identical to the parent cell, but are still included within the scope of the term “host cell” as used herein. In an embodiment, host cells include prokaryotic and eukaryotic cells selected from any of the kingdoms of life. In one aspect, eukaryotic cells include protist, fungal, plant and animal cells. In a further aspect, host cells include eukaryotic cells such as plant and/or animal cells. The cells can be mammalian cells, fish cells, insect cells, amphibian cells or avian cells. In a particular aspect, the host cell is a mammalian cell. A wide variety of mammalian cell lines suitable for growth in culture are available from the American Type Culture Collection (Manassas, Va.) and other depositories as well as commercial vendors. Cells that can be used in the processes of the invention include, but are not limited to, MK2.7 cells, PER-C6 cells, Chinese hamster ovary cells (CHO), such as CHO-K1 (ATCC CCL-61), (Chasin et al., 1986, Som. Cell Molec. Genet., 12:555-556; Kolkekar et al., 1997, Biochemistry, 36: 10901-10909; and WO 01/92337 A2), dihydrofolate reductase negative CHO cells (CHO/−DHFR, Urlaub and Chasin, 1980, Proc. Natl. Acad. Sci. USA, 77:4216), and dp12.CHO cells (U.S. Pat. No. 5,721,121); monkey kidney cells (CV1, ATCC CCL-70); monkey kidney CV1 cells transformed by SV40 (COS cells, COS-7, ATCC CRL-1651); HEK293 cells, Sp2/0 cells, 5L8 hybridoma cells, Daudi cells, EL4 cells, HeLa cells, HL-60 cells, K562 cells, Jurkat cells, THP-1 cells, Sp2/0 cells, primary epithelial cells (e.g., keratinocytes, cervical epithelial cells, bronchial epithelial cells, tracheal epithelial cells, kidney epithelial cells and retinal epithelial cells) and established cell lines and their strains (e.g., human embryonic kidney cells (e.g., HEK293 cells, or HEK293 cells subcloned for growth in suspension culture, Graham et al., 1977, J. Gen. Virol., 36:59); baby hamster kidney cells (BHK, ATCC CCL-10); mouse sertoli cells (TM4, Mather, 1980, Biol. Reprod., 23:243-251); human cervical carcinoma cells (HELA, ATCC CCL-2); canine kidney cells (MDCK, ATCC CCL-34); human lung cells (W138, ATCC CCL-75); human hepatoma cells (HEP-G2, HB 8065); mouse mammary tumor cells (MMT 060562, ATCC CCL-51); buffalo rat liver cells (BRL 3A, ATCC CRL-1442); TRI cells (Mather, 1982, Annals NY Acad. Sci., 383:44-68); MCR 5 cells; FS4 cells; PER-C6 retinal cells, MDBK (NBL-1) cells, 911 cells, CRFK cells, MDCK cells, BeWo cells, Chang cells, Detroit 562 cells, HeLa 229 cells, HeLa S3 cells, Hep-2 cells, KB cells, LS 180 cells, LS 174T cells, NCI-H-548 cells, RPMI 2650 cells, SW-13 cells, T24 cells, WI-28 VA13, 2RA cells, WISH cells, BS-C-I cells, LLC-MK₂ cells, Clone M-3 cells, 1-10 cells, RAG cells, TCMK-1 cells, Y-1 cells, LLC-PK₁ cells, PK(15) cells, GH₁ cells, GH₃ cells, L2 cells, LLC-RC 256 cells, MH₁C₁ cells, XC cells, MDOK cells, VSW cells, and TH-I, B1 cells, or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, MiCl₁ cells, CV-1 cells, COS-1 cells, COS-3 cells, COS-7 cells, African green monkey kidney cells (VERO-76, ATCC CRL-1587; VERO, ATCC CCL-81); DBS-FrhL-2 cells, BALB/3T3 cells, F9 cells, SV-T2 cells, M-MSV-BALB/3T3 cells, K-BALB cells, BLO-11 cells, NOR-10 cells, C₃H/IOTI/2 cells, HSDM₁C₃ cells, KLN205 cells, McCoy cells, Mouse L cells, Strain 2071 (Mouse L) cells, L-M strain (Mouse L) cells, L-MTK (Mouse L) cells, NCTC clones 2472 and 2555, SCC-PSA1 cells, Swiss/3T3 cells, Indian muntac cells, SIRC cells, C_{II} cells, and Jensen cells, or derivatives thereof) or any other cell type known to one skilled in the art. In some aspects, cells used in the invention are CHO cells, HEK293 cells, BHK cells, or derivatives thereof.

As used herein, the term “host cell proteins” (HCP) includes protein derived from a host cell and can be unrelated to the desired protein of interest. Host cell proteins can be a process-related impurity which can be derived from the manufacturing process and can include three major categories: cell substrate-derived, cell culture-derived and downstream derived. Cell substrate-derived impurities include, but are not limited to, proteins derived from a host organism and nucleic acid (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reverse phase liquid chromatography, ion-exchange chromatography, size exclusion chromatography, affinity chromatography, hydrophobic interaction chromatography, hydrophilic interaction chromatography, or mixed-mode chromatography. In some aspects, a sample comprising the at least one protein of interest or peptide digest can be subjected to any one of the aforementioned chromatographic methods or a combination thereof. Analytes separated using chromatography will feature distinctive retention times, reflecting the speed at which an analyte moves through the chromatographic column. Analytes may be compared using a chromatogram, which plots retention time on one axis and measured signal on another axis, where the measured signal may be produced from, for example, UV detection or fluorescence detection.

As used herein, the term “mass spectrometer” includes a device capable of identifying specific molecular species and measuring their accurate masses. The term is meant to include any molecular detector with which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application.

In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. MS/MS, or MS², can be performed by first selecting and isolating a precursor ion (MS′), and fragmenting it to obtain meaningful information. Tandem MS has been successfully performed with a wide variety of analyzer combinations. Which analyzers to combine for a certain application can be determined by several different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device.

The peptides identified by the mass spectrometer can be used as surrogate representatives of the intact protein and their post-translational modifications or other modifications. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications, or identifying post-translational modifications, or comparability analysis, or combinations thereof.

In some exemplary aspects, the mass spectrometer can work on nanoelectrospray or nanospray. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

In some exemplary embodiments, mass spectrometry can be performed under native conditions. As used herein, the term “native conditions” can include performing mass spectrometry under conditions that preserve non-covalent interactions in an analyte. For a detailed review on native MS, refer to the review: Elisabetta Boeri Erba & Carlo Petosa, The emerging role of native mass spectrometry in characterizing the structure and dynamics of macromolecular complexes, 24 PROTEIN SCIENCE 1176-1192 (2015).

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools.” Bioinformatics tools provide the capacity to search uninterpreted MS/MS spectra against all possible sequences in the database(s) and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com/proteinpilot), Phenyx (www.phenyx-ms.com), Sorcerer (www.sagenresearch.com), OMS SA (www.pubchem.ncbi.nlm.nih.gov/omssa/), X!Tandem (www.thegpm.org/TANDEM/), Protein Prospector (prospector.ucsfedu/prospector/mshome.htm), Byonic (www.proteinmetrics.com/products/byonic) or Sequest (fields.scripps.edu/sequest).

As used herein, the term “downstream process technology” refers to one or more techniques used after the upstream process technologies to produce, process, and/or purify a protein. Downstream process technology includes, for example, isolation of a protein product, using, for example, affinity chromatography, ion exchange chromatography, such as anion or cation exchange chromatography, hydrophobic interaction chromatography, or displacement chromatography.

As used herein, the term “ultrafiltration” or “UF” can include a membrane filtration process similar to reverse osmosis, using hydrostatic pressure to force water through a semi-permeable membrane. Ultrafiltration is described in detail in: LEOS J. ZEMAN & ANDREW L. ZYDNEY, MICROFILTRATION AND ULTRAFILTRATION: PRINCIPLES AND APPLICATIONS (1996), the entire teaching of which is herein incorporated. Filters with a pore size of smaller than 0.1 μm can be used for ultrafiltration. By employing filters having such small pore size, the volume of the sample can be reduced through permeation of the sample buffer through the filter while proteins are retained behind the filter.

As used herein, “diafiltration” or “DF” can include a method of using ultrafilters to remove and exchange salts, sugars, and non-aqueous solvents, to separate free from bound species, to remove low molecular weight material, and/or to cause the change of ionic and/or pH environments. Microsolutes are removed most efficiently by adding solvent to a solution being ultrafiltered at a rate approximately equal to the ultrafiltration rate. This washes microspecies from the solution at a constant volume. In certain exemplary embodiments of the present disclosure, a diafiltration step can be employed to exchange various buffers used in connection with the instant disclosure, for example, prior to chromatography or other production steps, as well as to remove impurities from the protein preparation.

As used herein, the term “formulation” refers to a pharmaceutical product, for example Dupilumab, that is formulated together with one or more pharmaceutically acceptable vehicles.

In terms of protein formulation, the term “stable,” as used herein refers to the protein of interest within the formulation being able to retain an acceptable degree of chemical structure or biological function after storage under exemplary conditions defined herein. A formulation may be stable even though the protein of interest contained therein does not maintain 100% of its chemical structure or biological function after storage for a defined amount of time. Under certain circumstances, maintenance of about 90%, about 95%, about 96%, about 97%, about 98% or about 99% of a protein's structure or function after storage for a defined amount of time may be regarded as “stable.”

The term “treat” or “treatment” refers to a therapeutic measure that reverses, stabilizes or eliminates an undesired disease or disorder (e.g., atopic dermatitis (eczema), eosinophilic or oral steroid dependent asthma, chronic rhinosinusitis with nasal polyposis (CRSwNP), eosinophilic esophagitis (EoE), inflammation), for example, by causing the regression, stabilization or elimination of one or more symptoms or indicia of such disease or disorder by any clinically measurable degree, for example, with regard to asthma it helps prevent severe asthma attacks (exacerbations), can improve breathing, and helps reduce the amount of oral corticosteroids needed.

As used herein, the term “critical quality attribute” (CQA) is used to describe a physical, chemical, biological, or microbiological property or characteristic that should be within an appropriate limit, range, or distribution to ensure the desired product quality. CQAs may include, for example, post-translational modifications.

As used herein, “viral filtration” can include filtration using suitable filters including, but not limited to, Planova 20N™, 50 N or BioEx from Asahi Kasei Pharma, Viresolve™ filters from EMD Millipore, ViroSart® CPV or Virosart® HF from Sartorius, or Ultipor DV20 or DV50™ or Pegasus™ Prime filter from Pall Corporation. It will be apparent to one of ordinary skill in the art to select a suitable filter to obtain desired filtration performance.

For biologics, the implementation of a robust and flexible upstream process is desirable. An efficient upstream process can lead to desirable production and scale-up of a protein of interest.

As used herein, a “sample” can be obtained from any step of a bioprocess, such as cell culture fluid (CCF), harvested cell culture fluid (HCCF), any step in the downstream processing, drug substance (DS), or a drug product (DP) comprising the final formulated product. In some specific exemplary embodiments, the sample can be selected from any step of the downstream process of clarification, chromatographic production, or filtration.

As used herein, the term “protein alkylating agent” or “alkylation agent” refers to an agent used for alkylating certain free amino acid residues in a protein. Non-limiting examples of protein alkylating agents are iodoacetamide (IOA/IAA), chloroacetamide (CAA), acrylamide (AA), N-ethylmaleimide (NEM), methyl methanethiosulfonate (MMTS), and 4-vinylpyridine or combinations thereof.

As used herein, “protein denaturing” or “denaturation” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT, SDS or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples of chaotropic agents include butanol, ethanol, guanidinium chloride, lithium perchlorate, lithium acetate, magnesium chloride, phenol, propanol, sodium dodecyl sulfate (SDS), thiourea, N-lauroylsarcosine, urea, and salts thereof. In some exemplary embodiments, denaturing agents may be used in the mobile phase in a chromatography analysis. In some exemplary embodiments, a denaturing agent used in a mobile phase may be acetonitrile (ACN). In some exemplary embodiments, a denaturing agent used in a mobile phase may be a surfactant.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion. Digestion of a protein into constituent peptides can produce a “peptide digest” that can further be analyzed using peptide mapping analysis.

As used herein, the term “hydrolyzing agent” refers to any one or combination of a large number of different agents that can perform digestion of a protein. Non-limiting examples of hydrolyzing agents that can carry out enzymatic digestion include protease from Aspergillus saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N(Asp-N), endoproteinase Arg-C(Arg-C), endoproteinase Glu-C(Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. Non-limiting examples of hydrolyzing agents that can carry out non-enzymatic digestion include the use of high temperature, microwave, ultrasound, high pressure, infrared, solvents (non-limiting examples are ethanol and acetonitrile), immobilized enzyme digestion (IMER), magnetic particle immobilized enzymes, and on-chip immobilized enzymes. For a recent review discussing the available techniques for protein digestion, see Switzar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 Journal of Proteome Research 1067-1077 (2013), the entire teachings of which are herein incorporated). One or a combination of hydrolyzing agents can cleave peptide bonds in a protein or polypeptide, in a sequence-specific manner, generating a predictable collection of shorter peptides. The ratio of hydrolyzing agent to protein and the time required for digestion can be appropriately selected to obtain optimal digestion of the protein. When the enzyme to substrate ratio is unsuitably high, the correspondingly high digestion rate will not allow sufficient time for the peptides to be analyzed by mass spectrometer, and sequence coverage will be compromised. On the other hand, a low E/S ratio would need long digestion and thus long data acquisition time. The enzyme to substrate ratio can range from about 1:0.5 to about 1:200.

As used herein, the term “protein reducing agent” or “reduction agent” refers to the agent used for reduction of disulfide bridges in a protein. Non-limiting examples of protein reducing agents used to reduce a protein are dithiothreitol (DTT), ß-mercaptoethanol, Ellman's reagent, hydroxylamine hydrochloride, sodium cyanoborohydride, tris(2-carboxyethyl)phosphine hydrochloride (TCEP-HCl), or combinations thereof.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, chimeric proteins, antibodies, monoclonal antibodies, polyclonal antibodies, and human antibodies. Proteins may be produced using recombinant cell-based production systems, such as the insect baculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated by reference). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST), myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, and IgE. In certain exemplary embodiments the antibody molecule is a full-length antibody (e.g., an IgG1) or alternatively the antibody can be a fragment (e.g., an Fc fragment or a Fab fragment).

The term “antibody” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the present disclosure, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, and single-chain antibody molecules. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

As used herein, a “protein pharmaceutical product,” “biopharmaceutical product” or “biotherapeutic” includes an active ingredient which can be fully or partially biological in nature. In one aspect, the protein pharmaceutical product can comprise a peptide, a protein, an antibody, an antigen, a peptide-drug conjugate, an antibody-drug conjugate, a protein-drug conjugate, cells, tissues, or combinations thereof. In another aspect, the protein pharmaceutical product can comprise a recombinant, engineered, modified, mutated, or truncated version of a peptide, a protein, an antibody, an antigen, vaccine, a peptide-drug conjugate, an antibody-drug conjugate, a protein-drug conjugate, cells, tissues, or combinations thereof.

### Exemplary Anti-IL-4Rα Antibodies

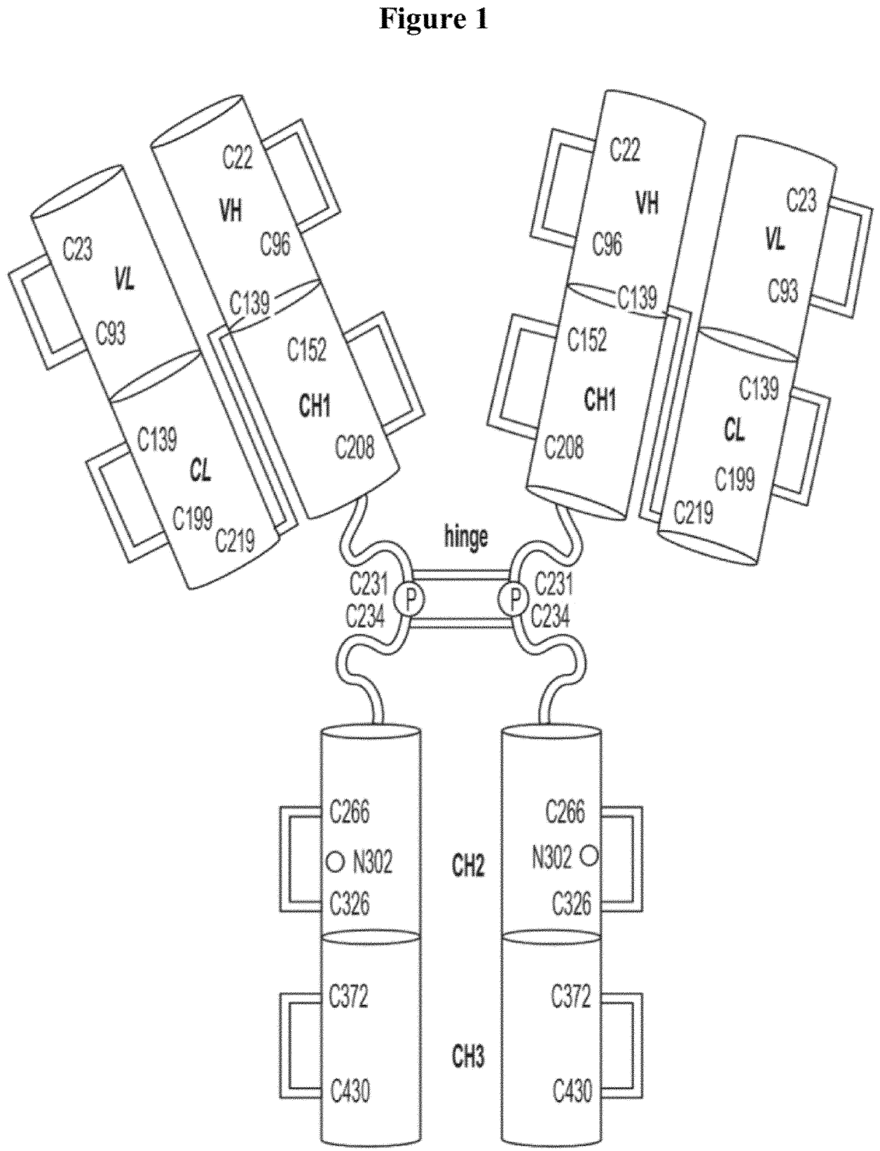

As used herein, Dupilumab (IgG4 isotype) is a covalent heterotetramer consisting of two disulfide-linked human heavy chains, each covalently linked through a disulfide bond to a human kappa light chain. Each heavy chain comprises a serine-to-proline mutation at amino acid 233, which is located in the hinge region of the Fc domain, to reduce the propensity of the antibody to form half-antibodies in solution. There is a single N-linked glycosylation site (Asn³⁰²) in each heavy chain, located within the CH2 domain of the Fc constant region in the molecule. The antibody, based on the primary sequence (in the absence of N-linked glycosylation), possesses a molecular weight of 146,897.0 Da, taking into account the formation of 16 disulfide bonds and removal of Lys⁴⁵² from each heavy chain C-terminus. The variable domains of the heavy and light chains combine to form complementarity-determining regions (CDRs) for the binding of Dupilumab to its target, interleukin-4 receptor α (IL-4Rα). A schematic representation of Dupilumab, including the location of N-linked glycosylation sites and disulfide bond structures, is presented in FIG. 1.

The manufacturing process can be used to produce a human antibody or antigen-binding fragment thereof that specifically binds IL-4R (such as IL-4Rα) and comprises the three heavy chain CDRs (HCDR1, HCDR2 and HCDR3) contained within a heavy chain variable region (HCVR) having an amino acid sequence of SEQ ID NO: 1. The antibody or antigen-binding fragment may comprise the three light chain CDRs (LCVR1, LCVR2, LCVR3) contained within a light chain variable region (LCVR) having an amino acid sequence of SEQ ID NO: 2. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In certain embodiments, the antibody or antigen-binding fragment thereof comprises the six CDRs (HCDR1, HCDR2, HCDR3, LCDR1, LCDR2 and LCDR3) from the heavy and light chain variable region amino acid sequence pairs (HCVR/LCVR) of SEQ ID NOs: 1 and 2.

In certain embodiments, the antibody or antigen-binding fragment thereof comprises six CDRs (HCDR1/HCDR2/HCDR3/LCDR1/LCDR2/LCDR3) having the amino acid sequences of SEQ ID NOs: 3/4/5/6/7/8.

In certain embodiments, the antibody or antigen-binding fragment thereof comprises HCVR/LCVR amino acid sequence pair of SEQ ID NOs: 1 and 2.

In certain embodiments, the antibody is Dupilumab, which comprises the HCVR/LCVR amino acid sequence pair of SEQ ID NOs: 1 and 2.

In certain embodiments, the antibody sequence is Dupilumab, which comprises the heavy chain/light chain amino acid sequence pair of SEQ ID NOs: 9 and 10.

Dupilumab HCVR amino acid sequence:
(SEQ ID NO: 1)
EVQLVESGGGLEQPGGSLRLSCAGSGFTFRDYAMTWVRQAPGKGLEWVS
SISGSGGNTYYADSVKGRFTISRDNSKNTLYLQMNSLRAEDTAVYYCAK
DRLSITIRPRYYGLDVWGQGTTVTVS.
Dupilumab LCVR amino acid sequence:
(SEQ ID NO: 2)
DIVMTQSPLSLPVTPGEPASISCRSSQSLLYSIGYNYLDWYLQKSGQSP
QLLIYLGSNRASGVPDRFSGSGSGTDFTLKISRVEAEDVGFYYCMQALQ
TPYTFGQGTKLEIK.
Dupilumab HCDR1 amino acid sequence:
(SEQ ID NO: 3)
GFTFRDYA.
Dupilumab HCDR2 amino acid sequence:
(SEQ ID NO: 4)
ISGSGGNT.
Dupilumab HCDR3 amino acid sequence:
(SEQ ID NO: 5)
AKDRLSITIRPRYYGL.
Dupilumab LCDR1 amino acid sequence:
(SEQ ID NO: 6)
QSLLYSIGYNY.
Dupilumab LCDR2 amino acid sequence:
(SEQ ID NO: 7)
LGS.
Dupilumab LCDR3 amino acid sequence:
(SEQ ID NO: 8)
MQALQTPYT.
Dupilumab HC amino acid sequence:
(SEQ ID NO: 9)
EVQLVESGGGLEQPGGSLRLSCAGSGFTFRDYAMTWVRQAPGKGLEWVS
SISGSGGNTYYADSVKGRFTISRDNSKNTLYLQMNSLRAEDTAVYYCAK
DRLSITIRPRYYGLDVWGQGTTVTVSSASTKGPSVFPLAPCSRSTSEST
AALGCLVKDYFPEPVTVSWNSGALTSGVHTFPAVLQSSGLYSLSSVVTV
PSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISK
AKGQPREPQVYTLPPSQEEMTKNQVSLTCLVKGFYPSDIAVEWESNGQP
ENNYKTTPPVLDSDGSFFLYSRLTVDKSRWQEGNVFSCSVMHEALHNHY
TQKSLSLSLG (amino acids 1-124 = HCVR; amino acids
125-451 = HC constant).
Dupilumab LC amino acid sequence:
(SEQ ID NO: 10)
DIVMTQSPLSLPVTPGEPASISCRSSQSLLYSIGYNYLDWYLQKSGQSP
QLLIYLGSNRASGVPDRFSGSGSGTDFTLKISRVEAEDVGFYYCMQALQ
TPYTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC (amino acids 1-112 =
LCVR; amino acids 112-219 = LC constant).

In certain embodiments, the manufacturing method can be used to produce an antibody or antigen-binding fragment thereof comprising light chain variable region (LCVR) and heavy chain variable region (HCVR) sequence pairs (LCVR/HCVR) selected from the group consisting of SCB-VL-39/SCB-VH-92; SCB-VL-40/SCB-VH-92; SCB-VL-41/SCB-VH-92; SCB-VL-42/SCB-VH-92; SCB-VL-43/SCB-VH-92; SCB-VL-44/SCB-VH-92; SCB-VL-44/SCB-VH-62; SCB-VL-44/SCB-VH-68; SCB-VL-44/SCB-VH-72; SCB-VL-44/SCB-VH-82; SCB-VL-44/SCB-VH-85; SCB-VL-44/SCB-VH-91; SCB-VL-44/SCB-VH-93; SCB-VL-45/SCB-VH-92; SCB-VL-46/SCB-VH-92; SCB-VL-47/SCB-VH-92; SCB-VL-48/SCB-VH-92; SCB-VL-49/SCB-VH-92; SCB-VL-50/SCB-VH-92; SCB-VL-51/SCB-VH-92; SCB-VL-51/SCB-VH-93; SCB-VL-52/SCB-VH-92; SCB-VL-52/SCB-VH-62; SCB-VL-52/SCB-VH-91; SCB-VL-53/SCB-VH-92; SCB-VL-54/SCB-VH-92; SCB-VL-54/SCB-VH-62; SCB-VL-54/SCB-VH-68; SCB-VL-54/SCB-VH-72; SCB-VL-54/SCB-VH-82; SCB-VL-54/SCB-VH-85; SCB-VL-54/SCB-VH-91; SCB-VL-55/SCB-VH-92; SCB-VL-55/SCB-VH-62; SCB-VL-55/SCB-VH-68; SCB-VL-55/SCB-VH-72; SCB-VL-55/SCB-VH-82; SCB-VL-55/SCB-VH-85; SCB-VL-55/SCB-VH-91; SCB-VL-56/SCB-VH-92; SCB-VL-57/SCB-VH-92; SCB-VL-57/SCB-VH-93; SCB-VL-57/SCB-VH-59; SCB-VL-57/SCB-VH-60; SCB-VL-57/SCB-VH-61; SCB-VL-57/SCB-VH-62; SCB-VL-57/SCB-VH-63; SCB-VL-57/SCB-VH-64; SCB-VL-57/SCB-VH-65; SCB-VL-57/SCB-VH-66; SCB-VL-57/SCB-VH-67; SCB-VL-57/SCB-VH-68; SCB-VL-57/SCB-VH-69; SCB-VL-57/SCB-VH-70; SCB-VL-57/SCB-VH-71; SCB-VL-57/SCB-VH-72; SCB-VL-57/SCB-VH-73; SCB-VL-57/SCB-VH-74; SCB-VL-57/SCB-VH-75; SCB-VL-57/SCB-VH-76; SCB-VL-57/SCB-VH-77; SCB-VL-57/SCB-VH-78; SCB-VL-57/SCB-VH-79; SCB-VL-57/SCB-VH-80; SCB-VL-57/SCB-VH-81; SCB-VL-57/SCB-VH-82; SCB-VL-57/SCB-VH-83; SCB-VL-57/SCB-VH-84; SCB-VL-57/SCB-VH-85; SCB-VL-57/SCB-VH-86; SCB-VL-57/SCB-VH-87; SCB-VL-57/SCB-VH-88; SCB-VL-57/SCB-VH-89; SCB-VL-57/SCB-VH-90; SCB-VL-57/SCB-VH-91; SCB-VL-58/SCB-VH-91; SCB-VL-58/SCB-VH-92; and SCB-VL-58/SCB-VH-93.

In certain embodiments, an antibody or antigen-binding fragment thereof comprises a LCVR/HCVR sequence pair of SCB-VL-44/SCB-VH-92.

In certain embodiments, an antibody or antigen-binding fragment thereof comprises a LCVR/HCVR sequence pair of SCB-VL-54/SCB-VH-92.

In certain embodiments, an antibody or antigen-binding fragment thereof comprises a LCVR/HCVR sequence pair of SCB-VL-55/SCB-VH-92.

In certain embodiments, an antibody or antigen-binding fragment thereof comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 sequence of SCB-55-LCDR1, an LCDR2 sequence of SCB-55-LCDR2, and an LCDR3 sequence of SCB-55-LCDR3.

In certain embodiments, an antibody or antigen-binding fragment thereof comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 sequence of SCB-55-LCDR1, an LCDR2 sequence of SCB-54-LCDR2, and an LCDR3 sequence of SCB-55-LCDR3.

In certain embodiments, an antibody or antigen-binding fragment thereof comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 sequence of SCB-55-LCDR1, an LCDR2 sequence of SCB-54-LCDR2, and an LCDR3 sequence of SCB-44-LCDR3.

The antibodies recited below in Table 1 are described in more detail in U.S. Pat. No. 10,774,141, incorporated herein by reference in its entirety for all purposes.

TABLE 1

| Sequence ID | Sequence |
|---|---|
| SCB-VL-39 | EIVLTQSPGTLSLSPGERATLSCRASQSVSNSYLAWYQQKPGQAPRLL |
| | IFGASSRATGIPDRFSGSGSGTDFTLTISRLEPEDFAVYYCQQYGSSPP |
| | WTFGQGTKVEIK (SEQ ID NO: 11) |
| SCB-VL-40 | EIVLTQSPGTLSLSPGERATLSCRASQSVSSSYLAWYQQKPGQAPRLL |
| | IYGASSRATGIPDRFSGSGSGTDFTLTISRLEPEDFAVYYCQQYGSSPP |
| | WTFGQGTKVEIK (SEQ ID NO: 12) |
| SCB-VL-41 | EIVLTQSPGTLSLSPGERATLSCRASQSVSSSYLAWYQQKPGQAPRLL |
| | IFGASSRAPGIPDRFSGSGSGTDFTLTISRLEPEDFAVYYCQQYGSSPP |
| | WTFGQGTKVEIK (SEQ ID NO: 13) |
| SCB-VL-42 | EIVLTQSPGTLSLSPGERATLSCRASQSVSNSYLAWYQQKPGQAPRLL |
| | IYGASSRATGIPDRFSGSGSGTDFTLTISRLEPEDFAVYYCQQYGSSPP |
| | WTFGQGTKVEIK (SEQ ID NO: 14) |
| SCB-VL-43 | EIVLTQSPGTLSLSPGERATLSCRASQSVSNSYLAWYQQKPGQAPRLL |
| | IFGASSRAPGIPDRFSGSGSGTDFTLTISRLEPEDFAVYYCQQYGSSPP |
| | WTFGQGTKVEIK (SEQ ID NO: 15) |
| SCB-VL-44 | EIVLTQSPGTLSLSPGERATLSCRASQSVSSSYLAWYQQKPGQAPRLL |
| | IYGASSRAPGIPDRFSGSGSGTDFTLTISRLEPEDFAVYYCQQYGSSPP |
| | WTFGQGTKVEIK (SEQ ID NO: 16) |
| SCB-VL-45 | EIVLTQSPGTLSLSPGERATLSCRASQSVSSSYLAWYQQKPGQAPRLL |
| | IFGASSRATGIPDRFSGSGSGTDFTLTISRLEPEDFAVYYCQQYDHSPP |
| | WTFGQGTKVEIK (SEQ ID NO: 17) |
| SCB-VL-46 | EIVLTQSPGTLSLSPGERATLSCRASQSVSSSYLAWYQQKPGQAPRLL |
| | IFGASSRATGIPDRFSGSGSGTDFTLTISRLEPEDFAVYYCQQYGSSAG |
| | WTFGQGTKVEIK (SEQ ID NO: 18) |
| SCB-VL-47 | EIVLTQSPGTLSLSPGERATLSCRASQSVSSSYLAWYQQKPGQAPRLL |
| | IFGASSRATGIPDRFSGSGSGTDFTLTISRLEPEDFAVYYCQQYDHSAG |
| | WTFGQGTKVEIK (SEQ ID NO: 19) |
| SCB-VL-48 | EIVLTQSPGTLSLSPGERATLSCRASQSVSNSYLAWYQQKPGQAPRLL |
| | IFGASSRATGIPDRFSGSGSGTDFTLTISRLEPEDFAVYYCQQYDHSPP |
| | WTFGQGTKVEIK (SEQ ID NO: 20) |
| SCB-VL-49 | EIVLTQSPGTLSLSPGERATLSCRASQSVSSSYLAWYQQKPGQAPRLL |
| | IYGASSRATGIPDRFSGSGSGTDFTLTISRLEPEDFAVYYCQQYDHSPP |
| | WTFGQGTKVEIK (SEQ ID NO: 21) |
| SCB-VL-50 | EIVLTQSPGTLSLSPGERATLSCRASQSVSSSYLAWYQQKPGQAPRLL |
| | IFGASSRAPGIPDRFSGSGSGTDFTLTISRLEPEDFAVYYCQQYDHSPP |
| | WTFGQGTKVEIK (SEQ ID NO: 22) |
| SCB-VL-51 | EIVLTQSPGTLSLSPGERATLSCRASQSVSSSYLAWYQQKPGQAPRLL |
| | IYGASSRAPGIPDRFSGSGSGTDFTLTISRLEPEDFAVYYCQQYDHSA |
| | GWTFGQGTKVEIK (SEQ ID NO: 23) |
| SCB-VL-52 | EIVLTQSPGTLSLSPGERATLSCRASQSVSNSYLAWYQQKPGQAPRLL |
| | IFGASSRAPGIPDRFSGSGSGTDFTLTISRLEPEDFAVYYCQQYDHSAG |
| | WTFGQGTKVEIK (SEQ ID NO: 24) |
| SCB-VL-53 | EIVLTQSPGTLSLSPGERATLSCRASQSVSNSYLAWYQQKPGQAPRLL |
| | IYGASSRATGIPDRFSGSGSGTDFTLTISRLEPEDFAVYYCQQYDHSA |
| | GWTFGQGTKVEIK (SEQ ID NO: 25) |
| SCB-VL-54 | EIVLTQSPGTLSLSPGERATLSCRASQSVSSSYLAWYQQKPGQAPRLL |
| | IFGASSRAPGIPDRFSGSGSGTDFTLTISRLEPEDFAVYYCQQYDHSAG |
| | WTFGQGTKVEIK (SEQ ID NO: 26) |
| SCB-VL-55 | EIVLTQSPGTLSLSPGERATLSCRASQSVSSSYLAWYQQKPGQAPRLL |
| | IYGASSRATGIPDRFSGSGSGTDFTLTISRLEPEDFAVYYCQQYDHSA |
| | GWTFGQGTKVEIK (SEQ ID NO: 27) |
| SCB-VL-56 | EIVLTQSPGTLSLSPGERATLSCRASQSVSNSYLAWYQQKPGQAPRLL |
| | IFGASSRATGIPDRFSGSGSGTDFTLTISRLEPEDFAVYYCQQYDHSAG |
| | WTFGQGTKVEIK (SEQ ID NO: 28) |
| SCB-VL-57 | EIVLTQSPGTLSLSPGERATLSCRASQSVSSSYLAWYQQKPGQAPRLL |
| | IFGASSRATGIPDRFSGSGSGTDFTLTISRLEPEDFAVYYCQQYGSSPP |
| | WTFGQGTKVEIK (SEQ ID NO: 29) |
| SCB-VL-58 | EIVLTQSPGTLSLSPGERATLSCRASQSVSNSYLAWYQQKPGQAPRLL |
| | IYGASSRAPGIPDRFSGSGSGTDFTLTISRLEPEDFAVYYCQQYDHSA |
| | GWTFGQGTKVEIK (SEQ ID NO: 30) |
| SCB-VH-59 | EVQLVESGGGLVHPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS (SEQ ID NO: 31) |
| SCB-VH-60 | EVQLVQSGGGLVQPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS (SEQ ID NO: 32) |
| SCB-VH-61 | EVQLVQSGGGLVHPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS (SEQ ID NO: 33) |
| SCB-VH-62 | EVQLVQSGGGLVHPGGSLRLSCAGSGFTFSRNAMFWVRQAPGKGLE |
| | WVSGIGTGGATSYADSVKGRFTISRDNAKNSLYLQMNSLRAEDMAV |
| | YYCARGRYYFDYWGQGTLVTVSS (SEQ ID NO: 34) |
| SCB-VH-63 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAEDTAV |
| | YYCARGRYYFDYWGQGTLVTVSS (SEQ ID NO: 35) |
| SCB-VH-64 | EVQLVESGGGLVQPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS (SEQ ID NO: 36) |
| SCB-VH-65 | EVQLVESGGGLVHPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS (SEQ ID NO: 37) |
| SCB-VH-66 | EVQLVQSGGGLVQPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS (SEQ ID NO: 38) |
| SCB-VH-67 | EVQLVQSGGGLVHPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFDYWGQGTLVTVSS (SEQ ID NO: 39) |
| SCB-VH-68 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS (SEQ ID NO: 40) |
| SCB-VH-69 | EVQLVESGGGLVHPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS (SEQ ID NO: 41) |
| SCB-VH-70 | EVQLVQSGGGLVQPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS (SEQ ID NO: 42) |
| SCB-VH-71 | EVQLVQSGGGLVHPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS (SEQ ID NO: 43) |
| SCB-VH-72 | EVQLVQSGGGLVHPGGSLRLSCAGSGFTFSRNAMFWVRQAPGKGLE |
| | WVSGIGTGGATSYADSVKGRFTISRDNAKNSLYLQMNSLRAEDMAV |
| | YYCARGRYYFPWWGQGTLVTVSS (SEQ ID NO: 44) |
| SCB-VH-73 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAEDTAV |
| | YYCARGRYYFPWWGQGTLVTVSS (SEQ ID NO: 45) |
| SCB-VH-74 | EVQLVQSGGGLVHPGRSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS (SEQ ID NO: 46) |
| SCB-VH-75 | EVQLVQSGGGLVHPGGSLRLTCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS (SEQ ID NO: 47) |
| SCB-VH-76 | EVQLVQSGGGLVHPGGSLRLSCAGSGFTFSRNAMHWVRQAPGKGL |
| | EWVSGIGTGGATNYADSVKGRFTISRDNAKNSLYLQMNSLRAEDMA |
| | VYYCARGRYYFDYWGQGTLVTVSS (SEQ ID NO: 48) |
| SCB-VH-77 | EVQLVQSGGGLVHPGGSLRLSCAGSGFTFSRNAMFWVRQAPGEGLE |
| | WVSGIGTGGATNYADSVKGRFTISRDNAKNSLYLQMNSLRAEDMA |
| | VYYCARGRYYFDYWGQGTLVTVSS (SEQ ID NO: 49) |
| SCB-VH-78 | EVQLVQSGGGLVHPGGSLRLSCAGSGFTFSRNAMFWVRQAPGKGLE |
| | WVSGIGTGGATNYADSVKGRFTISRDEAKNSLYLQMNSLRAEDMAV |
| | YYCARGRYYFDYWGQGTLVTVSS (SEQ ID NO: 50) |
| SCB-VH-79 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAGDMA |
| | VYYCARGRYYFDYWGQGTLVTVSS (SEQ ID NO: 51) |
| SCB-VH-80 | EVQLVQSGGGLVHPGGSLRLSCAGSGFTFDDYAMFWVRQAPGKGL |
| | EWVSGIGTGGATNYADSVKGRFTISRDNAKNSLYLQMNSLRAEDMA |
| | VYYCARGRYYFDYWGQGTLVTVSS (SEQ ID NO: 52) |
| SCB-VH-81 | EVQLVQSGGGLVQPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS (SEQ ID NO: 53) |
| SCB-VH-82 | EVOLVESGGGLVHPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS (SEQ ID NO: 54) |
| SCB-VH-83 | EVQLVESGGGLVQPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS (SEQ ID NO: 55) |
| SCB-VH-84 | EVQLVESGGGLVQPGGSLRLSCAASGFTFSRNAMFWVRQAPGKGLE |
| | WVSGIGTGGATNYADSVKGRFTISRDNAKNSLYLQMNSLRAEDTAV |
| | YYCARGRYYFPWWGQGTLVTVSS (SEQ ID NO: 56) |
| SCB-VH-85 | EVQLVESGGGLVQPGGSLRLSCAASGFTFSRNAMFWVRQAPGKGLE |
| | WVSGIGTGGATSYADSVKGRFTISRDNAKNSLYLQMNSLRAEDMAV |
| | YYCARGRYYFPWWGQGTLVTVSS (SEQ ID NO: 57) |
| SCB-VH-86 | EVQLVQSGGGLVHPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS (SEQ ID NO: 58) |
| SCB-VH-87 | EVQLVQSGGGLVQPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS (SEQ ID NO: 59) |
| SCB-VH-88 | EVQLVESGGGLVHPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS (SEQ ID NO: 60) |
| SCB-VH-89 | EVQLVQSGGGLVHPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS (SEQ ID NO: 61) |
| SCB-VH-90 | EVQLVESGGGLVQPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS (SEQ ID NO: 62) |
| SCB-VH-91 | EVQLVESGGGLVQPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFDYWGQGTLVTVSS (SEQ ID NO: 63) |
| SCB-VH-92 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS (SEQ ID NO: 64) |
| SCB-VH-93 | EVQLVESGGGLVQPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS (SEQ ID NO: 65) |
| SCB-92- | RNAMF (SEQ ID NO: 66) |
| HCDR1 | |
| SCB-92- | GIGTGGATSYADSVKG (SEQ ID NO: 67) |
| HCDR3 | |
| SCB-92- | GRYYFDY (SEQ ID NO: 68) |
| HCDR3 | |
| SCB-55- | RASQSVSSSYLA (SEQ ID NO: 69) |
| LCDR1 | |
| SCB-55- | GASSRAT (SEQ ID NO: 70) |
| LCDR2 | |
| SCB-55- | QQYDHSAGWT (SEQ ID NO: 71) |
| LCDR3 | |
| SCB-54- | GASSRAP (SEQ ID NO: 72) |
| LCDR2 | |
| SCB-44- | QQYGSSPPWT (SEQ ID NO: 73) |
| LCDR3 | |

In certain embodiments, the manufacturing method can be used to produce an antibody or antigen-binding fragment thereof comprising light chain variable region (LCVR) and heavy chain variable region (HCVR) sequence pairs (LCVR/HCVR) selected from the group consisting of MEDI-1-VL/MEDI-1-VH through MEDI-42-VL/MEDI-42-VH.

In certain embodiments, an antibody or antigen-binding fragment thereof comprises a LCVR/HCVR sequence pair of MEDI-37GL-VL/MEDI-37GL-VH.

In certain embodiments, an antibody or antigen-binding fragment thereof comprises an HCVR comprising an HCDR1 sequence of MEDI-37GL-HCDR1, an HCDR2 sequence of MEDI-37GL-HCDR2, and an HCDR3 sequence of MEDI-37GL-HCDR3, and an LCVR comprising an LCDR1 sequence of MEDI-37GL-LCDR1, an LCDR2 sequence of MEDI-37GL-LCDR2, and an LCDR3 sequence of MEDI-37GL-LCDR3.

The antibodies recited below in Table 2 are described in more detail in U.S. Pat. No. 8,877,189, incorporated herein by reference in its entirety for all purposes.

TABLE 2

| Sequence ID | Sequence |
|---|---|
| MEDI-1-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLDYWGKGTLVTVSS (SEQ ID NO: 74) |
| MEDI-1-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SLSANYVFGTGTKLTVL (SEQ ID NO: 75) |
| MEDI-2-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS (SEQ ID NO: 80) |
| MEDI-2-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SQPPNPLFGTGTKLTVL (SEQ ID NO: 76) |
| MEDI-3-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKLLKNPWGKGTLVTVSS (SEQ ID NO: 77) |
| MEDI-3-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWFG |
| | TPASNYVFGTGTKLTVL (SEQ ID NO: 78) |
| MEDI-4-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS (SEQ ID NO: 80) |
| MEDI-4-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SSPPQPIFGTGTKLTVL (SEQ ID NO: 79) |
| MEDI-5-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYDWGKGTLVTVSS (SEQ ID NO: 80) |
| MEDI-5-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SSPPQPIFGTGTKLTVL (SEQ ID NO: 79) |
| MEDI-6-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 81) |
| MEDI-6-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTYHPIFGTGTKLTVL (SEQ ID NO: 82) |
| MEDI-7-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQYWGKGTLVTVSS (SEQ ID NO: 83) |
| MEDI-7-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SSPPQPIFGTGTKLTVL (SEQ ID NO: 79) |
| MEDI-8-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQYWGKGTLVTVSS (SEQ ID NO: 83) |
| MEDI-8-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTYHPIFGTGTKLTVL (SEQ ID NO: 82) |
| MEDI-9-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS (SEQ ID NO: 80) |
| MEDI-9-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTMYPLFGTGTKLTVL (SEQ ID NO: 84) |
| MEDI-10-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYDWGKGTLVTVSS (SEQ ID NO: 80) |
| MEDI-10-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STVLTPIFGTGTKLTVL (SEQ ID NO: 85) |
| MEDI-11-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWFYDWGKGTLVTVSS (SEQ ID NO: 86) |
| MEDI-11-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPSMIPLFGTGTKLTVL (SEQ ID NO: 87) |
| MEDI-12-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWFYDWGKGTLVTVSS (SEQ ID NO: 86) |
| MEDI-12-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTMYPLFGTGTKLTVL (SEQ ID NO: 84) |
| MEDI-13-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYDWGKGTLVTVSS (SEQ ID NO: 80) |
| MEDI-13-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTLQPLFGTGTKLTVL (SEQ ID NO: 88) |
| MEDI-14-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS (SEQ ID NO: 80) |
| MEDI-14-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPPTKPLFGTGTKLTVL (SEQ ID NO: 89) |
| MEDI-15-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS (SEQ ID NO: 80) |
| MEDI-15-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STHRHPLFGTGTKLTVL (SEQ ID NO: 90) |
| MEDI-16-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS (SEQ ID NO: 80) |
| MEDI-16-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTYHPIFGTGTKLTVL (SEQ ID NO: 82) |
| MEDI-17-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQHWGKGTLVTVSS (SEQ ID NO: 91) |
| MEDI-17-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPVDRPIFGTGTKLTVL (SEQ ID NO: 92) |
| MEDI-18-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQHWGKGTLVTVSS (SEQ ID NO: 91) |
| MEDI-18-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTPMPVFGTGTKLTVL (SEQ ID NO: 93) |
| MEDI-19-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQHWGKGTLVTVSS (SEQ ID NO: 91) |
| MEDI-19-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTYHPIFGTGTKLTVL (SEQ ID NO: 82) |
| MEDI-20-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 81) |
| MEDI-20-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STVWEWPFGTGTKLTVL (SEQ ID NO: 94) |
| MEDI-21-VH | QVQLVQSGAEVKKPGASVKVSCKASGYAFTSYYMHWARQAPGQG |
| | LEWMGIINPSGGSASYAQKFQGRVTMTRDTSTSTVYMELSSLRSED |
| | TAVYYCARGKYWMYDWGKGTLVTVSS (SEQ ID NO: 99) |
| MEDI-21-VL | QSVLTQPPSVSAAPGQKVTISCSGGSSNIGNSYVSWYQQLPGTAPKL |
| | LIYDNNKRPSGIPDRFSGSKSGTSATLAITGLQTGDEAVYFCGTWDT |
| | STVWEWPFGTGTKLTVL (SEQ ID NO: 95) |
| MEDI-22-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 81) |
| MEDI-22-VL | QPVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL (SEQ ID NO: 96) |
| MEDI-23-VH | QVQLVQSGAEVRKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 97) |
| MEDI-23-VL | QSVLTQPPSVSAAPGQKVTISCSGGSSNIGNNYVSWYQQLPGTAPKL |
| | LIYDNNKRPPGIPDRFSGSKSGTSATLAITGLQTGDEADYYCGTWDT |
| | STVWEWPFGTGTKLTVL (SEQ ID NO: 98) |
| MEDI-24-VH | QVQLVQSGAEVKKPGASVKVSCKASGYAFTSYYMHWARQAPGQG |
| | LEWMGIINPRGGSTSYAQKFQGRVTMTRDTSTSTVYMELSSLRSED |
| | TAVYYCARGKYWMYDWGKGTLVTVSS (SEQ ID NO: 99) |
| MEDI-24-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL (SEQ ID NO: 100) |
| MEDI-25-VH | QVQLVQSGAEVKKPGASVKVSCKASGYAFTSYYMHWARQAPGQG |
| | LEWMGIINPRGGSASYAQKFQGRVSMTRDTSTSTVYMELSSLRSED |
| | TAVYYCARGKYWMYDWGKGTLVTVSS (SEQ ID NO: 101) |
| MEDI-25-VL | QSVLTQPPSVSAAPGQKVTISCSGGSSNIGNSYVSWYQQLPGTAPKL |
| | LIYDNNKRPSGIPDRFSGSKSGTTATLAITGLQTGDEADYYCGTWVT |
| | STVWEWPFGTGTKLTVL (SEQ ID NO: 102) |
| MEDI-26-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 81) |
| MEDI-26-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL (SEQ ID NO: 100) |
| MEDI-27-VH | QVQLVQSGAEVRKPGASVKVSCKASGYAFTSYYMHWARQAPGQG |
| | LEWMGIINPSGGSTSYAQKFQGRVTMTRDTSTSTVYMELSSLRPED |
| | TAVYYCARGKYWMYDWGKGTQVTVSS (SEQ ID NO: 103) |
| MEDI-27-VL | QSVLTQPPLVSAAPGQKVTISCSGGSSNIGNSYVSWYQRLPGTAPKL |
| | LIYDNNKRPSGIPDRFSGSKSGTSATLAITGLQTGDEADYYCGTWDT |
| | STVWEWPFGTGTKLTVL (SEQ ID NO: 104) |
| MEDI-28-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGNGTLVTVSS (SEQ ID NO: 105) |
| MEDI-28-VL | LPVLTQPPSVSAAPGQKVTISCSGGSSSIGNSYVSWYQQLPGAAPKL |
| | LIYDNNKRPSGIPDRFSGFRSGTSATLAITGLQTGDEADYYCGTWDT |
| | SPVWEWPFGTGTKLTVL (SEQ ID NO: 106) |
| MEDI-29-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTRVTVSS (SEQ ID NO: 107) |
| MEDI-29-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPVWEWPFGTGTKLTVL (SEQ ID NO: 108) |
| MEDI-30-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 81) |
| MEDI-30-VL | QSVLTQPPSVSAAPGQKVTISCSGGSSNIGNSYVSWYQRLPGAAPKL |
| | LIYDNNKRPSGIPDRFSGSKSGTSATLAITGLQTGDEADYYCGTWDT |
| | STVWEWPFGTGTKLTVL (SEQ ID NO: 109) |
| MEDI-31-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 81) |
| MEDI-31-VL | QSVLTQPPSVSAAPGQKVTISCSGGSSSIGNSYVSWYQQLPGTAPKL |
| | LIYDNNKRPSGIPDRFSGSKSGTSATLAITGLQTGDEADYYCGTWAT |
| | SPVWEWPFGTGTKLTVL (SEQ ID NO: 110) |
| MEDI-32-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 81) |
| MEDI-32-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STAWEWPFGTGTKLTVL (SEQ ID NO: 111) |
| MEDI-33-VH | QVQLVQSGAEEKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 112) |
| MEDI-33-VL | QSALTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL (SEQ ID NO: 113) |
| MEDI-34-VH | QVQLVQSGAEVKKPGASVKVSCKASGYAFTSYYMHWARQAPGQG |
| | LEWMGIINPSGGSTSYAQKFQGRVSMTRDTSTSTVYMELSSLRSEDT |
| | AVYYCARGKYWMYDWGKGTLVTVSS (SEQ ID NO: 114) |
| MEDI-34-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL (SEQ ID NO: 100) |
| MEDI-35-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 81) |
| MEDI-35-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPVWEWPFGTGTKLTVL (SEQ ID NO: 108) |
| MEDI-36-VH | QVQLVQSGAEVKKPGASVKVSCKASGYAFTSYYMHWARQAPGQG |
| | LEWMGIINPSGGSASYAQKFQGRVTMTRDTSTSTVYMELSSLRSED |
| | TAVYYCARGKYWMYDWGKGTLVTVSS (SEQ ID NO: 99) |
| MEDI-36-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDS |
| | STVWEWPFGTGTKLTVL (SEQ ID NO: 100) |
| MEDI-37-VH | QVQLVQSGAEVKKPGASVKVSCKASGYAFTSYYMHWARQAPGQG |
| | LEWMGIINPRGGSTSYAQKFQGRVAMTRDTSTSTVYMELSSLRPED |
| | TAVYYCARGKYWMYDWGKGTLVTVSS (SEQ ID NO: 115) |
| MEDI-37-VL | QSVLTQPPSVSAAPGQKVTISCSGGGSSIGNSYVSWYQQLPGTAPKL |
| | LIYDNNKRPSGVPDRFSGSKSGTSATLAITGLQTGDEADYYCGTWD |
| | TSPVWEWPFGTGTKLTVL (SEQ ID NO: 116) |
| MEDI-38-VH | QVQLVQSGAEVKKPGASVKVSCKASGYAFTSYYMHWARQAPGQG |
| | LEWMGIINPSGGSASYAQKFQGRVTMTRDTSTSTVYMELSSLRSED |
| | TAVYYCARGKYWMYDWGKGTLVTVSS (SEQ ID NO: 99) |
| MEDI-38-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL (SEQ ID NO: 100) |
| MEDI-39-VH | QVQLVQSGAEVKKPGASVKVSCKASGYAFTSYYMHWARQAPGQG |
| | LEWMGIINPRGGSTSYAQKFQGRVTMTRDTSTSTVYMELSSLRSED |
| | TAVYYCARGKYWMYDWGKGTLVTVSS (SEQ ID NO: 99) |
| MEDI-39-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STAWEWPFGTGTKLTVL (SEQ ID NO: 117) |
| MEDI-40-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS (SEQ ID NO: 81) |
| MEDI-40-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDS |
| | STVWEWPFGTGTKLTVL (SEQ ID NO: 100) |
| MEDI-41-VH | QVQLVQSGAEVRKPGASVKVSCKASGYAFTSYYMHWARQAPGQG |
| | LEWMGIINPSGGSTSYAQKFQGRVTMTRDTSTSTVYMELSSLRPED |
| | TAVYYCARGKYWMYDWGKGTLVTVSG (SEQ ID NO: 118) |
| MEDI-41-VL | QSVLTQPPSVSAAPGQKVTISCSGGSTNIGNSYVSWYQRLPGTAPKL |
| | LIYDNNKRPPGIPDRFSGSKSGTSATLAITGLQTGDEADYYCGTWDT |
| | STVWEWPFGTGTKLTVL (SEQ ID NO: 119) |
| MEDI-42-VH | QVQLVQSGAEVKKPGASVKVSCKASGYAFTSYYMHWARQAPGQG |
| | LEWVGIINPSGGSTSYAQKFQGRVTMTRDTSTSTVYMELSSLRSGDT |
| | AVYYCARGKYWMYDWGKGTLVTVSS (SEQ ID NO: 120) |
| MEDI-42-VL | QAVLTQPPSVSAAPGQKVTISCSGGSSNIGNSYVSWYQRLPGAAPKL |
| | LIYDNNKRPSGIPDRFSGSKSGTSATLAITGLQTGDEADYYCGTWDT |
| | STGWEWPFGTGTKLTVL (SEQ ID NO: 121) |
| MEDI-37GL- | QVQLVQSGAEVKKPGASVKVSCKASGYAFTSYYMHWVRQAPGQG |
| VH | LEWMGIINPRGGSTSYAQKFQGRVTMTRDTSTSTVYMELSSLRSED |
| | TAVYYCARGKYWMYDWGKGTLVTVSS (SEQ ID NO: 122) |
| MEDI-37GL- | QSVLTQPPSVSAAPGQKVTISCSGGGSSIGNSYVSWYQQLPGTAPKL |
| VL | LIYDNNKRPSGIPDRFSGSKSGTSATLGITGLQTGDEADYYCGTWDT |
| | SPVWEWPFGTGTKLTVL (SEQ ID NO: 123) |
| MEDI-37GL- | SYYMH (SEQ ID NO: 124) |
| HCDR1 | |
| MEDI-37GL- | IINPRGGSTSYAQKFQG (SEQ ID NO: 125) |
| HCDR2 | |
| MEDI-37GL- | GKYWMYD (SEQ ID NO: 126) |
| HCDR3 | |
| MEDI-37GL- | SGGGSSIGNSYVS (SEQ ID NO: 127) |
| LCDR1 | |
| MEDI-37GL- | DNNKRPS (SEQ ID NO: 128) |
| LCDR2 | |
| MEDI-37GL- | GTWDTSPVWEWP (SEQ ID NO: 129) |
| LCDR3 | |

In certain embodiments, an antibody or antigen-binding fragment thereof comprises a LCVR/HCVR sequence pair of AJOU-90-VL/AJOU-83-VH.

In certain embodiments, an antibody or antigen-binding fragment thereof comprises an HCVR comprising an HCDR1 sequence of AJOU-84-HCDR1, an HCDR2 sequence of AJOU-85-HCDR2, and an HCDR3 sequence of AJOU-32-HCDR3, and an LCVR comprising an LCDR1 sequence of AJOU-96-LCDR1, an LCDR2 sequence of AJOU-60-LCDR2, and an LCDR3 sequence of AJOU-68-LCDR3.

The antibodies recited below in Table 3 are described in more detail in WO2020/096381 and Kim et al. (Scientific Reports. 9: 7772. 2019), incorporated herein by reference in their entireties for all purposes.

TABLE 3

| Sequence ID | Sequence |
|---|---|
| AJOU-1-VH | EVQLLESGGGLVQPGGSLRLSCAVSGFTFSNYAMSWVRQAPGKGL |
| | EWVSAISSGGGNIYYADSVKGRFTISRDNSKNTLYLQMNSLRAEDT |
| | AVYYCAKLRRYFDYWGQGTLVTVSS (SEQ ID NO: 130) |
| AJOU-2-VH | EVQLLESGGGLVQPGGSLRLSCAASGFTFSDYAMSWVRQAPGKGL |
| | EWVSAISSGGSSIYYADSVKGRFTISRDNSKNTLHLQMNSLRAEDT |
| | AVYYCARGPQRSATAVFDYWGQGTLVTVSS (SEQ ID NO: 131) |
| AJOU-3-VH | EVQLLESGGGLVQPGGSLRLSCAASGFTFSNYAMSWVRQAPGKGL |
| | EWVSWISPNSGNIYYADSVKGRFTISRDNSKNTLYLQMNSLRAEDT |
| | AVYYCARRPLSAAWSHSSYYNAMDVWGQGTLVTVSS (SEQ ID |
| | NO: 132) |
| AJOU-4-VH | EVQLLESGGGLVQPGGSLRLSCAASGFTFSGYAMSWVRQAPGKGL |
| | EWVSLISHSGSNTYYADSVKGRFTISRDNSKNTLYLQMNSLRAEDT |
| | AVYYCARPHRAFDYWGQGTLVTVSS (SEQ ID NO: 133) |
| AJOU-5-VH | EVQLLESGGGLVQPGGSLRLSCAASGFTFSNYAMSWVRQAPGKGL |
| | EWVSGISHGSGSIYYADSVKGRFTISRDNSKNTLYLQMNSLRAEDT |
| | AVYYCARPHRAFDYWGQGTLVTVSS (SEQ ID NO: 134) |
| AJOU-6-VH | EVQLLESGGGLVQPGGSLRLSCAASGFTFSNYAMSWVRQAPGKGL |
| | EWVSGISHGNGSIYYADSVKGRFTISRDNSKNTLYLQMNSLRAEDT |
| | AVYYCAKTGRHFDYWGQGTLVTVSS (SEQ ID NO: 135) |
| AJOU-7-VH | EVQLLESGGGLVQPGGSLRLSCAASGFTFSNYAMSWVRQAPGKGL |
| | EWVSSISPSGSSIYYADSVKGRFTISRDNSKNTLYLQMNSLRAEDTA |
| | VYYCARSYRAFDYWGQGTLVTVSS (SEQ ID NO: 136) |
| AJOU-8-VH | EVQLLESGGGLVQPGGSLRLSCAASGFTFSNYAMSWVRQAPGKGL |
| | EWVSAISPSGGSIYYADSVKGRFTISRDNSKNTLYLQMNSLRAEDT |
| | AVYYCARAKRAFDYWGQGTLVTVSS (SEQ ID NO: 137) |
| AJOU-9-VH | EVQLLESGGGLVQPGGSLRLSCAASGFTFSNYAMSWVRQAPGKGL |
| | EWVSAISPGSGSTYYADSVKGRFTISRDNSKNTLYLQMNSLRAEDT |
| | AVYYCAKFRRHFDYWGQGTLVTVSS (SEQ ID NO: 138) |
| AJOU-10-VH | EVQLLESGGGLVQPGGSLRLSCAASGFTFSNYAMSWVRQAPGKGL |
| | EWVSAISSGGGNIYYADSVKGRFTISRDNSKNTLYLQMNSLRAEDT |
| | AVYYCARVHRAFDYWGQGTLVTVSS (SEQ ID NO: 139) |
| AJOU-69-VH | EVQLLESGGGLVQPGGSLRLSCAASGFTFSNYAMSWVRQAPGKGL |
| | EWVSAITSSGRSIYYADSVKGRFTISRDNSKNTLYLQMNSLRAEDT |
| | AVYYCARVHRAFDYWGQGTLVTVSS (SEQ ID NO: 140) |
| AJOU-70-VH | EVQLLESGGGLVQPGGSLRLSCAASGFTFSNYAMSWVRQAPGKGL |
| | EWVSAITSSGANIYYADSVKGRFTISRDNSKNTLYLQMNSLRAEDT |
| | AVYYCARVHRAFDYWGQGTLVTVSS (SEQ ID NO: 141) |
| AJOU-71-VH | EVQLLESGGGLVQPGGSLRLSCAASGFTFSNYAMSWVRQAPGKGL |
| | EWVSAITSSGGNIYYADSVKGRFTISRDNSKNTLYLQMNSLRAEDT |
| | AVYYCARVHRAFDYWGQGTLVTVSS (SEQ ID NO: 142) |
| AJOU-72-VH | EVQLLESGGGLVQPGGSLRLSCAASGFTFSNYAMSWVRQAPGKGL |
| | EWVSAITAGGGSIYYADSVKGRFTISRDNSKNTLYLQMNSLRAEDT |
| | AVYYCARVHRAFDYWGQGTLVTVSS (SEQ ID NO: 143) |
| AJOU-83-VH | EVQLLESGGGLVQPGGSLRLSCAASGFTFSRHAMAWVRQAPGKGL |
| | EWVSAITSSGRSIYYADSVKGRFTISRDNSKNTLYLQMNSLRAEDT |
| | AVYYCARVHRAFDYWGQGTLVTVSS (SEQ ID NO: 144) |
| AJOU-33-VL | QSVLTQPPSASGTPGQRVTISCSGSSSNIGNNYVNWYQQLPGTAPK |
| | LLIYDNSHRPSGVPDRFSGSKSGTSASLAISGLRSEDEADYYCGTW |
| | DASLSAYVFGGGTKLTVL (SEQ ID NO: 145) |
| AJOU-34-VL | QSVLTQPPSASGTPGQRVTISCSGSSSNIGNNNVSWYQQLPGTAPKL |
| | LIYANSKRPSGVPDRFSGSKSGTSASLAISGLRSEDEADYYCGSWD |
| | DSLSAYVFGGGTKLTVL (SEQ ID NO: 146) |
| AJOU-35-VL | QSVLTQPPSAPGTPGQRVTISCTGSSSNIGSNSVNWYQQLPGTAPKL |
| | LIYDDSHRPSGVPDRFSGSKSGTSASLAISGLRSEDEADYYCDAWD |
| | SSLSAYVFGGGTKLTVL (SEQ ID NO: 147) |
| AJOU-36-VL | QSVLTQPPSASGTPGQRVTLSCTGSSSNIGSNYVSWYQQLPGTAPK |
| | LLIYADSQRPSGVPDRFSGSKSGTSASLAISGLRSEDEADYYCGTW |
| | DDSLSGYVFGGGTKLTVL (SEQ ID NO: 148) |
| AJOU-37-VL | QSVLTQPPSASGTPGQRVTISCSSSSSNIGSNYVSWYQQLPGTAPKL |
| | LIYSDSHRPSGVPDRFSGSKSGTSASLAISGLRSEDEADYYCGSWDY |
| | SLSAYVFGGGTKLTVL (SEQ ID NO: 149) |
| AJOU-38-VL | QSVLTQPPSASGTPGQRVTISCTGSSSNIGNNTVSWYQQLPGTAPKL |
| | LIYDNSHRPSGVPDRFSGSKSGTSASLAISGLQSEDEADYYCGSWD |
| | YSLSAYVFGGGTKLTVL (SEQ ID NO: 150) |
| AJOU-39-VL | QSVLTQPPSASGTPGQRVTISCTGSSSNIGNNDVNWYQQLPGTAPK |
| | LLIYYDSQRPSGVPDRFSGSKSGTSASLAISGLRSEDEADYYCATW |
| | DASLSAYVFGGGTKLTVL (SEQ ID NO: 151) |
| AJOU-40-VL | QSVLTQPPSASGTPGQRVTISCSGSSSNIGSNAVNWYQQLPGTAPKL |
| | LIYYDNQRPSGVPDRFSGSKSGTSASLAISGLRSEDEADYYCGTWD |
| | DSLNGYVFGGGTKLTVL (SEQ ID NO: 152) |
| AJOU-41-VL | QSVLTQPPSASGTPGQRVTISCSGSSSNIGNNAVTWYQQLPGTAPK |
| | LLIYDDSHRPSGVPDRFSGSKSGTSASLAISGLRSEDEADYYCGSW |
| | DYSLSAYVFGGGTKLTVL (SEQ ID NO: 153) |
| AJOU-42-VL | QSVLTQPPSASGTPGQRVTISCSGSSSNIGSNTFNWYQQLPGTAPKL |
| | LIYADSHRPSGVPDRFSGSKSGTSASLAISGLRSEDEADYYCGTWD |
| | YSLSGYVLGGGTKLTVL (SEQ ID NO: 154) |
| AJOU-77-VL | QSVLTQPPSASGTPGQRVTISCSGSSSNIGSNTFNWYQQLPGTAPKL |
| | LIYADSHRPSGVPDRFSGSKSGTSASLAISGLRSEDEADYYCGTWD |
| | YSLSGYVLGGGTKLTVL (SEQ ID NO: 154) |
| AJOU-78-VL | QSVLTQPPSASGTPGQRVTISCSGSSSNIGSNTFNWYQQLPGTAPKL |
| | LIYADSHRPSGVPDRFSGSKSGTSASLAISGLRSEDEADYYCGTWD |
| | YSLRGYVLGGGTKLTVL (SEQ ID NO: 155) |
| AJOU-79-VL | QSVLTQPPSASGTPGQRVTISCSGSSSNIGSNTFNWYQQLPGTAPKL |
| | LIYADSHRPSGVPDRFSGSKSGTSASLAISGLRSEDEADYYCGYWD |
| | YSLSGYVLGGGTKLTVL (SEQ ID NO: 156) |
| AJOU-80-VL | QSVLTQPPSASGTPGQRVTISCSGSSSNIGSNTFNWYQQLPGTAPKL |
| | LIYADSHRPSGVPDRFSGSKSGTSASLAISGLRSEDEADYYCGTWD |
| | YSLSGYVLGGGTKLTVL (SEQ ID NO: 154) |
| AJOU-86-VL | QSVLTQPPSASGTPGQRVTISCSGSSANSRTDGFNWYQQLPGTAPK |
| | LLIYADSHRPSGVPDRFSGSKSGTSASLAISGLRSEDEADYYCGTW |
| | DYSLSGYVLGGGTKLTVLG (SEQ ID NO: 157) |
| AJOU-87-VL | QSVLTQPPSASGTPGQRVTISCSGSAQFGSRDNFNWYQQLPGTAPK |
| | LLIYADSHRPSGVPDRFSGSKSGTSASLAISGLRSEDEADYYCGTW |
| | DYSLSGYVLGGGTKLTVLG (SEQ ID NO: 158) |
| AJOU-88-VL | QSVLTQPPSASGTPGQRVTISCSGSTKQMHNYQFNWYQQLPGTAP |
| | KLLIYADSHRPSGVPDRFSGSKSGTSASLAISGLRSEDEADYYCGT |
| | WDYSLSGYVLGGGTKLTVLG (SEQ ID NO: 159) |
| AJOU-89-VL | QSVLTQPPSASGTPGQRVTISCSGSLLRGENLQFNWYQQLPGTAPK |
| | LLIYADSHRPSGVPDRFSGSKSGTSASLAISGLRSEDEADYYCGTW |
| | DYSLSGYVLGGGTKLTVLG (SEQ ID NO: 160) |
| AJOU-90-VL | QSVLTQPPSASGTPGQRVTISCSGSPLFPDSGSFNWYQQLPGTAPKL |
| | LIYADSHRPSGVPDRFSGSKSGTSASLAISGLRSEDEADYYCGTWD |
| | YSLSGYVLGGGTKLTVLG (SEQ ID NO: 161) |
| AJOU-91-VL | QSVLTQPPSASGTPGQRVTISCSGSAALDLSPSFNWYQQLPGTAPKL |
| | LIYADSHRPSGVPDRFSGSKSGTSASLAISGLRSEDEADYYCGTWD |
| | YSLSGYVLGGGTKLTVLG (SEQ ID NO: 162) |
| AJOU-84- | RHAMA (SEQ ID NO: 163) |
| HCDR1 | |
| AJOU-85- | AITSSGRSIYYADSVKG (SEQ ID NO: 164) |
| HCDR2 | |
| AJOU-32- | VHRAFDY (SEQ ID NO: 165) |
| HCDR3 | |
| AJOU-96- | SGSPLFPDSGSFN (SEQ ID NO: 166) |
| LCDR1 | |
| AJOU-60- | ADSHRPS (SEQ ID NO: 167) |
| LCDR2 | |
| AJOU-68- | GTWDYSLSGYV (SEQ ID NO: 168) |
| LCDR3 | |

In certain embodiments, an antibody or antigen-binding fragment thereof of the disclosure comprises light chain variable region (LCVR) and heavy chain variable region (HCVR) sequence pairs (LCVR/HCVR) selected from the group of sequences listed in Table 4 consisting of 11/3, 27/19, 43/35, 59/51, 75/67, 91/83, 107/99, 123/115, 155/147, and 171/163.

The antibodies recited below in Table 4 are described in more detail in U.S. Pat. Nos. 7,605,237 and 7,608,693, incorporated herein by reference in their entireties for all purposes.

TABLE 4

| Sequence ID | Sequence |
|---|---|
| REGN-VH-3 | QVQLVQSGAEVKKPGASVKVSCKASGYTFTNYGISWVRQAPGQG |
| | LEWMGWISVYNGKTNYAQKLQGRVTMTTDTSTTTAYMEMRSLR |
| | SDDTAVYYCARGSGYDLDYWGQGTLVSVSS (SEQ ID NO: 169) |
| REGN-VH-19 | EVQLVESGGGLVQPGGSLRLSCAASGFTFSSFWMTWVRQAPGKG |
| | LEWVANIKQDGSEKYYVDSVKGRFTISRDNAKNSLYLQMNSLRA |
| | EDTAVYYCARDPGRTMVRGGIRYYYGMDVWGQGTTVTVSS |
| | (SEQ ID NO: 170) |
| REGN-VH-35 | EVKLAESGGGLVQPGGSLRLSCAASGFTFSSHWMNWVRQAPGKG |
| | LEWVANIKQDGSDKYYVDSVKGRFTISRDNAKNSLYLQLNSLIAE |
| | DTAVYYCARDRGVRPPRGAFDIWGQGTMVTVSS (SEQ ID NO: |
| | 171) |
| REGN-VH-51 | QVQLVQSGAEVKKPGASVKVSCKASGYTFNSYGISWVRQAPGQG |
| | LEWMGWIRTYNGNTNYAQKLQGRVTMTTDTSTSTAYMELRSLR |
| | SDDTAVYYCARDEARIVVAGTTPYYYGMDVWGQGTTVTVSS |
| | (SEQ ID NO: 172) |
| REGN-VH-67 | QVQLVESGGGLVQPGGSLRLSCAVSGFTISDHYMSWIRQAPGKGL |
| | EWISYISSSGSKIYYADSVKGRFTISRDNAKNSLFLQMNSLRAEDT |
| | AVYYCARTRQLVGDYWGQGTLVTVSS (SEQ ID NO: 173) |
| REGN-VH-83 | EVQLVESGGGLVQPGRSLRLSCAASGFTFDNYAMHWVRQAPGK |
| | GLEWVSGIRWNSGSIGYADSVKGRFTISRDNAKNSLYLQMNSLRA |
| | EDTALYYCAKEGGYSGYRPGPFFDYWGQGTLVTVSS (SEQ ID |
| | NO: 174) |
| REGN-VH-99 | QVQLVQSGAEVKKPGASVKVSCKASGYTFTNYGISWVRQAPGQG |
| | LEWMGWISVYNGHTNYAQKLQGRVTMTTDTSTSTAYMELRSLR |
| | SDDTAVYYCARGSGYDFDSWGQGTLVTVSS (SEQ ID NO: 175) |
| REGN-VH-115 | QVQLVQSGAEVKKPGASVKVSCKASRYTFTSYDINWVRQATGQG |
| | LEWMGWMNPNSGNTGYAQKFQGRVTMTRNTSTSTAYMELSSLR |
| | SEDTAVYYCARVRRFFDYWGQGTLVTVSS (SEQ ID NO: 176) |
| REGN-VH-147 | QVQLVQSGPEVKKPGASVKVSCKASGYTFTNYGISWVRQAPGQG |
| | LEWMGWISVYNGNINYAQKLQGRVTMTTDTSTSTAYMDLRSLRS |
| | DDTAVYYCARGSGYDFDYWGQGTLVTVSS (SEQ ID NO: 177) |
| REGN-VH-163 | QVQLVQSGAEVKKPGASVKVSCKDSAYTFNRYGISWVRQAPGQG |
| | LEWMGWISAYTGNTVYAQKLQGRVTMTTDNSTSTAYMELRSLR |
| | SDDTAVYYCARDKSIFGVVRGFDYWGQGTLVTVSS (SEQ ID NO: |
| | 178) |
| REGN-VL-11 | AIQMTQSPSSLSASVGDRVTITCRASQGIRNALGWYQQKPGKAPK |
| | LLIYAASSLQSGVPSRFSGSGSGTDFTLTFSSLQPEDFATYYCLQDF |
| | NYPYTFGQGTKLEIK (SEQ ID NO: 179) |
| REGN-VL-27 | DIQMTQSPSSVSASVGDRVTISCRASQGVSSWLAWYQQKPGNAP |
| | KLLISAASSIQSGVPSRFSGSGSGTDFTLTISSLQPEDFATYYCQQA |
| | NSFPLTFGGGTKVEIK (SEQ ID NO: 180) |
| REGN-VL-43 | DIQMTQSPSSVSASVGDRVTITCRASQGISSWLAWYQQKPGKAPK |
| | LLIYAASSFQSGVPSRFSGSGSGTDFTLTISSLQPEDFATYFCQQAN |
| | SFPLTFGGGTTVEIK (SEQ ID NO: 181) |
| REGN-VL-59 | DIQMTQSPSSVSASVGDRVTITCRASQDISIWLAWYQQSPGKAPKL |
| | LINVASRLQSGVPSRFSGSGSGTDFTLTINSLQPEDFVTYYCQQAN |
| | SFPITFGQGTRLATK (SEQ ID NO: 182) |
| REGN-VL-75 | DIQLTQSPSFLSASVGDRVTITCWASQGISSYLAWYQQKPGKAPKL |
| | LIFAASTLQSGVPSRFSGSGSGTEFTLTISSLQPEDFATYYCQQLNS |
| | YPLTFGGGTKVEIR (SEQ ID NO: 183) |
| REGN-VL-91 | EIVMTQSPATLSVSPGERATLSCRASQSVNYNLAWYQHKPGQAPR |
| | LLIYGASTRATGIPARFSGSGSGTEFTLTISSLQSEDFAVYYCQQYN |
| | NWPLTFGGGTKVEIK (SEQ ID NO: 184) |
| REGN-VL-107 | AIQMTQSSSSLSASVGDRVTITCRASQAIRNALGWYQQKPGKAPK |
| | VLIYAASSLQSGIPSRFSGSGSGTDFTLTISSLQPEDFATYYCLQDY |
| | DYPYTFGQGTKLEIK (SEQ ID NO: 185) |
| REGN-VL-123 | DIQLTQSPSFLSASVGDRVTITCWASQGIISYLAWYQQKPGKAPKL |
| | LIYAASTLHSGVPSRFSGSGSGTEFTLTISSLQPEDFATYYCHQLKS |
| | YPITFGQGTRLEIK (SEQ ID NO: 186) |
| REGN-VL-155 | AIQMTQSPSSLSASVGDRVTITCRASQDIRNALGWYQQKPGKAPK |
| | LLIYAASSLQSGVPSRFSGSASGTDFTLTISSLQPEDFAAYYCLQDY |
| | NYPYTFGQGTKLEIK (SEQ ID NO: 187) |
| REGN-VL-171 | EIVMTQSPVTLSLSPGERATLPCRASQSVSSSLAWYQQKAGQSPRL |
| | LIYGASTRATGIPARFSGSGSGTEFTLTISNLQSEDFAVYYCQQYN |
| | NWPLTFGGGTKVEIK (SEQ ID NO: 188) |

It is understood that the present disclosure is not limited to any of the aforesaid protein(s), protein(s) of interest, antibody(s), cell culture media, host cell protein(s), protein alkylating agent(s), protein denaturing agent(s), protein reducing agent(s), digestive enzyme(s), treatment(s), process(es), sample(s), surfactant(s), detergent(s), chromatographic method(s), filtration method(s), mass spectrometer(s), database(s), bioinformatics tool(s), pH, temperature(s), or concentration(s), and any protein(s), protein(s) of interest, antibody(s), cell culture media, host cell protein(s), protein alkylating agent(s), protein denaturing agent(s), protein reducing agent(s), digestive enzyme(s), treatment(s), process(es), sample(s), surfactant(s), detergent(s), chromatographic method(s), filtration method(s), mass spectrometer(s), database(s), bioinformatics tool(s), pH, temperature(s), or concentration(s) can be selected by any suitable means.

The present disclosure will be more fully understood by reference to the following examples. They should not, however, be construed as limiting the scope of the invention.

### Cell Culture Media and Additives

The elimination of serum and reduction or elimination of hydrolysates from cell culture media, while reducing lot-to-lot variability and enhancing downstream processing steps, unfortunately diminishes cell growth, viability and protein expression. Thus, chemically defined serum-free and low-to-no-hydrolysate media requires additional ingredients to improve cell growth and protein production. The cell culture media of the invention may be supplemented with additional ingredients such as polyamines or increased concentrations of components like amino acids, salts, sugars, vitamins, hormones, growth factors, buffers, antibiotics, lipids, trace elements and the like, depending on the requirements of the cells to be cultured or the desired cell culture parameters.

In some embodiments, the cell culture medium disclosed herein is supplemented with polyamines (“PS media”) such as ornithine, putrescine, spermine, spermidine, or combinations to improve cell growth, cell viability, and recombinant protein production.

In one aspect, the PS media comprises ornithine at a concentration (expressed in micromoles per liter) of at least about 90, 100, 150, 200, 250, 300, 350, 400, 450, 500, 540, 545, 550, 555, 560, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 625, 630, 635, 640, 645, 650, 700, 750, 800, 850, or 900±14 μM. In one aspect, the PS media comprises ornithine at a concentration between about 0.09 and 0.9 mM.

In one embodiment and in addition to the inclusion of ornithine or putrescine, the media comprises a mixture of nucleosides in a cumulative concentration of at least 50 μM, at least 60 μM, at least 70 μM, at least 80 μM, at least 90 μM, at least 100 μM, at least 110 μM, at least 115 μM, at least 120 μM, at least 125 μM, at least 130 μM, at least 135 μM, at least 140 μM, at least 145 μM, at least 150 μM, at least 155 μM, at least 160 μM, at least 165 μM, or at least 170 μM.

In one embodiment, the media comprises about 174 μM±26 μM nucleoside. In one embodiment, the media comprises purine derivatives in a cumulative concentration of at least 40 μM, at least 45 μM, at least 50 μM, at least 55 μM, at least 60 μM, at least 65 μM, at least 70 μM, at least 75 μM, at least 80 μM, at least 85 μM, at least 90 μM, at least 95 μM, at least 100 μM, or at least 105 μM. In one embodiment, the media comprises about 106 μM±5 μM of purine derivatives. Purine derivatives include hypoxanthine and the nucleosides adenosine and guanosine. In one embodiment, the media comprises pyrimidine derivatives in a cumulative concentration of at least 30 μM, at least 35 μM, at least 40 μM, at least 45 μM, at least 50 μM, at least 55 μM, at least 60 μM, or at least 65 μM. In one embodiment, the media comprises about 68 μM±5 μM of pyrimidine derivatives. Pyrimidine derivatives include the nucleosides thymidine, uridine, and cytidine. In one particular embodiment, the media comprises adenosine, guanosine, cytidine, uridine, thymidine and hypoxanthine.

In addition to the inclusion of ornithine or putrescine, in one embodiment, the media also comprises amino acids in a cumulative concentration of at least 40 mM, wherein the amount of glutamine is not included in the calculation of the cumulative total. In one embodiment, glutamine is not included in the media, but may be supplied as a “point-of-use addition” to the media during the culturing of cells, such as during the production of protein. Thus, in some embodiments, such as in the method to culture cells or the method to produce a protein of interest, the media may be supplemented with glutamine as a point-of-use addition. In one such embodiment, glutamine is added in an amount less than about 40 mM, less than about 35 mM, less than about 30 mM, less than about 25 mM, less than about 20 mM, less than about 15 mM, less than about 10 mM, less than about 8 mM, less than about 7 mM, less than about 6 mM, less than about 5 mM, less than about 4 mM, less than about 3 mM, or less than about 2.5 mM. In one embodiment, the amount of glutamine in the media that was supplemented with glutamine is about 2 mM±0.5 mM.

In one embodiment, in addition to the inclusion of ornithine or a combination of both ornithine and putrescine, the media also comprises amino acids having a non-polar side group in a concentration of at least 15 mM, at least 24 mM, at least 25 mM, at least 26 mM, at least 27 mM, at least 28 mM, at least 29 mM, or at least 30 mM. In one embodiment, the media comprises about 30 mM of amino acids having a non-polar side group. In one embodiment, of the total amount of amino acids by mole contained within the media, at least 32%, at least 33%, at least 34%, at least 35%, at least 36%, at least 37%, at least 38%, at least 39%, at least 40%, or at least 41% are amino acids having non-polar side groups. In one embodiment, about 42%±1% by mole of the amino acids in the media are amino acids having a non-polar side group. Amino acids having a non-polar side group include alanine, valine, leucine, isoleucine, proline, phenylalanine, tryptophan, and methionine. In one embodiment, the optional mixture of amino acid supplements are selected from the group consisting of the amino acids in Table 5 below:

TABLE 5

| Amino acid | RANGE mM (mmol/L) | RANGE (g/L) |
|---|---|---|
| Alanine | 0-11.2 | 0-1 |
| Arginine | 2.4-11.9 | 0.5-2.5 |
| Asparagine | 1.3-33.3 | 0.2-5 |
| Aspartic Acid | 1.5-93.9 | 0.2-12.5 |
| Cysteine | 1.1-19.9 | 0.2-3.5 |
| Glutamic acid | 1.4-47.6 | 0.2-7 |
| Glutamine | 0-23.9 | 0-3.5 |
| Glycine | 0-16.7 | 0-1.25 |
| Histidine | 1-9.5 | 0.2-2 |
| Isoleucine | 1.5-22.9 | 0.2-3 |
| Leucine | 1.5-38.1 | 0.2-5 |
| Lysine | 2.7-24.6 | 0.5-4.5 |
| Methionine | 1.3-13.4 | 0.2-2 |
| Phenylalanine | 1.2-18.2 | 0.2-3 |
| Proline | 1.7-26.1 | 0.2-3 |
| Serine | 1.9-57.1 | 0.2-6 |
| Threonine | 1.7-33.6 | 0.2-4 |
| Tryptophan | 0.5-14.7 | 0.1-3 |
| Tyrosine | 0.9-22.2 | 0.2-5 |
| Valine | 1.7-34.1 | 0.2-4 |

In one embodiment, in addition to the inclusion of ornithine or a combination of both ornithine and putrescine, the media also comprises amino acids having an uncharged polar side group in a concentration of about 10 mM to 34 mM, about 11 mM to 33 mM, about 12 mM to 32 mM, about 13 mM to 31 mM, about 14 mM to 30 mM, about 15 mM to 29 mM, about 16 mM to 28 mM, about 17 mM to 27 mM, about 18 mM to 26 mM, about 19 mM to 25 mM, about 20 mM to 24 mM, about 21 mM to 23 mM, about 22 mM, about 23 mM, about 24 mM, or about 25 mM. In one embodiment, the medium comprises about 22 mM of amino acids having an uncharged polar side group. In another embodiment, the medium comprises about 12 mM amino acids having an uncharged polar side group. In one embodiment, of the total amount by mole of amino acids contained within the media, about 14% to 46%, about 15% to 45%, about 16% to 44%, about 17% to 43%, about 18% to 42%, about 19% to 41%, about 20% to 40%, about 21% to 39%, about 22% to 38%, about 23% to 37%, about 24% to 36%, about 25% to 35%, about 26% to 34%, about 27% to 33%, about 28% to 32%, about 29% to 31%, or about 30% are amino acids having uncharged polar side groups. In one embodiment, about 30%±3% by mole of the amino acids in the media are amino acids having an uncharged polar side group. Amino acids having an uncharged polar side group include glycine, serine, threonine, cysteine, tyrosine, asparagine, and glutamine.

In one embodiment, in addition to the inclusion of ornithine or a combination of both ornithine and putrescine, the media also comprises amino acids having a negative charge at pH 6 (e.g., acidic amino acids) in a concentration of about 4 mM to 14 mM, about 5 mM to 13 mM, about 6 mM to 12 mM, about 7 mM to 11 mM, about 8 mM to 10 mM, about 9 mM, or about 4 mM. In one embodiment, the media comprises about 9 mM of acidic amino acids. In one embodiment, the media comprises 9 mM±1 mM of acidic amino acids. In one embodiment, of the total amount by mole of amino acids contained within the media, about 8% to 18%, about 9% to 17%, about 10% to 16%, about 11% to 15%, about 12% to 14%, or about 13% are acidic amino acids. In one embodiment, about 12.6%±1% by mole of the amino acids in the media are acidic amino acids. Acidic amino acids include aspartic acid and glutamic acid.

In one embodiment, in addition to the inclusion of ornithine or a combination of both ornithine and putrescine, the media also comprises amino acids having a positive charge at pH 6 (e.g., basic amino acids) in a concentration of at least 3.5 mM, at least 4 mM, at least 5 mM, at least 6 mM, at least 7 mM, at least 8 mM, at least 9 mM, at least 10 mM, or at least 11 mM. In one embodiment, the media comprises about 11 mM of basic amino acids. In one embodiment, the media comprises about 11.42 mM±1 mM of basic amino acids. In one embodiment, of the total amount by mole of amino acids contained within the media, at least 5%, at least 6%, at least 7%, at least 8%, at least 9%, at least 10%, at least 11%, at least 12%, at least 13%, at least 14%, or at least 15% are basic amino acids. In one embodiment, about 16% by mole of the amino acids in the media are basic amino acids. In one embodiment, about 15.8%±2.4% by mole of the amino acids in the media are basic amino acids. In one embodiment, about 21%±3.2% by mole of the amino acids in the media are basic amino acids. Basic amino acids include lysine, arginine, and histidine.

In one embodiment, in addition to the inclusion of ornithine or a combination of both ornithine and putrescine, the media also comprises about 30 mM non-polar amino acids, about 22 mM uncharged polar amino acids, about 9 mM acidic amino acids, and about 11 mM basic amino acids. In one embodiment, of the amino acids in the media, about 42% by mole are non-polar amino acids, about 30% by mole are uncharged polar amino acids, about 13% by mole are acidic amino acids, and about 16% by mole are basic amino acids.

In addition to the inclusion of ornithine or a combination of both ornithine and putrescine, in one embodiment, the media comprises micromolar amounts of fatty acids (or fatty acid derivatives) and tocopherol. In one embodiment, the fatty acids include any one or more of linoleic acid, linolenic acid, thioctic acid, oleic acid, palmitic acid, stearic acid, arachidic acid, arachidonic acid, lauric acid, behenic acid, decanoic acid, dodecanoic acid, hexanoic acid, lignoceric acid, myristic acid, and octanoic acid. In one embodiment, the media comprises tocopherol, linoleic acid, and thioctic acid.

In one embodiment, the media also comprises a mixture of vitamins, which includes other nutrients and essential nutrients, at a cumulative concentration of at least about 700 μM or at least about 2 mM. In one embodiment, the mixture of vitamins comprises one or more of D-biotin, choline chloride, folic acid, myo-inositol, niacinamide, pyridoxine HCl, D-pantothenic acid (hemiCa), riboflavin, thiamine HCl, vitamin B12, and the like. In one embodiment, the mixture of vitamins includes all of D-biotin, choline chloride, folic acid, myo-inositol, niacinamide, pyridoxine HCl, D-pantothenic acid (hemiCa), riboflavin, thiamine HCl, and vitamin B12.

In one embodiment, the media also comprises taurine, hypotaurine, or combinations thereof. Taurine is present in many tissues of humans and other mammalian species, e.g. brain, retina, myocardium, skeletal and smooth muscle, platelets and neutrophils. Taurine may help with osmoregulation, membrane stabilization and anti-inflammation, and also regulates mitochondrial protein synthesis through enhanced electron transport chain activity that protects against superoxide generation (Jong et al., 2010, Journal of Biomedical Science 17(Suppl 1): S25; Jong et al., 2012, Amino Acids 42:2223-2232sw). In one aspect, a cell culture medium which is serum-free may comprise about 0.1 mM to about 10 mM taurine, about 0.1 mM to about 1 mM taurine, about 0.4 mM to about 8 mM taurine, about 1 mM to about 6 mM taurine, about 2 mM to about 5 mM taurine, or about 3 to about 4 mM taurine. Taurine has also been found to increase cellular specific productivity and allows for lower ammonia byproduct by those cells to improve the production of recombinant proteins. See U.S. Pat. No. 10,927,342, which is incorporated by reference in its entirety.

In one aspect, a fed-batch production method of making Dupilumab comprises culturing Chinese Hamster Ovary (CHO) cells comprising a nucleic acid encoding said Dupilumab in a cell culture production medium in large-scale, wherein insulin in said medium is supplemented on days 2 and 4 at a concentration of about 7.5 mg/L.

In one embodiment, said Dupilumab is produced at a titer of at least 1.5 g/L in said cell culture production medium by day 4. In another embodiment, said Dupilumab is produced at a titer of at least 2 g/L in said cell culture production medium by day 4.

In one embodiment, said culturing lasts for about 10-18 days. In another embodiment, said culturing lasts for about 10 days, about 12 days, about 14 days, about 16 days, or about 18 days.

In one embodiment, said cell culture production medium is a serum-free medium. In some embodiments, said serum-free medium comprises a recombinant growth factor, an osmolarity regulator, a pH buffer agent, glutamine, and a cell protectant.

In one embodiment, said CHO cells are cultured at a temperature ranging from about 32° C. to about 38° C.

In one embodiment, said Dupilumab is produced at a titer of at least about 0.5 g/L in said cell culture production medium on day 4. In another embodiment, said Dupilumab is so produced such that the viability of said cells is at least about 95% in said cell culture production medium on day 4. In yet another embodiment, said Dupilumab is so produced such that the concentration of ammonia in said cell culture production medium on day 4 is less than about 5 mM.

In one aspect, a fed-batch production method of making Dupilumab comprises culturing Chinese Hamster Ovary (CHO) cells comprising a nucleic acid encoding said Dupilumab in a cell culture production medium in large-scale, wherein tyrosine in said medium is supplemented on day 3 at a concentration of about 2 g/L.

In one embodiment, said Dupilumab is produced at a titer of at least about 8 g/L in said cell culture production medium on day 14.

In one embodiment, said Dupilumab is produced at a titer of at least 9 g/L in said cell culture production medium at days 10-14. In another embodiment, said Dupilumab is produced at a titer of at least 10 g/L in said cell culture production medium at days 10-14.

In one embodiment, said cell culture production medium is a serum-free medium. In some embodiments, said serum-free medium comprises a recombinant growth factor, an osmolarity regulator, a pH buffer agent, glutamine, methotrexate, and a cell protectant.

In one embodiment, said CHO cells are cultured at a temperature ranging from about 32° C. to about 38° C.

In one embodiment, the concentration of ammonia in said cell culture production medium on day 14 is less than about 10 mM. In another embodiment, the concentration of ammonia in said cell culture production medium on day 14 is less than about 8 mM.

In one aspect, a fed-batch production method of making Dupilumab comprises culturing Chinese Hamster Ovary (CHO) cells comprising a nucleic acid encoding said Dupilumab in a cell culture production medium in large-scale, wherein tyrosine in said medium is supplemented on day 3 and day 7 at a concentration of about 2 g/L.

In one aspect, a fed-batch production method of making Dupilumab comprises culturing Chinese Hamster Ovary (CHO) cells comprising a nucleic acid encoding said Dupilumab in a cell culture production medium in large-scale, wherein tyrosine in said medium is supplemented on day 3 and day 7 at a concentration of about 1 g/L.

In one embodiment, said Dupilumab is produced at a titer of at least about 8 g/L in said cell culture production medium on day 14. In another embodiment, said Dupilumab is produced at a titer of at least 9 g/L in said cell culture production medium. In yet another embodiment, said Dupilumab is produced at a titer of at least 10 g/L in said cell culture production medium.

In one embodiment, said cell culture production medium is a serum-free medium. In some embodiments, said serum-free medium comprises a recombinant growth factor, an osmolarity regulator, a pH buffer agent, glutamine, methotrexate, and a cell protectant.

In one embodiment, said CHO cells are cultured at a temperature ranging from about 32° C. to about 38° C.

In one aspect, a fed-batch production method of making Dupilumab comprises culturing Chinese Hamster Ovary (CHO) cells comprising a nucleic acid encoding said Dupilumab in a cell culture production medium in large-scale, wherein sodium phosphate in said medium is supplemented on days 2 and 4, or at days 0, 2, 4, 6, and/or 8, to a concentration of about 200-550, about 200-275, about 250-325, about 300-375, about 350-425, about 400-475, about 450-525, or about 500-550 mg/L, such that titer of said Dupilumab in said cell culture production medium on day 10 is about 5 g/L, about 6 g/L, about 7 g/L, or about 8 g/L. In another aspect, titer of said Dupilumab in said cell culture production medium between days 10 and 14 is about 4 g/L, about 5 g/L, about 6 g/L, about 7 g/L, about 8 g/L, about 9 g/L or about 10 g/L.

In one embodiment, said cell culture production medium is a serum-free medium. In some embodiments, said serum-free medium comprises a recombinant growth factor, an osmolarity regulator, a pH buffer agent, glutamine, methotrexate, and a cell protectant.

In one embodiment, said CHO cells are cultured at a temperature ranging from about 32° C. to about 38° C.

In one embodiment, said sodium phosphate in said medium is supplemented on days 2 and 4, or at days 0, 2, 4, 6, and/or 8, at a concentration of about 200-550, about 200-275, about 250-325, about 300-375, about 350-425, about 400-475, or about 450-525 mg/L.

In one aspect, a fed-batch production method of making Dupilumab comprises culturing Chinese Hamster Ovary (CHO) cells comprising a nucleic acid encoding said Dupilumab in a cell culture production medium in large-scale, wherein sodium phosphate in said medium is supplemented on days 2 and 4, or on days 0, 2, 4, 6, and 8, to a concentration of about 200-550, about 200-275, about 250-325, about 300-375, about 350-425, about 400-475, about 450-525, or about 500-550 mg/L, respectively, wherein tyrosine in said medium is supplemented on day 3 at a concentration of about 2 g/L, and wherein insulin in said medium is supplemented on days 2 and 4 at a concentration of about 7.5 mg/L such that titer of said Dupilumab in said cell culture production medium on day 10 is at least 5 g/L.

In one aspect, a fed-batch production method of making Dupilumab comprises culturing Chinese Hamster Ovary (CHO) cells comprising a nucleic acid encoding said Dupilumab in a cell culture production medium in large-scale, wherein sodium phosphate in said medium is supplemented on days 2 and 4, or on days 0, 2, 4, 6, and 8, to a concentration of about 200-550, about 200-275, about 250-325, about 300-375, about 350-425, about 400-475, about 450-525, or about 500-550 mg/L, respectively, wherein tyrosine in said medium is supplemented on day 3 and day 7 at a concentration of about 1 g/L, and wherein insulin in said medium is supplemented on days 2 and 4 at a concentration of about 7.5 mg/L, such that titer of said Dupilumab in said cell culture production medium on day 10 is at least 5 g/L.

In one embodiment, said cell culture production medium is a serum-free medium. In some embodiments, said serum-free medium comprises a recombinant growth factor, an osmolarity regulator, a pH buffer agent, glutamine, methotrexate, and a cell protectant.

In one embodiment, said CHO cells are cultured at a temperature ranging from about 32° C. to about 38° C.

In one embodiment, sodium phosphate in said medium is supplemented on days 2 and 4, or on days 0, 2, 4, 6, and 8, to a concentration of 200-550, about 200-275, about 250-325, about 300-375, about 350-425, about 400-475, or about 450-525 mg/L.

In one embodiment, said culturing lasts about 10 days.

Various embodiments of the media of the invention include any of the combinations of the above described embodiments, including chemically defined, hydrolysate-free serum-free media comprising ornithine or putrescine in the indicated amounts, plus inter alia (a) amino acids; (b) optionally nucleosides; (c) salts of divalent cations; (d) fatty acids and tocopherol; and (e) vitamins. In some embodiments, small amounts of hydrolysates may be added to the PS media.

The applicants envision that in the practice of this invention any one or more of a variety of base media or combinations thereof, to which the ornithine or a combination of both ornithine and putrescine are added, may be used. Base media are generally known in the art and include inter alia Eagle's MEME (minimal essential media) (Eagle, Science, 1955, 112(3168):501-504), Ham's F12 (Ham, Proc. Nat'l. Acad. Sci. USA, 1965, 53:288-293), F-12 K medium, Dulbecco's medium, Dulbecco's Modified Eagle Medium (Proc. Natl. Acad. Sci. USA., 1952 August; 38(8): 747-752), DMEM/Ham's F12 1:1, Trowell's T8, A2 media Holmes and Wolf, Biophys. Biochem. Cytol., 1961, 10:389-401), Waymouth media (Davidson and Waymouth, Biochem. J., 1945, 39(2):188-199), Williams E media (William's et al., Exp. Cell Res., 1971, 69:105 et seq.), RPMI 1640 (Moore et al., J. Amer. Med. Assoc., 1967, 199:519524), MCDB 104/110 media (Bettger et al., Proc. Nat'l. Acad. Sci. USA, 1981, 78(9):5588-5592), Ventrex HL-1 media, albumin-globulin media (Orr et al., Appl. Microbiol., 1973, 25(1):4954), RPMI-1640 Medium, RPMI-1641 Medium, Iscove's Modified Dulbecco's Medium, McCoy's 5 A Medium, Leibovitz's L-15 Medium, and serum-free media such as EX-CELL™ 300 Series (JRH Biosciences, Lenexa, Kansas), protamine-zinc-insulin media (Weiss et al., 1974, U.S. Pat. No. 4,072,565), biotin-folate media (Cartaya, 1978, U.S. Re30,985), Transferrin-fatty acid media (Baker, 1982, U.S. Pat. No. 4,560,655), transferrin-EGF media (Hasegawa, 1982, U.S. Pat. No. 4,615,977; Chessebeuf, 1984, U.S. Pat. No. 4,786,599), and other media permutations (see Inlow, U.S. Pat. No. 6,048,728; Drapeau, U.S. Pat. No. 7,294,484; Mather, U.S. Pat. No. 5,122,469; Furukawa, U.S. Pat. No. 5,976,833; Chen, U.S. Pat. No. 6,180,401; Chen, U.S. Pat. No. 5,856,179; Etcheverry, U.S. Pat. No. 5,705,364; Etcheverry, U.S. Pat. No. 7,666,416; Ryll, U.S. Pat. No. 6,528,286; Singh, U.S. Pat. No. 6,924,124; Luan, U.S. Pat. No. 7,429,491; and the like).

In a particular embodiment, the media is chemically defined and comprises in addition to the ornithine or combination of both ornithine and putrescine: CaCl₂ 2H₂O; HEPES buffer, KCL; MgSO₄; NaCl; Na₂HPO₄ or other phosphate salts; pyruvate; L-alanine; L-arginine HCl; L-asparagine H₂O; L-aspartic acid; L-cysteine HCl H₂O; L-glutamic acid; Glycine; L-histidine HCl H₂O; L-isoleucine; L-leucine; L-lysine HCl; L-methionine; L-ornithine HCl; L-phenylalanine; L-proline; L-serine; L-threonine; L-tryptophan; L-tyrosine 2Na 2 H₂O; L-valine; D-biotin; choline chloride; folic acid; myo-inositol; niacinamide; pyridoxine HCl; D-pantothenic acid; riboflavin; thiamine HCl; vitamin B12; p-aminobenzoic acid; ethanolamine HCl; poloxamer 188; DL-a-tocopherol phosphate; linoleic acid; Na₂SeO₃; thioctic acid; and glucose; and optionally adenosine; guanosine; cytidine; uridine; thymidine; and hypoxanthine 2Na.

In one embodiment, the starting osmolarity of the media is 200-500, 250-400, 275-350, or about 300 mOsm. During growth of the cells in the media, and in particular following any feedings according to a fed batch protocol, the osmolarity of the culture may increase up to about 350, 400, 450, or as high as 500 mOsm.

In some embodiments wherein the osmolarity of the defined medium is less than about 300, the osmolarity is brought to about 300 with the addition of one or more salts in excess of the amount specified. In one embodiment, osmolarity is increased to a desired level by adding one or more of an osmolyte selected from sodium chloride, potassium chloride, a magnesium salt, a calcium salt, an amino acid salt, a salt of a fatty acid, sodium bicarbonate, sodium carbonate, potassium carbonate, a chelator that is a salt, a sugar (e.g., galactose, glucose, sucrose, fructose, fucose, etc.), and a combination thereof. In one embodiment, the osmolyte is added over and above its concentration in a component already present in the defined medium (e.g., a sugar is added over and above the concentration specified for a sugar component).

The present disclosure provides a cell culture comprising a cell line expressing a protein of interest in an OS medium as described above. In one embodiment, the cell culture comprises insulin, which can be added as a point-of-use ingredient to the media, or can be included in the media formulation. In one embodiment, the cell line comprises cells capable of producing a biotherapeutic protein. Examples of cell lines that are routinely used to produce protein biotherapeutics include inter alia primary cells, BSC cells, HeLa cells, HepG2 cells, LLC-MK cells, CV-1 cells, COS cells, VERO cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, CHO cells, CHO-K1 cells, NS-1 cells, MRC-5 cells, WI-38 cells, 3T3 cells, HEK293 cells, RK cells, Per.C6 cells and chicken embryo cells. In one embodiment, the cell line is a CHO cell line or one or more of several specific CHO cell variants optimized for large-scale protein production, e.g., CHO-K1.

Animal cells, such as CHO cells, may be cultured in small scale cultures, such as in 125 mL containers having about 25 mL of media, 250 mL containers having about 50 to 100 mL of media, or 500 mL containers having about 100 to 200 mL of media. Alternatively, the cultures can be large scale, such as, for example, 1000 mL containers having about 300 to 1000 mL of media, 3000 mL containers having about 500 mL to 3000 mL of media, 8000 mL containers having about 2000 mL to 8000 mL of media, and 15000 mL containers having about 4000 mL to 15000 mL of media. Cultures for manufacturing can comprise large-scale 10,000 L of media or more. Large scale cell cultures, such as for clinical manufacturing of protein therapeutics, are typically maintained for days, or even weeks, while the cells produce the desired protein(s). During this time, the culture can be supplemented with a concentrated feed medium comprising components, such as nutrients and amino acids, which are consumed during the course of the culture. Concentrated feed medium may be based on any cell culture media formulation. Such a concentrated feed medium can comprise most of the components of the cell culture medium at, for example, about 5×, 6×, 7×, 8×, 9×, 10×, 12×, 14×, 16×, 20×, 30×, 50×, 100×, 200×, 400×, 600×, 800×, or even about 1000× of their normal useful amount. Concentrated feed media are often used in fed batch culture processes.

In some embodiments, the cell culture media is supplemented with “point-of-use additions,” also known as additions, point-of-use ingredients, or point-of-use chemicals, during the course of cell growth or protein production. Point-of-use additions include any one or more of a growth factor or other proteins, a buffer, an energy source, a salt, an amino acid, a metal, and a chelator. Other proteins include transferrin and albumin. Growth factors, which include cytokines and chemokines, are generally known in the art and are known to stimulate cell growth, or in some cases, cellular differentiation. A growth factor is usually a protein (e.g., insulin), a small peptide, or a steroid hormone, such as estrogen, DHEA, testosterone, and the like. In some cases, a growth factor may be a non-natural chemical that promotes cell proliferation or protein production, such as e.g., tetrahydrofolate (THF), methotrexate, and the like. Non-limiting examples of protein and peptide growth factors include angiopoietins, bone morphogenetic proteins (BMPs), brain-derived neurotrophic factor (BDNF), epidermal growth factor (EGF), erythropoietin (EPO), fibroblast growth factor (FGF), glial cell line-derived neurotrophic factor (GDNF), granulocyte colony-stimulating factor (G-CSF), granulocyte macrophage colony-stimulating factor (GM-CSF), growth differentiation factor-9 (GDF9), hepatocyte growth factor (HGF), hepatoma-derived growth factor (HDGF), insulin, insulin-like growth factor (IGF), migration-stimulating factor, myostatin (GDF-8), nerve growth factor (NGF) and other neurotrophins, platelet-derived growth factor (PDGF), thrombopoietin (TPO), transforming growth factor α (TGF-α), transforming growth factor beta (TGF-β), tumor necrosis factor-α (TNF-α), vascular endothelial growth factor (VEGF), Wnt signaling pathway agonists, placental growth factor (PIGF), fetal Bovine somatotrophin (FBS), interleukin-1 (IL-1), IL-2, IL-3, IL-4, IL-5, IL-6, IL-7, and the like. In one embodiment, the cell culture media is supplemented with the point-of-use addition growth factor insulin. In one embodiment, the concentration of insulin in the media, e.g., the amount of insulin in the cell culture media after addition, is from about 0.1 μM to 10 μM. One or more of the point-of-use additions can also be included in the media formulation of some embodiments.

Buffers are generally known in the art. The invention is not restricted to any particular buffer or buffers, and any one of ordinary skill in the art can select an appropriate buffer or buffer system for use with a particular cell line producing a particular protein. In one embodiment, a point-of-use addition buffer is NaHCO₃. In one embodiment, the point-of-use addition buffer comprises NaHCO₃. In another embodiment, the buffer is HEPES.

Energy sources for use as a point-of-use addition in cell culture are also well known in the art. Without limitation, in one embodiment, the point-of-use addition energy source is glucose. Given the particular and specific requirements of a particular cell line and the protein to be produced, in one embodiment the glucose can be added to a cumulative concentration of about 1 to 35 mM in the media. In some cases, glucose can be added at high levels up to 10 g/L.

Chelators are likewise well known in the art of cell culture and protein production. Tetrasodium EDTA dihydrate and citrate are two common chelators used in the art, although other chelators may be employed in the practice of this invention. In one embodiment, a point-of-use addition chelator is tetrasodium EDTA dihydrate. In one embodiment, a point-of-use addition chelator is citrate, such as Na₃C₆H₅O₇.

In one embodiment, the cell culture may be supplemented with one or more point-of-use addition amino acids, such as e.g., glutamine. In one embodiment, the cell culture media is supplemented with the point-of-use addition glutamine at a final concentration of about 1 mM to 13 mM.

Other point-of-use additions include one or more of various amino acids and metal salts, such as salts of iron, nickel, zinc and copper. In one embodiment, the cell culture media is supplemented with any one or more of copper sulfate, zinc sulfate, ferric chloride, and nickel sulfate.

In one embodiment, the cell culture media is supplemented with any one or more or all of the following point-of-use additions: about 25-30 mM NaHCO₃, about 1.5-2 mM glutamine, about 0.75-0.9 μM insulin, about 10.75-11.25 mM glucose, about 6.25-6.75 μM zinc sulfate, about 0.15-0.17 μM copper sulfate, about 70-80 μM ferric chloride, about 0.6-0.7 μM nickel sulfate, about 80-90 μM EDTA, and about 45-60 μM citrate.

In one embodiment, the media is supplemented at intervals during cell culture according to a fed-batch process. Fed-batch culturing is generally known in the art and employed to optimized protein production (see Y. M. Huang et al., Biotechnol Prog. 2010 September-October; 26(5):1400-10).

Cell viability, viable cell density, and cell doubling are improved relative to cells grown in culture without ornithine or putrescine. Regarding cell viability, cells grown in PS media show a viability that is at least 10%, at least 15%, at least 20%, at least 25%, at least 30%, at least 35%, at least 40%, at least 45%, at least 50%, at least, 55%, at least 60%, at least 65%, at least 70%, at least 75%, at least 80%, at least 85%, at least 90%, at least 95%, at least 99%, at least 100%, or at least 3-fold greater than the viability of similar or identical cells grown in non-PS media.

In some embodiments, the doubling rate of viable mammalian cells in PS media is at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 35%, at least 40%, at least 45%, at least 50%, at least 55%, at least 60%, at least 70%, at least 80%, at least 90%, at least 100%, or at least 3-fold greater than the doubling rate of mammalian cells cultured in non-PS media. In some embodiments, the doubling rate of viable mammalian cells in PS media is about 10%, 11%, 12%, 13%, 14%, 15%, 16%, 17%, 18%, 19%, 20%, 21%, 22%, 23%, 24%, 25%, 26%, 27%, 28%, 29%, or 30% greater than the doubling rate of mammalian cells in non-PS media.

In some embodiments, the doubling time of actively cycling mammalian cells is less than 30 hours, less than 29 hours, less than 28 hours, less than 27 hours, less than 26 hours, less than 25 hours, less than 24 hours, less than 23 hours, less than 22 hours, less than 21 hours, less than 20 hours, less than 19 hours, or less than 18 hours in PS media. In some embodiments, the doubling time of actively growing mammalian cells is less than 28 hours in PS media. In some embodiments, the doubling time of mammalian cells is about 27±1 hours, about 26±1 hours, about 25±1 hours, about 24±1 hours, about 23±1 hours, about 22±1 hours, or about 21±1 hours in PS media. In some embodiments, the doubling time of actively cycling mammalian cells is about 24±1 hours in PS media. In some embodiments, the doubling time of actively dividing cells cultured in PS media is at least 15%, at least 16%, at least 17%, at least 18%, at least 19%, at least 20%, or at least 25% shorter than the doubling time of actively cycling cells cultured in a non-PS media.

### Protein Production

In addition to chemically defined PS media and methods of culturing cells in PS media, the present disclosure provides methods of producing a protein, such as a therapeutically effective antibody or other biopharmaceutical drug substance, in a cell cultured in PS media.

In some embodiments, the rate of production of protein by mammalian cells cultured in PS media is at least 5%, 10%, 15%, or 20% greater than the rate of production of protein by an identical mammalian cell cultured in non-PS media. In some embodiments, the rate of production of protein in cells cultured in PS media is at least 1 pg/cell/day (“PCD”), at least 2 PCD, at least 3 PCD, at least 4 PCD, at least 5 PCD, at least 6 PCD, at least 7 PCD, at least 8 PCD, at least 9 PCD, at least 10 PCD, at least 15 PCD, at least 20 PCD, at least 25 PCD, at least 30 PCD, at least 35 PCD, at least 40 PCD, at least 45 PCD, at least 50 PCD, at least 75 PCD, or at least 100 PCD.

In some embodiments the protein production yield or titer, which can be expressed in grams of protein product per liter of culture media, from cells cultured in PS media is at least 100 mg/L, at least 1 g/L, at least 1.2 g/L, at least 1.4 g/L, at least 1.6 g/L, at least 1.8 g/L, at least 2 g/L, at least 2.5 g/L, at least 3 g/L, at least, 3.5 g/L, at least 4 g/L, at least 4.5 g/L, at least 5 g/L, at least 5.5 g/L, at least 6 g/L, at least 6.5 g/L, at least 7 g/L, at least 7.5 g/L, at least 8 g/L, at least 8.5 g/L, at least 9 g/L, at least 9.5 g/L, at least 10 g/L, or at least 20 g/L.

In some embodiments, the protein product (protein of interest) is an antibody, a human antibody, a humanized antibody, a chimeric antibody, a monoclonal antibody, an antigen-binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, or an IgG4 antibody. In one embodiment, the antibody is an IgG1 antibody. In one embodiment, the antibody is an IgG2 antibody. In one embodiment, the antibody is an IgG4 antibody.

In some embodiments, the protein of interest is a recombinant protein that comprises an Fc moiety and another domain. In some embodiments, the Fc moiety comprises a hinge region followed by a CH2 and CH3 domain of an IgG.

The present invention is not limited to any particular type of cell for protein production. Examples of cell types suitable for protein production include mammalian cells, primate cells, insect cells, avian cells, bacterial cells, and yeast cells. The cells may be stem cells or recombinant cells transformed with a vector for recombinant gene expression, or cells transfected with a virus for producing viral products. The cells may comprise a recombinant heterologous polynucleotide construct that encodes a protein of interest. That construct can be an episome or it can be an element that is physically integrated into the genome of the cell. The cells may also produce a protein of interest without having that protein encoded on a heterologous polypeptide construct. In other words, the cell may naturally encode the protein of interest, such as a B-cell producing an antibody. The cells may also be primary cells, such as chicken embryo cells, or primary cell lines. Examples of useful cells include BSC cells, LLC-MK cells, CV-1 cells, COS cells, VERO cells, MDBK cells, MDCK cells, CRFK cells, RAF cells, RK cells, TCMK-1 cells, LLCPK cells, PK15 cells, LLC-RK cells, MDOK cells, BHK-21 cells, chicken embryo cells, NS-1 cells, MRC-5 cells, WI-38 cells, BHK cells, HEK293 cells, RK cells, Per.C6 cells and CHO cells. In various embodiments, the cell line is a CHO cell derivative, such as CHO-K1, CHO DUX B-11, Veggie-CHO, GS-CHO, S-CHO, or CHO lec mutant lines.

In one embodiment, the cell, which is a CHO cell, ectopically expresses a protein. In one embodiment, the protein comprises an immunoglobulin heavy chain region, such as a CH1, CH2, or CH3 region. In one embodiment, the protein comprises a human or rodent immunoglobulin CH2 and CH3 region. In one embodiment, the protein comprises a human or rodent immunoglobulin CH1, CH2, and CH3 region. In one embodiment, the protein comprises a hinge region and a CH1, CH2, and CH3 region. In some embodiments, the protein comprises an immunoglobulin heavy chain variable domain. In some embodiments, the protein comprises an immunoglobulin light chain variable domain. In some embodiments, the protein comprises an immunoglobulin heavy chain variable domain and an immunoglobulin light chain variable domain. In some embodiments, the protein is an antibody, such as a human antibody, a rodent antibody, or a chimeric human/rodent antibody (e.g., human/mouse, human/rat, or human hamster).

A production phase can be conducted at any scale of culture, from flasks or wave bags, to one-liter bioreactors, and to large scale industrial bioreactors. A large-scale process can be conducted in a volume of about 1,000 liters to 25,000 liters or more. One or more of several means may be used to control protein production, such as temperature shift or chemical induction. A growth phase may occur at a higher temperature than a production phase. For example, a growth phase may occur at a first temperature of about 35° C. to 38° C., and a production phase may occur at a second temperature of about 29° C. to 37° C., optionally from about 30° C. to 36° C. or from about 30° C. to 34° C. In addition, chemical inducers of protein production, such as caffeine, butyrate, tamoxifen, estrogen, tetracycline, doxycycline, and hexamethylene bisacetamide (HMBA), may be added concurrent with, before, or after a temperature shift. If inducers are added after a temperature shift, they can be added from one hour to five days after the temperature shift, such as from one to two days after the temperature shift. Production cell cultures may be run as continuous feed culture system, as in a chemostat (see C. Altamirano et al., Biotechnol Prog. 2001 November-December; 17(6):1032-41), or according to a fed-batch process (Huang, 2010).

The invention is useful for improving protein production via cell culture processes. The cell lines used in the invention can be genetically engineered to express a polypeptide of commercial or scientific interest. Genetically engineering the cell line involves transfecting, transforming or transducing the cells with a recombinant polynucleotide molecule, or otherwise altering (e.g., by homologous recombination and gene activation or fusion of a recombinant cell with a non-recombinant cell) so as to cause the host cell to express a desired recombinant polypeptide. Methods and vectors for genetically engineering cells or cell lines to express a polypeptide of interest are well known to those of skill in the art; for example, various techniques are illustrated in Current Protocols in Molecular Biology. Ausubel et al., eds. (Wiley & Sons, New York, 1988, and quarterly updates); Sambrook et al., Molecular Cloning: A Laboratory Manual (Cold Spring Laboratory Press, 1989); Kaufman, R. J., Large Scale Mammalian Cell Culture, 1990, pp. 15-69. A wide variety of cell lines suitable for growth in culture are available from the American Type Culture Collection (Manassas, Va.) and commercial vendors. Examples of cell lines commonly used in the industry include VERO, BHK, HeLa, CVI (including Cos), MDCK, HEK293, 3T3, myeloma cell lines (e.g., NSO, NSI), PC12, W138 cells, and Chinese hamster ovary (CHO) cells. CHO cells are widely used for the production of complex recombinant proteins, such as cytokines, clotting factors, and antibodies (Brasel et al. (1996), Blood 88:2004-2012; Kaufman et al. (1988), J. Biol Chem 263:6352-6362; McKinnon et al. (1991), J Mot Endocrinol 6:231-239; Wood et al. (1990), J Immunol. 145:3011-3016). The dihydrofolate reductase (DHFR)-deficient mutant cell line (Urlaub et al. (1980), Proc Natl Acad Sci USA 77: 4216-4220) DXBI 1 is a desirable CHO host cell line because the efficient DHFR selectable and amplifiable gene expression system allows high level recombinant protein expression in these cells (Kaufman R J. (1990), Meth Enzymol 185:537-566). In addition, these cells are easy to manipulate as adherent or suspension cultures and exhibit relatively good genetic stability. CHO cells and the proteins recombinantly expressed by them have been extensively characterized and have been approved for use in commercial manufacturing. In some embodiments, the CHO cell lines are cell lines as described in U.S. Patent Application Publications No. 2010/0304436 A1, 2009/0162901 A1 and 2009/0137416 A1, and U.S. Pat. Nos. 7,455,988 B2, 7,435,553 B2, and 7,105,348 B2.

The present disclosure is not limited in scope by the specific embodiments described herein, which are intended as illustrations of individual aspects or embodiments of the invention. Functionally equivalent methods and components are within the scope of the invention. Various modifications of the invention, in addition to those described here, are apparent to those skilled in the art from the foregoing description and accompanying drawings. Such modifications fall within the scope of the invention.

The disclosure is based, in part, on the discovery that addition of ornithine or a combination of ornithine and putrescine to serum-free cell culture media results in increased cell growth, viability and polypeptide production from a recombinantly engineered animal cell line (or natural cell) expressing a protein of interest, thereby enhancing culture robustness, improving the yield of the polypeptide of interest.

### Seed Train Optimization and Measurement

As described herein, improved methods have been developed for optimization of large-scale production of proteins in cell culture by implementing a seed train process that forces and biases cells to increase viable cell density (VCD) in the large-scale production vessel. In U.S. application Ser. No. 16/984,581 titled Metabolically Optimized Cell Culture, incorporated by reference, it had previously been observed that peak lactate consumption in the seed train could be used to improve large scale production of proteins. As described herein, a new seed-train process has been developed that further enhances cell metabolism and productivity by modulating the initial VCD and without impacting observed lower peak lactate consumption, evidencing a new and different mechanism for enhancing protein production from that described in U.S. application Ser. No. 16/984,581.

By way of background, it is well-known that manufacture of proteins via cell culture is customarily performed using a batch or fed-batch process. Early stages of inoculum growth after vial thaw include culturing cells in a seed culture. Culturing vessels include, but are not limited to, well plates, wave bags, T-flasks, shake flasks, stirred vessels, spinner flasks, hollow fiber, air lift bioreactors, and the like. A suitable cell culturing vessel is a bioreactor. A bioreactor refers to any culturing vessel that is manufactured or engineered to manipulate or control environmental conditions. Such culturing vessels are well known in the art. Typically, cells are grown at an exponential growth rate, such as in seed train bioreactors, in order to progressively increase size and/or volume of the cell population. After cell mass is scaled up through several bioreactor stages, cells are then transferred to a production bioreactor while the cells are still in exponential growth (log phase) (Gambhir, A. et al., 2003, J Bioscience Bioeng 95(4):317-327). It is generally considered undesirable to allow cells in batch culture, for example seed culture, to go past the log phase into stationary phase. It has been recommended that cultures should be passaged while they are in log phase, before, cells, e.g. adherent cells, reach confluence due to contact inhibition or accumulation of waste products inhibits cell growth, among other reasons (Cell Culture Basics, Gibco/Invitrogen Online Handbook, www.invitrogen.com; ATCC® Animal Cell Culture Guide, www.atcc.org).

Following transfer to fed-batch culture, cells are cultured for a period of time whereas the composition of the medium is monitored and controlled to allow production of the protein or polypeptide of interest. After a particular yield is reached or cell viability, waste accumulation or nutrient depletion determines that the culture should be terminated, the produced protein or polypeptide is isolated. Efforts particularly related to reducing the output of metabolic waste products, such as accumulation of lactate, in cell culture have improved the overall quantity of final protein titers. These efforts are focused on controlled glucose or nutrient-limited fed-batch processes (see e.g. WO2004104186; U.S. Pat. No. 8,192,951B2), improved cell culture medium conditions (e.g. U.S. Pat. No. 7,390,660; Zagari, et al., 2013, New Biotechnol., 30(2):238-45), or cellular engineering, including targeting enzymes in the glycolysis pathway (e.g. Kim, S. H. and Lee, G. M., 2007, Appl. Microbiol. Biotechnol. 74, 152-159; Kim, S. H. and Lee, G. M., 2007, Appl. Microbiol. Biotechnol. 76, 659-665; Wlaschin, K. F. and Hu, W-S., 2007, J. Biotechnol. 131, 168-176).

Controlled feeding of cells is utilized in an effort to reach a more efficient metabolic phenotype (Europa, A. F., et al., 2000, Biotechnol. Bioeng. 67:25-34; Cruz et al., 1999, Biotechnol Bioeng, 66(2):104-113; Zhou et al., 1997, Cytotechnology 24, 99-108; Xie and Wang, 1994, Biotechnol Bioeng, 43:1174-89). However, this is complicated by the fact that nutrient deprivation as well as rapid changes in, for example, ammonia concentration seen at high cell density fed-batch culture can induce apoptosis (“programmed cell death”) (Newland et al., 1994, Biotechnol, Bioeng. 43(5):434-8). Hence, a common optimization approach is to grow cells to moderately high density in fed-batch and then deliberately induce a prolonged, productive stationary phase by, e.g., a temperature or pH change (Quek et al., 2010, Metab Eng 12(2):161-71. doi: 10.1016/j.ymben.2009.09.002. Epub 2009 Oct. 13).

In some embodiments, the initial VCD in the seed-train process is modulated to enhance protein production in batch or fed-batch process. In some aspects the initial VCD is increased to about 3.5×10⁵ to 5.43×10⁵ cells/mL compared to an acceptable standard initial VCD ranging from 1.7×10⁵ to 2.3×10⁵ cells/mL at each step (from N-5 to N-1). In some aspects the initial VCD is about 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, or 3.0× greater than an alternative initial VCD in an acceptable standard seed train.

In some embodiments, increasing the initial VCD in N-5 to N-1 vessels results in no change to the final VCD of the N-1 compared to a standard seed-train. In one aspect, the optimized seed train results in an increased final VCD in N-5 to N-2 vessels compared to a standard seed-train. In one aspect there is no substantial difference in peak lactate observed in the final production vessel compared to a standard seed-train. In one aspect the sustained biomass of the optimized seed-train is increased. In one aspect, the optimized seed train resulted in a 1%, 2%, 3%, 4%, 5%, 6%, 7%, 8%, 9%, 10%, 11%, 12%, 13%, 14%, 15%, 16%, 17%, 18%, 19%, or 20% increase in final titer (g/L).

Example 22 is provided to describe to those of ordinary skill in the art how to use the optimized seed-train, and is not intended to limit the scope of what the inventors regard as their invention.

Because of the importance of the VCD in the seed train process and in-process controls, more efficient and improved methods for measuring VCD are needed. Currently, assays used to monitor suspension mammalian cell line cell culture VCD are based on off-line analyses (Ritacco, Biotechnology Progress, 34(6):1407-1426, 2018). For example, the trypan blue exclusion test (TBET) can visually distinguish viable cells from non-viable cells using a manual hemocytometer or an automated bioanalyzer; however, off-line assays are not compatible with the Food and Drug Administration (FDA) regulatory framework for implementation of a mechanism to design, analyze and control pharmaceutical manufacturing processes through the in-line or on-line measurement of critical process parameters (CPPs) that affect critical quality attributes (CQAs) (e.g., process analytical technology/(PAT)) (Metze, Bioprocess and Biosystems Engineering, 43(2):193-205, 2020). Off-line assays also introduce delays between sampling as well as responding to process changes and require more labor and resources.

It will be appreciated that a need exists for in-line and/or on-line methods and systems for measuring VCD to optimize cell culture therapeutic protein production and adhere to the regulatory framework outlined by the FDA for process analytical technology implementation.

Dielectric spectroscopy is a promising technique for real-time analysis of cell culture and biomass properties. Dielectric spectroscopy is a non-destructive, non-invasive continuous process monitoring tool that can generate continuous process data for both suspension and adherent cell cultures. Nevertheless, implementation of a biomass on-line monitoring technique remains challenging because of the associated complex calibration processes, integration difficulties and insufficient model accuracy and transferability (Carvell, Cytotechnology, 50:35-48, 2006, Kell et al., J. Bioelectricity, 4(2):317-348, 1990). Accordingly, the present application provides the benefit of dielectric spectroscopy systems and methods that can accurately determine on-line viable cell density predictions during cell culture.

In one embodiment, the present disclosure provides methods for culturing a cell by measuring a first property of the cell culture, using the first property to predict a second property of the cell culture, and adjusting a culturing condition based on the predicted second property of the cell culture.

In one embodiment, a capacitance probe can be a capacitance or dielectric sensor that produces alternating dielectric fields and measures the permittivity of a surrounding medium. A capacitance probe may produce alternating electric fields using at least one electrode. An electrode within a capacitance probe may comprise platinum, aluminum, gold, or other suitable metal.

In one aspect, an on-line sensor is used to measure a first property of a cell culture.

In one aspect, a correlation equation is used to predict a second property of the cell culture based on the measured first property of the cell culture.

In one aspect, the cell culture property can be a capacitance value, viable cell density, permittivity, and/or conductivity.

In one embodiment, the present disclosure provides methods for measuring VCD of cells cultured in a bioreactor by applying an electric field to cells cultured in a bioreactor, measuring the capacitance and correlating the measured capacitance to viable cell density.

In one embodiment, the present disclosure provides methods for culturing a cell by using an on-line sensor to measure a first property of a cell culture, using the measure of the first property of the cell culture and a correlation equation to predict at least one measure of a second property of the cell culture, and adjusting a culturing condition based on the at least one predicted measure of the second property of the cell culture to culture the cell.

In one aspect, an off-line assay is used to measure a second property of the first cell culture.

In one aspect, the cell is from a cell line that is the same as a cell line used to derive the correlation equation.

In one aspect, the cell is from a cell line that is different from a cell line used to derive the correlation equation.

In one aspect, the correlation equation is derived using more than one cell line.

In one aspect, more than one first capacitance value of the first cell culture is measured.

In one aspect, more than one first VCD value of the first cell culture is measured.

In one aspect, at least 50 percent of variability in the first VCD values is due to variance in the first capacitance values.

In one aspect, the correlation equation is produced using multivariate data analysis.

In an embodiment, a bioanalyzer measurement of a cell culture sample can be performed using a bioanalyzer system. A bioanalyzer system can comprise at least one 96-well plate, syringe, 24-position external sample tray, lab-on-a-chip or a combination thereof. A bioanalyzer system for performing a bioanalyzer measurement of a cell culture sample can be automated. A bioanalyzer system can analyze a single sample by performing between about 1 and about 20 assays simultaneously. Exemplary, non-limiting bioanalyzer measurements that may be performed by a bioanalyzer system include quantitating pH, the partial pressure of CO₂ (pCO₂), the partial pressure of O₂, glucose, lactate, glutamine, glutamate, ammonium, sodium, potassium, calcium, osmolality, total cell density, viable cell density, cell viability, or a combination thereof. A bioanalyzer system can determine viable cell density using the trypan blue exclusion test. A cell culture sample within a lab-on-a-chip can be analyzed using at least one electrophoretic separation, flow cytometry or a combination thereof. Electrophoretic separations can be used to perform DNA, RNA or protein assays, or a combination thereof. A DNA assay can determine the size or quantity of DNA, or the size and quantity of DNA within a cell culture sample. An RNA assay can quantitate total RNA, mRNA or RNA and mRNA within a sample, determine the size of at least one small RNA within a sample, determine the purity and integrity of a sample or a combination thereof. A protein assay can determine the size or quantity of at least one protein within a sample. Flow cytometry can be used to analyze protein expression, apoptosis, gene silencing transfection monitoring, intracellular staining, extracellular staining or a combination thereof.

In one aspect, cell culture nutrients can include, for example, glucose, glutamine, glutamate, sodium, potassium, calcium, O₂, or a combination thereof.

In one aspect, cell culture metabolites can include, for example, acetic acid, acetate, lactic acid, lactate, ammonia, ethanol, lactic acid, CO₂, or a combination thereof.

In one aspect, viable cell density refers to the number of live cells in a given volume of culture medium, as determined by measuring the permittivity of a surrounding medium, standard viability assays, including the trypan blue dye exclusion test, or a combination thereof. Cell density refers to the number of cells in a given volume of culture medium.

Example 23 is provided to describe to those of ordinary skill in the art how to use improved capacitance probes for measuring VCD, and is not intended to limit the scope of what the inventors regard as their invention.

### Vessel and Bioreactor Agitation, Sparging and Measurement Optimization

Optimizing the starting volume, air sparge, and agitation setpoints in vessels and bioreactors is important because during the growth and production phases, the cell culture is exposed to shear stress from agitator impellers and sparge gassing, which are required to maintain sufficient oxygen transport and stripping of dissolved carbon dioxide for maintained cell health. It has been found that these processes can be optimized by the use of stepped or varying agitation and air sparging rates. For example, it has been found that stepped or varying agitation and air sparging rates provide greater improvements in VCD. Dissolved oxygen and carbon dioxide can be controlled to a setpoint via sparging on a cascade control loop.

It has also been found that stepped or varying agitation and air sparging rates can reduce heterogeneity by lowering the amount of acidic and basic species of the recombinant protein of interest. These variants can arise from deamidation, sialylation, glycation and fragmentation, which can alter the stability, activity and potency of proteins that comprise Fc moieties (portions from the fragment crystallizable region of antibodies). Sissolak et al., J. Indust. Microbiol. Biotech. 46: 1167-78 (2019). Basic variants can cause increased binding of antibodies to Fc receptors. Hintersteiner et al., MABS 8: 1458-60 (2016).

Fc glycans can play a role in immunogenicity, bioactivity, pharmacodynamics and pharmacokinetics. Reusch and Tejada, Glycobiol. 25: 1325-34 (2015). A phenomenon that can occur is known as non-glycosylated heavy chain (NGHC). NGHC variation can alter effector functions, such as opsonization. Opsonization concerns the Fc portions that are involved in ADCC (antibody-dependent cellular cytotoxicity), ADCP (antibody-dependent cellular phagocytosis) and CDC (complement-dependent cytotoxicity). Thus, depending on the mode of action, there may be a need to control charge variation and NGHC in proteins that comprise Fc moieties.

In one exemplary embodiment, agitation of the culture is increased by 25%, 50%, 75%, 100%, 125%, 150%, 175% or 200% at day 0.5, 1, 1.5, 2, 2.5, 3, 3.5, 4, 4.5, 5, 5.5, 6, 6.5, 7, 7.5, 8, 8.5, 9, 9.5, 10, 10.5, or 11. In one aspect, the initial agitation rate is between 20-150 rpm and is increased to 40-300 rpm and then again to 80 to 600 rpm. The agitation rpm may also be correlated to power per unit volume using known models. For example, in a 10,000 L bioreactor with two impellers rotating at 40 rpm with 10,000 L, the power per unit volume is equal to 0.076 hp/1000 L; when rotating at 22 rpm with 7,500 L, the power per unit volume is equal to 0.017 hp/1000 L. In a 3,000 L bioreactor with two impellers rotating at 40 rpm with 3,000 L, the power per unit volume is equal to 0.033 hp/1000 L; when rotating at 40 rpm with 2,500 L, the power per unit volume is equal to 0.04 hp/1000 L. In a 10,000 L bioreactor with a single impeller rotating at 40 rpm with 10,000 L, the power per unit volume is equal to 0.005 hp/1000 L; when rotating at 22 rpm with 7,500 L, the power per unit volume is equal to 0.001 hp/1000 L.

In another aspect, the initial agitation rate is about 75 rpm and then increased to 150 rpm and then increased to 225 rpm. In one exemplary embodiment, air sparging rates of the culture are increased by 25%, 50%, 75%, 100%, 125%, 150%, 175%, 200%, 225%, 250%, 275%, 300%, 325%, 350%, 375%, 400%, 425%, 450%, 475%, or 500% at day 0.5, 1, 1.5, 2, 2.5, 3, 3.5, 4, 4.5, 5, 5.5, 6, 6.5, 7, 7.5, 8, 8.5, 9, 9.5, 10, 10.5, or 11. In one aspect, the initial sparging rate is between 50-75 slpm and is increased to about 225-275 slpm at day 2. In one aspect the spargers have between 146 and 292 holes that are sized between 0.5 mm and 2 mm and can include, for example, spargers with 146×0.5 mm holes, 146×1.0 mm holes, 146×1.5 mm holes, 146×2.0 mm holes, 292×0.5 mm holes, 292×1.0 mm holes, 292×1.5 mm holes, and 292×2.0 mm holes.

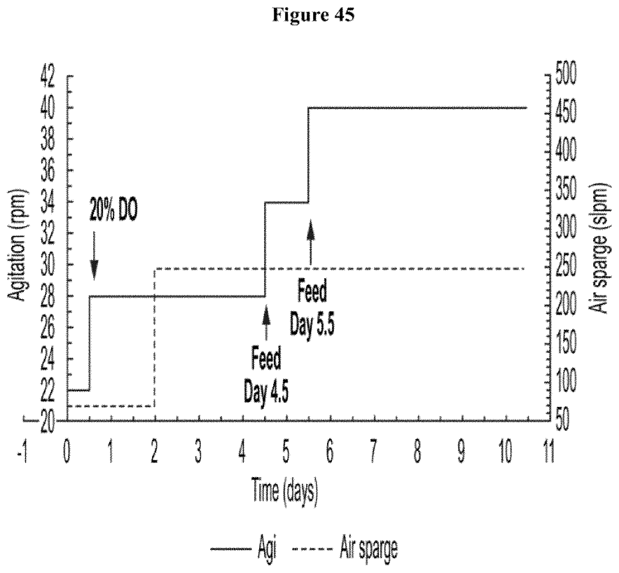

In one aspect the agitation rate and air sparging rates are increased on the same days. In another aspect the agitation rate and air sparging rates are increased on different days, where the agitation rate may be increased on feed days as shown in FIG. 45.

In one exemplary embodiment, the starting volume of the vessel or bioreactor may be optimized to reduce agitation-related surface effects. The initial working volume inside a vessel or bioreactor is typically set at a level with sufficient empty space to provide room for adjustments in volume, including subsequent feeds. It was unexpectedly found that increasing the initial working volume increased biomass and final titer. It was found that by increasing the working volume it reduced agitation-related surface effects caused by impellers in bioreactors, including air cascade effects, and provided better control over the concentration of dissolved oxygen.

In one aspect, the initial working volume of the vessel or bioreactor is adjusted to ensure the uppermost impeller (if more than one) is covered by the cell culture media at the start of the culturing process. Shown in Table 6 below are examples of different vessels and optimized volumes (kg) to reduce agitation-related surface effects.

TABLE 6

| | | Original Volume (kg) | Optimized Volume (kg) |
|---|---|---|---|
| Unit Operation | | (range) | (range) |
| 50 | L | 40-45 | 45-50 |
| 500 | L | 425-460 | 460-480 |
| 3,000 | L | 1,775-1,825 | 1,850-1,900 |
| 10,000 | L | 7,000-7,400 | 7,400-7,800 |

In one embodiment, the levels of dissolved oxygen are periodically measured. In one aspect, the periods for measuring the levels of dissolved oxygen can be selected by a user. In another aspect, the levels of dissolved oxygen can be automatically measured at set time periods. In one aspect, the sparging rate can be configured to automatically adjust the sparging rate based on the measured levels of dissolved oxygen to maintain a set level of dissolved oxygen, which can be constant, varied or stepped as described above using a cascade control.

As described above, dissolved oxygen and carbon dioxide concentrations are critical process parameters that must be carefully controlled, which also requires optimized systems and methods for measuring dissolved gasses. In one embodiment, improved probes for measuring dissolved gasses in large-scale vessels and bioreactors that are used for culturing cells in manufacturing therapeutic protein products in the biopharmaceutical industry are disclosed. Improved data processing methods were developed through comparison of data from two optical probes (termed Optical Probe A and Optical Probe B) alongside electrochemical probe data in a large-scale industrial bioreactor. As shown in Examples 19 and 21 below, methods of calibration were developed to account for offsets observed between both optical probes, with respect to the electrochemical probe (Optical Probe A: ˜+1.6% saturation, Optical Probe B: ˜−3.0% saturation), to achieve the goals of obtaining the necessary accuracy while minimizing maintenance and reducing or eliminating unwanted excursion events outside of normal operating ranges. Notably, the optical probes demonstrated 100% elimination of excursions above the normal operating range observed on the electrochemical probe, and an approximate twofold reduction in noise compared with the electrochemical probe (based on a standard deviation comparison).

In addition, as shown in Example 20, improved signal processing methods, including filtering and smoothing data, were developed for electrochemical probes to reduce excursion events determined to be false positives. In one aspect, the signal processing is applied in real-time.

In one embodiment, cells are cultured in a large-scale vessel or bioreactor where levels of dissolved gasses, including oxygen, are periodically measured. In one aspect, the periods for measuring the levels of dissolved oxygen can be selected by a user. In another aspect, the levels of dissolved oxygen can be automatically measured at set time periods.

In one embodiment, a vessel or bioreactor with reduced maintenance requirements is configured to use one or more optical probes to measure dissolved oxygen. Bioreactors and vessels may also be configured with one or more optical probes to measure other gasses such as carbon dioxide. In one aspect, the data from the optical probes is processed based on a fixed offset. In one aspect, the offset is based on a correlation of data from an optical probe and an electrochemical probe. In another aspect, the offset is based on a correlation with an optical probe and a known standard. In another aspect, the data from the optical probes is processed based on an offset that varies over time during the culturing process. In one aspect, the offset is applied in real-time.

In another embodiment, a vessel or bioreactor is configured to use one or more electrochemical probes with optimized data processing by filtering and smoothing the signals from the electrochemical probe. In one aspect, the sampling window for the data is smoothed between a 10-minute window and a 33-minute window.

It is believed that the same methods for improving accuracy and reducing signal noise used for optical probes and electrochemical probes for measuring dissolved oxygen may also be applied to measuring carbon dioxide and other dissolved gases.

In one embodiment, cells are cultured under proper pCO₂ conditions which can control the heterogeneity of antibodies, and derivatives and fragments of both recombinantly produced in mammalian cells, and is described more fully in U.S. Pat. Appln. No. 63/246,047 (Methods of Controlling Antibody Heterogeneity), incorporated by reference herein. The cells can be any suitable mammalian cell, including CHO, BHK, HEK293, HeLa, Human Amniotic, Per.C6 and Sp2/0 cells. Although not bound by any theory, it is believed that increasing CO₂ levels in media leads to increase in intracellular CO₂, which may be responsible for charge and glycosylation variation. This effect is separate from any decrease in pH possibly due to the formation of carbonic acid.

Carbon dioxide concentration can be increased using CO₂ sparging or by lowering air sparging. Reducing pressure in production bioreactors results in reduced solubility of oxygen; this in turn requires greater sparging of greater oxygen to maintain a dissolved oxygen (DO) set point and increased gas flow rate, which drives off pCO2 from the culture medium. Carbon dioxide concentration can be measured using a CO₂ electrode, also referred to as a Severinghaus electrode. More advanced systems are commercially available, such as the BioProfile® FLEX and FLEX 2 Analyzers. Charge variants can be measured using Imaged Capillary Isoelectric Focusing (iCIEF) and ion exchange chromatography with elution by salt gradient. NGHC can be measured by reduced capillary electrophoresis (CE)-SDS.

The cells can be cultured for about 10-15 days. In one embodiment, the cells can be cultured for about 14 days. The pCO₂ conditions can be between about 30 mmHg and about 210 mmHg, 50 mmHg to 200 mmHg, 60 mmHg to 190 mmHg, 70 mmHg to 180 mmHg, 80 mmHg to 170 mmHg, 90 mmHg to 160 mmHg, 100 mmHg to 150 mmHg, 110 mmHg to 140 mmHg, 120 mmHg to 140 mmHg, 120 mmHg to 130 mmHg, or any value within these ranges during the culturing, which is preferably maintained by CO2 sparging, and can be measured using a CO2 electrode.

The methods can comprise seeding media with mammalian cells that produce an Fc-containing protein, such as antibodies capable of binding IL-4 receptors; and culturing the cells under pCO2 conditions that allow the mammalian cells to produce Fc-containing proteins capable of binding IL-4 receptors. Preferably, the proteins are human monoclonal antibodies, preferably IgG antibodies, including subclasses such as IgG1 and IgG4.

In another aspect, the main peak form of an Fc-containing protein, such as antibodies capable of binding IL-4 receptors, produced by the cells may comprise between about 38% to about 65% of total Fc-containing protein; the acidic variant of the Fc-containing protein, such as antibodies capable of binding IL-4 receptors, may comprise between about 20% to about 47% of total Fc-containing protein; and the basic variant of the Fc-containing protein, such as antibodies capable of binding IL-4 receptors, may comprise up to about 36% of total Fc-containing proteins. The percentage of Fc-containing proteins with non-glycosylated heavy chains may comprise about 3 to about 8%, 4%-7%, 5%-7% and 5%-6.5%, 5%-6%, 5%-5.75%, 5%-5.5% or any whole or fractional value within these ranges.

In another aspect, the main peak form of Fc-containing protein, such as antibodies capable of binding IL-4 receptors, produced by the cells may comprise between about 50% to about 70%, the acidic variant of the Fc-containing proteins may comprise between about 20% to about 47%, and the basic variant of the Fc-containing proteins may comprise up to about 15% of the total Fc-containing protein, such as antibodies capable of binding IL-4 receptors, total antibodies, antibody derivatives or antibody fragments. The percentage of Fc-containing protein, such as antibodies capable of binding IL-4 receptors, with non-glycosylated heavy chains may comprise about 3 to about 8%, 4%-7%, 5%-7% and 5%-6.5%, 5%-6%, 5%-5.75%, 5%-5.5% or any whole or fractional value within these ranges.

In another aspect, the main peak form of Fc-containing protein, such as antibodies capable of binding IL-4 receptors, produced by the cells may comprise between about 50% to about 70%, the acidic variant of the Fc-containing protein, such as antibodies capable of binding IL-4 receptors, may comprise between about 20% to about 47%, and the basic variant of the Fc-containing protein, such as antibodies capable of binding IL-4 receptors, may comprise up to about 6%, about 8% or about 10% of the total Fc-containing protein, such as antibodies capable of binding IL-4 receptors, total antibodies, antibody derivatives or antibody fragments. The percentage of Fc-containing protein, such as antibodies capable of binding IL-4 receptors, with non-glycosylated heavy chains may comprise about 3 to about 8%, 4%-7%, 5%-7% and 5%-6.5%, 5%-6%, 5%-5.75%, 5%-5.5% or any whole or fractional value within these ranges.

In another aspect, the main peak form of Fc-containing protein, such as antibodies capable of binding IL-4 receptors, produced by the cells may comprise between about 50% to about 65%, the acidic variant of the Fc-containing protein, such as antibodies capable of binding IL-4 receptors, may comprise between about 23% to about 46%, the basic variant of the Fc-containing protein may comprise up to about 15% of the total Fc-containing protein, such as antibodies capable of binding IL-4 receptors, total antibodies, antibody derivatives or antibody fragments. The percentage of Fc-containing protein, such as antibodies capable of binding IL-4 receptors, with non-glycosylated heavy chains may comprise about 3 to about 8%, 4%-7%, 5%-7% and 5%-6.5%, 5%-6%, 5%-5.75%, 5%-5.5% or any whole or fractional value within these ranges.

In another aspect, the main peak form of Fc-containing protein, such as antibodies capable of binding IL-4 receptors, produced by the cells may comprise between about 50% to about 65%, the acidic variant of the Fc-containing protein, such as antibodies capable of binding IL-4 receptors, may comprise between about 31% to about 46%, and the basic variant of the Fc-containing protein may comprise up to about 15% of the total Fc-containing protein, such as antibodies capable of binding IL-4 receptors, total antibodies, antibody derivatives or antibody fragments. The percentage of Fc-containing protein, such as antibodies capable of binding IL-4 receptors, with non-glycosylated heavy chains may comprise about 3 to about 8%, 4%-7%, 5%-7% and 5%-6.5%, 5%-6%, 5%-5.75%, 5%-5.5% or any whole or fractional value within these ranges.

In another aspect, the main peak form of Fc-containing protein, such as antibodies capable of binding IL-4 receptors, produced by the cells may comprise between about 50% to about 65%, the acidic variant of the Fc-containing protein, such as antibodies capable of binding IL-4 receptors, may comprise between about 23% to about 39%, and the basic variant of the Fc-containing protein, such as antibodies capable of binding IL-4 receptors, may comprise up to about 15% of the total Fc-containing protein, such as antibodies capable of binding IL-4 receptors, total antibodies, antibody derivatives or antibody fragments. The percentage of Fc-containing protein, such as antibodies capable of binding IL-4 receptors, with non-glycosylated heavy chains may comprise about 3 to about 8%, 4%-7%, 5%-7% and 5%-6.5%, 5%-6%, 5%-5.75%, 5%-5.5% or any whole or fractional value within these ranges.

In another aspect, the main peak form of Fc-containing protein, such as antibodies capable of binding IL-4 receptors, produced by the cells may comprise between about 35% to about 60%, the acidic variant of the Fc-containing protein, such as antibodies capable of binding IL-4 receptors, may comprise between about 20% to about 40%, and the basic variant of the Fc-containing protein, such as antibodies capable of binding IL-4 receptors, may comprise between about 10% and about 40% of the total Fc-containing protein, such as antibodies capable of binding IL-4 receptors, total antibodies, antibody derivatives or antibody fragments. The percentage of Fc-containing protein, such as antibodies capable of binding IL-4 receptors, with non-glycosylated heavy chains may comprise about 3 to about 8%, 4%-7%, 5%-7% and 5%-6.5%, 5%-6%, 5%-5.75%, 5%-5.5% or any whole or fractional value within these ranges.

In another aspect, the main peak form of Fc-containing protein, such as antibodies capable of binding IL-4 receptors, produced by the cells may comprise between about 39% to about 50%, the acidic variant of the Fc-containing protein, such as antibodies capable of binding IL-4 receptors, may comprise between about 22% to about 38%, and the basic variant of the Fc-containing protein, such as antibodies capable of binding IL-4 receptors, may comprise between about 14% and about 36% of the total Fc-containing protein, such as antibodies capable of binding IL-4 receptors, total antibodies, antibody derivatives or antibody fragments. The percentage of Fc-containing protein, such as antibodies capable of binding IL-4 receptors, with non-glycosylated heavy chains may comprise about 3 to about 8%, 4%-7%, 5%-7% and 5%-6.5%, 5%-6%, 5%-5.75%, 5%-5.5% or any whole or fractional value within these ranges.

Additional details are further described by Example 18 below based on an Fc-containing protein, such as antibodies capable of binding IL-4 receptors, which are illustrative of the various aspects of the invention, but do not limit the inventions in any manner and may be applied to cells that are cultured under proper pCO₂ conditions for controlling acidic species and non-glycosylated heavy chains for Dupilumab.

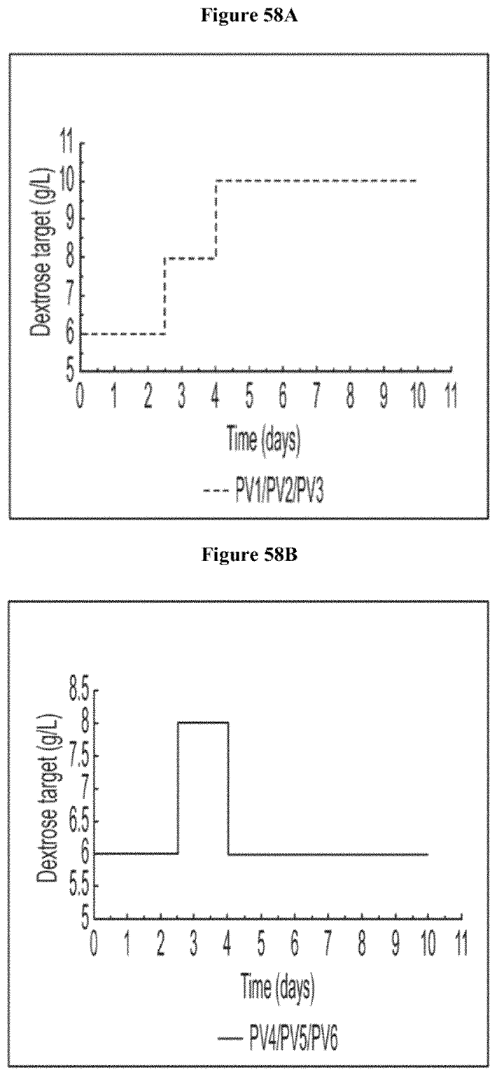

In one exemplary embodiment glucose feeding strategies are also optimized to prevent high osmolality conditions. It was found that glucose concentration influences osmotic conditions which can impact the cell culture performance. To improve cell culture condition and performance, the lower dextrose target was reduced to prevent high osmolality conditions. In one aspect, the dextrose target was reduced by 10%, 20%, 30%, 40%, 50%, 60%, or 70%. In one aspect, the dextrose target was stepped up on day 2, 3, 4, 5, or 6. In one aspect, the dextrose target was stepped up and then reduced after 0.5, 1, 1.5, 2, 2.5, or 3 days. In one exemplary embodiment, the initial dextrose target is between 5 g/L and 7 g/L, and the stepped-up dextrose target is between 7 g/L and 11 g/L. In one embodiment, the dextrose target is stepped up and stepped down one or more times. Exemplary dextrose targets and adjustments over time are shown in FIG. 58A and FIG. 58B.

In another aspect, High Temperature Short Time (HTST) treatment at 102° C. for 10 seconds of seed train and production medium and feeds can be performed. In another aspect, HTST treatment is performed at temperature of about 101° C., 102° C., 103° C., 104° C., 105° C., or about 106° C. for period of 8 seconds, 9 seconds, 11 seconds, 12 seconds, 13 seconds, 14 seconds, or 15 seconds. HTST can function as a flash pasteurization of cell culture media and feeds that may be used in manufacturing as a viral barrier for upstream processing and to mitigate adventitious agent risk, resulting in equivalent process performance and product quality, including titer.

### Centrifugation, Depth and Polish Filtration

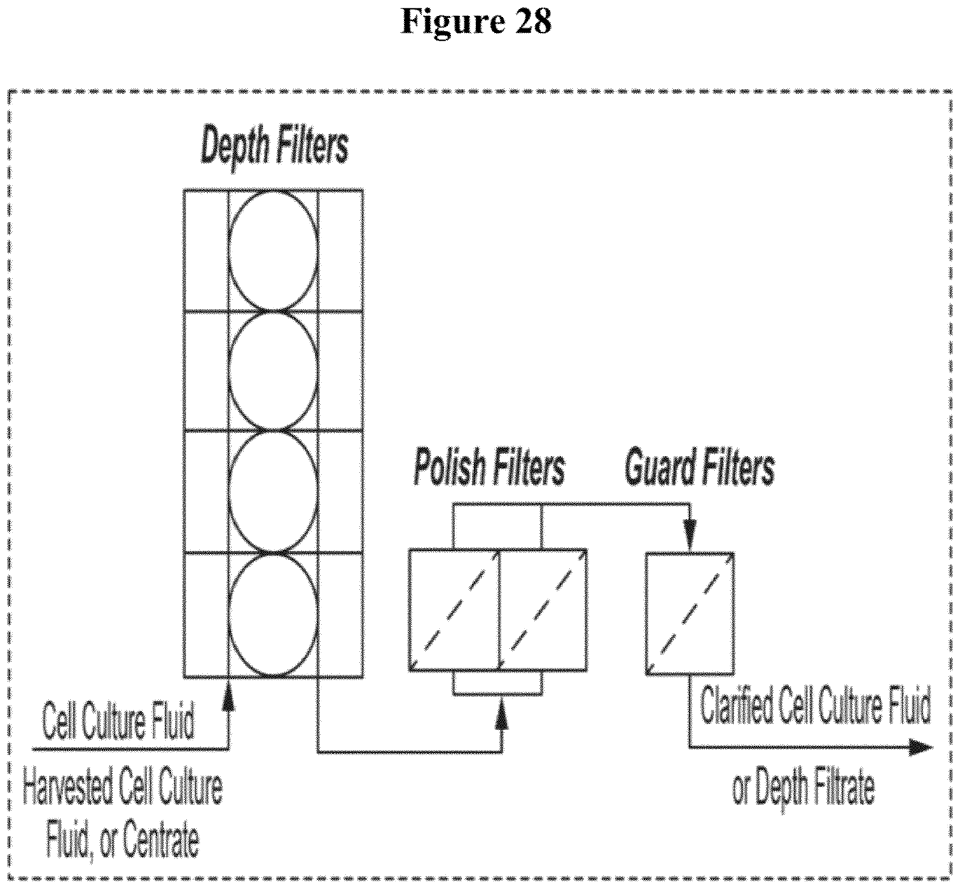

In certain exemplary embodiments, the primary recovery may include one or more centrifugation steps, including disc-stack centrifugation and depth, polish, and guard filtration or direct depth and polish filtrations without centrifugation, to separate the protein of interest from a host cell and attendant cellular debris. Shown in FIG. 28 is a schematic of an exemplary outline regarding the use of depth filters, polish filter and guard filters.

Centrifugation of the sample can be performed at, for example, but not by way of limitation, 7,000×g to approximately 12,750×g. A 60-80% solids fill of the bowl is recommended for calculation of the discharge interval. The centrate is then processed by in-line depth, polish, and guard filtration. As a result, the filter flux is a function of the centrifuge feed flow rate and the areas of the depth, polish, and guard filters. In some embodiments, a bowl speed of 5550 rpm (7000 g) and feed flow rate of about 1630 (L/h) may be used. The filter flux and discharge interval can be calculated as shown in Table 7 below:

TABLE 7

| Parameter | Specification | |
|---|---|---|
| Bowl Speed (rpm) | 5550 | (7000 g) |
| Feed Flow Rate (L/h) | 1630 | |
| Q/Σ(m/s) | 1.06 × 10⁸ | (Q/cΣ = 2.6 × 10⁻⁸ m/s) |

| | |
|---|---|
| Discharge Interval (seconds) | 145 |

g, standard gravity

It has been found that the bowl speed may impact the turbidity by changing the settling rate for particles of various sizes. In the context of large-scale production, such centrifugation can occur on-line with a flow rate set to achieve, for example, a turbidity level of 150 NTU in the resulting supernatant.

The cell culture fluid or centrate may need to be adjusted to a particular pH and conductivity in order to obtain desired impurity removal and product recovery from the depth filtration step.

To reduce lot-to-lot variability, centrate turbidity should be measured at the midpoint between discharges, known as the bowl midpoint (t=½ discharge interval) to determine whether adjustments to bowl speed and feed flow rate need to be made. It has been found that a faster flow rate results in a shorter residence time, which may increase turbidity and lower turbidity clearance over the filters. It was found that centrifugal performance cannot be predicted by use of linear extrapolation using sigma concept alone due to alterations in level of centrifuge-induced shear and therefore particle size distribution with alterations in bowl speed.

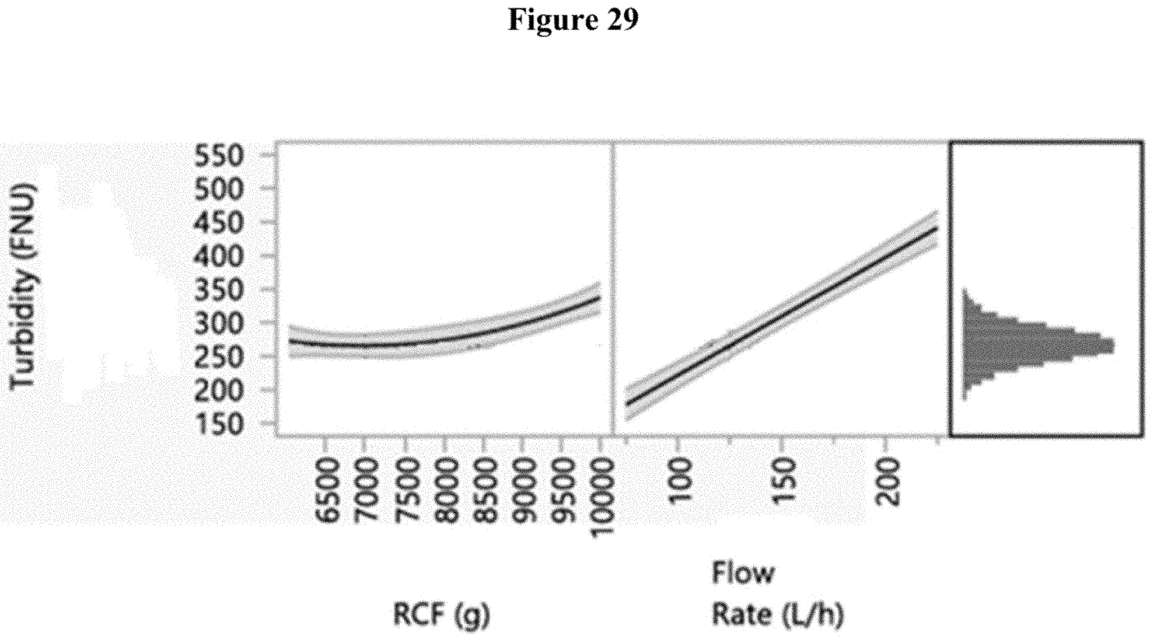

To overcome these limitations, a prediction profiler was generated in order to visualize the factors and response as reflected in FIG. 29.

The resultant turbidity model is significant (p<0.0001) with an R2adj of 0.95 and an RMSE of 25.0 FNU. The main effect and quadratic of relative centrifugal force, the main effect of flow rate, and the interaction of relative centrifugal force and flow rate were identified as significant factors. Flow rate had the largest effect on centrate turbidity. In general, with an RCF of less than 8000 g, the centrate turbidity was only a function of flow rate.

Such centrate can then be collected for further processing or in-line filtered through one or more depth filters for further clarification of the sample. The most frequently used depth filters for bioprocessing consist of diatomaceous earth, cellulose fibers, and a positively charged resin binder. Depth filters, unlike membrane filters, retain particles throughout the porous filter media, allowing for retention of particles both larger and smaller than the pore size. Particle retention is believed to involve both size exclusion and adsorption through ionic, hydrophobic, and other mechanisms. The filter fouling mechanism may include pore blockage, cake formation, and pore constriction. Non-limiting examples of depth filters that can be used in the context of the instant disclosure include the Millistak+X0HC, F0HC, D0HC, C0HC, A1HC, B1HC depth filters (EMD Millipore), 3M™ model 30/60ZA, 60/90 ZA, VR05, VR07, delipid depth filters (3M Corp.). A 0.2 μm filter such as Sartorius's 0.45/0.2 μm Sartopore™ bi-layer or Millipore's Express SHR or SHC filter cartridges typically follows the depth filters. The Emphaze AEX Hybrid Purifier multi-mechanism filter may also be used to filter the depth filtered material. Other filters well known to the skilled artisan can also be used.

After depth filtration, polish filtration is used to ensure final solids removal prior to column chromatography. Polish filtration can include specially designed filters that increase impurity absorption through ionic mechanisms and therefore reduce the overall impurity level applied to the primary column chromatography step. Guard filtration after polish filtration is used for bioburden control only.

To accommodate high titer and increased protein production, pore size of filters for these polish and guard filters may range from 0.15 μm-0.25 μm, and in one aspect may be 0.2 μm. For an average 10,000 L bioreactor culture, polish filter loading can range from 255 L/m² to 270 L/m², and in one aspect about 265 to 270 L/m² (e.g., 33.6 m² per 10,000 L batch). Guard filter loading can range from 700 L/m² to 720 L/m².

### Protein A Chromatography

In certain exemplary embodiments, it may be advantageous to subject a biological sample to affinity chromatography for production of a protein of interest. The chromatographic material is capable of selectively or specifically binding to or interacting with the protein of interest. Non-limiting examples of such chromatographic material include: Protein A and Protein G. Also included is chromatographic material comprising, for example, a protein or portion thereof capable of binding to or interacting with the protein of interest.

Affinity chromatography can involve subjecting a biological sample to a column comprising a suitable Protein A resin. When used herein, the term “Protein A” encompasses Protein A recovered from a native source thereof, Protein A produced synthetically (e.g., by peptide synthesis or by recombinant techniques), and variants thereof which retain the ability to bind proteins which have a C_{H}2/C_{H}3 region. Protein A may additionally bind to human IgG molecules containing IgG F(ab′)2 fragments from the human VH3 gene family (Roben et al., J Immunol. 1995 154(12):6437-45). In certain aspects, Protein A resin is useful for affinity-based production and isolation of a variety of antibody isotypes by interacting specifically with the Fc portion of a molecule should it possess that region.

There are several commercial sources for Protein A resin. Suitable resins include, but are not limited to, MabSelect PrismA, MabSelect SuRe, MabSelect SuRe LX, MabSelect, Mab Select SuRe pcc, Mab Select Xtra, rProtein A Sepharose from Cytiva, ProSep HC, ProSep Ultra, ProSep Ultra Plus from EMD Millipore, MabCapture from ThermoFisher, and Amsphere™ A3 from JSR Life Sciences.

An affinity column can be equilibrated with a suitable buffer prior to sample loading. A pH of a Protein A load may be, for example, between about 6 and about 8, between about 6 and about 7, between about 7 and about 8, or about 6. Following loading of the column, the column can be washed one or multiple times using a suitable wash buffer. The column can then be eluted using an appropriate elution buffer, for example, glycine-HCl, acetic acid, or citric acid. The eluate can be monitored using techniques well known to those skilled in the art such as a UV detector. The eluted fractions of interest can be collected and then prepared for further processing.

A Protein A wash buffer may be selected on the basis of its ability to disrupt protein-protein interactions, for example interactions between a protein of interest and impurities such as HCPs, without disrupting interactions between the protein of interest and the chromatographic material. Suitable wash buffers for HCP removal may comprise, for example, salts (e.g. sodium-containing salts such as sodium phosphate, potassium-containing salts such as potassium sorbate, magnesium-containing salts, hydrochloride-containing salts such as guanidine hydrochloride, Tris-containing salts), surfactants (e.g. polysorbate 20, polysorbate 80), polar materials (e.g. isopropanol, ethanol), or amino acids (e.g. arginine). Suitable wash buffers for HCP removal may have a pH between about 5 and about 9, about 5, about 5.5, about 6, about 6.5, about 7, about 7.5, about 8, about 8.5, or about 9.

A suitable wash buffer may be selected on the basis of removing a particular HCP of concern for a manufacturing process, for example by selecting a pH of the wash buffer corresponding to a pI of an HCP of concern. A wash buffer may be selected on the basis of, for example, disrupting electrostatic interactions of an HCP of concern and a protein of interest, hydrophobic interactions of an HCP of concern and a protein of interest, or both. The specific nature of an HCP of concern can be used to inform the selection of buffer components as shown, for example, in Table 8. An HCP of concern may be identified using HCP profiling, for example by using mass spectrometry to identify and quantify HCPs that associate with a protein of interest. A suitable wash buffer may be selected on the basis of ensuring an acceptable yield of a protein of interest by disrupting binding of a protein of interest to a chromatographic resin as little as possible.

TABLE 8
Exemplary buffer components and pH to disrupt protein-
protein interactions during affinity capture

| pH | Components | Rationale |
|---|---|---|
| <8.0 | 100-450 mM Arginine, 20 mM | Molecular structure of arginine can mimic HCP, many |
| | Tris, pH 6.0 | HCPs have an isoelectric point around 6 |
| | 100 mM-1.2M Potassium Sorbate, | Reported to reduce HCP as a wash component in the |
| | pH 7.2 | literature |
| | 0.5-1.0M Sodium Benzoate, pH | Reported to reduce HCP as a wash component in the |
| | 6.0 | literature |
| 8.0 | 100-450 mM Arginine, 30 mM | Molecular structure of arginine can mimic HCP, many |
| | Tris, pH 8.0 | HCPs have an isoelectric point around 8 |
| | 0.5-1.0M Guanidine, 0.05-0.5M | Guanidine is a kosmotrope and should weaken |
| | NaCl, pH 8.0 | hydrophobic interactions |
| >8.0 | 25 mM Tris, 1%-20% | Urea at high concentrations can denature proteins. In |
| | Isopropanol, 0.5-0.6M Urea, pH | combination with a polar solvent may generate |
| | 9.0 | combinatorial effects |
| | 10-500 mM Sodium Carbonate, | A more alkaline condition may denature HCP and decrease |
| | pH 10.0 | interaction |

An affinity wash buffer may further be selected on the basis of its ability to remove HMW species of a protein of interest. A suitable wash buffer may be selected that results in a substantial decrease in HMW species while preserving a yield of a protein of interest at acceptable levels.

Reduction of HCP levels and/or HMW species using an affinity wash buffer may make it possible to achieve appropriate drug substance quality with fewer downstream chromatographic steps, for example, using a Protein A column and an AEX column without a CEX column or a HIC column; or using a Protein A column followed by a mixed-mode column and an AEX column, without a CEX column or a HIC column. If the impurity levels achievable with additional columns are not matched through affinity capture wash buffer optimization alone, additional contributions can be had by using a charged filter media during a filtration step. This is typically done following a viral inactivation step, during which several pH adjustments are already being made and targeting a specific condition for filtration is easily integrated into the downstream process. Filter media with multiple mechanisms of removal are commercially available, including cation, anion, hydrophobic, and mixed mode mechanisms. Operating filters under conditions where a product of interest does not bind increases the likelihood of impurity removal and a high recovery, as the charge density on filters is typically lower when compared to packed bed chromatography.

### Low pH Hold

In certain exemplary embodiments, the primary recovery process can also be a point to reduce or inactivate viruses that can be present in a biological sample. Any one or more of a variety of methods of viral reduction/inactivation can be used during the primary recovery phase of production including heat inactivation (pasteurization), pH inactivation, buffer/detergent treatment, UV and γ-ray irradiation and the addition of certain chemical inactivating agents such as β-propiolactone or, for example, copper phenanthroline as described in U.S. Pat. No. 4,534,972, the entire teaching of which is incorporated herein by reference. In another aspect, virus-retentive filtration as a dedicated viral clearance step may also be used and is discussed below.

In those exemplary embodiments where viral reduction/inactivation is employed, in one aspect a biological sample can be adjusted, as needed, for further production steps. For example, following low pH viral inactivation, the pH of the sample may be adjusted to a more neutral pH, for example, from about 4.5 to about 8.5, prior to continuing the production process. In another aspect, the mixture may also be diluted with water for injection (WFI) to obtain a desired conductivity.

In another aspect, the eluate may be subjected to viral inactivation, for example, either by detergent or low pH. A suitable detergent concentration or pH (and time) can be selected to obtain a desired viral inactivation result.

In another aspect, after viral inactivation, the eluate may be pH and/or conductivity adjusted for subsequent production steps, including anion exchange chromatography, cationic exchange chromatography, mixed-mode chromatography, hydrophobic interaction chromatography, affinity chromatography (e.g., Protein A), and size-exclusion chromatography, among others.

### Anion Exchange Chromatography

In certain exemplary embodiments, a protein of interest is produced by subjecting a biological sample to at least one anion exchange (AEX) separation step. In one scenario, the anion exchange step can occur following an affinity chromatography procedure (e.g., Protein A affinity). In other scenarios, the anion exchange step can occur before the affinity chromatography step. In some embodiments, the AEX separation is the second of four chromatography unit operations and is performed downstream of affinity capture and viral inactivation by low pH hold operations. In yet other embodiments, the AEX separation is preceded by an ion exchange step. Alternatively, the AEX separation can be followed by another ion exchange procedure.

Anion exchange packed bed chromatography is based on ionic interactions between the binding entity (target protein or impurity) and the functional group immobilized on the chromatographic media. Performance may be a function of the mobile phase, the functional group, and the resin backbone. In some embodiments, a particular objective of an AEX step is to reduce the levels of DNA, HCPs, HMW species, and, if present, virus.

The use of an anionic exchange material versus a cationic exchange material is based, in part, on the local charges of the protein of interest. Anion exchange chromatography can be used in combination with other chromatographic procedures such as affinity chromatography, size exclusion chromatography, hydrophobic interaction chromatography, as well as other modes of chromatography known to the skilled artisan.

In performing a separation, the initial protein composition (biological sample) can be placed in contact with an anion exchange material by using any of a variety of techniques, for example, using a batch production technique or a chromatographic technique.

In the context of batch production, anion exchange material is prepared in, or equilibrated to, a desired starting buffer. Upon preparation, a slurry of the anion exchange material may be obtained. The biological sample may be contacted with the slurry to allow for protein adsorption to the anion exchange material. A solution comprising acidic species that do not bind to the AEX material may be separated from the slurry by allowing the slurry to settle and removing the supernatant. The slurry can be subjected to one or more washing steps and/or elution steps.

In the context of chromatographic separation, a packed bed chromatographic column is used to house chromatographic support material (resin or solid phase). A sample comprising a protein of interest is loaded onto a particular chromatographic column. The column can then be subjected to one or more wash steps using a suitable wash buffer. Components of a sample that have not adsorbed onto the resin will likely flow through the column. Components that have adsorbed to the resin can be differentially eluted using an appropriate elution buffer.

In some embodiments, a quantity of protein loaded on an AEX resin (e.g., gram protein per liter resin) is between about 50 g/L and about 200 g/L, between about 100 g/L and about 150 g/L, less than about 120 g/L, about 50 g/L, about 55 g/L, about 60 g/L, about 65 g/L, about 70 g/L, about 75 g/L, about 80 g/L, about 85 g/L, about 90 g/L, about 95 g/L, about 100 g/L, about 105 g/L, about 110 g/L, about 115 g/L, about 120 g/L, about 125 g/L, about 130 g/L, about 135 g/L, about 140 g/L, about 145 g/L, about 150 g/L, about 155 g/L, about 160 g/L, about 165 g/L, about 170 g/L, about 175 g/L, about 180 g/L, about 185 g/L, about 190 g/L, about 195 g/L, or about 200 g/L.

In some embodiments, a sample may be neutralized before contact with an AEX material. A sample, for example a viral inactivated pool, may be neutralized to a pH between about 7.40 and about 8.30, between about 7.50 and about 7.70, between about 7.55 and about 7.65, about 7.40, about 7.45, about 7.50, about 7.51, about 7.52, about 7.53, about 7.54, about 7.55, about 7.56, about 7.57, about 7.58, about 7.59, about 7.60, about 7.61, about 7.62, about 7.63, about 7.64, about 7.65, about 7.66, about 7.67, about 7.68, about 7.69, about 7.70, about 7.75, about 7.80, about 7.85, about 7.90, about 7.95, about 8.00, about 8.05, about 8.10, about 8.15, about 8.20, about 8.25, or about 8.30.

In some embodiments, a sample may be further adjusted after neutralizing and before contacting an AEX material. A sample, for example a viral inactivated pool, may be adjusted to between about 3.00 mS/cm and about 6.00 mS/cm, between about 3.00 mS/cm and about 4.00 mS/cm, between about 3.40 mS/cm and about 3.60 mS/cm, about 3.00 mS/cm, about 3.10 mS/cm, about 3.20 mS/cm, about 3.30 mS/cm, about 3.40 mS/cm, about 3.50 mS/cm, about 3.60 mS/cm, about 3.70 mS/cm, about 3.80 mS/cm, about 3.90 mS/cm, about 4.00 mS/cm, about 4.10 mS/cm, about 4.20 mS/cm, about 4.30 mS/cm, about 4.40 mS/cm, about 4.50 mS/cm, about 4.60 mS/cm, about 4.70 mS/cm, about 4.80 mS/cm, about 4.90 mS/cm, about 5.00 mS/cm, about 5.10 mS/cm, about 5.20 mS/cm, about 5.30 mS/cm, about 5.40 mS/cm, about 5.50 mS/cm, about 5.60 mS/cm, about 5.70 mS/cm, about 5.80 mS/cm, about 5.90 mS/cm, or about 6.00 mS/cm. A sample may be adjusted using, for example, about 2 M sodium acetate, about 2 M Tris base, or a combination thereof.

In some embodiments, an AEX column is subjected to a pre-equilibration step prior to contact with a sample. A pre-equilibration step replaces the strongly-bound hydroxide ions on the AEX column and facilitates faster equilibration. In some exemplary embodiments, a pre-equilibration buffer (or “mobile phase”) comprises about 2 M sodium chloride, WFI, or a combination thereof. In some exemplary embodiments, a linear velocity of a pre-equilibration buffer is between about 100 and about 300 cm/hr, between about 150 and about 250 cm/hr, about 100 cm/hr, about 110 cm/hr, about 120 cm/hr, about 130 cm/hr, about 140 cm/hr, about 150 cm/hr, about 160 cm/hr, about 170 cm/hr, about 180 cm/hr, about 190 cm/hr, about 200 cm/hr, about 210 cm/hr, about 220 cm/hr, about 230 cm/hr, about 240 cm/hr, about 250 cm/hr, about 260 cm/hr, about 270 cm/hr, about 280 cm/hr, about 290 cm/hr, or about 300 cm/hr. In some exemplary embodiments, about two column volumes of pre-equilibration buffer are used.

In some embodiments, an AEX column is subjected to an equilibration step prior to contact with a sample, and optionally following a pre-equilibration step. An equilibration step changes the mobile phase to a pH and conductivity that matches the buffer excipients present in the load material (sample). In some embodiments, an equilibration buffer may comprise about 50 mM Tris and about 60 mM acetate, at a pH between about 7.50 and about 7.70, and a conductivity between about 3.00 mS/cm and about 4.00 mS/cm. In some embodiments, a pH of an equilibration buffer is between about 7.40 and about 8.30, between about 7.50 and about 7.70, between about 7.55 and about 7.65, about 7.40, about 7.45, about 7.50, about 7.51, about 7.52, about 7.53, about 7.54, about 7.55, about 7.56, about 7.57, about 7.58, about 7.59, about 7.60, about 7.61, about 7.62, about 7.63, about 7.64, about 7.65, about 7.66, about 7.67, about 7.68, about 7.69, about 7.70, about 7.75, about 7.80, about 7.85, about 7.90, about 7.95, about 8.00, about 8.05, about 8.10, about 8.15, about 8.20, about 8.25, or about 8.30. In some embodiments, a conductivity of an equilibration buffer is between about 3.00 mS/cm and about 6.00 mS/cm, between about 3.00 mS/cm and about 4.00 mS/cm, between about 3.40 mS/cm and about 3.60 mS/cm, about 3.00 mS/cm, about 3.10 mS/cm, about 3.20 mS/cm, about 3.30 mS/cm, about 3.40 mS/cm, about 3.50 mS/cm, about 3.60 mS/cm, about 3.70 mS/cm, about 3.80 mS/cm, about 3.90 mS/cm, about 4.00 mS/cm, about 4.10 mS/cm, about 4.20 mS/cm, about 4.30 mS/cm, about 4.40 mS/cm, about 4.50 mS/cm, about 4.60 mS/cm, about 4.70 mS/cm, about 4.80 mS/cm, about 4.90 mS/cm, about 5.00 mS/cm, about 5.10 mS/cm, about 5.20 mS/cm, about 5.30 mS/cm, about 5.40 mS/cm, about 5.50 mS/cm, about 5.60 mS/cm, about 5.70 mS/cm, about 5.80 mS/cm, about 5.90 mS/cm, or about 6.00 mS/cm. In some exemplary embodiments, a linear velocity of an equilibration buffer is between about 100 and about 300 cm/hr, between about 150 and about 250 cm/hr, about 100 cm/hr, about 110 cm/hr, about 120 cm/hr, about 130 cm/hr, about 140 cm/hr, about 150 cm/hr, about 160 cm/hr, about 170 cm/hr, about 180 cm/hr, about 190 cm/hr, about 200 cm/hr, about 210 cm/hr, about 220 cm/hr, about 230 cm/hr, about 240 cm/hr, about 250 cm/hr, about 260 cm/hr, about 270 cm/hr, about 280 cm/hr, about 290 cm/hr, or about 300 cm/hr. In some exemplary embodiments, about three column volumes of an equilibration buffer are used.

In some embodiments, a concentration of protein loaded onto an AEX column (an “AEX load”, or grams protein per liter solution) is between about 10.0 g/L and about 30.0 g/L, between about 12 g/L and about 25 g/L, about 10.0 g/L, about 11.0 g/L, about 12.0 g/L, about 13.0 g/L, about 14.0 g/L, about 15.0 g/L, about 16.0 g/L, about 17.0 g/L, about 18.0 g/L, about 19.0 g/L, about 20.0 g/L, about 21.0 g/L, about 22.0 g/L, about 23.0 g/L, about 24.0 g/L, about 25.0 g/L, about 26.0 g/L, about 27.0 g/L, about 28.0 g/L, about 29.0 g/L, or about 30.0 g/L. In some exemplary embodiments, a linear velocity of the AEX load is between about 100 and about 300 cm/hr, between about 150 and about 250 cm/hr, about 100 cm/hr, about 110 cm/hr, about 120 cm/hr, about 130 cm/hr, about 140 cm/hr, about 150 cm/hr, about 160 cm/hr, about 170 cm/hr, about 180 cm/hr, about 190 cm/hr, about 200 cm/hr, about 210 cm/hr, about 220 cm/hr, about 230 cm/hr, about 240 cm/hr, about 250 cm/hr, about 260 cm/hr, about 270 cm/hr, about 280 cm/hr, about 290 cm/hr, or about 300 cm/hr.

In some embodiments, flowthrough is collected when UV absorbance at 280 nm reaches 0.2 AU on a 5 mm flow path, approximately 1.2 column volumes into the load step. This is followed by a wash step to increase yield.

A wash step is typically performed in AEX chromatography using conditions similar to the load conditions or alternatively by decreasing the pH and/or increasing the ionic strength/conductivity of the wash in a step wise or linear gradient manner. In one aspect, the aqueous salt solution used in both the loading and wash buffer has a pH that is at or near the isoelectric point (pI) of the protein of interest. Typically, the pH is about 0 to 2 units higher or lower than the pI of the protein of interest, however it may be in the range of 0 to 0.5 units higher or lower. It may also be at the pI of the protein of interest.

In some embodiments, a wash buffer comprises about 50 mM Tris and about 60 mM acetate, at a pH between about 7.50 and about 7.70, and a conductivity between about 3.00 mS/cm and about 4.00 mS/cm. In some embodiments, a pH of a wash buffer is between about 7.40 and about 8.30, between about 7.50 and about 7.70, between about 7.55 and about 7.65, about 7.40, about 7.45, about 7.50, about 7.51, about 7.52, about 7.53, about 7.54, about 7.55, about 7.56, about 7.57, about 7.58, about 7.59, about 7.60, about 7.61, about 7.62, about 7.63, about 7.64, about 7.65, about 7.66, about 7.67, about 7.68, about 7.69, about 7.70, about 7.75, about 7.80, about 7.85, about 7.90, about 7.95, about 8.00, about 8.05, about 8.10, about 8.15, about 8.20, about 8.25, or about 8.30. In some embodiments, a conductivity of a wash buffer is between about 3.00 mS/cm and about 6.00 mS/cm, between about 3.00 mS/cm and about 4.00 mS/cm, between about 3.40 mS/cm and about 3.60 mS/cm, about 3.00 mS/cm, about 3.10 mS/cm, about 3.20 mS/cm, about 3.30 mS/cm, about 3.40 mS/cm, about 3.50 mS/cm, about 3.60 mS/cm, about 3.70 mS/cm, about 3.80 mS/cm, about 3.90 mS/cm, about 4.00 mS/cm, about 4.10 mS/cm, about 4.20 mS/cm, about 4.30 mS/cm, about 4.40 mS/cm, about 4.50 mS/cm, about 4.60 mS/cm, about 4.70 mS/cm, about 4.80 mS/cm, about 4.90 mS/cm, about 5.00 mS/cm, about 5.10 mS/cm, about 5.20 mS/cm, about 5.30 mS/cm, about 5.40 mS/cm, about 5.50 mS/cm, about 5.60 mS/cm, about 5.70 mS/cm, about 5.80 mS/cm, about 5.90 mS/cm, or about 6.00 mS/cm. In some embodiments, a linear velocity of a wash buffer is between about 100 and about 300 cm/hr, between about 150 and about 250 cm/hr, about 100 cm/hr, about 110 cm/hr, about 120 cm/hr, about 130 cm/hr, about 140 cm/hr, about 150 cm/hr, about 160 cm/hr, about 170 cm/hr, about 180 cm/hr, about 190 cm/hr, about 200 cm/hr, about 210 cm/hr, about 220 cm/hr, about 230 cm/hr, about 240 cm/hr, about 250 cm/hr, about 260 cm/hr, about 270 cm/hr, about 280 cm/hr, about 290 cm/hr, or about 300 cm/hr. In some embodiments, a wash length is between about 3 and about 5 column volumes (CVs), about 3 CVs, about 3.5 CVs, about 4 CVs, about 4.5 CVs, or about 5 CVs.

In some embodiments, an AEX column may be regenerated using one or more strip buffers. In some embodiments, a first strip buffer comprises 2 M sodium chloride (NaCl) and a second strip buffer comprises 1 N sodium hydroxide (NaOH). In some embodiments, an AEX column may further be soaked in 1 N NaOH for at least about 30 minutes.

In some embodiments, an AEX resin lifetime is between about 1 and about 100 cycles, between about 50 and about 90 cycles, less than about 100 cycles, about 10 cycles, about 20 cycles, about 40 cycles, about 50 cycles, about 60 cycles, about 70 cycles, about 80 cycles, about 90 cycles, or about 100 cycles. In some embodiments, an AEX resin lifetime may reach 200 cycles.

An anionic agent may be selected from the group consisting of acetate, chloride, formate and combinations thereof. A cationic agent may be selected from the group consisting of Tris, arginine, sodium and combinations thereof. A buffer may be selected from the group consisting of pyridine, piperazine, L-histidine, Bis-Tris, Bis-Tris propane, imidazole, N-ethylmorpholine, TEA (triethanolamine), Tris, morpholine, N-methyldiethanolamine, AMPD (2-amino-2-methyl-1,3-propanediol), diethanolamine, ethanolamine, AMP (2-amino-2-methyl-1-propaol), 1,3-diaminopropane and piperidine.

A packed anion-exchange chromatography column, anion-exchange membrane device, anion-exchange monolithic device, or depth filter media can be operated either in bind-elute mode, flowthrough mode, or a hybrid mode wherein proteins exhibit binding to the chromatographic material and yet can be washed from such material using a buffer that is the same or substantially similar to the loading buffer.

In the bind-elute mode, a column or membrane device is first conditioned with a buffer with appropriate ionic strength and pH under conditions where certain proteins will adsorb to the resin-based matrix. For example, during the feed load, a protein of interest can be adsorbed to the resin due to electrostatic attraction. After washing the column or the membrane device with the equilibration buffer or another buffer with a different pH and/or conductivity, the product recovery is achieved by increasing the ionic strength (e.g., conductivity) of the elution buffer to compete with the solute for the charged sites of the anion exchange matrix. Changing the pH and thereby altering the charge of the solute is another way to achieve elution of the solute. The change in conductivity or pH may be gradual (gradient elution) or stepwise (step elution).

In the flowthrough mode, a column or membrane device is operated at a selected pH and conductivity such that the protein of interest does not bind to the resin or the membrane while the acidic species will either be retained on the column or will have a distinct elution profile as compared to the protein of interest. In the context of this strategy, acidic species will interact with or bind to the chromatographic material under suitable conditions while the protein of interest and certain aggregates and/or fragments of the protein of interest will flow through the column.

In some embodiments, an AEX step is performed in negative mode (flowthrough mode), where negatively charged process related impurities are adsorbed to the immobilized, positively charged ligand, and the protein of interest flows through.

Non-limiting examples of anionic exchange resins include diethylaminoethyl (DEAE), quaternary aminoethyl (QAE) and quaternary amine (Q) groups. Additional non-limiting examples include: Poros 50PI and Poros 50HQ, which are a rigid polymeric bead with a backbone consisting of cross-linked poly[styrene-divinylbenzene]; Poros 50XQ; Capto Q Impres and Capto DEAE, which are a high flow agarose bead; Capto Adhere; Q Sepharose Fast Flow; Toyopearl QAE-550, Toyopearl DEAE-650, and Toyopearl GigaCap Q-650, which are a polymeric base bead; Fractogel® EMD TMAE Hicap, which is a synthetic polymeric resin with a tentacle ion exchanger; Sartobind STIC® PA nano, which is a salt-tolerant chromatographic membrane with a primary amine ligand; Sartobind Q nano, which is a strong anion exchange chromatographic membrane; CUNO BioCap, which is a zeta-plus depth filter media constructed from inorganic filter aids, refined cellulose, and an ion exchange resin; XOHC, which is a depth-filter media constructed from inorganic filter aid, cellulose, and mixed cellulose esters; and Unosphere Q. In some embodiments, a resin is chosen with a relatively larger pore size, for increased surface area exposed to negatively charged species.

In some embodiments, a sample (batch) may be split subsequent to an AEX step, with split batches further processed in parallel or in sequence. In some embodiments, a batch may not be split subsequent to an AEX step.

Additives such as polyethylene glycol (PEG), detergents, amino acids, sugars, chaotropic agents can be added to enhance the performance of the separation to achieve better separation, recovery and/or product quality.

### Cation Exchange Chromatography

The method may comprise subjecting a biological sample comprising a protein of interest to at least one cation exchange (CEX) step. In certain exemplary embodiments, the CEX step will be in addition to an AEX step and occur either before or after the AEX step. In some embodiments, CEX is the third chromatography unit operation in the purification process.

In performing cation exchange, a sample comprising a protein of interest can be contacted with a cation exchange material by using any of a variety of techniques, for example, using a batch production technique or a chromatographic technique, as described above for AEX. Cation exchange packed bed chromatography is based on ionic interactions between the binding entity (target protein or impurity) and the functional group immobilized on the chromatographic media. Performance may be a function of, for example, the mobile phase, elution conditions, the functional group, and the resin backbone. In some embodiments, a particular objective of a CEX step is to reduce the levels of HCPs and HMW product related impurities, for example, protein aggregates.

In some embodiments, a concentration of protein loaded on a CEX resin (e.g., grams protein per liter resin) is between about 40 g/L and about 110 g/L, less than about 100 g/L, about 40 g/L, about 45 g/L, about 50 g/L, about 55 g/L, about 60 g/L, about 65 g/L, about 70 g/L, about 75 g/L, about 80 g/L, about 85 g/L, about 90 g/L, about 95 g/L, about 100 g/L, about 105 g/L, or about 110 g/L.

In some embodiments, a CEX column is subjected to a pre-strip step to remove any bound impurities before beginning a new separation. In some embodiments, a pre-strip buffer comprises about 2 M sodium chloride (NaCl). In some embodiments, about two column volumes of pre-strip buffer are used. In some embodiments, a pre-strip step is used only for the first cycle of a batch.

In some embodiments, a CEX column is subjected to an equilibration step to change the mobile phase pH and conductivity to favor adsorption. In some embodiments, an equilibration buffer comprises about 40 mM sodium acetate at a pH between about 5.90 and about 6.10, and a conductivity between about 2.00 mS/cm and about 4.00 mS/cm. In some embodiments, an equilibration buffer comprises a pH between about 4.00 and about 6.50, between about 5.00 and about 6.00, between about 5.90 and about 6.10, about 4.00, about 4.10, about 4.20, about 4.30, about 4.40, about 4.50, about 4.60, about 4.70, about 4.80, about 4.90, about 5.00, about 5.10, about 5.20, about 5.30, about 5.40, about 5.50, about 5.60, about 5.70, about 5.80, about 5.90, about 6.00, about 6.10, about 6.20, about 6.30, about 6.40, or about 6.50. In some embodiments, a conductivity of an equilibration buffer is between about 2.00 mS/cm and about 6.00 mS/cm, between about 2.00 mS/cm and about 4.00 mS/cm, between about 4.00 mS/cm and about 6.00 mS/cm, between about 3.00 mS/cm and about 4.00 mS/cm, about 2.00 mS/cm, about 2.10 mS/cm, about 2.20 mS/cm, about 2.30 mS/cm, about 2.40 mS/cm, about 2.50 mS/cm, about 2.60 mS/cm, about 2.70 mS/cm, about 2.80 mS/cm, about 2.90 mS/cm, about 3.00 mS/cm, about 3.10 mS/cm, about 3.20 mS/cm, about 3.30 mS/cm, about 3.40 mS/cm, about 3.50 mS/cm, about 3.60 mS/cm, about 3.70 mS/cm, about 3.80 mS/cm, about 3.90 mS/cm, about 4.00 mS/cm, about 4.10 mS/cm, about 4.20 mS/cm, about 4.30 mS/cm, about 4.40 mS/cm, about 4.50 mS/cm, about 4.60 mS/cm, about 4.70 mS/cm, about 4.80 mS/cm, about 4.90 mS/cm, about 5.00 mS/cm, about 5.10 mS/cm, about 5.20 mS/cm, about 5.30 mS/cm, about 5.40 mS/cm, about 5.50 mS/cm, about 5.60 mS/cm, about 5.70 mS/cm, about 5.80 mS/cm, about 5.90 mS/cm, or about 6.00 mS/cm. In some embodiments, about three column volumes of equilibration buffer are used.

A sample (CEX load) may be adjusted prior to contact with a CEX material. In some embodiments, a sample, for example an AEX pool, is adjusted to a pH between about 4.00 and about 6.50, between about 5.00 and about 6.00, between about 5.90 and about 6.10, about 4.00, about 4.10, about 4.20, about 4.30, about 4.40, about 4.50, about 4.60, about 4.70, about 4.80, about 4.90, about 5.00, about 5.10, about 5.20, about 5.30, about 5.40, about 5.50, about 5.60, about 5.70, about 5.80, about 5.90, about 6.00, about 6.10, about 6.20, about 6.30, about 6.40, or about 6.50. In some embodiments, a sample is adjusted using about 2 M acetic acid.

In some embodiments, a CEX column is subjected to a wash step to remove weakly bound impurities. In some embodiments, an equilibration buffer is also used as a wash buffer. In some embodiments, a wash buffer comprises about 40 mM sodium acetate at a pH between about 5.90 and about 6.10, and a conductivity between about 2.00 mS/cm and about 4.00 mS/cm. In some embodiments, a wash buffer comprises a pH between about 4.00 and about 6.50, between about 5.00 and about 6.00, between about 5.90 and about 6.10, about 4.00, about 4.10, about 4.20, about 4.30, about 4.40, about 4.50, about 4.60, about 4.70, about 4.80, about 4.90, about 5.00, about 5.10, about 5.20, about 5.30, about 5.40, about 5.50, about 5.60, about 5.70, about 5.80, about 5.90, about 6.00, about 6.10, about 6.20, about 6.30, about 6.40, or about 6.50. In some embodiments, a conductivity of a wash buffer is between about 2.00 mS/cm and about 6.00 mS/cm, between about 2.00 mS/cm and about 4.00 mS/cm, between about 4.00 mS/cm and about 6.00 mS/cm, between about 3.00 mS/cm and about 4.00 mS/cm, about 2.00 mS/cm, about 2.10 mS/cm, about 2.20 mS/cm, about 2.30 mS/cm, about 2.40 mS/cm, about 2.50 mS/cm, about 2.60 mS/cm, about 2.70 mS/cm, about 2.80 mS/cm, about 2.90 mS/cm, about 3.00 mS/cm, about 3.10 mS/cm, about 3.20 mS/cm, about 3.30 mS/cm, about 3.40 mS/cm, about 3.50 mS/cm, about 3.60 mS/cm, about 3.70 mS/cm, about 3.80 mS/cm, about 3.90 mS/cm, about 4.00 mS/cm, about 4.10 mS/cm, about 4.20 mS/cm, about 4.30 mS/cm, about 4.40 mS/cm, about 4.50 mS/cm, about 4.60 mS/cm, about 4.70 mS/cm, about 4.80 mS/cm, about 4.90 mS/cm, about 5.00 mS/cm, about 5.10 mS/cm, about 5.20 mS/cm, about 5.30 mS/cm, about 5.40 mS/cm, about 5.50 mS/cm, about 5.60 mS/cm, about 5.70 mS/cm, about 5.80 mS/cm, about 5.90 mS/cm, or about 6.00 mS/cm. In some embodiments, about two column volumes of wash buffer are used. In some embodiments, a wash buffer comprises potassium sorbate.

Following a wash step, a protein of interest may be eluted through an increase in conductivity. In some embodiments, CEX pool collection begins at 0.2 AU, using a 2 mm UV path, and ends after about 5 column volumes (CVs) of pool collection. In some embodiments, an elution buffer comprises about 20 mM Tris and about 120 mM sodium acetate at a pH between about 5.90 and about 6.20, and a conductivity between about 9.00 mS/cm and about 11.00 mS/cm. In some embodiments, an elution buffer comprises a pH between about 5.70 and about 7.00, between about 5.90 and about 6.20, about 5.70, about 5.80, about 5.90, about 6.00, about 6.10, about 6.20, about 6.30, about 6.40, about 6.50, about 6.60, about 6.70, about 6.80, about 6.90, or about 7.00. In some embodiments, a conductivity of an equilibration buffer is between about 7.00 mS/cm and about 11.00 mS/cm, between about 9.00 mS/cm and about 11.00 mS/cm, between about 10.00 mS/cm and about 11.00 mS/cm, about 7.00 mS/cm, about 7.10 mS/cm, about 7.20 mS/cm, about 7.30 mS/cm, about 7.40 mS/cm, about 7.50 mS/cm, about 7.60 mS/cm, about 7.70 mS/cm, about 7.80 mS/cm, about 7.90 mS/cm, about 8.00 mS/cm, about 8.10 mS/cm, about 8.20 mS/cm, about 8.30 mS/cm, about 8.40 mS/cm, about 8.50 mS/cm, about 8.60 mS/cm, about 8.70 mS/cm, about 8.80 mS/cm, about 8.90 mS/cm, about 9.00 mS/cm, about 9.10 mS/cm, about 9.20 mS/cm, about 9.30 mS/cm, about 9.40 mS/cm, about 9.50 mS/cm, about 9.60 mS/cm, about 9.70 mS/cm, about 9.80 mS/cm, about 9.90 mS/cm, about 10.00 mS/cm, about 10.10 mS/cm, about 10.20 mS/cm, about 10.30 mS/cm, about 10.40 mS/cm, about 10.50 mS/cm, about 10.60 mS/cm, about 10.70 mS/cm, about 10.80 mS/cm, about 10.90 mS/cm, or about 11.00 mS/cm. In some embodiments, about five column volumes of elution buffer are used.

In some embodiments, after elution a CEX column is subjected to a high ionic strength strip followed by a caustic strip to remove bound impurities and prepare the resin for additional cycles or storage. In some embodiments, a first strip buffer comprises about 2 M sodium chloride (NaCl) and a second strip buffer comprises about 1 N sodium hydroxide (NaOH).

In some embodiments, each phase of the separation except elution is performed at a linear velocity between about 100 and about 300 cm/hr, between about 150 and about 250 cm/hr, about 100 cm/hr, about 110 cm/hr, about 120 cm/hr, about 130 cm/hr, about 140 cm/hr, about 150 cm/hr, about 160 cm/hr, about 170 cm/hr, about 180 cm/hr, about 190 cm/hr, about 200 cm/hr, about 210 cm/hr, about 220 cm/hr, about 230 cm/hr, about 240 cm/hr, about 250 cm/hr, about 260 cm/hr, about 270 cm/hr, about 280 cm/hr, about 290 cm/hr, or about 300 cm/hr. In an exemplary embodiment, each phase of the separation except for elution is performed at a linear velocity of 200 cm/hr. In an exemplary embodiment, the elution step is performed at a linear velocity of 100 cm/hr.

In some embodiments, a CEX resin lifetime is between about 1 and about 100 cycles, between about 50 and about 90 cycles, less than about 100 cycles, about 10 cycles, about 20 cycles, about 30 cycles, about 40 cycles, about 50 cycles, about 60 cycles, about 70 cycles, about 80 cycles, about 90 cycles, or about 100 cycles. In some embodiments, an AEX resin lifetime may reach 200 cycles.

An aqueous salt solution may be used as both a loading and wash buffer having a pH that is lower than the isoelectric point (pI) of the protein of interest. In one aspect, the pH is about 0 to 5 units lower than the pI of the protein. In another aspect, it is in the range of 1 to 2 units lower than the pI of the protein. In yet another aspect, it is in the range of 1 to 1.5 units lower than the pI of the protein.

In certain exemplary embodiments, the concentration of the anionic agent in aqueous salt solution is increased or decreased to achieve a pH of between about 3.5 and about 10.5, or between about 4 and about 10, or between about 4.5 and about 9.5, or between about 5 and about 9, or between about 5.5 and about 8.5, or between about 6 and about 8, or between about 6.5 and about 7.5. In one aspect, the concentration of anionic agent is increased or decreased in the aqueous salt solution in order to achieve a pH of 5, or 5.5, or 6, or 6.5, or 6.8, or 7.5. Buffer systems suitable for use in the CEX methods include, but are not limited to, Tris formate, Tris acetate, ammonium sulfate, sodium chloride, and sodium sulfate.

In certain exemplary embodiments, the conductivity and pH of the aqueous salt solution is adjusted by increasing or decreasing the concentration of a cationic agent. In one aspect, the cationic agent is maintained at a concentration ranging from about 20 mM to about 500 mM, about 50 mM to about 350 mM, about 100 mM to about 300 mM, or about 100 mM to about 200 mM. Non-limiting examples of the cationic agent can be selected from the group consisting of sodium, Tris, triethylamine, ammonium, arginine, and combinations thereof.

A packed cation-exchange chromatography column or a cation-exchange membrane device can be operated either in bind-elute mode, flowthrough mode, or a hybrid mode wherein the product exhibits binding to or interacting with a chromatographic material yet can be washed from such material using a buffer that is the same or substantially similar to the loading buffer (details of these modes are outlined above). In some embodiments, a CEX step is performed in positive (bind-elute) mode, where positively charged protein of interest and impurities are adsorbed to the immobilized, negatively charged stationary phase. The protein of interest is then eluted while many of the impurities remain bound to the stationary phase. A series of regeneration steps remove bound impurities and prepare the CEX column for subsequent cycles.

Cationic substituents include carboxymethyl (CM), sulfoethyl (SE), sulfopropyl (SP), phosphate (P) and sulfonate (S). Additional cationic materials include, but are not limited to: Capto SP ImpRes, which is a high flow agarose bead; Capto S ImpAct; CM Hyper D grade F, which is a ceramic bead coated and permeated with a functionalized hydrogel, 250-400 ionic groups μeq/mL; Eshmuno S, which is a hydrophilic polyvinyl ether base matrix with 50-100 μeq/mL ionic capacity; Nuvia C Prime, which is a hydrophobic cation exchange media composed of a macroporous highly crosslinked hydrophilic polymer matrix 55-75 με/mL; Nuvia S, which has a UNOsphere base matrix with 90-150 με/mL ionic groups; Poros HS, which is a rigid polymeric bead with a backbone consisting of cross-linked poly[styrene-divinylbenzene]; Poros XS, which is a rigid polymetric bead with a backbone consisting of cross-linked poly[styrene divinyl-benzene]; Toyo Pearl Giga Cap CM 650M, which is a polymeric base bead with 0.225 meq/mL ionic capacity; Toyo Pearl Giga Cap S 650M, which is a polymeric base bead; Toyo Pearl MX TRP, which is a polymeric base bead; and Fractogel® EMD SE Hicap. It is noted that CEX chromatography can be used with MM resins, described herein. In some embodiments, a CEX resin is selected for relatively higher capacity, for example for loading up to about 100 g/L of resin. In some embodiments, a CEX resin is selected for relatively higher caustic stability, for example to prevent hydrolysis of the cationic substrate.

Additives such as polyethylene glycol, detergents, amino acids, sugars, and chaotropic agents can be added to enhance the performance of the separation so as to achieve better separation, recovery and/or product quality.

### Mixed-Mode Chromatography

Mixed-mode (“MM”) chromatography may also be used in the process of the invention. MM chromatography, also referred to herein as “multimodal chromatography” or “MMC”, is a chromatographic strategy that utilizes a support comprising a ligand that is capable of providing at least two different interactions with an analyte or protein of interest from a sample. One of these sites provides an attractive type of charge-charge interaction between the ligand and the protein of interest, and the other site provides for electron acceptor-donor interaction and/or hydrophobic and/or hydrophilic interactions. Electron donor-acceptor interactions include interactions such as hydrogen-bonding, π-π, cation-π, charge transfer, dipole-dipole, induced dipole, etc.

The column resin employed for a mixed-mode separation can be Capto Adhere. Capto Adhere is a strong anion exchanger with multimodal functionality. Its base matrix is a highly cross-linked agarose with a ligand (N-benzyl-N-methyl ethanol amine) that exhibits different functionalities for interaction, such as ionic interaction, hydrogen bonding and hydrophobic interaction. In certain aspects, the resin employed for a mixed-mode separation is selected from PPA-HyperCel and HEA-HyperCel. The base matrices of PPA-HyperCel and HEA-HyperCel are high porosity cross-linked cellulose. Their ligands are phenylpropylamine and hexylamine, respectively. Phenylpropylamine and hexylamine offer different selectivity and hydrophobicity options for protein separations. Additional mixed-mode chromatographic supports include, but are not limited to, Capto MMC, MEP-HyperCel, MBI HyperCel, CMM HyperCel, Capto Adhere ImpRes, Capto Core 700, Nuvia C Prime, Toyo Pearl MX Trp 650M, and Eshmuno® HCX. In certain aspects, the mixed-mode chromatography resin is comprised of ligands coupled to an organic or inorganic support, sometimes denoted by a base matrix, directly or via a spacer. The support may be in the form of particles, such as essentially spherical particles, a monolith, filter, membrane, surface, capillaries, and the like. In certain aspects, the support is prepared from a native polymer, such as cross-linked carbohydrate material, such as agarose, agar, cellulose, dextran, chitosan, konjac, carrageenan, gellan, alginate and the like. To obtain high adsorption capacities, the support can be porous, and ligands are then coupled to the external surfaces as well as to the pore surfaces. Such native polymer supports can be prepared according to standard methods, such as inverse suspension gelation (S Hjerten: Biochim Biophys Acta 79(2), 393-398 (1964), the entire teaching of which is incorporated herein by reference). Alternatively, the support can be prepared from a synthetic polymer, such as cross-linked synthetic polymers, for example, styrene or styrene derivatives, divinylbenzene, acrylamides, acrylate esters, methacrylate esters, vinyl esters, vinyl amides, and the like. Such synthetic polymers can be produced according to standard methods, see “Styrene based polymer supports developed by suspension polymerization” (R Arshady: Chimica e L'Industria 70(9), 70-75 (1988), the entire teaching of which is incorporated herein by reference). Porous native or synthetic polymer supports are also available from commercial sources, such as Cytiva, Uppsala, Sweden. MMC may be operated in flowthrough mode or bind and elute mode depending on the resin used; for example, AEX/HIC hybrid resins may preferably be operated in flowthrough mode while CEX/HIC hybrid resins may preferably be operated in bind and elute mode.

Additives such as polyethylene glycol, detergents, amino acids, sugars, chaotropic agents can be added to enhance the performance of the separation, so as to achieve better separation, recovery and/or product quality.

The methods of the disclosure can also be implemented in a continuous chromatography mode. In this mode, at least two columns are employed (referred to as a “first” column and a “second” column). In certain embodiments, this continuous chromatography mode can be performed such that the eluted fractions and/or stripped fractions can then be loaded subsequently or concurrently onto the second column (with or without dilution).

In one embodiment, the media choice for continuous mode can be one of various chromatographic resins with pendant hydrophobic and anion exchange functional groups, monolithic media, membrane adsorbent media or depth filtration media.

In some exemplary embodiments, MMC is the second chromatographic separation in the process, following affinity capture chromatography and optionally followed by an AEX step. In other embodiments, MMC is the third chromatographic separation in the process, following affinity capture chromatography and an AEX step. In some embodiments, the method of the present disclosure may include MMC and not CEX or HIC steps. In some embodiments, a process for producing a recombinant protein such as Dupilumab includes two, three, or four chromatography modalities, including affinity capture chromatography, MMC, and optionally AEX, CEX, HIC, or an additional MMC step, in any order before or after MMC. MMC may be operated in flowthrough mode or in bind and elute mode regardless of an order of chromatography steps. MMC may be operated in a plate-based format, for example a 96-well plate-based format, or a robocolumn-based format.

In some embodiments, a MMC column is subjected to an equilibration step to change the mobile phase pH and conductivity to favor adsorption. In some embodiments, an equilibration buffer comprises about 100 mM NaCl at a pH of about 5. In some embodiments, an equilibration buffer comprises a pH between about 4.50 and about 9.00, between about 4.50 and about 8.00, between about 4.50 and about 5.50, between about 5.00 and about 6.00, about 4.50, about 4.60, about 4.70, about 4.80, about 4.90, about 5.00, about 5.10, about 5.20, about 5.30, about 5.40, about 5.50, about 5.60, about 5.70, about 5.80, about 5.90, about 6.00, about 6.10, about 6.20, about 6.30, about 6.40, about 6.50, about 6.60, about 6.70, about 6.80, about 6.90, about 7.00, about 7.10, about 7.20, about 7.30, about 7.40, about 7.50, about 7.60, about 7.70, about 7.80, about 7.90, about 8.00, about 8.10, about 8.20, about 8.30, about 8.40, about 8.50, about 8.60, about 8.70, about 8.80, about 8.90, or about 9.00.

In some embodiments, an equilibration buffer comprises NaCl at between about 100 mM and about 500 mM, between about 100 mM and about 250 mM, between about 100 mM and about 150 mM, between about 80 mM and about 120 mM, between about 95 mM and about 105 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 175 mM, about 200 mM, about 225 mM, about 250 mM, about 275 mM, about 300 mM, about 325 mM, about 350 mM, about 375 mM, about 400 mM, about 425 mM, about 450 mM, about 475 mM, or about 500 mM. In some embodiments, an equilibration buffer comprises NaCl between about 0 mM and about 100 mM. In some embodiments, an equilibration buffer comprises citrate or arginine.

In some embodiments, a wash buffer may comprise any of the pH values, NaCl, concentrations, or buffer salts of the equilibration buffer described above.

In some embodiments, a quantity of protein loaded on an MMC resin (e.g., gram protein per liter resin) is about 100 g/L or about 110 g/L. In some embodiments, a quantity of protein loaded on an MMC resin is between about 50 g/L and about 200 g/L, between about 100 g/L and about 150 g/L, between about 100 g/L and about 110 g/L, less than about 120 g/L, about 50 g/L, about 55 g/L, about 60 g/L, about 65 g/L, about 70 g/L, about 75 g/L, about 80 g/L, about 85 g/L, about 90 g/L, about 95 g/L, about 100 g/L, about 105 g/L, about 110 g/L, about 115 g/L, about 120 g/L, about 125 g/L, about 130 g/L, about 135 g/L, about 140 g/L, about 145 g/L, about 150 g/L, about 155 g/L, about 160 g/L, about 165 g/L, about 170 g/L, about 175 g/L, about 180 g/L, about 185 g/L, about 190 g/L, about 195 g/L, or about 200 g/L. In some embodiments, a quantity of protein loaded on an MMC resin may be between about 10 g/L and about 80 g/L, about 10 g/L, about 15 g/L, about 20 g/L, about 25 g/L, about 30 g/L, about 35 g/L, about 40 g/L, about 45 g/L, about 50 g/L, about 55 g/L, about 60 g/L, about 65 g/L, about 70 g/L, about 75 g/L, or about 80 g/L.

In some embodiments, MMC is operated in bind and elute mode, and further includes the use of an elution buffer. In some embodiments, an elution buffer comprises a pH between about 4.50 and about 9.00, between about 4.50 and about 8.00, between about 4.50 and about 5.50, between about 5.00 and about 6.00, about 4.50, about 4.60, about 4.70, about 4.80, about 4.90, about 5.00, about 5.10, about 5.20, about 5.30, about 5.40, about 5.50, about 5.60, about 5.70, about 5.80, about 5.90, about 6.00, about 6.10, about 6.20, about 6.30, about 6.40, about 6.50, about 6.60, about 6.70, about 6.80, about 6.90, about 7.00, about 7.10, about 7.20, about 7.30, about 7.40, about 7.50, about 7.60, about 7.70, about 7.80, about 7.90, about 8.00, about 8.10, about 8.20, about 8.30, about 8.40, about 8.50, about 8.60, about 8.70, about 8.80, about 8.90, or about 9.00.

In some embodiments, an elution buffer comprises NaCl at between about 0 mM and about 500 mM, between about 100 mM and about 250 mM, between about 100 mM and about 150 mM, about 0 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 175 mM, about 200 mM, about 225 mM, about 250 mM, about 275 mM, about 300 mM, about 325 mM, about 350 mM, about 375 mM, about 400 mM, about 425 mM, about 450 mM, about 475 mM, or about 500 mM. In some embodiments, an elution buffer comprises citrate or arginine.

### Hydrophobic Interaction Chromatography

The method of the present disclosure may comprise subjecting a biological sample comprising a protein of interest to at least one hydrophobic interaction chromatography (HIC) step. In performing the separation, a biological sample is contacted with a HIC material, for example, using a batch production technique or using a column or membrane chromatography. Prior to HIC processing it may be desirable to adjust the concentration of the salt buffer to achieve desired protein binding/interaction to the resin or the membrane. In some embodiments, HIC separation is a fourth and final chromatographic separation in the process and is performed downstream of a CEX step.

In some embodiments, a particular objective of a HIC step may be, for example, to reduce the levels of HCPs or HMW product related impurities, including PLBD2. PLBD2 levels in the HIC load may be present at 100-300 ppm and may be reduced by about 40×, about 50×, about 60×, about 70×, about 80×, about 90×, about 100×, about 110×, about 120×, about 130×, about 140×, about 150×, about 160×, about 170×, about 180×, about 190×, about 200×, about 210×, about 220×, about 230×, about 240×, about 250×, about 260×, about 270×, about 280×, about 290×, about 300×, or about 310×, below 100 ppm, below 30 ppm, below 4 ppm, or below 1 ppm. The use of HIC to remove PLBD2 is also described in more detail in U.S. Pat. No. 10,774,141, and is incorporated herein by reference in its entirety for all purposes.

In some embodiments, a batch of sample may be split into two sublots that are individually processed through a HIC step and subsequent steps, for example virus-retentive filtration and UF/DF, before being recombined, for example prior to third stage concentration.

Whereas ion exchange chromatography relies on the local charge of the protein of interest for selective separation, hydrophobic interaction chromatography exploits the hydrophobic properties of proteins to achieve selective separation. HIC resins are typically functionalized with aromatic or aliphatic hydrocarbon ligands. Hydrophobic groups on or within a protein interact with hydrophobic groups of chromatography resin or a membrane. Typically, under suitable conditions, the more hydrophobic a protein is (or portions of a protein) the stronger it will interact with the column or the membrane. Thus, under suitable conditions, HIC can be used to facilitate the separation of process-related impurities (e.g., HCPs) as well as product-related substances (e.g., aggregates and fragments) from a protein of interest in a sample.

Like ion exchange chromatography, a HIC column or a HIC membrane device can also be operated in an elution mode, a flowthrough, or a hybrid mode wherein the product exhibits binding to or interacting with a chromatographic material yet can be washed from such material using a buffer that is the same or substantially similar to the loading buffer. (The details of these modes are outlined above in connection with AEX processing.) In some embodiments, a HIC step is performed in negative mode where process-related impurities bind to an immobilized ligand, and the protein of interest flows through.

As hydrophobic interactions are strongest at high ionic strength, this form of separation is conveniently performed following a salt elution step such as those typically used in connection with ion exchange chromatography. Alternatively, salts can be added to a sample before employing a HIC step. Adsorption of a protein to a HIC column is favored by high salt concentrations, but the actual concentrations can vary over a wide range depending on the nature of the protein of interest, salt type and the particular HIC ligand chosen. Various ions can be arranged in a so-called soluphobic series depending on whether they promote hydrophobic interactions (salting-out effects) or disrupt the structure of water (chaotropic effect) and lead to the weakening of the hydrophobic interaction. Cations are ranked in terms of increasing salting out effect as Ba²⁺; Ca²⁺; Mg²⁺; Li⁺; Cs⁺; Na⁺; K⁺; Rb⁺; NH4⁺, while anions may be ranked in terms of increasing chaotropic effect as PO₄³⁻; SO₄²⁻; CH₃CO₃⁻; CI⁻; Br⁻; NO₃⁻; ClO₄⁻; I⁻; SCN⁻.

HIC media normally comprise a base matrix (e.g., cross-linked agarose or synthetic copolymer material) to which hydrophobic ligands (e.g., alkyl or aryl groups) are coupled. A suitable HIC media comprises an agarose resin or a membrane functionalized with phenyl groups (e.g., a Phenyl Sepharose™ from Cytiva or a Phenyl Membrane from Sartorius). Various HIC resins are available commercially. Examples include, but are not limited to, Capto Phenyl, Capto Butyl, Phenyl Sepharose™ 6 Fast Flow with low or high substitution, Phenyl Sepharose™ High Performance, Octyl Sepharose™ High Performance (GE Healthcare); Fractogel™ EMD Propyl or Fractogel™ EMD Phenyl (E. Merck, Germany); Macro-Prep™ Methyl or Macro-Prep™ t-Butyl columns (Bio-Rad, California); WP HI-Propyl (C3)™ (J. T. Baker, New Jersey); and Toyopearl™ ether, phenyl or butyl (TosoHaas, PA); Toyo PPG; Toyo Phenyl; Toyo Butyl; and Toyo Hexyl.

Because the pH selected for any particular production process must be compatible with protein stability and activity, particular pH conditions may be specific for each application. However, because at pH 5.0-8.5 particular pH values have very little significance on the final selectivity and resolution of a HIC separation, such conditions may be favored. An increase in pH weakens hydrophobic interactions and retention of proteins changes more drastically at pH values above 8.5 or below 5.0. In addition, changes in ionic strength, the presence of organic solvents, temperature and pH (especially at the isoelectric point, pI, when there is no net surface charge) can impact protein structure and solubility and, consequently, the interaction with other hydrophobic surfaces, such as those in HIC media and hence, in certain embodiments, the present disclosure incorporates production strategies wherein one or more of the foregoing are adjusted to achieve the desired reduction in process-related impurities and/or product-related substances.

In certain exemplary embodiments, spectroscopy methods such as UV, NIR, FTIR, Fluorescence, and Raman may be used to monitor the protein of interest and impurities in an on-line, at-line or in-line mode, which can then be used to control the level of aggregates in the pooled material collected from the HIC adsorbent effluent. In other exemplary embodiments, on-line, at-line or in-line monitoring methods can be used either on the effluent line of the chromatography step or in the collection vessel, to enable achievement of the desired product quality/recovery. In yet other exemplary embodiments, the UV signal can be used as a surrogate to achieve an appropriate product quality/recovery, wherein the UV signal can be processed appropriately, including, but not limited to, such processing techniques as integration, differentiation, and moving average, such that normal process variability can be addressed and the target product quality can be achieved. In certain exemplary embodiments, such measurements can be combined with in-line dilution methods such that ion concentration/conductivity of the load/wash can be controlled by feedback and hence facilitate product quality control.

In some embodiments, a concentration of protein loaded on a HIC resin (e.g., gram protein per liter resin) is between about 60 g/L and about 180 g/L, between about 100 g/L and about 150 g/L, between about 180 g/L and about 200 g/L, less than about 180 g/L, about 60 g/L, about 65 g/L, about 70 g/L, about 75 g/L, about 80 g/L, about 85 g/L, about 90 g/L, about 95 g/L, about 100 g/L, about 105 g/L, about 110 g/L, about 115 g/L, about 120 g/L, about 125 g/L, about 130 g/L, about 135 g/L, about 140 g/L, about 145 g/L, about 150 g/L, about 155 g/L, about 160 g/L, about 165 g/L, about 170 g/L, about 175 g/L, about 175 g/L, about 180 g/L, about 185 g/L, about 190 g/L, about 195 g/L, or about 200 g/L.

In some embodiments, a sample may be adjusted before contacting a HIC material, which may be referred to as load adjustment. In some embodiments, a sample, for example a CEX pool, is adjusted to about 40 mM citrate using a single bolus addition of about 1.2 M sodium citrate. Following the adjustment, the pH of the sample (HIC load) is between about 6.30 and about 6.70, and the conductivity of the sample is between about 14.00 and about 17.00 mS/cm. In some embodiments, a HIC load comprises a pH between about 5.50 and about 7.00, between about 6.30 and about 6.70, about 5.50, about 5.60, about 5.70, about 5.80, about 5.90, about 6.00, about 6.10, about 6.20, about 6.30, about 6.40, about 6.50, about 6.60, about 6.70, about 6.80, about 6.90, or about 7.00. In some embodiments, a HIC load comprises a conductivity between about 14.00 mS/cm and about 17.00 mS/cm, about 14.00 mS/cm, about 14.10 mS/cm, about 14.20 mS/cm, about 14.30 mS/cm, about 14.40 mS/cm, about 14.50 mS/cm, about 14.60 mS/cm, about 14.70 mS/cm, about 14.80 mS/cm, about 14.90 mS/cm, about 15.00 mS/cm, about 15.10 mS/cm, about 15.20 mS/cm, about 15.30 mS/cm, about 15.40 mS/cm, about 15.50 mS/cm, about 15.60 mS/cm, about 15.70 mS/cm, about 15.80 mS/cm, about 15.90 mS/cm, about 16.00 mS/cm, about 16.10 mS/cm, about 16.20 mS/cm, about 16.30 mS/cm, about 16.40 mS/cm, about 16.50 mS/cm, about 16.60 mS/cm, about 16.70 mS/cm, about 16.80 mS/cm, about 16.90 mS/cm, or about 17.00 mS/cm. In some embodiments, a concentration of citrate in a HIC load is between about 10 mM and about 50 mM, between about 30 mM and about 40 mM, about 10 mM, about 15 mM, about 20 mM, about 25 mM, about 30 mM, about 31 mM, about 32 mM, about 33 mM, about 34 mM, about 35 mM, about 36 mM, about 37 mM, about 38 mM, about 39 mM, about 40 mM, about 45 mM, or about 50 mM.

In some embodiments, a HIC column is subjected to a pre-equilibration step prior to contact with a sample. A pre-equilibration step removes any residual bound impurities from the column. In some embodiments, a pre-equilibration step is only performed for the first cycle of a batch. In some exemplary embodiments, a pre-equilibration buffer comprises water for injection (WFI). In some exemplary embodiments, a linear velocity of a pre-equilibration buffer is between about 100 and about 300 cm/hr, between about 150 and about 250 cm/hr, about 100 cm/hr, about 110 cm/hr, about 120 cm/hr, about 130 cm/hr, about 140 cm/hr, about 150 cm/hr, about 160 cm/hr, about 170 cm/hr, about 180 cm/hr, about 190 cm/hr, about 200 cm/hr, about 210 cm/hr, about 220 cm/hr, about 230 cm/hr, about 240 cm/hr, about 250 cm/hr, about 260 cm/hr, about 270 cm/hr, about 280 cm/hr, about 290 cm/hr, or about 300 cm/hr. In an exemplary embodiment, the linear velocity is about 200 cm/hr. In some exemplary embodiments, about three column volumes of pre-equilibration buffer are used.

In some embodiments, a HIC column is subjected to an equilibration step prior to contact with a sample, and optionally following a pre-equilibration step. An equilibration step changes the mobile phase to a pH and conductivity that matches the buffer excipients present in the load material (sample). In some embodiments, an equilibration buffer may comprise about 40 mM Tris and about 40 mM sodium citrate, at a pH between about 6.30 and about 6.70, and a conductivity between about 8.50 mS/cm and about 12.00 mS/cm. In some embodiments, a linear velocity of an equilibration buffer is between about 100 and about 300 cm/hr, between about 150 and about 250 cm/hr, about 100 cm/hr, about 110 cm/hr, about 120 cm/hr, about 130 cm/hr, about 140 cm/hr, about 150 cm/hr, about 160 cm/hr, about 170 cm/hr, about 180 cm/hr, about 190 cm/hr, about 200 cm/hr, about 210 cm/hr, about 220 cm/hr, about 230 cm/hr, about 240 cm/hr, about 250 cm/hr, about 260 cm/hr, about 270 cm/hr, about 280 cm/hr, about 290 cm/hr, or about 300 cm/hr. In an exemplary embodiment, the linear velocity is about 200 cm/hr. In some exemplary embodiments, about two column volumes of an equilibration buffer are used.

In some embodiments, a protein concentration of a HIC load is between about 10.0 g/L and about 20.0 g/L, between about 12.0 g/L and about 18.0 g/L, about 10.0 g/L, about 10.5 g/L, about 11.0 g/L, about 11.5 g/L, about 12.0 g/L, about 12.5 g/L, about 13.0 g/L, about 13.5 g/L, about 14.0 g/L, about 14.5 g/L, about 15.0 g/L, about 15.5 g/L, about 16.0 g/L, about 16.5 g/L, about 17.0 g/L, about 17.5 g/L, about 18.0 g/L, about 18.5 g/L, about 19.0 g/L, about 19.5 g/L, or about 20.0 g/L. In some embodiments, a linear velocity of the HIC load is between about 100 and about 300 cm/hr, between about 150 and about 250 cm/hr, about 100 cm/hr, about 110 cm/hr, about 120 cm/hr, about 130 cm/hr, about 140 cm/hr, about 150 cm/hr, about 160 cm/hr, about 170 cm/hr, about 180 cm/hr, about 190 cm/hr, about 200 cm/hr, about 210 cm/hr, about 220 cm/hr, about 230 cm/hr, about 240 cm/hr, about 250 cm/hr, about 260 cm/hr, about 270 cm/hr, about 280 cm/hr, about 290 cm/hr, or about 300 cm/hr. In an exemplary embodiment, the linear velocity is about 150 cm/hr.

In some embodiments, flowthrough is collected when UV absorbance at 280 nm reaches 0.2 AU on a 5 mm flow path, approximately 3 to 4 column volumes into the load step. This is followed by a wash step to increase yield.

In some embodiments, an equilibration buffer is also used as a wash buffer. In some embodiments, a wash buffer comprises about 40 mM Tris and about 40 mM sodium citrate, at a pH between about 6.30 and about 6.70, and a conductivity between about 8.50 mS/cm and about 12.00 mS/cm. In some embodiments, a linear velocity of a wash buffer is between about 100 and about 300 cm/hr, between about 150 and about 250 cm/hr, about 100 cm/hr, about 110 cm/hr, about 120 cm/hr, about 130 cm/hr, about 140 cm/hr, about 150 cm/hr, about 160 cm/hr, about 170 cm/hr, about 180 cm/hr, about 190 cm/hr, about 200 cm/hr, about 210 cm/hr, about 220 cm/hr, about 230 cm/hr, about 240 cm/hr, about 250 cm/hr, about 260 cm/hr, about 270 cm/hr, about 280 cm/hr, about 290 cm/hr, or about 300 cm/hr. In an exemplary embodiment, the linear velocity is about 150 cm/hr. In some embodiments, a wash length is between about 6 and about 8 column volumes (CVs), about 6 CVs, about 6.5 CVs, about 7 CVs, about 7.5 CVs, or about 8 CVs. In an exemplary embodiment, the wash length is about 8 CVs.

In some embodiments, a HIC column may be regenerated using one or more strip buffers. In some embodiments, a first strip buffer comprises about two column volumes of WFI, a second strip buffer comprises about two column volumes of 1 N sodium hydroxide, and a third strip buffer comprises about two column volumes of WFI. A strip buffer may comprise 5 mM sodium hydroxide. Additional steps may be performed for the final cycle of a batch. In some embodiments, the final cycle of a batch includes an additional strip step using about two column volumes of about 20% ethanol, a further strip step using about two column volumes of WFI, and a final equilibration step performed as described above.

In some embodiments, a HIC resin lifetime is between about 1 and about 100 cycles, between about 50 and about 90 cycles, less than about 100 cycles, about 10 cycles, about 20 cycles, about 40 cycles, about 50 cycles, about 60 cycles, about 70 cycles, about 80 cycles, about 90 cycles, or about 100 cycles. In some embodiments, an AEX resin lifetime may reach 200 cycles.

### Size Exclusion Chromatography

Size exclusion chromatography or gel filtration relies on the separation of components as a function of their molecular size. Separation depends on the amount of time that the substances spend in the porous stationary phase as compared to time in the fluid. The probability that a molecule will reside in a pore depends on the size of the molecule and the pore. In addition, the ability of a substance to permeate into pores is determined by the diffusion mobility of macromolecules which is higher for small macromolecules. Very large macromolecules may not penetrate the pores of the stationary phase at all; and, for very small macromolecules the probability of penetration is close to unity. While components of larger molecular size move more quickly past the stationary phase, components of small molecular size have a longer path length through the pores of the stationary phase and are thus retained longer in the stationary phase.

The chromatographic material can comprise a size exclusion material wherein the size exclusion material is a resin or membrane. The matrix used for size exclusion is preferably an inert gel medium which can be a composite of cross-linked polysaccharides, for example, cross-linked agarose and/or dextran in the form of spherical beads. The degree of cross-linking determines the size of pores that are present in the swollen gel beads. Molecules greater than a certain size do not enter the gel beads and thus move through the chromatographic bed the fastest. Smaller molecules, such as detergent, protein, DNA and the like, which enter the gel beads to varying extent depending on their size and shape, are retarded in their passage through the bed. Molecules are thus generally eluted in the order of decreasing molecular size.

Porous chromatographic resins appropriate for size-exclusion chromatography of viruses may be made of dextrose, agarose, polyacrylamide, or silica which have different physical characteristics. Polymer combinations can also be also used. Most commonly used are those under the tradename, “SEPHADEX” available from Amersham Biosciences. Other size exclusion supports from different materials of construction are also appropriate, for example Toyopearl 55F (polymethacrylate, from Tosoh Bioscience, Montgomery Pa.) and Bio-Gel P-30 Fine (BioRad Laboratories, Hercules, CA).

### Virus Retentive Filtration

Viral filtration is a dedicated viral reduction step in a production process. This step is usually performed after chromatographic polishing. Virus reduction by virus retentive membranes is based on hydrodynamic radii differences by which a protein of interest, for example a monoclonal antibody (about 10 nm in hydrodynamic radius) passes through the filter while the larger virus (greater than 18 nm in hydrodynamic radius) is retained by the membrane.

Viral reduction can be achieved via the use of suitable filters including, but not limited to, Planova 20N™, 50 N or BioEx from Asahi Kasei Pharma, Viresolve™ filters from EMD Millipore, ViroSart® CPV or Virosart® HF from Sartorius, or Ultipor DV20 filter, DV50™ or Pegasus Prime filter from Pall Corporation. It will be apparent to one of ordinary skill in the art to select a suitable filter to obtain desired filtration performance.

A viral retentive filtration step may additionally comprise a pre-filter step. Virus retentive filters are vulnerable to premature fouling due to pore plugging by impurities present in feed streams. Fouling is undesirable as the associated virus retentive filter flux decay has been correlated to virus breakthrough for certain virus retentive filters. Studies have reported use of guard or pre-filters to ameliorate virus filter fouling. Chemically functionalized pre-filters, including HIC, CEX and AEX modalities, have been found by the inventors to improve virus retentive filter capacity. Pre-filtering can be achieved by the use of suitable pre-filters including, but not limited to, Viresolve Shield, Viresolve Shield H, Millistak+HC Pro X0SP (MilliporeSigma), or Pegasus Protect from Pall Corporation. It will be apparent to one of ordinary skill in the art to select a suitable pre-filter to obtain desired filtration performance.

In some embodiments, a virus retentive filtration (VRF) step is performed immediately following a HIC step. In some embodiments, load material is conditioned with three pre-filters immediately before three filters.

In some exemplary embodiments, the VRF process comprises the six following steps. The filter assembly is first primed and wetted with WFI, until the device is filled and properly vented, at an operating transmembrane pressure (TMP) of 5±2 psi. The second step comprises flushing with greater than 50 L/m² WFI, at an operating TMP of 25±5 psi. This is followed by a pre-use integrity test, using sterile oil-free air or nitrogen gas. The fourth step comprises a buffer flush and flux verification: the assembly is equilibrated with a buffer comprising about 40 mM Tris and about 40 mM sodium citrate, pH 6.5, with a volume greater than 20 L/m² at 25±5 psi TMP.

The sample, for example HIC pool, is then loaded at a constant 25±5 psi TMP, with a volume of less than 900 L/m². Pool collection begins immediately upon the start of load and ends once load material is depleted. In some embodiments, a sample volume is between about 700 L/m² and about 1300 L/m², about 700 L/m², about 750 L/m², about 800 L/m², about 850 L/m², about 900 L/m², about 950 L/m², about 1000 L/m², about 1050 L/m², about 1100 L/m², about 1150 L/m², about 1200 L/m², about 1250 L/m², about 1300 L/m², about 1350 L/m², about 1400 L/m², about 1500 L/m², about 1600 L/m², about 1700 L/m², about 1800 L/m², about 1900 L/m² or about 2000 L/m². In some embodiments, a load conductivity is between about 7.5 mS/cm and about 13.5 mS/cm, about 7.5 mS/cm, about 8.0 mS/cm, about 8.5 mS/cm, about 9.0 mS/cm, about 9.5 mS/cm, about 10.0 mS/cm, about 10.5 mS/cm, about 11.0 mS/cm, about 11.5 mS/cm, about 12.0 mS/cm, about 12.5 mS/cm, about 13.0 mS/cm, or about 13.5 mS/cm. In some embodiments, a load pH is between about 4.5 and about 7.5, between about 6.3 and about 6.7, about 4.5, about 5.0, about 5.5, about 6.0, about 6.5, about 7.0, or about 7.5. In some embodiments, a load protein concentration is between about 4.5 g/L and about 13 g/L, between about 8 g/L and about 10 g/L, about 4.5 g/L, about 5.0 g/L, about 5.5 g/L, about 6.0 g/L, about 6.5 g/L, about 7.0 g/L, about 7.5 g/L, about 8.0 g/L, about 8.5 g/L, about 9.0 g/L, about 9.5 g/L, about 10.0 g/L, about 10.5 g/L, about 11.0 g/L, about 11.5 g/L, about 12.0 g/L, about 12.5 g/L, or about 13.0 g/L.

Finally, the filter is flushed with WFI in preparation for a final, post-use integrity test using sterile oil-free air or nitrogen gas.

### Concentration and Diafiltration

Certain exemplary embodiments of the present disclosure employ ultrafiltration and diafiltration (UF/DF), also referred to as concentration and diafiltration, to further concentrate and formulate a protein of interest. UF/DF conditions a drug substance to achieve pH, excipient content, and protein concentration conducive to long term storage and addition of stabilizing excipients to generate final drug substance (FDS). During processing, protein solution is pumped tangentially across the surface of a semi-permeable, parallel, flat sheet membrane. The membrane may be of a pore size, for example about 50 kDa, that is permeable to water and buffer salts but generally impermeable to a protein of interest, for example a monoclonal antibody. The driving force for permeation is applied transmembrane pressure (TMP) induced by flow restriction at the outlet of the membrane flow channel.

Ultrafiltration is described in detail in: Microfiltration and Ultrafiltration: Principles and Applications, L. Zeman and A. Zydney (Marcel Dekker, Inc., New York, N.Y., 1996); and in: Ultrafiltration Handbook, Munir Cheryan (Technomic Publishing, 1986; ISBN No. 87762-456-9); the entire teachings of which are incorporated herein by reference. One filtration process is Tangential Flow Filtration as described in the Millipore catalogue entitled “Pharmaceutical Process Filtration Catalogue” pp. 177-202 (Bedford, Mass., 1995/96), the entire teaching of which is incorporated herein by reference. Ultrafiltration is generally considered to mean filtration us ing filters with a pore size of smaller than 0.1 μ [symbol] . By employing filters having such a small pore size, the volume of sample can be reduced through permeation of the sample buffer through the filter membrane pores while proteins are retained above the membrane surface.

There are typically three to four stages of UF/DF: initial concentration, diafiltration, secondary concentration, and final concentration. During initial concentration, TMP drives water and salts across the permeable membrane, which reduces the liquid volume and thus increases protein concentration. The extent of concentration in initial concentration may be optimized to balance throughput and protein stability.

During the diafiltration stage, permeable solutes are replaced as new buffer is washed into the product stream. When new buffer is added at the same rate as permeate is removed from the system, the sum of the retentate tank and skid volume defines the system volume. One turn-over volume (TOV) is defined as an amount of diafiltration buffer added to the UF/DF process, and matches the system volume. Typically, replacement of 8-times system volume (8 TOV) assures >99.9% buffer exchange. The diafiltration buffer is intended to condition the protein to a stable pH and excipient concentration that compensates for high product concentration.

Once adequate buffer exchange has taken place, secondary concentration further reduces product volume to facilitate storage and formulation. If additional reduction in product volume is required, for example due to plant fit considerations, higher final protein concentration requirements, or required adjustment with viscosity modulators, final concentration is performed.

One of ordinary skill in the art can select an appropriate membrane filter device for the UF/DF operation. Examples of membrane cassettes suitable for the present invention include, but are not limited to, Pellicon 2 or Pellicon 3 cassettes with 10 kD, 30 kD or 50 kD membranes from EMD Millipore, Kvick 10 kD, 30 kD or 50 kD membrane cassettes from Cytiva, and Centramate or Centrasette 10 kD, 30 kD or 50 kD cassettes from Pall Corporation.

Exemplary steps are described in more detail here. A first UF/DF step comprises a WFI flush, with a volume of about 12 L/m² with respect to the retentate and 40 L/m² with respect to the permeate. A feed flowrate is between about 200 and 300 L/hr/m² (LMH), with a pressure of about 25±5 psi. Next, a pre-use leak test is performed with WFI, with an inlet pressure of 8-12 psi. Equilibration is then performed with about 4 mM acetate at about pH 4.10±0.10. A volume of equilibration buffer is greater than or about 5 L/m², and the flowrate is between about 200 and 300 LMH.

The sample to be concentrated (load) is then adjusted for compatibility with the final drug substance composition. A load adjustment solution may be, for example, about 10% (w/v) super refined polysorbate 80 or polysorbate 20, added as about 50 μL/L of load.

Next, the initial concentration step is performed with the load, for example a VRF pool. The load is added at a volume of about or less than 970 g/m², with a flowrate of between about 200 and about 350 LMH, and a pressure of about 20±10 psi. In an exemplary embodiment, a flow rate is about 300 LMH. A protein concentration after initial concentration may be between about 60 and 80 g/L. In an exemplary embodiment, a protein concentration at this step is about 70 g/L.

Following the initial concentration step is a diafiltration step. A diafiltration buffer may comprise about 4 mM acetate at a pH between about 4.00 and 4.20. In some embodiments, a concentration of acetate in a diafiltration buffer is between about 3 mM and about 10 mM, between about 3 mM and about 5 mM, about 3 mM, about 3.5 mM, about 4 mM, about 4.5 mM, about 5 mM, about 5.5 mM, about 6 mM, about 6.5 mM, about 7 mM, about 7.5 mM, about 8 mM, about 8.5 mM, about 9 mM, about 9.5 mM, or about 10 mM. In some embodiments, a pH of a diafiltration buffer is between about 4.00 and about 4.50, about 4.00, about 4.05, about 4.10, about 4.15, about 4.20, about 4.25, about 4.30, about 4.35, about 4.40, about 4.45, or about 4.50. In an exemplary embodiment, a pH of a diafiltration buffer is about 4.10.

A volume of diafiltration buffer used may be about or more than 8 TOV, at a flowrate between about 200 and 350 LMH, and a pressure between about 10 and 30 psi. In an exemplary embodiment, a flow rate for diafiltration is about 300 LMH. In an exemplary embodiment, a pressure for diafiltration is about 20 psi. A protein concentration after diafiltration may be between about 60 and about 80 g/L. In an exemplary embodiment, a protein concentration after diafiltration is about 70 g/L.

After diafiltration is a secondary concentration step. A product quantity used for secondary concentration is about or less than 970 g/m². A flowrate for secondary concentration is between about 100 and about 350 LMH. In an exemplary embodiment, a flowrate is about 300 LMH. A pressure for secondary concentration is between about 10 and 30 psi, with less than 60 psi inlet pressure. A protein concentration after secondary concentration may be between about 80 and 100 g/L. In an exemplary embodiment, a protein concentration after secondary concentration is about 90 g/L.

Following secondary concentration is a membrane depolarization step. This process may have a duration of between about 5 and 15 minutes, with a flow rate of about or less than 350 LMH, at a pressure of less than 30 psi. For this step, the permeate valve is 0% open while the retentate valve is 100% open.

Next is a final, or third, concentration step. A quantity of final concentration load used may be about or less than 1800 g/m², added at a flow rate of between about 5 and 350 LMH and an inlet pressure of less than 60 psi. In some embodiments, a final protein concentration is between about 180 g/L and about 260 g/L, between about 225 g/L and about 255 g/L, between about 228 g/L and about 232 g/L, about 180 g/L, about 185 g/L, about 190 g/L, about 195 g/L, about 200 g/L, about 205 g/L, about 210 g/L, about 215 g/L, about 220 g/L, about 225 g/L, about 230 g/L, about 235 g/L, about 240 g/L, about 245 g/L, about 250 g/L, about 255 g/L, or about 260 g/L. In an exemplary embodiment, a final protein concentration is about 232 g/L. In another exemplary embodiment, a final protein concentration is about 240 g/L.

Following final concentration, the process includes another membrane depolarization step conducted for between about 15 and 35 minutes. In an exemplary embodiment, this step has a duration of about 25 minutes. A feed flowrate is about or less than 350 LMH, with a pressure below 30 psi, the permeate valve 0% open, and the retentate valve 100% open.

Finally, following membrane polarization there is a WFI rinse step, with an application of 10 L/m² WFI at a flowrate between about 200 and about 250 LMH.

### Formulation

Certain exemplary embodiments comprise further adjusting a final concentrated pool (FCP) protein following a concentration and diafiltration step. A concentrated protein may be adjusted to the desired concentration and with the desired excipients for a final drug substance (FDS).

If a filtered FCP protein concentration is above a specified target, it is diluted with FCP dilution buffer to adjust it to a target concentration. FCP dilution buffer may comprise about 10 mM sodium acetate, about 25 mM arginine hydrochloride, and about 5% sucrose, at a pH of about 5.3. In some embodiments, a concentration of sodium acetate in FCP dilution buffer can be between about 2.0 mM to about 14.0 mM, between about 6.0 mM to about 12.0 mM, between about 8.0 mM to about 10.0 mM, about 2.0 mM, about 2.5 mM, about 3.0 mM, about 3.5 mM, about 4.0 mM, about 4.5 mM, about 5 mM, about 5.5 mM, about 6.0 mM, about 6.5 mM, about 7.0 mM, about 7.5 mM, about 8.0 mM, about 8.5 mM, about 9.0 mM, about 9.5 mM, about 10.0 mM, about 10.5 mM, about 11.0 mM, about 11.5 mM, about 12.0 mM, about 12.5 mM, about 13.0 mM, about 13.5 mM or about 14.0 mM. In some embodiments, a concentration of arginine hydrochloride in FCP dilution buffer can be between about 10 mM and about 40 mM, between about 15 mM and about 35 mM, between about 20 mM and about 30 mM, about 15 mM, about 20 mM, about 25 mM, about 30 mM, about 35 mM or about 40 mM. In some embodiments, a concentration of sucrose in FCP dilution buffer can be between about 1% and 10%, between about 2% and 9%, between about 3% and 8%, about 1%, about 2%, about 3%, about 4%, about 5%, about 6%, about 7%, about 8%, about 9% or about 10%. In some embodiments, a pH of FCP dilution buffer can be between about 4.0 and about 6.5, between about 4.5 and about 6.0, between about 5.0 and about 5.5, about 4.0, about 4.5, about 5.0, about 5.5, about 6.0, about 6.5, or about 7.0.

In some embodiments, FCP may be further combined with an excipient concentrate buffer both to achieve a target dilution of the FCP protein, and to achieve a target excipient concentration. An FCP excipient concentration buffer may comprise about 10 mM acetate, about 350 mM arginine hydrochloride, and about 70% (w/v) sucrose, at a pH of about 5.3. In some embodiments, a concentration of acetate in an excipient concentrate buffer can be between about 2.0 mM to about 14.0 mM, between about 6.0 mM to about 12.0 mM, between about 8.0 mM to about 10.0 mM, about 2.0 mM, about 2.5 mM, about 3.0 mM, about 3.5 mM, about 4.0 mM, about 4.5 mM, about 5.0 mM, about 5.5 mM, about 6.0 mM, about 6.5 mM, about 7.0 mM, about 7.5 mM, about 8.0 mM, about 8.5 mM, about 9.0 mM, about 9.5 mM, about 10.0 mM, about 10.5 mM, about 11.0 mM, about 11.5 mM, about 12.0 mM, about 12.5 mM, about 13.0 mM, about 13.5 mM or about 14.0 mM. In some embodiments, a concentration of arginine hydrochloride in an excipient concentrate buffer can be between about 200 mM and about 500 mM, between about 250 mM and about 450 mM, between about 300 mM and about 400 mM, about 200 mM, about 250 mM, about 300 mM, about 350 mM, about 400 mM, about 450 mM, or about 500 mM. In some embodiments, a concentration of sucrose in an excipient concentrate buffer can be between 50% and 90%, between about 55% and 85%, between about 60% and 80%, about 50%, about 55%, about 60%, about 65%, about 70%, about 75%, about 80%, or about 85%. In some embodiments, a pH of an excipient concentrate buffer can be between about 4.0 and about 6.5, between about 4.5 and about 6.0, between about 5.0 and about 5.5, about 4.0, about 4.5, about 5.0, about 5.5, about 6.0, about 6.5, or about 7.0.

Following dilution with dilution buffer and combination with excipient concentrate buffer, an adjusted FCP may be filtered into a single-use bag and mixed. The sample may be referred to as filtered drug substance (DS). Filtered DS may be further diluted to a target concentration with FDS dilution buffer and compounded with excipient buffer solution to a final concentration and composition.

A target concentration may be about 200.0 mg/mL. In some embodiments, a target concentration is between about 160.0 mg/mL and about 240.0 mg/mL, between about 180.0 mg/mL and about 220.0 mg/mL, between about 190.0 mg/mL and about 210.0 mg/mL, between about 195.0 mg/mL and about 205.0 mg/mL, between about 199.0 mg/mL and about 201.0 mg/mL, between about 199.5 mg/mL and about 200.5 mg/mL, between about 199.9 mg/mL and about 200.1 mg/mL, about 160.0 mg/mL, about 165.0 mg/mL, about 170.0 mg/mL, about 175.0 mg/mL, about 180.0 mg/mL, about 185.0 mg/mL, about 190.0 mg/mL, about 195.0 mg/mL, about 199.0 mg/mL, about 199.5 mg/mL, about 199.9 mg/mL, about 200.0 mg/mL, about 200.1 mg/mL, about 200.5 mg/mL, about 201.0 mg/mL, about 205.0 mg/mL, about 210.0 mg/mL, about 215.0 mg/mL, about 220.0 mg/mL, about 225.0 mg/mL, about 230.0 mg/mL, about 235.0 mg/mL, or about 240.0 mg/mL.

FDS dilution buffer may comprise about 10 mM sodium acetate, about 25 mM arginine hydrochloride, and about 5% sucrose, at a pH of about 5.3. In some embodiments, a concentration of sodium acetate in FDS dilution buffer can be between about 2.0 mM to about 14.0 mM, between about 6.0 mM to about 12.0 mM, between about 8.0 mM to about 10.0 mM, about 2.0 mM, about 2.5 mM, about 3.0 mM, about 3.5 mM, about 4.0 mM, about 4.5 mM, about 5 mM, about 5.5 mM, about 6.0 mM, about 6.5 mM, about 7.0 mM, about 7.5 mM, about 8.0 mM, about 8.5 mM, about 9.0 mM, about 9.5 mM, about 10.0 mM, about 10.5 mM, about 11.0 mM, about 11.5 mM, about 12.0 mM, about 12.5 mM, about 13.0 mM, about 13.5 mM or about 14.0 mM. In some embodiments, a concentration of arginine hydrochloride in FDS dilution buffer can be between about 10 mM and about 40 mM, between about 15 mM and about 35 mM, between about 20 mM and about 30 mM, about 15 mM, about 20 mM, about 25 mM, about 30 mM, about 35 mM or about 40 mM. In some embodiments, a concentration of sucrose in FDS dilution buffer can be between about 1% and 10%, between about 2% and 9%, between about 3% and 8%, about 1%, about 2%, about 3%, about 4%, about 5%, about 6%, about 7%, about 8%, about 9% or about 10%. In some embodiments, a pH of FDS dilution buffer can be between about 4.0 and about 6.5, between about 4.5 and about 6.0, between about 5.0 and about 5.5, about 4.0, about 4.5, about 5.0, about 5.5, about 6.0, about 6.5, or about 7.0.

A composition of excipient buffer solution may be selected depending on the target concentration and composition of a final drug substance (FDS).

An excipient buffer solution for a target FDS concentration of 150 mg/mL may comprise about 20 mM sodium acetate, about 80 mM L-histidine, about 25 mM L-arginine hydrochloride, about 5% sucrose, and about 0.8% polysorbate 80, at a pH of about 6.7. In some embodiments, a concentration of sodium acetate in FDS excipient buffer solution (for a target FDS concentration of 150 mg/mL) can be between about 5 mM and about 35 mM, between about 10 mM and about 30 mM, between about 15 mM and about 25 mM, about 10 mM, about 15 mM, about 20 mM, about 25 mM, about 30 mM, or about 35 mM. In some embodiments, a concentration of L-histidine in FDS excipient buffer solution (for a target FDS concentration of 150 mg/mL) can be between about 60 mM and about 100 mM, between about 65 mM and about 95 mM, between about 70 mM and about 90 mM, between about 75 mM and about 85 mM, about 60 mM, about 70 mM, about 80 mM, or about 90 mM. In some embodiments, a concentration of L-arginine hydrochloride in FDS excipient buffer solution (for a target FDS concentration of 150 mg/mL) can be between about 10 mM and about 40 mM, between about 15 mM and about 35 mM, between about 20 mM and about 30 mM, about 15 mM, about 20 mM, about 25 mM, about 30 mM, about 35 mM or about 40 mM. In some embodiments, a concentration of sucrose in FDS excipient buffer solution (for a target FDS concentration of 150 mg/mL) can be between about 1% and 10%, between about 2% and 9%, between about 3% and 8%, about 1%, about 2%, about 3%, about 4%, about 5%, about 6%, about 7%, about 8%, about 9% or about 10%. In some embodiments, a concentration of polysorbate 80 in FDS excipient buffer solution (for a target FDS concentration of 150 mg/mL) can be between about 0.1% and 1.6%, between about 0.3% and 1.3%, between about 0.6% and 1.1%, between about 0.8% and 0.9%, about 0.2%, about 0.4%, about 0.6%, about 0.8%, about 1.0%, about 1.2%, about 1.4%, or about 1.6%. In some embodiments, a pH of FDS excipient buffer solution (for a target FDS concentration of 150 mg/mL) can be between about 5.0 and about 8.0, between about 5.5 and about 7.5, between about 6.0 and about 7.0, about 5.0, about 5.5, about 6.0, about 6.5, about 7.0, or about 7.5.

An excipient buffer solution for a target FDS concentration of 175 mg/mL may comprise about 30 mM sodium acetate, about 160 mM L-histidine, about 225 mM L-arginine hydrochloride, about 5% (w/v) sucrose, and about 1.6% (w/v) polysorbate 80, at a pH of about 7.0. In some embodiments, a concentration of sodium acetate in FDS excipient buffer solution (for a target FDS concentration of 175 mg/mL) can be between about 15 mM and about 45 mM, between about 20 mM and about 40 mM, between about 25 mM and about 35 mM, about 20 mM, about 25 mM, about 30 mM, about 35 mM, about 40 mM, or about 45 mM. In some embodiments, a concentration of L-histidine in FDS excipient buffer solution (for a target FDS concentration of 175 mg/mL) can be can be between about 120 mM and about 200 mM, between about 130 mM and about 190 mM, between about 140 mM and about 180 mM, between about 150 mM and about 170 mM, about 140 mM, about 150 mM, about 160 mM, about 170 mM, or about 180 mM. In some embodiments, a concentration of L-arginine hydrochloride in FDS excipient buffer solution (for a target FDS concentration of 175 mg/mL) can be between about 125 mM and about 325 mM, between about 150 mM and about 300 mM, between about 175 mM and about 275 mM, between about 200 mM and about 250 mM, about 150 mM, about 175 mM, about 200 mM, about 225, mM, about 275 mM, about 300 mM, or about 325 mM. In some embodiments, a concentration of sucrose in FDS excipient buffer solution (for a target FDS concentration of 175 mg/mL) can be between about 1% and 10%, between about 2% and 9%, between about 3% and 8%, about 1%, about 2%, about 3%, about 4%, about 5%, about 6%, about 7%, about 8%, about 9% or about 10%. In some embodiments, a concentration of polysorbate 80 in FDS excipient buffer solution (for a target FDS concentration of 175 mg/mL) can be between about 0.8% and 2.4%, between about 1.0% and 2.2%, between about 1.2% and 2.0%, between about 1.4% and 1.8%, about 0.8%, about 1.2%, about 1.4%, about 1.6%, about 1.8%, about 2.0%, about 2.2%, or about 2.4%. In some embodiments, a pH of FDS excipient buffer solution (for a target FDS concentration of 175 mg/mL) can be between about 5.5 and about 8.5, between about 6.0 and about 8.0, between about 6.5 and about 7.5, about 5.5, about 6.0, about 6.5, about 7.0, about 7.5. or about 8.0.

### EXAMPLES

### Example 1. Insulin Supplementation

Total insulin concentration (0 mg/L, 5 mg/L, 10 mg/L, 15 mg/L) was assessed in combination with an insulin schedule (Total on Day 4; Split between Days 2, 4; Split between days 2, 4, 6, 8).

A modified seed train using normal operating conditions in flasks and bioreactors was used to inoculate the custom D-optimal design as shown in the table below (Table 9). A 15-day fed batch process (Table 10) was run in 2 L bioreactors with CDM1B comprising taurine and sodium phosphate.

TABLE 9
(Insulin Supplementation Experiment Conditions)

| Experiment Factor | Factor Type | Level 1 | Level 2 | Level 3 | Level 4 |
|---|---|---|---|---|---|
| Inoculation Density | Continuous | 4.5 × 10⁶ viable | 5.5 × 10⁶ viable | 8.0 × 10⁶ viable | |
| | | cells/mL | cells/mL | cells/mL | |
| Insulin Schedule | Categorical | Day 4 | Day 2, 4 | Day 2, 4, 6, 8 | |
| Insulin Concentration | Continuous | 0 mg/L | 5 mg/L | 10 mg/L | 15 mg/L |

TABLE 10
(Insulin Supplementation 2 L Bioreactor Conditions)
Culture Conditions

| | |
|---|---|
| Insulin Feed | 0 mg/L or |
| | 5 mg/L or |
| | 10 mg/L or |
| | 15 mg/L |
| Insulin Feed Day | None or |
| | Day 4 or |
| | Days 2, 4 or |
| | Days 2, 4, 6, 8 |

Bioreactor samples were measured on the Nova Flex analyzer for cell count and metabolites. Upon completion of the production, samples were analyzed for titer analysis. While the production duration was 15 days, day 13 bioreactor samples were analyzed for product quality analyses.

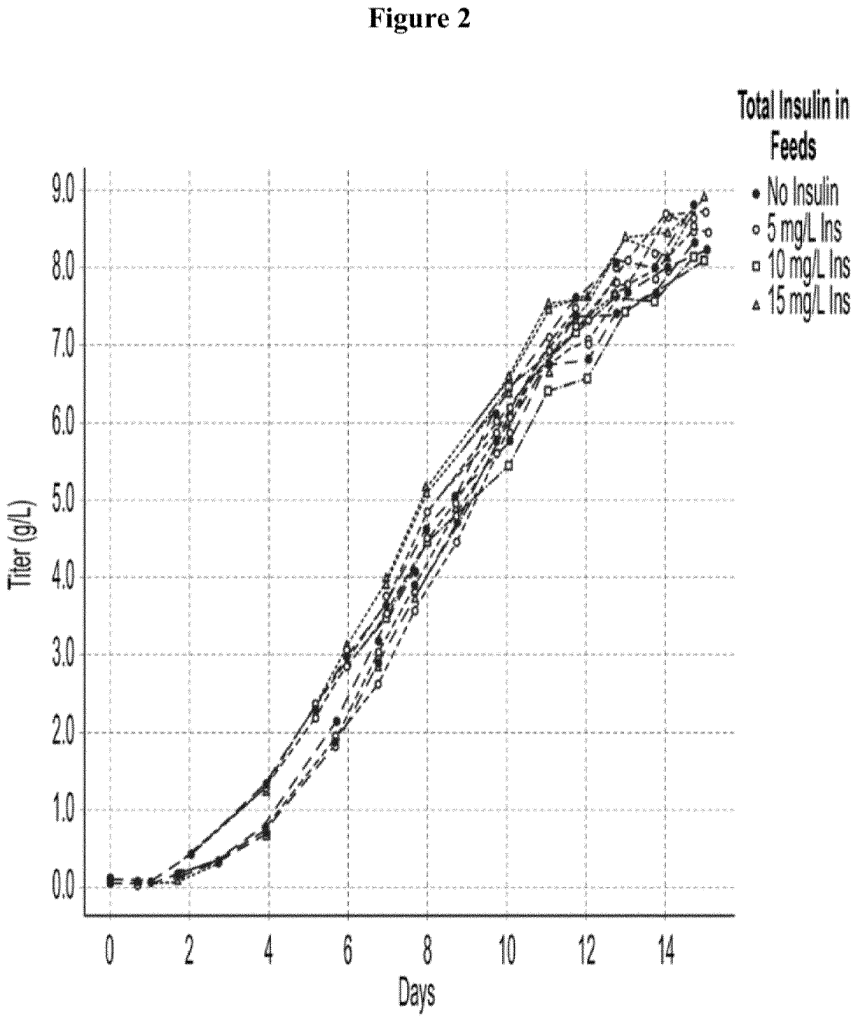

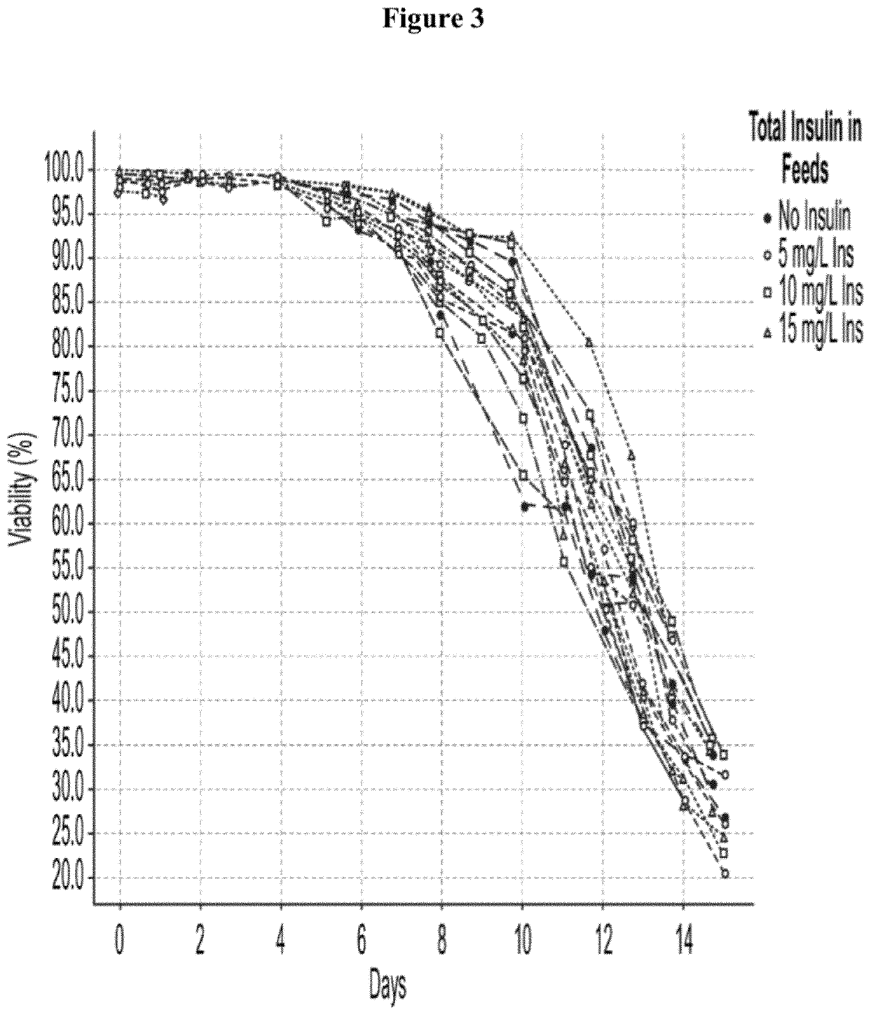

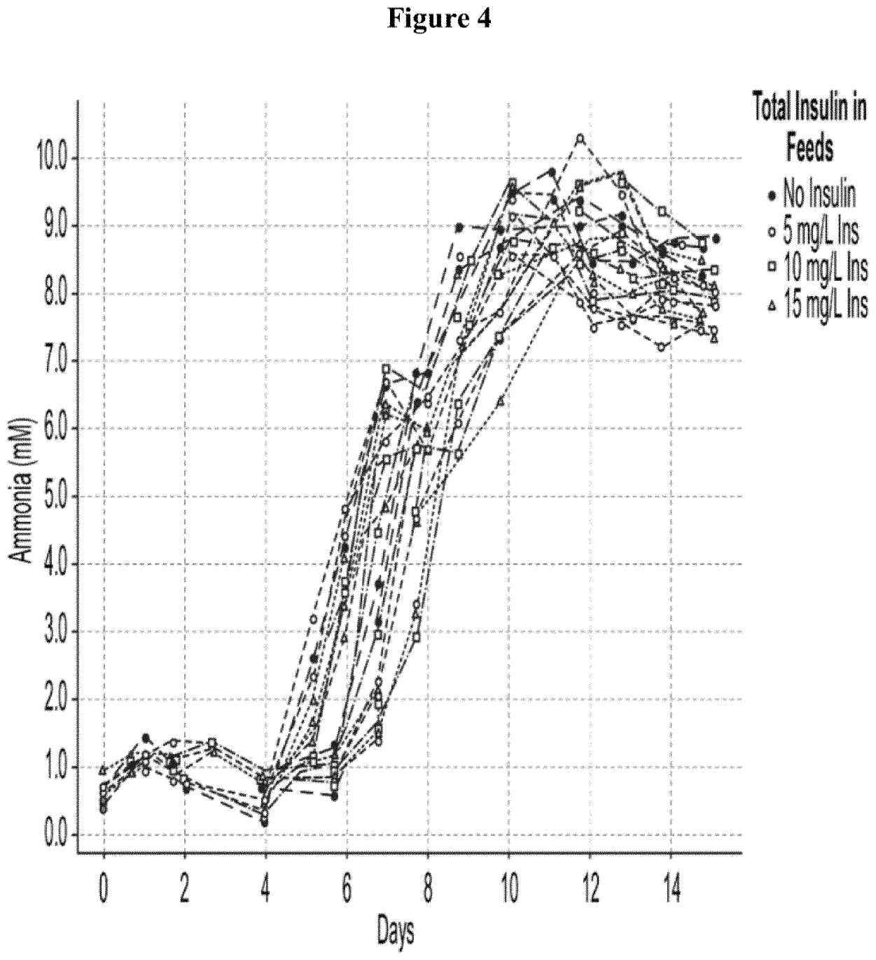

Increased insulin resulted in higher productivity (FIG. 2), higher viability (FIG. 3), and overall reduced ammonia (FIG. 4) in some of the bioreactors. Higher inoculation densities also impacted the titer curve parameters and resulted in lower ammonia and viability.

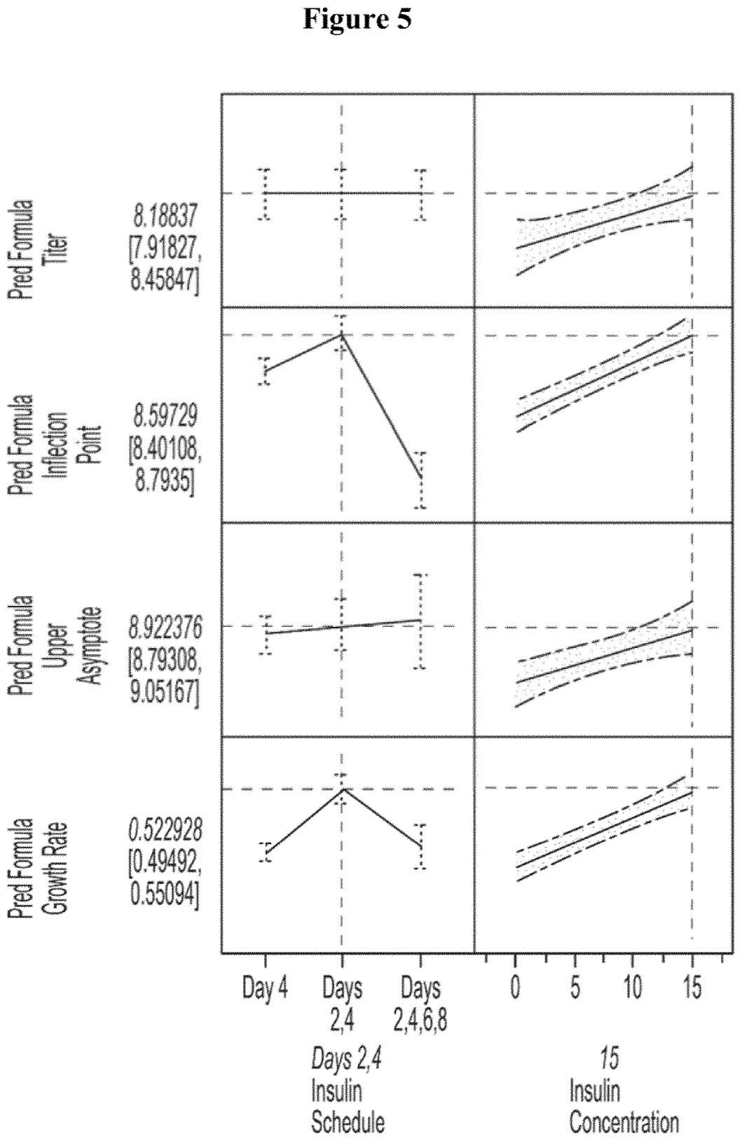

From the JMP optimization (FIG. 5), it was determined to increase the insulin concentration to 15 mg/L. Providing the insulin on day 2 and 4 was preferential for maximum titer. As a result, the insulin feed strategy of 7.5 mg/L on days 2 and 4 was found optimal.

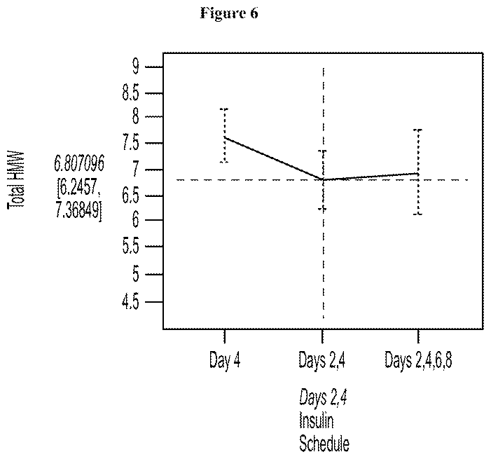

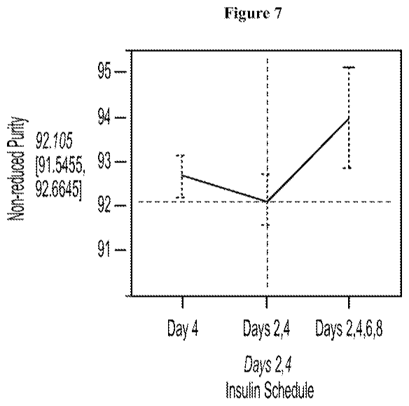

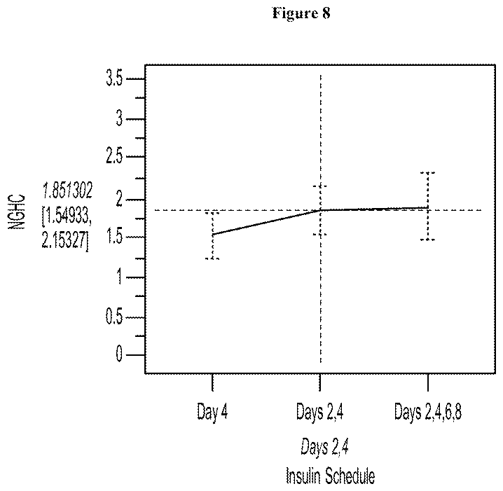

Product quality was periodically monitored throughout medium and feed development. Day 13 bioreactor harvest samples were submitted for product quality analysis. Galactosylation and fucosylation were not impacted by process variables. The impacts of insulin feeding schedule were assessed for aggregate (FIG. 6), non-reduced purity (FIG. 7), and NGHC (FIG. 8). Impacts to these product quality attributes from insulin feeding schedule were minor and within development targets.

### Example 2. Tyrosine Supplementation

It was noted that several amino acids were depleted in the increased productivity process. As a result, additional tyrosine feeding was studied to address the depletions.

A modified seed train using normal operating conditions in flasks was used to inoculate the flask custom D-optimal design as shown in the table below (Table 11). A 15-day fed batch process was run in 250 mL flasks with cell culture medium comprising sodium phosphate and taurine. Tyrosine was supplemented on day 3 of production. Tyrosine concentration varied from 1.8 g/L to 2.2 g/L, as per the experimental design (Table 11).

TABLE 11

| Experiment | | | | | |
|---|---|---|---|---|---|
| Factor | Factor Type | Level 1 | Level 2 | Level 3 | Level 4 |
| Tyrosine | Continuous | 1.8 g/L | 2.0 g/L | 2.2 g/L | N/A |
| Concentration | | | | | |

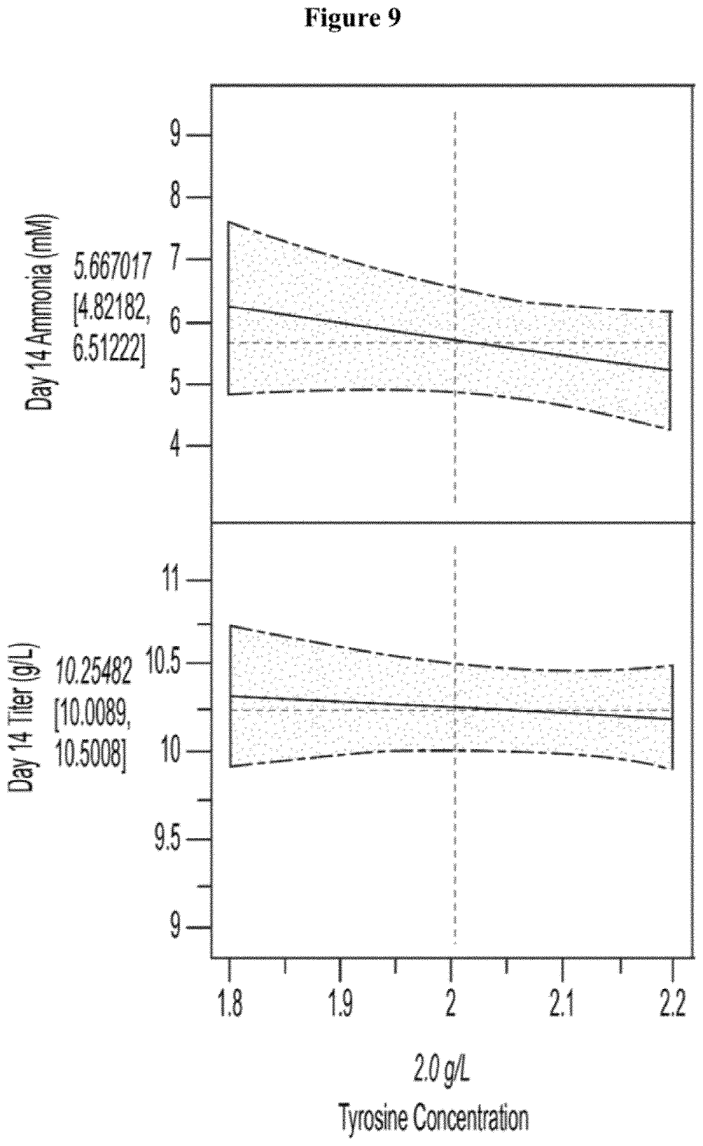

Feeding additional tyrosine to cell production resulted in decreased ammonia and had minimal impact to productivity, as shown in FIG. 9.

### Example 3. Phosphate Redistribution

Phosphate is essential for energy transfer in the cell and synthesis of nucleic acids and phospholipids. Phosphate depletions can cause a decrease in cell viability and accumulation of unwanted cellular metabolic by-products. Initial small-scale model development experiments indicated a potential phosphate depletion by Day 2. As a result, phosphate redistribution, including increased basal medium concentrations, was studied.

Phosphate feeding schedules were evaluated in the production flask model. Avoiding depletion of key nutrients, phosphate included, is fundamental and necessary prior to starting more complex medium and feed studies.

A modified seed train was used to inoculate the production flask experiment that utilized flasks and seed train bioreactors. A fed batch process with the chosen cell line was run in flasks with the starting production medium comprising taurine. Levels of sodium phosphate were varied in the starting medium, as per the experiment design. Sodium phosphate was also varied in the four feeds throughout production (n=2 for each condition, see Table 12).

TABLE 12

| | Experimental Factor: Sodium Phosphate | | | |
|---|---|---|---|---|
| Production Medium | (Feed Concentration (mg/L)) | | | |
| Concentration (mg/L)* | Day n | Day 2n | Day 3n | Day 4n |
| 0 | 525 | 525 | 350 | 350 |
| 267 (1.88 mM) | 525 | 525 | 250 | 250 |
| 267 (1.88 mM) | 525 | 525 | 0 | 0 |
| 267 (1.88 mM) | 350 | 350 | 200 | 150 |

*Component level defined as concentration prior to inoculation

Daily samples were analyzed on the Nova CDV for cell count, the Nova BP400 for metabolites, and the Advanced Instruments Model 2020 Osmometer for osmolality. Samples were also submitted for titer analysis.

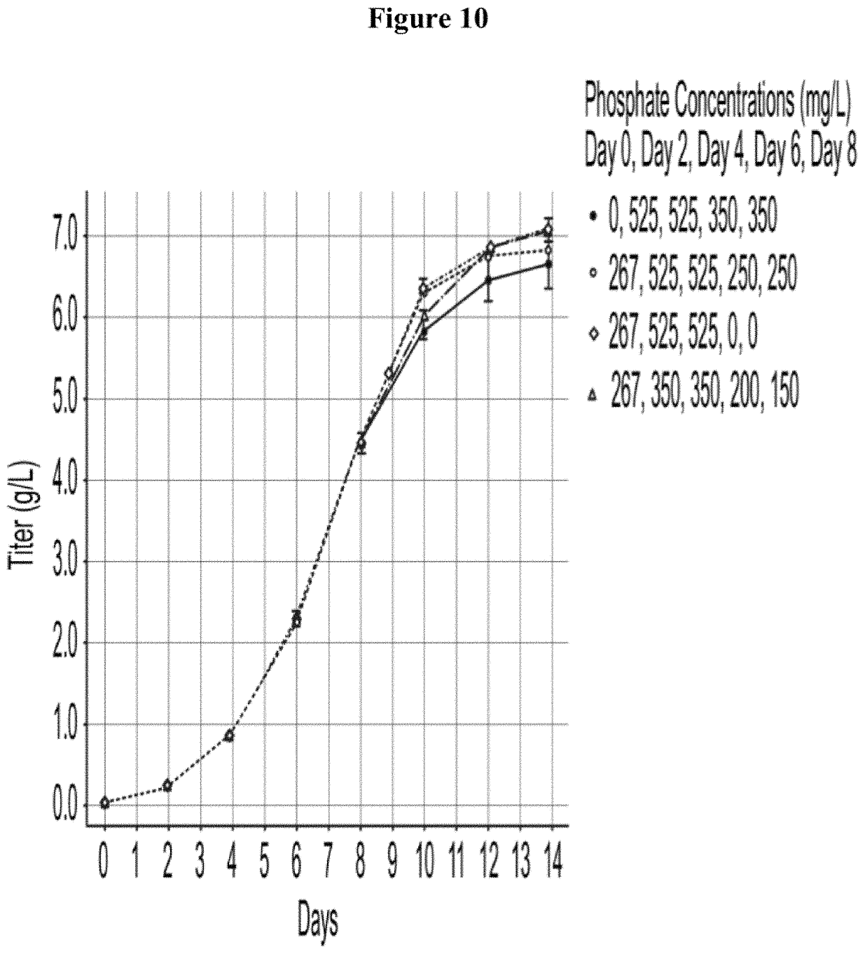

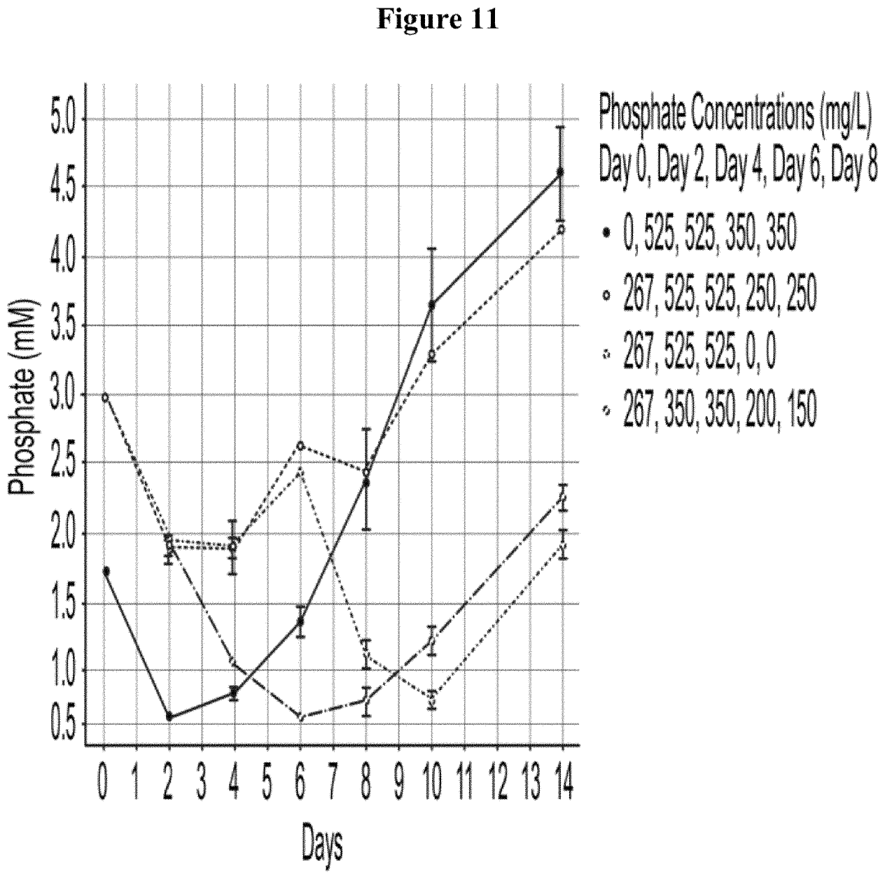

The phosphate redistribution strategies resulted in minor increases in final titer (FIG. 10). The different feeding paradigms changed the shapes of the phosphate concentration curves, and three conditions neared depletion at various production days (FIG. 11). The condition with increased phosphate early in the batch and continued phosphate feeding through the end of the batch (267, 525, 525, 250, 250 mg/L) did not deplete of phosphate during production; this feeding strategy was implemented for the upstream process.

### Example 4. Production Bioreactor Conditions

Cell culture parameters in the production bioreactor are expected to have the most influence on both product quality and process productivity since most of the protein is produced in this unit operation.

A culture from a seed train inoculated two N-3 10 L stirred tank bioreactors. After the N-3 final density was reached, three N-2 10 L stirred tank bioreactors were inoculated. Four N-1 10 L stirred tank bioreactors were inoculated after the N-2 final density was reached. Appropriate split ratios were used throughout the seed train expansion. At the three specified final N-1 density targets, 2 L fed batch production bioreactors were inoculated. Three of the four N-1 reactors were utilized to inoculate the production bioreactors. In addition to the inoculation relevant variable of density, temperature and pH Upper Limit were set according to experiment design, and the pH lower limit was held constant.

Daily samples were analyzed on the Nova FLEX for cell count and metabolites. Upon completion of the production batch, Day 10, 12, and/or 13 samples were analyzed for titer analysis and quality testing. Sixteen samples from Day 10, 12 and/or 13 were also analyzed for sequence variants.

It was determined that the harvest day had a large impact on preliminary general quality attributes, process attributes, and sequence variants (titer, total fucosylation, total galactosylation, viability, total Pro-Ala SV, Pro-Ala Site P12). Harvest day is a timing-dependent upstream process parameter where an operating range is defined considering the manufacturing facility capabilities and flexibility. Monte Carlo simulations were performed to determine the combination of ranges. The optimal profiler, based on the IMP desirability maximization algorithm, determined Day 10.0 as the optimal set point.

Monte Carlo simulations were performed on N-1 Density to characterize failure rates on process and quality attributes for process ranges tested in combination. Ideal process ranges were then chosen based on achieving the most desirable manufacturing process with an emphasis on minimizing failure rates (target failure rate of <100 ppm, 0.01%). Failures primarily occurred in meeting Cys-Tyr Substitution, SDS LMW, SDS Purity, SDS Purity (HC+LC+NGHC), SEC HMW Aggregate Dimer, and SEC HMW Aggregate Higher Order targets. Increasing the N-1 Density upper limit minimally impacted quality attributes with the large impact (2.0% increase) to Cys-Tyr Substitution. The N-1 Density range was set to 4.35−5.45×10⁵ cells/mL to balance quality attribute responses and manufacturing flexibility.

Increasing the upper limit of harvest day increases NR SDS LMW failure by 70% and decreases SEC HMW Aggregate Higher Order by 22%. The increase of NR SDS LMW does at the same time decrease SDS LMW with harvest length due to decreasing trisulfides. Due to this discrepancy and to increase ease of manufacturing, the harvest day range was set to 10-11 days. Final harvest day range and N-1 density range of 5 standard deviations were chosen for the final process to balance tighter control (lower failure rates) and manufacturing flexibility. A summary of simulation conditions can be found in Table 13 (Production Simulation Conditions Summary).

TABLE 13
(Production Simulation Conditions Summary)

| Factor | Range 1 | Range 2 |
|---|---|---|
| Harvest Day (Day) | 10.0-10.5 | 10.0-11.0 |
| N-1 Density (1 × 10⁶ cell/mL) | 4.35-5.23 | 4.35-5.45 |

### Harvest Overview

The harvesting process may begin at the separation step after the recombinant protein has been produced using upstream production methods described above and/or by alternative production methods conventional in the art. When using the cell culture techniques of the instant disclosure, a protein of interest can be produced intracellularly, in the periplasmic space, or directly secreted into the medium. In embodiments where the protein of interest is produced intracellularly, particulate debris—either host cells or lysed cells (e.g., resulting from homogenization) can be removed by a variety of means, including, but not limited to, centrifugation or ultrafiltration. Where the protein of interest is secreted into the medium, supernatants from such expression systems can be first concentrated using a commercially available protein concentration filter, for example, using an Amicon™ or Millipore Pellicon™ ultrafiltration unit. In one aspect, the protein of interest may be harvested by centrifugation followed by depth filtration and then affinity capture chromatography.

A variety of different production techniques, including, but not limited to, affinity, ion exchange, mixed-mode, size exclusion, and hydrophobic interaction chromatography, singularly or in combination, are envisaged to be within the scope of the present invention. These chromatographic steps separate mixtures of proteins of a biological sample on the basis of their charge, degree of hydrophobicity, or size, or a combination thereof, depending on the particular form of separation. Several different chromatography resins are available for each of the techniques alluded to supra, allowing accurate tailoring of the production scheme to a particular protein involved. Each separation method results in the protein traversing at different rates through a column to achieve a physical separation that increases as they pass further through the column or adhere selectively to a separation medium. The proteins are then either (i) differentially eluted using an appropriate elution buffer and/or (ii) collected from flowthrough fractions obtained from the column used, optionally, from washing the column with an appropriate equilibration buffer. In some cases, the protein of interest is separated from impurities (HCPs, protein variants, etc.) when the impurities preferentially adhere to the column and the protein of interest less so, e.g., the protein of interest does not adsorb to the solid phase of a particular column and thus flows through the column. In some cases, the impurities are separated from the protein of interest when they fail to adsorb to the column and thus flow through the column.

The harvesting process may begin at the separation step after the recombinant protein has been produced using upstream production methods described above and/or by alternative production methods conventional in the art. Once a clarified solution or mixture comprising the protein of interest, for example, Dupilumab, has been obtained, separation of the protein of interest from process-related impurities (such as the other proteins produced by the cell (like HCPs), as well as product-related substances, such acidic or basic variants) is performed. A combination of one or more different production techniques, including affinity, ion exchange (e.g., CEX, AEX), mixed-mode (MM), and/or hydrophobic interaction chromatography can be employed. Such production steps separate mixtures of components within a biological sample on the basis of their, for example, charge, degree of hydrophobicity, and/or apparent size. Numerous chromatography resins are commercially available for each of the chromatography techniques mentioned herein, allowing accurate tailoring of the production scheme to a particular protein involved. Each of the separation methods allow proteins to either traverse at different rates through a column achieving a physical separation that increases as they pass further through the column or to adsorb selectively to a separation resin (or medium). The proteins can then be differentially collected. In some cases, the protein of interest is separated from components of a biological sample when other components specifically adsorb to a column's resin while the protein of interest does not.

Column loading and washing steps can be controlled by in-line, at-line or off-line measurement of the product-related impurity/substance levels, either in the column effluent, or the collected pool, or both, so as to achieve a particular target product quality and/or yield. In certain embodiments, the loading concentration can be dynamically controlled by in-line or batch or continuous dilutions with buffers or other solutions to achieve the partitioning necessary to improve the separation efficiency and/or yield.

### Harvest Pre-Treatments

In certain exemplary embodiments, the harvest process can include an initial step for retarding enzymatic processes and reducing formations of aggregates. Primary recovery may be combined with the use of pre-treatments. Pre-treatments are designed to improve the harvest step through the use of additives that aid in cell removal and help reduce process related impurity levels, thus lowering the burden on the downstream chromatographic steps. Pre-treatments include flocculation or precipitation, and both approaches promote association or clumping of smaller particulates to form larger solids that can be removed more effectively. Non-limiting examples of these pre-treatments include polymeric flocculants, Chitosan, Poly(ethyleneimine) and cationic salts such as calcium phosphate.

Examples of harvest pre-treatments that may be used in the present invention are described more fully in the Examples below. In one aspect, this step can be performed by the process of cooling the bioreactor to about (±3° C.) 13.5° C., 14.5° C., 15.5° C., 16.5° C., 17.5° C., 18.5° C., 19.5° C., 20.5° C., 21.5° C., 22.5° C. or 23.5° C. Maintaining a narrow temperature range has also been shown to limit changes to impurity solubility or polish filter binging in the proceeding steps.

### Example 5: Chitosan Flocculant Pre-Treatment

In one example, Chitosan was solubilized in 1% (w/v) acetic acid, whilst a 125 mM stock solution of calcium phosphate was created from equal parts of 500 mM sodium phosphate and 800 mM calcium chloride. Cell culture fluid for the polyethylenimine (PEI) and Chitosan originated from BRX161-10128 and material for the calcium phosphate originated from two developmental bioreactors, TD29-10437 and TD32-10438. Pools of cell culture fluid were adjusted to three different pH values using acetic acid: native pH, pH 6.5 and the midpoint pH. From these three different pools, aliquots of each pH were adjusted to three different conductivity ranges; native, 1.5× native and 0.5× native, either by the addition of NaCl or dilution using RODI.

An overview of the samples tested can be seen in Table 14, Table 15, and Table 16 below.

TABLE 14
(Overview of flocculation central composite design (CCD) design of experiments
(DoE) study performed for PEI, CF = clearance factor (Load_{HCP}/Pool_{HCP})

| | [PEI] | | | Conductivity | | | |
|---|---|---|---|---|---|---|---|
| | (% w/v) | Mixing Time (s) | pH | (×control) | NTU Normalized | Yield | HCP (CF) |
| P-1 | 0.1 | 120 | 7.38 | 1.5 | 3.05 | 98.8 | 0.6 |
| P-2 | 0.1 | 65 | 6.5 | 1 | 3.41 | 99.2 | 0.7 |
| P-3 | 0.1 | 10 | 6.5 | 1.5 | 3.44 | 102.2 | 0.6 |
| P-4 | 0.55 | 65 | 6.94 | 1 | 2.54 | 85.4 | 0.5 |
| P-5 | 1 | 10 | 6.5 | 1 | 1.32 | 69.6 | 0.7 |
| P-6 | 1 | 10 | 7.38 | 0.5 | 0.04 | 79.4 | 0.9 |
| P-7 | 0.1 | 120 | 6.5 | 0.5 | 0.01 | 113.2 | 1.0 |
| P-8 | 0.55 | 65 | 6.94 | 1 | 2.78 | 86.0 | 0.6 |
| P-9 | 0.55 | 120 | 6.94 | 1.5 | 3.01 | 85.3 | 0.5 |
| P-10 | 1 | 65 | 6.94 | 0.5 | 0.14 | 121.0 | 0.6 |
| P-11 | 0.55 | 65 | 6.94 | 1 | 2.46 | 88.1 | 0.6 |
| P-12 | 0.1 | 10 | 7.38 | 1.5 | 3.15 | 100.1 | 0.7 |
| P-13 | 0.55 | 10 | 7.38 | 1 | 2.29 | 85.0 | 0.6 |
| P-14 | 0.55 | 65 | 6.94 | 1 | 2.42 | 84.2 | 0.5 |
| P-15 | 0.1 | 10 | 6.94 | 0.5 | 0.14 | 167.0 | 0.5 |
| P-16 | 1 | 65 | 7.38 | 1.5 | 1.52 | 68.3 | 0.7 |
| P-17 | 1 | 120 | 6.5 | 0.5 | 0.04 | 79.3 | 1.4 |
| P-18 | 1 | 120 | 6.5 | 1.5 | 2.36 | 68.0 | 0.7 |
| P-19 | 0.55 | 120 | 7.38 | 0.5 | 0.29 | 83.2 | 0.9 |
| P-20 | 0.1 | 65 | 7.38 | 0.5 | 0.17 | 98.4 | 0.9 |
| P-21 | 0.55 | 10 | 6.5 | 0.5 | 0.02 | 96.2 | 1.1 |
| P-22 | 0.1 | 120 | 6.94 | 1 | 4.79 | 95.2 | 0.7 |
| P-23 | 1 | 120 | 7.38 | 1 | 1.52 | 69.8 | 0.7 |
| P-24 | 0.55 | 65 | 6.5 | 1.5 | 3.26 | 85.5 | 0.5 |
| P-25 | 1 | 10 | 6.94 | 1.5 | 2.65 | 69.2 | 0.8 |

TABLE 15
(Overview of flocculation CCD DoE study performed for
chitosan, CF = clearance factor (Load_{HCP}/Pool_{HCP})

| | [Chitosan] | | | Conductivity | | | |
|---|---|---|---|---|---|---|---|
| | (% w/v) | Mixing Time (s) | pH | (×control) | NTU Normalized | Yield | HCP (CF) |
| C-1 | 0.04 | 65 | 7.38 | 1 | 1.10 | 101.1 | 1.15 |
| C-2 | 0.08 | 10 | 6.94 | 1.5 | 1.02 | 95.4 | 1.43 |
| C-3 | 0.04 | 120 | 7.38 | 0.5 | 0.04 | 94.9 | 1.74 |
| C-4 | 0.04 | 10 | 7.38 | 1.5 | 1.19 | 99.9 | 1.08 |
| C-5 | 0.08 | 120 | 6.5 | 0.5 | 4.34 | 108.8 | 2.35 |
| C-6 | 0.12 | 10 | 6.5 | 0.5 | 2.69 | 103.2 | 3.78 |
| C-7 | 0.08 | 65 | 6.94 | 1 | 0.26 | 94.9 | 0.74 |
| C-8 | 0.04 | 10 | 6.94 | 0.5 | 0.03 | 168.7 | 1.05 |
| C-9 | 0.04 | 120 | 6.5 | 0.5 | 0.01 | 113.0 | 0.96 |
| C-10 | 0.12 | 120 | 6.5 | 1.5 | 1.35 | 90.9 | 0.87 |
| C-11 | 0.08 | 120 | 7.38 | 1.5 | 1.66 | 99.6 | 1.13 |
| C-12 | 0.04 | 10 | 6.5 | 1 | 0.15 | 100.0 | 1.13 |
| C-13 | 0.08 | 65 | 6.94 | 1 | 0.29 | 95.3 | 1.04 |
| C-14 | 0.08 | 10 | 7.38 | 0.5 | 4.30 | 95.2 | 2.38 |
| C-15 | 0.12 | 120 | 6.94 | 1 | 0.08 | 92.3 | 1.15 |
| C-16 | 0.08 | 65 | 6.94 | 1 | 0.27 | 96.4 | 1.43 |
| C-17 | 0.08 | 65 | 6.94 | 1 | 0.23 | 95.2 | 1.32 |
| C-18 | 0.12 | 65 | 6.94 | 0.5 | 6.85 | 151.3 | 1.36 |
| C-19 | 0.12 | 120 | 7.38 | 0.5 | 4.72 | 37.7 | 1.51 |
| C-20 | 0.12 | 10 | 7.38 | 1 | 0.83 | 90.9 | 1.36 |
| C-21 | 0.12 | 65 | 7.38 | 1.5 | 1.20 | 101.8 | 1.05 |
| C-22 | 0.04 | 120 | 6.94 | 1.5 | 1.16 | 98.9 | 1.32 |
| C-23 | 0.04 | 65 | 6.5 | 1.5 | 1.19 | 97.8 | 1.33 |
| C-24 | 0.08 | 65 | 6.5 | 1 | 0.01 | 95.1 | 1.60 |
| C-25 | 0.12 | 10 | 6.5 | 1.5 | 1.74 | 90.1 | 1.34 |

TABLE 16
(Overview of flocculation CCD DoE study performed for calcium
phosphate, CF = clearance factor (Load_{HCP}/Pool_{HCP})

| | [Calcium | | | | | | |
|---|---|---|---|---|---|---|---|
| | Phosphate] | | | Conductivity | | | |
| | (mM) | Mixing Time (s) | pH | (×control) | NTU Normalized | Yield | HCP (CF) |
| A-1 | 4.63 | 120 | 7.31 | 1.5 | 0.94 | 97.1 | 3.47 |
| A-2 | 4.63 | 65 | 6.5 | 1 | 0.97 | 95.4 | 6.85 |
| A-3 | 4.63 | 10 | 6.5 | 1.5 | 1.01 | 95.1 | 4.77 |
| A-4 | 9.26 | 65 | 6.91 | 1 | 0.82 | 91.6 | 5.69 |
| A-5 | 13.89 | 10 | 6.5 | 1 | 0.80 | 83.4 | 3.76 |
| A-6 | 13.89 | 10 | 7.31 | 0.5 | 0.44 | 58.0 | . |
| A-7 | 4.63 | 120 | 6.5 | 0.5 | 0.83 | 87.9 | . |
| A-8 | 9.26 | 65 | 6.91 | 1 | 0.77 | 88.6 | 1.95 |
| A-9 | 9.26 | 120 | 6.91 | 1.5 | 0.54 | 89.3 | 2.37 |
| A-10 | 13.89 | 65 | 6.91 | 0.5 | 0.05 | 53.8 | 5.20 |
| A-11 | 9.26 | 65 | 6.91 | 1 | 0.82 | 90.8 | 2.42 |
| A-12 | 4.63 | 10 | 7.31 | 1.5 | 1.03 | 94.3 | 1.56 |
| A-13 | 9.26 | 10 | 7.31 | 1 | 0.93 | 89.5 | 2.47 |
| A-14 | 9.26 | 65 | 6.91 | 1 | 0.76 | 90.3 | 1.52 |
| A-15 | 4.63 | 10 | 6.91 | 0.5 | 1.00 | 87.1 | 2.35 |
| A-16 | 13.89 | 65 | 7.31 | 1.5 | 0.44 | 84.2 | 2.27 |
| A-17 | 13.89 | 120 | 6.5 | 0.5 | 0.11 | 54.4 | 4.70 |
| A-18 | 13.89 | 120 | 6.5 | 1.5 | 0.17 | 83.0 | 2.03 |
| A-19 | 9.26 | 120 | 7.31 | 0.5 | 0.09 | 72.2 | 4.73 |
| A-20 | 4.63 | 65 | 7.31 | 0.5 | 0.94 | 89.8 | 3.17 |
| A-21 | 9.26 | 10 | 6.5 | 0.5 | 0.82 | 70.8 | 5.64 |
| A-22 | 4.63 | 120 | 6.91 | 1 | 0.97 | 94.7 | 2.40 |
| A-23 | 13.89 | 120 | 7.31 | 1 | 0.26 | 85.0 | 3.86 |
| A-24 | 9.26 | 65 | 6.5 | 1.5 | 0.66 | 88.3 | 3.51 |
| A-25 | 13.89 | 10 | 6.91 | 1.5 | 0.77 | 81.9 | 2.11 |

Varying amounts of flocculant were added to 20 mL of each aliquot in a 50 mL conical tube. PEI was previously tested at additions from 0 to 0.2% (w/v), with higher levels of flocculant resulting in a higher DNA log reduction value (LRV), therefore PEI was evaluated in from 0.1% to 1% (w/v). Chitosan was also previously evaluated between 0-0.05% (w/v) with higher levels of Chitosan resulting in lower turbidity, higher DNA LRV, and high yields. Due to this, Chitosan levels between 0.4-0.12% were evaluated. Calcium Phosphate was evaluated in GE Healthcare application note 28-9403-48 AB with 9.26 mM CaPi used as the flocculant. A range of 4.63-13.89 mM was evaluated to surround this value.

After vigorously mixing the samples, 14 mL of each tube was placed in a 15 mL conical tube and incubated whilst rocking for the amount of time specified in the DoE. Control samples of each diluted pool as well as a native sample were also incubated. Tubes were then spun down in an Allegra X-22 (Beckman Coulter) centrifuge using rotor SX4250 at 4200 rpm for 10 minutes and the supernatant was analyzed for titer, turbidity, DNA and HCP. HCP was measured using the Bradford assay, which measures total protein in a sample. The difference between the total protein value and titer value is considered to be HCP. All reported values were normalized to control values. Two sets of controls were performed for each DoE, a native control where no dilutions were performed and then a control for the samples in which the conductivity was reduced by dilution with RODI. The controls were run in parallel and tested for the same responses. These values were then used to normalize the data, with all of the samples at 1.0× and 1.5× conductivity normalized to the native control and the diluted samples normalized to diluted control, as shown in Table 17 below.

TABLE 17
(Overview of control values performed for the flocculation DoE)

| | | | Titer | |
|---|---|---|---|---|
| | | N TU | (g/L) | HCP (ppm) |
| PEI/ | Native Control (no dilution) | 124 | 3.62 | 1.13E+06 |
| Chitosan | 0.5× conductivity Control | 67.2 | 1.37 | 1.59E+06 |
| Calcium | Native Control (no dilution) | 102.2 | 3.20 | 6.67E+05 |
| Phosphate | 0.5× conductivity Control | 56.9 | 1.42 | 7.67E+05 |

Due to the large sample volume and low through-put of the assay used for DNA quantification, select samples were initially analyzed for DNA content. The data attained was inconclusive and further DNA analysis was therefore not included when analyzing the DoE.

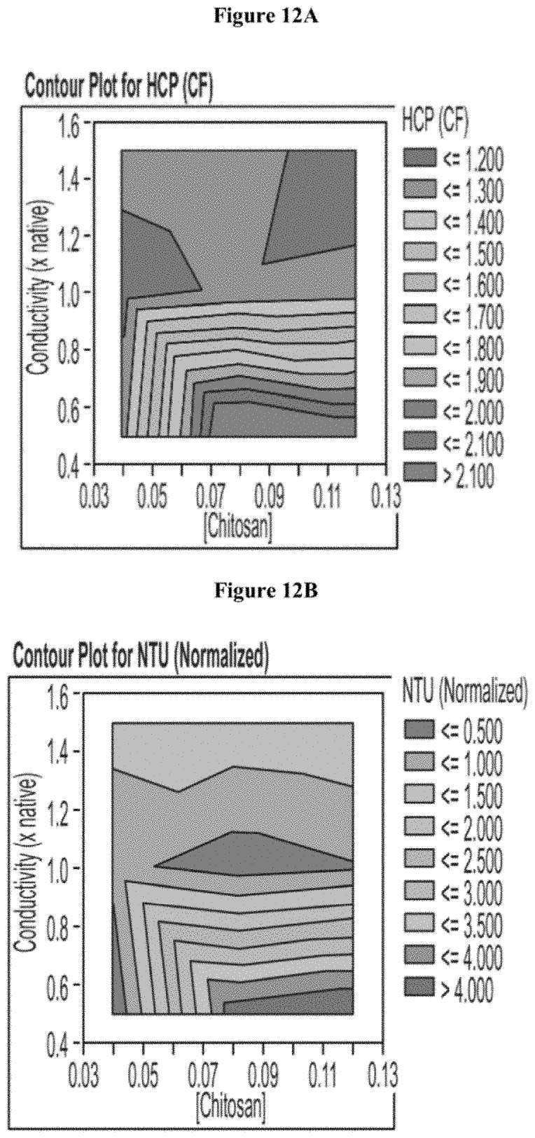

For chitosan, HCP and supernatant turbidity were affected by conductivity and chitosan concentration: an increase in HCP clearance (FIG. 12A) and supernatant turbidity (NTU) (FIG. 12B) at higher concentrations and lower conductivities was observed, with a minimum NTU (50% control) occurring at a native conductivity and spanning the concentrations tested. An increase in NTU is believed to decrease filter performance due to higher levels of particulates and is thus undesirable. The flocculation step yield was highest at low concentrations as well as low conductivity. However, the yield for most chitosan samples tested greater than 90 percent. HCP clearance (load/pool) was generally less than 2, which in and of itself is insufficient to merit this additional unit operation.

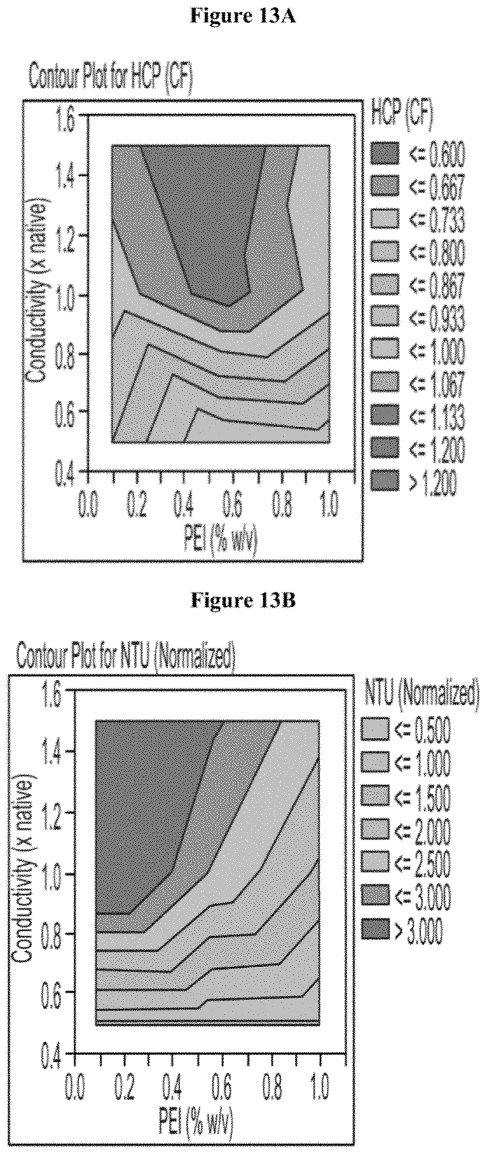

Lower concentrations of PEI and higher conductivities yielded in the lowest turbidity measurements (ca. 1 NTU), although most of the samples had turbidities higher than the control (FIG. 13A and FIG. 13B). Lower conductivities (0.5× native) resulted in higher HCP clearance (clearance factor (CF) 1.0-1.2; clearance factor=load impurity/pool impurity) as well as improved yield (>90%); unfortunately, most of the samples tested using PEI reported higher HCP levels than in the control (CF<1). PEI can strongly interfere with commonly employed protein estimation assays, such as the Bradford assay. Due to this interference, PEI absorbance was measured, converted to protein concentration, and factored into the results. The high HCP values measured in the PEI samples most likely are due to flaws in this evaluation, skewing the results higher than the actual value. Despite these drawbacks, it was found that it is best to operate PEI flocculation at a lower conductivity, resulting in higher yields (>90%), lower NTU values (4-20% of control) and a host cell protein clearance factor ca. 1-1.2. pH and mixing time were not found to be significant factors for either chitosan or PEI flocculation. Overall, PEI did not reduce HCP from the control and NTU was increased in most of the samples, which does not merit this additional unit operation.

Considering calcium phosphate flocculation, lower conductivities generally resulted in higher HCP clearance (CF>6), with some interaction between conductivity and calcium phosphate concentration. Lower concentrations of calcium phosphate (<8 mM) resulted in higher NTU values (70-80% of control), yet all NTU values for calcium phosphate flocculation were below the controls. While lower conductivities and higher concentrations provided better impurity clearance, yield was sacrificed at these conditions (50-70%), with the average yields <90% and both PEI and chitosan averaging yields >90%. Since calcium phosphate is a salt, clearance is easily achieved in either a positive mode chromatography step or tangential flow filtration. Calcium phosphate is also the only flocculant tested that achieved both relevant HCP clearance and lower turbidity and will therefore be pursued further.

### Example 6: Reduced pH: Acid Precipitation

In addition to the use of flocculant additives such as Chitosan and cationic salts, it has been found that reducing the pH may be used to cause particle precipitation that can result in the removal of cellular contaminants, such as cellular proteins or DNA. Lowering the pH too quickly, too low, or for too long, however, may have negative effects on the target protein (e.g., Dupilumab) and formation of high molecular weight (HMW) impurities, which include dimer species and higher order aggregates (HOA). To address these problems, it has been found that suitable buffers and acids for reducing pH levels should initially be added at reduced concentrations over an extended period of time. Nonlimiting examples of suitable buffers and concentrations include 1 M phosphoric acid, 1 M citric acid, and 1.75 M acetic acid. Based on the foregoing, it expected that a buffer or acid solution having a pKa range between 2 to 5 may also be added at a concentration of 1 M to 2 M.

### Example 7. Phosphoric and Acetic Acid used for Acid Precipitation

To determine the ability of acid adjustment in lowering contaminants in the soluble/liquid phase of bioreactor material prior to centrifugation, two exemplary acid precipitation were tested. 5 mL aliquots of Dupilumab cell culture fluid from a development bioreactor were adjusted down to various pH values ranging from 3.3-7.2, using either 1 M phosphoric acid or 1.75 M acetic acid. After 60 minutes of exposure to the various pH values, the aliquots were centrifuged for 10 minutes at 2200 rpm on an Allegra 6R (Beckman Coulter) bench centrifuge using rotor GH-3.8. The supernatant was filtered, neutralized and assayed for titer by analytical rProA (100 μL POROS MabCaptureA, 1 mL/min; equilibrate and wash in 10 mM NaPi, 500 mM NaCl, pH 7.2; elute in 500 mM acetic acid) and total protein using a Bradford Assay with Bovine Gamma Globulin (BGG) as a standard. The difference between the total protein content per sample and titer value was determined to be the host cell protein content. No DNA quantification was conducted.

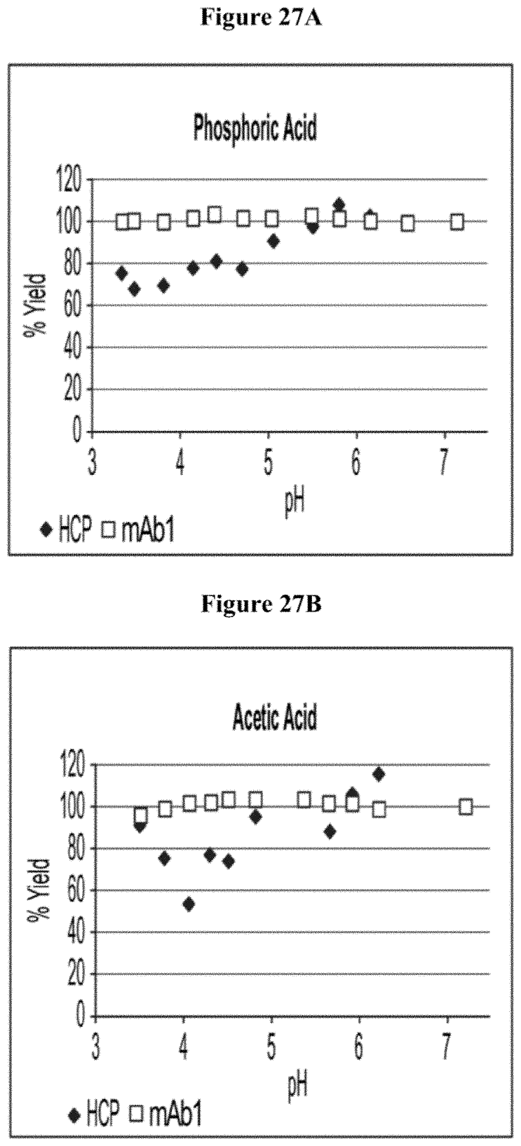

For both acids used for acid adjustment, the Dupilumab yield remained constant near 100% over the range of pH values tested, with lower (80%) HCP values achieved at more acidic pH values, as shown in FIG. 27A and FIG. 27B. Lower pH values result in an up to 80% HCP reduction as measured by the Bradford Assay whilst yield remains constant throughout the pH range tested.

Consistent with the data shown in FIG. 27A and FIG. 27B, it was found that the volume of 1 M phosphoric acid necessary to achieve pH adjustment was much less than 1.75 M acetic acid and that more acidic pH values result in the lowest (80%) HCP values at high (100%) yields.

To minimize potential degradation of the target protein and formation of HMW impurities, suitable buffers and acids may slowly be added over a period of time ranging from 5 minutes to 30 minutes, depending the buffer or acid used, in combination with an agitation rate of 35-50 rpm, where the agitation rate corresponds to an energy dissipation rate of 0.00114-0.0504 m²/s³. Energy dissipation rates predicted from computational fluid dynamics models are show in Table 18 below:

TABLE 18

| | | Energy Dissipation |
|---|---|---|
| Bioreactor Size and Type | Agitation (rpm) | Rate (m²/s³) |
| 40 L SSB | 60 | 0.00114 |
| 50 L SUB | 90 | 0.063843 |
| 50 L SUB | 100 | 0.064621 |
| 50 L SUB | 200 | 0.090896 |
| 500 L SUB | 110 | 0.072555 |
| 500 L SUB | 40 | 0.004796 |
| 500 L SUB | 75 | 0.033706 |
| 2,00 L SUB | 100 | 0.073414 |
| 10,000 L SUB | 43 | 0.0591 |

SSB, stainless steel bioreactor; SUB, single-use bioreactor

The energy dissipation rates can be calculated using, for example, the following energy dissipation rate equations used for a 500 L and 10,000 L stainless steel bioreactor shown below (respectively):

$$- 0.007137878 + \frac{( 0.2824278049 - ( - 0.007137878 ) )}{( 1 + Exp ( - 0.038290628 \cdot ( Agitation ( rpm ) - 122.18055984 ) ) )}$$

$$54233.304537 + \frac{( - 0.005810041 - 54233.304537 )}{( 1 + Exp ( 0.0579476751 \cdot ( Agitation ( rpm ) - 278.3075455 ) ) )}$$

To further minimize potential degradation of the target protein from lowered pH levels, the amount of time the harvest, including the target protein, is held at a reduced pH level should be limited and the pH level should then be raised. Suitable hold times can range from 30 minutes to 80 minutes depending on the pH. In an exemplary embodiment, the hold time ranges from 30 minutes to 60 minutes where the pH ranges from 4.3 to 5.0. To raise the pH, suitable buffers and bases can be used, including Tris base, sodium phosphate, potassium phosphate, sodium hydroxide. The pH may be raised to from about 5.5 to 6.5, or to about 6.0.

The temporary reduction in pH can be performed after cooling the bioreactor or prior to cooling of the bioreactor. Cooling the bioreactor prior to the temporary reduction in pH can further reduce interchain disulfide reduction of the molecule and limit the generation of aggregates in the target protein (e.g., Dupilumab) during the transient pH treatment.

As noted above, the process of temporarily reducing the pH causes process related impurities to aggregate or precipitate similar to a flocculant, and in certain exemplary embodiments described below, the impurities may then be removed using various filtration steps described below.

### Example 8. Design of a Novel Manufacturing Process for Antibody Drug Product

This Example sets forth the overall design of the novel manufacturing processes further described in detail in the preceding and following Examples. In order to accommodate increased productivity of an antibody drug product, a new production process was developed, as will be further described in detail in the Examples below. The optimized process was designed to accommodate increased productivity and to incorporate additional product and process understanding. The novel process delivers a greater than two-fold increase in productivity compared to an alternative commercially approved process.

The optimized process also had to accommodate various process-related considerations. One was ensuring comparable quality to an existing alternative process. Another was enhancing recombinant antibody productivity and recovery. A third consideration was characterizing scale-up and repeatability of the preliminary process in preparation for GMP production. Another consideration was optimizing each unit operation to minimize the impact of process variation on product quality.

The overall process steps for cell expansion, protein production, and purification have been redeveloped to allow for increased protein titer and production yield, while using similarly sized equipment and processing facilities. The production process maintains equivalent control throughout the manufacturing process to ensure product quality and monitor process consistency. The control strategy of the method incorporates enhanced process understanding from alternative manufacturing processes.

The optimized process described herein is based on fed batch suspension culture of recombinant Chinese Hamster Ovary (CHO) cells engineered to express an antibody drug product that are expanded through a seed train until inoculation of the production bioreactor. Other than the cell lines, no animal derived raw materials are used in the process. Cell banks for the optimized process are cryopreserved and thawed in chemically defined medium. All stages of the seed train as well as the production bioreactor feature chemically defined production cell culture medium and nutrient feeds. The novel process includes additional nutrient feeds compared to an alternative process to increase productivity and control product quality. Production bioreactor duration is approximately 10 days, a substantial time saving compared to an alternative process. Cell culture is terminated by harvest pre-treatment featuring reduced temperature and a novel pH titration step, followed by centrifugation and depth filtration. Harvest pre-treatment increases viral inactivated pool filterability.

The harvesting and purification process include Protein A affinity chromatography, cation (CEX) and anion (AEX) exchange chromatography, and hydrophobic interaction chromatography (HIC). This process includes high-capacity chromatography resins to accommodate increased productivity while maintaining manufacturing plant fit. The process uses low pH viral inactivation and virus-retentive filtration as dedicated viral clearance steps.

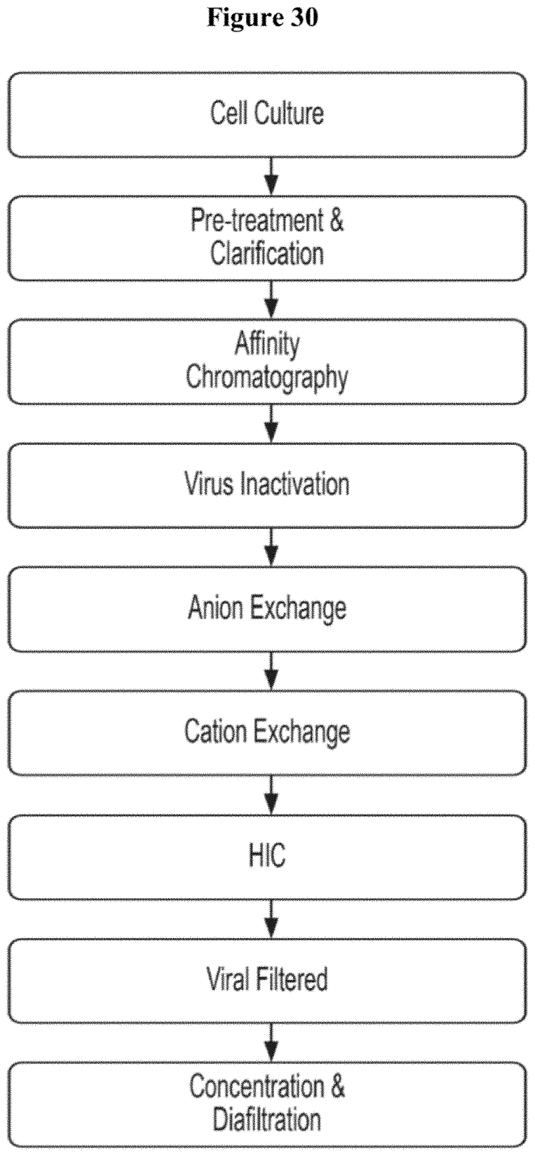

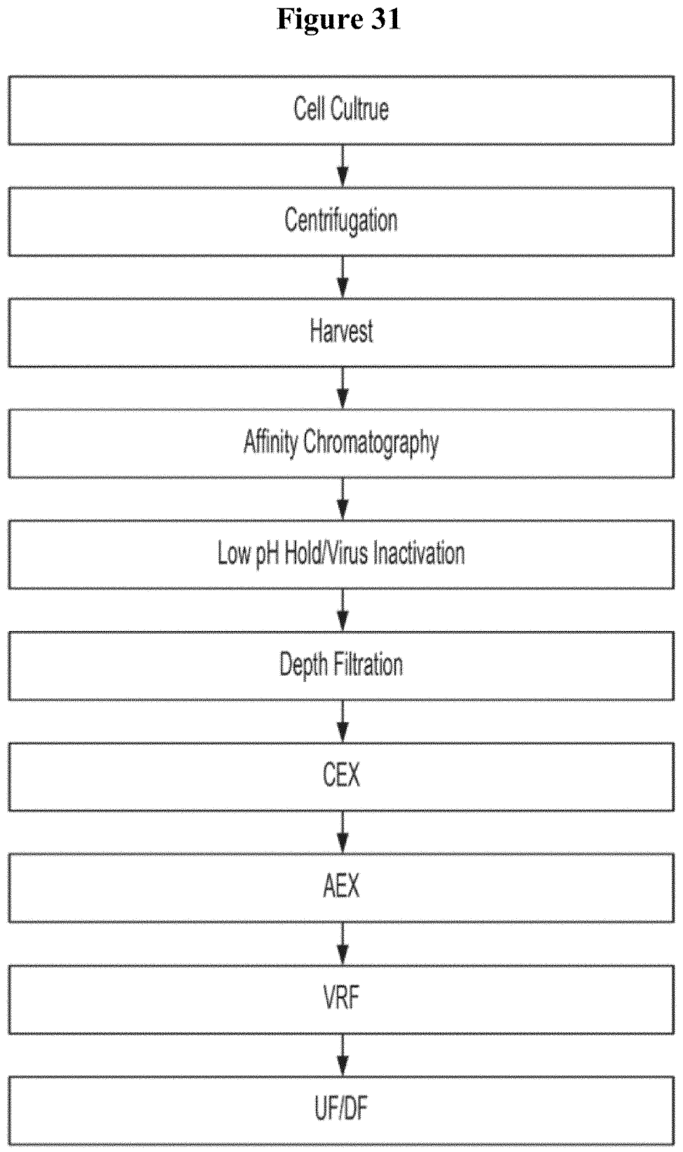

At completion of purification, final concentrated pool (FCP) is prepared by concentration/diafiltration (UF/DF). A process flow diagram showing exemplary steps from thaw to FDS is presented in FIG. 30 and FIG. 31. Exemplary novel features of the process of the present disclosure that provide advantages in the harvesting and purification of drug product are described in more detail below.

The chemically defined media used has been optimized compared to an alternative method, for example using the addition of poloxamer, taurine, and additional sodium phosphate. Poloxamer provides additional protection against shear stress in the production bioreactor. Taurine is present as an additional amino acid for improved cell productivity. Additional sodium phosphate contributes to improved cell growth.

As described above, the production bioreactor expansion time has been optimized, with a reduced time of about 10 days. This shorter batch duration improves overall production cadence and maintains product quality.

The bioreactor feed was additionally optimized in the novel method. The number of total feed events was increased to six, compared to an alternative method, and modifications were made to the composition and timing of production bioreactor feed formulations and production bioreactor duration. The improved feed strategy was developed to provide additional amino acids and nutrients based on higher cell density and overall increased cell productivity.

The optimized process includes a novel pre-treatment step prior to harvesting, including reduced temperature and pH titration followed by centrifugation and depth filtration. Transient pH pre-treatment aids in host cell and debris removal by mechanical centrifugation and filtration. Improved harvest pool quality leads to additional product stability during harvest hold. The depth filter at this step is further flushed to increase protein yield.

Following harvest, the polish filter was improved compared to alternative methods, with the introduction of a hybrid purifier multi-mechanism filter device. The EMP770 functionalized anion exchange harvest filter provides additional impurity clearance.

The Protein A chromatography resin used for the optimized process was selected to take advantage of modernized Protein A technology, allowing for greater binding capacity, which is useful for handling an increased drug titer.

During the low pH hold, the viral inactivation acid adjustment buffer was changed compared to alternative processes, from 1 M phosphoric acid to 0.25 M phosphoric acid, allowing for improved acid dispersion in the higher concentration protein A eluate pool. The target hold pH was changed from between 3.5-3.7 to between 3.45-3.65, which showed improvement in a viral clearance study. The post hold pH was changed from between 5.4-5.8 pH to between 5.8-6.2 pH, which was optimized for the change in the next chromatography modality.

Alternative processes may additionally feature a depth and polish filtration step at this point. Due to the pH pre-treatment step applied in the optimized process, a depth and polish filtration step is not necessary for the process of the present disclosure, and the sample may be advanced directly to the next chromatography step.

Following viral inactivation, existing alternative processes feature the steps of CEX, in bind/elute mode, followed by AEX, in flowthrough mode. Conventionally, AEX is used after CEX in order to limit the amount of impurities passing through the AEX column, since overloading the AEX column will result in impurities being passed in the flowthrough along with the drug substance. A disadvantage of this conventional process is that, because AEX is operated in flowthrough mode, drug substance is diluted after the AEX step, which is unsuitable for handling the high drug titer of the process of the present invention.

An advantage of the process includes the use of a functionalized anion exchange harvest filter as described above, which reduces impurities such as host cell DNA. This in turn leads to a lower amount of relevant impurities at the point where the sample is injected into the AEX column, preventing the AEX column from being overloaded with impurities even without a preceding CEX step. Therefore, the optimized process involves subjecting the sample to AEX before CEX instead of the reverse. The CEX step, operating in bind-elute mode, concentrates the flowthrough from the AEX step, improving the ability of the drug batch to fit into the processing plant.

Additionally, due in part to the lower number of impurities in the sample prior to the AEX step, the optimized process includes loading a greater amount of protein onto the AEX and CEX columns compared to alternative methods. Furthermore, the AEX resin and the CEX resin were each also optimized for increased binding capacity, in order to allow loading of more product over the column.

The HIC step was also improved over alternative methods. The amount of sample loaded onto the column was optimized, increasing from between about 80-100 grams of protein per liter of HIC media to between about 180-200 grams of protein per liter of HIC media, to improve yield without compromising quality. A lower citrate concentration for equilibration and wash of the column was optimized to ensure efficient impurity removal, and to increase yield. An additional advantage of using buffers with a low sodium citrate concentration, for example about 40 mM or about 30 mM sodium citrate, is the minimization of electrostatic interference between the buffer and a subsequent charged virus retentive pre-filter step.

The HIC strip 1 solution was additionally optimized for improved column regeneration developed to accommodate a higher throughput. It was found that a strip buffer comprising 5 mM NaOH improves solubility and removal of any residual contaminants compared to an alternative method.

Virus retentive filtration (VRF) was optimized through the use of a new pre-filter selected during development to allow for increased viral loading. As described above, the optimization of the HIC wash and equilibration buffers with a relatively low citrate concentration allowed for an improvement in the virus retentive filtration pre-filter, because a charged filter could be used without undue electrostatic interactions with sodium citrate.

During concentration and diafiltration steps (UF/DF), concentration and diafiltration set points were optimized to accommodate higher protein throughput. With the optimized process of the present invention, the amount of protein that could be processed using the same equipment as an alternative method was about twice as much. The load adjustment solution was changed to align with an optimized excipient concentration in the final formulation. The diafiltration buffer was also changed from 10 mM sodium acetate, pH 4.8±0.10 in an alternative process to 4 mM acetate, pH 4.10±0.10 in the optimized process to adjust for the removal of arginine from the process.

The arginine adjustment solution used in existing alternative processes was entirely removed from the optimized process, due to the addition of enhanced concentration controls that allowed for the optimization of viscosity without the addition of a viscosity-reducing agent. The protein concentration range for the final concentrated pool after UF/DF was optimized in order to increase product recovery by slightly reducing viscosity, from a range between 228 to 255 g/L (target 240 g/L) to between 224-255 g/L (target 232 g/L). This reduced viscosity allowed for improved pump function that led to improved protein recovery without the addition of a viscosity-reducing agent such as arginine.

Finally, drug substance adjustment was further optimized: an optional dilution step was introduced in the optimized process to accommodate the increased protein throughput, resulting in higher DS concentration. DS adjustment buffers were also optimized to compensate for the removal of the arginine adjustment step.

Based on comparability testing, the optimized process shows substantial improvement over alternative processes. In one example, an existing alternative process was used to process a harvested protein batch which achieved a 50% downstream yield over the course of harvesting and purification steps. In contrast, the optimized process was used to process a harvested protein batch comprising 2.6 to 3 times more protein for the same sized batch and achieved a 60-65% downstream yield over the course of harvesting and purification steps. Thus, the optimized process is preferred for greater net yield of antibody product.

Further details on optimized steps of the optimized process are described below.

### Example 9. Affinity Capture and Viral Inactivation Development

An affinity chromatographic step captures a protein of interest, for example Dupilumab, from clarified conditioned medium, thereby reducing process related impurities such as DNA, HCP, and cell culture components, and increasing protein concentration.

In exemplary embodiments, affinity chromatography and subsequent viral inactivation by low pH hold occurs after harvest of the bioreactor, and as such, serves as the first chromatography step in the optimized process of the present invention. During the capture, a protein of interest, for example Dupilumab, is bound to the affinity resin and then washed to remove non-specifically bound impurities. The protein is subsequently eluted at low pH. Following elution from the column, the protein is subjected to a low pH hold (LPH) for viral inactivation (VI), and then adjusted to a higher pH in preparation for subsequent filtration and packed bed chromatographic polishing steps. Low pH hold conditions included viral inactivation at a pH from about 3 to about 4 followed by titration to a pH from about 5 to about 8. The viral inactivated pool (VIP) is sterile filtered, for example using a LifeASSURE™ PDA filter (3M).

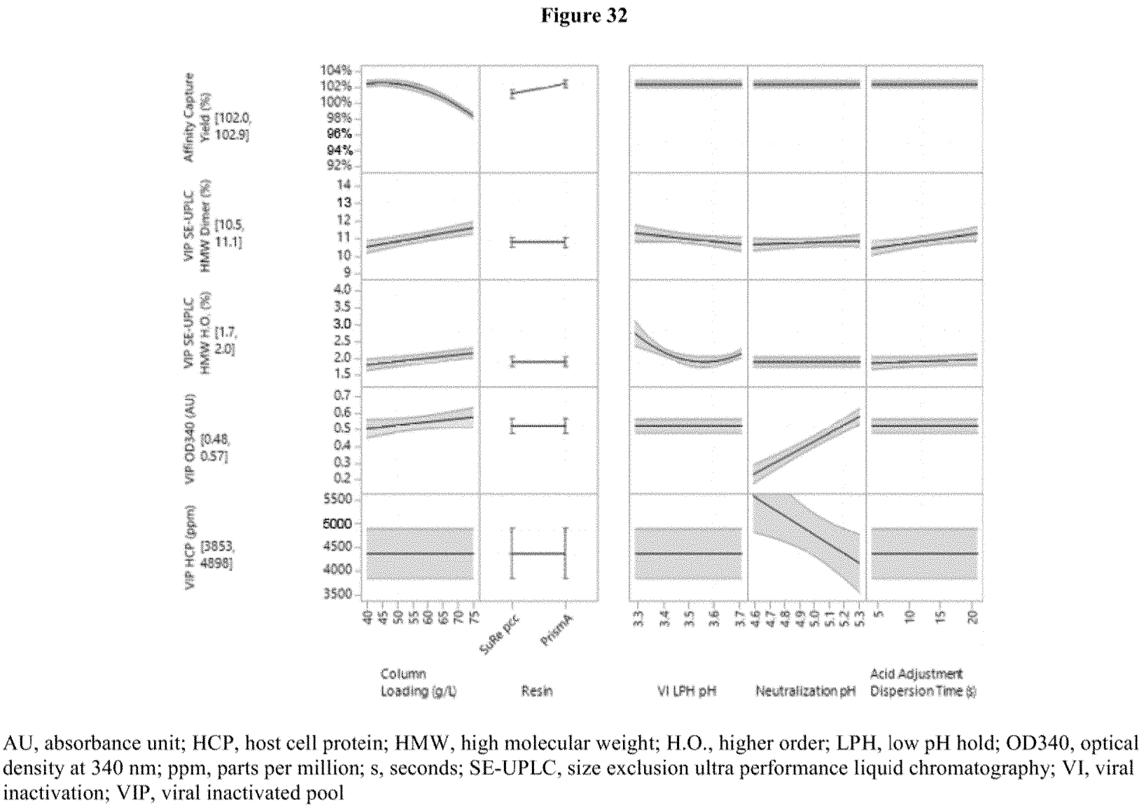

Affinity capture and viral inactivation by low pH hold process performance during confirmation batches were compared to small-scale model predictions derived from the aforementioned Monte Carlo simulations. Comparison of the confirmation batches to the Monte Carlo simulations showed that Pool HMW and VIP HCP levels were equal to or less than the predicted ranges generated from scale-down multivariate models, demonstrating the utility of Monte Carlo simulations as a conservative prediction model. Confirmation batches resulted in affinity capture yield (%) of about 95%-103%, VIP SE-UPLC HMW dimer of about 9.8%-11.3%, and VIP SE-UPLC HMW higher order (%) of about 0.7%-2.0%, demonstrating effective control of these attributes and scalability of the process. Multivariate studies of affinity capture and viral inactivation risk factors and responses were performed as shown in FIG. 32.

Additionally, variability in HCP content in the confirmation batches as compared to Monte Carlo simulations was attributed to use of a single batch of load material used at small scale that had been stored frozen prior to studies and resulting simulations, while the pilot-scale productions used load material derived from diverse batches without a freeze/thaw cycle. All confirmation batches demonstrated VIP HCP levels of 2900 (ppm)-4500 (ppm), with further HCP clearance provided downstream, demonstrating effective control of this attribute. The successful scale-up from bench scale to 500 L scale provides support and confidence for successful scale-up to 10,000 L scale.

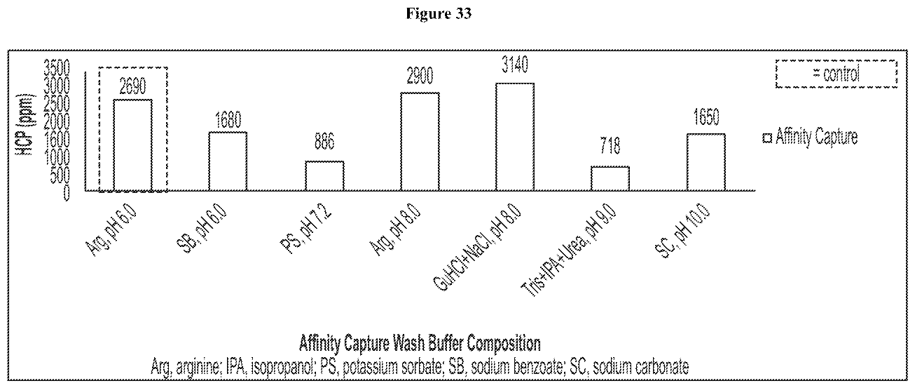

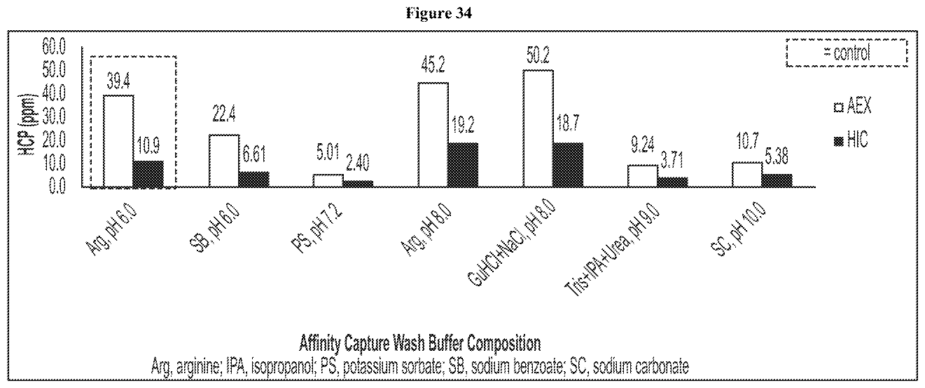

The use of Protein A wash buffers for removing HCPs associated with the protein of interest was further investigated. The effects of the buffers described in Table 8 were assessed for a monoclonal antibody, using a buffer comprising 450 mM arginine and 20 mM tris at pH 6.0 as a control. The HCP in the affinity pool is shown in FIG. 33. The HCP of the subsequent pools following either AEX alone or HIC alone, without a CEX step, are shown in FIG. 34.

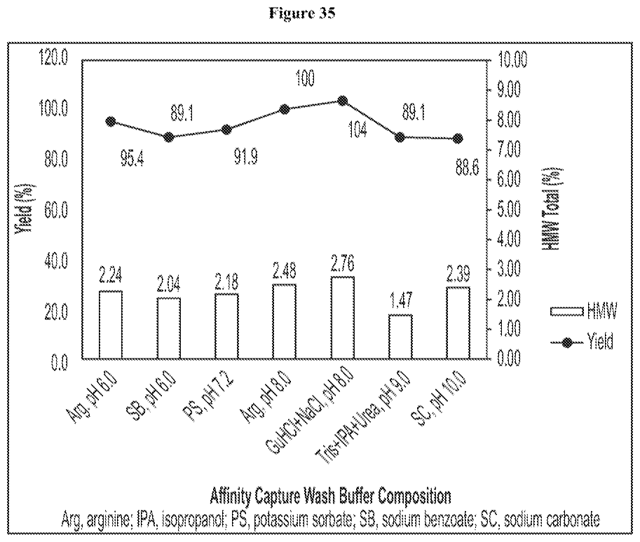

The same buffers were further investigated for their ability to reduce BMW species in the Protein A pool. A quantification of yield and protein A pool HMW, as compared to the control buffer, is shown in FIG. 35.

The results of this screening show that several of the screened affinity wash buffers may be suitable for an appropriate drug substance quality, for example, yielding below 30 ppm HCP. The selection of an appropriate wash buffer yielded a suitable product quality even when omitting downstream steps such as CEX, AEX, or HIC. The use of a wash buffer selected for removing HCPs and/or BMW species may make it desirable to produce Dupilumab in the absence of a HIC processing step, for example using only the chromatography steps of Protein A, CEX and AEX; or Protein A, MMC and AEX, as further detailed in Example 24.

### Example 10. Anion Exchange Chromatography Development

An anion exchange (AEX) chromatography unit operation reduces levels of CHO DNA, CHO HCP, HMW product related impurities, and diverse model viruses. In exemplary embodiments, AEX is the second chromatography step in the optimized process and is performed downstream of affinity capture and low pH hold virus inactivation steps. This unit operation is performed in flow through mode where negatively charged impurities are adsorbed to the immobilized, positively charged ligand (column), and the product flows through.

An AEX chromatographic media for the optimized process may be selected, for example, for superior removal of macromolecules like DNA, and superior flow properties, compared to alternative AEX media.

Repeated use of chromatography resin was studied through univariate studies to demonstrate minimal change to quality attributes or process performance following 100 cycles. Wash length was also evaluated through univariate studies and was not found to impact process performance, except for yield. Load protein concentration was studied retrospectively to demonstrate minimal change to quality attributes or process performance with variable feed concentration. Raw material lot-to-lot variability was considered in this risk assessment, especially variability of the chromatography resin. However, raw material variation was identified as low risk due to platform experience with exemplary AEX resins with other monoclonal antibody processes, and therefore not directly investigated for Dupilumab in multivariate or univariate studies.

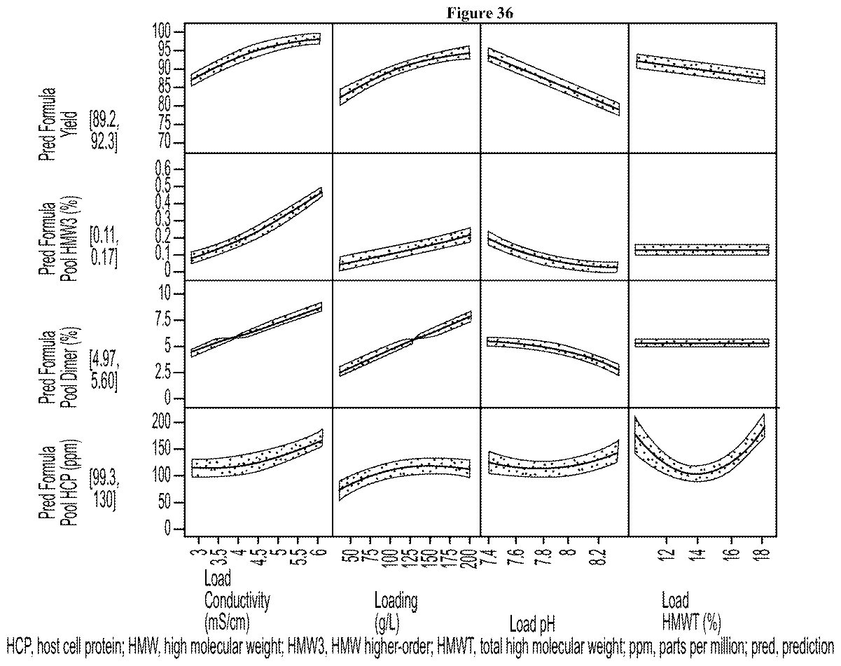

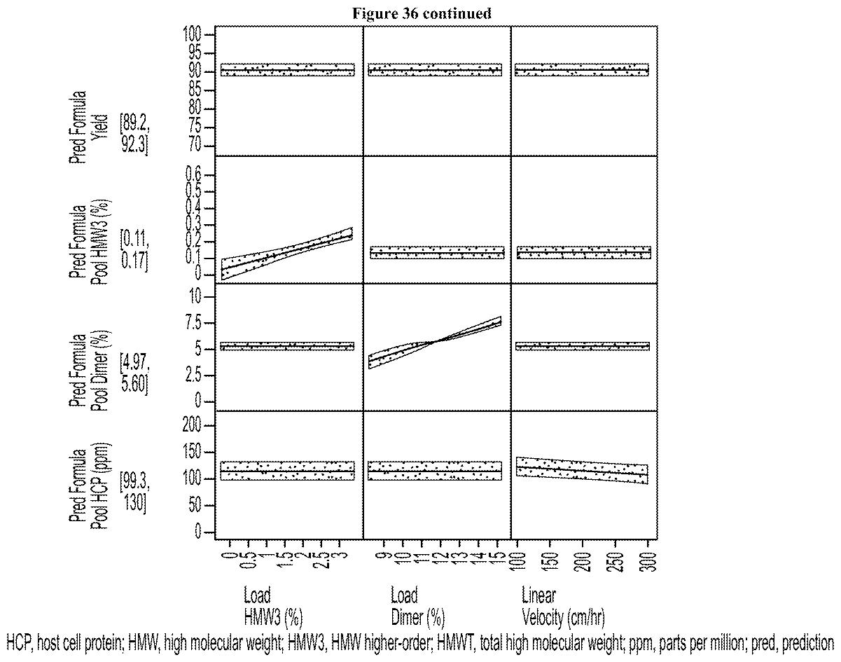

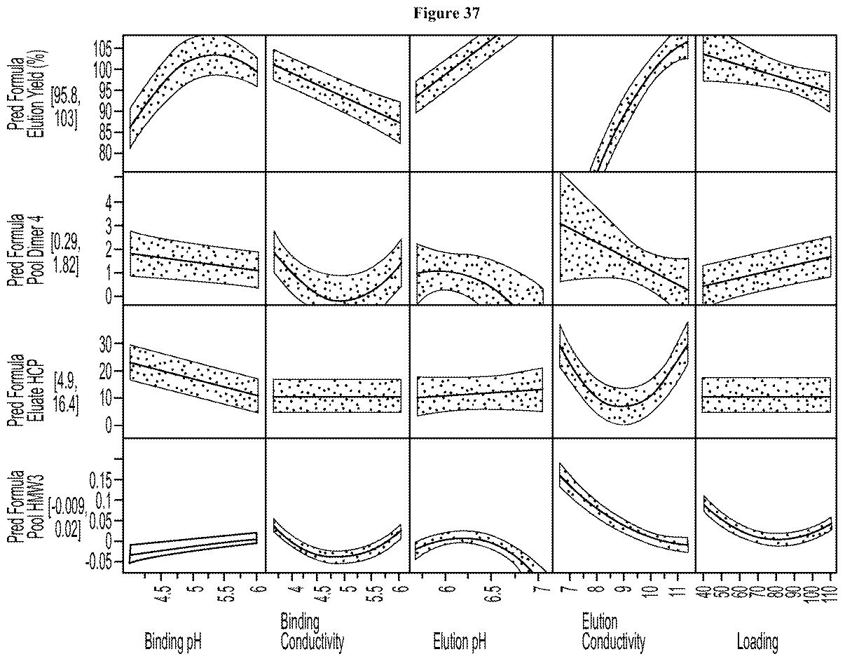

Anion exchange (AEX) process performance during confirmation batches was compared to small-scale model predictions derived from Monte Carlo simulations of the process run at set point with estimated variation in input parameters. Comparison of the confirmation batch responses with Monte Carlo simulations showed that the predicted range generated from the scale-down multivariate model for Dupilumab produced was very similar to the confirmation batches using the optimized process of the present invention, illustrating process robustness to scale-up and appropriateness of the small-scale model to predict pilot scale performance. Confirmation batches resulted in AEX yield (%) of about 88%-91%, AEX pool SE-UPLC UMW higher order (%) of about 0.01%-0.08%, AEX pool SE-UPLC HMW dimer (%) of about 4.25%-5.75%, and AEX pool HCP (ppm) of about 110 (ppm)-170 (ppm). The successful scale-up from bench scale to 500 L scale provides support and confidence of successful scale-up to 10,000 L scale. Multivariate studies of AEX risk factors and responses were performed as shown in FIG. 36.

### Example 11. Cation Exchange Chromatography Development

A cation exchange (CEX) chromatography unit operation reduces levels of CHO HCP and HMW product related impurities. In exemplary embodiments, CEX is the third chromatography step in the optimized process and is performed downstream of an anion exchange chromatography step. This unit operation is performed in positive (bind-elute) mode, where positively charged product and impurities are adsorbed to the immobilized, negatively charged stationary phase. The product is then eluted through an increase in conductivity, while many of the impurities remain bound to the stationary phase. A series of regeneration steps then remove bound impurities and prepare the CEX column for subsequent cycles.

A CEX chromatographic media for the optimized process may be selected, for example, for higher Dupilumab binding capacity compared to alternative CEX media and reduction in process volume because the step operates in bind/elute mode. High binding capacity, combined with placement of CEX after the dilutive AEX step, allows a batch produced using the optimized process to be processed in existing plant infrastructure.

Repeated use of chromatography resin was studied through univariate studies to demonstrate minimal change to quality attributes or process performance following 100 cycles. Performance was verified in four 500 L confirmation batches that were produced with the intended commercial upstream process. Compared to the first three confirmation batches, for the fourth confirmation batch the CEX process was adjusted slightly with a reduction in elution flow rate. Reduction in elution flow rate was intended to reduce pressure increase in columns of diameter exceeding 20 cm, based on observed pressure increase in 10,000 L scale processes using the same chromatography resin and bed height. While this flow rate was not characterized in multivariate studies, elution flow rate has not been identified as affecting product quality in similar monoclonal antibody processes, and the change was considered to pose low risk to product quality.

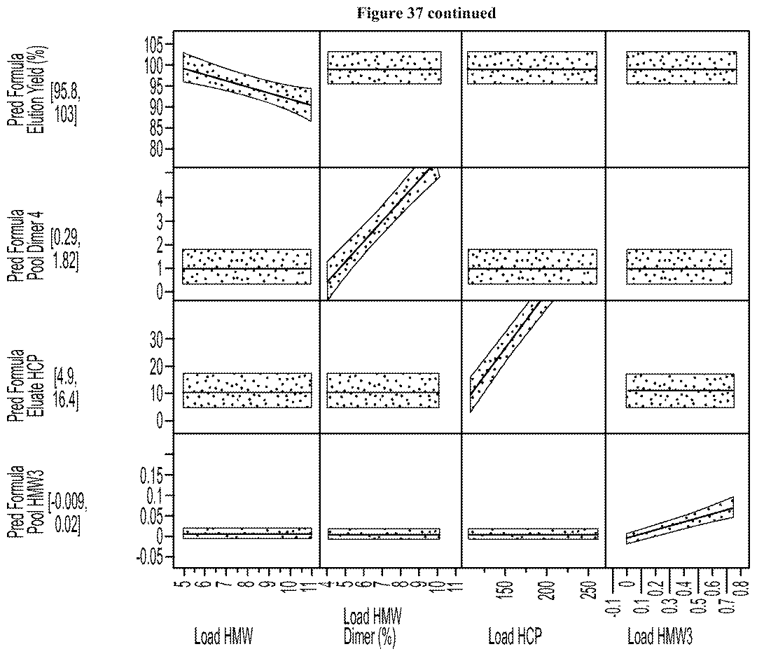

Cation exchange process performance during confirmation batches was compared to small-scale model predictions derived from Monte Carlo simulations of the process run at set point with estimated variation in input parameters. Comparison of the confirmation batch responses with Monte Carlo simulations showed that the predicted range generated from the scale-down multivariate model for Dupilumab produced was very similar to the confirmation batches using the optimized process of the present invention, illustrating process robustness to scale-up and appropriateness of the small-scale model to predict pilot scale performance. All confirmation batch quality was similar and resulted in CEX yield (%) of about 93%-98%, CEX pool SE-UPLC HMW dimer (%) of about 1%-1.5%, CEX pool HCP (ppm) 12 (ppm)-23 (ppm). The successful scale-up from bench scale to 500 L scale provides support and confidence of successful scale-up to 10,000 L scale. Multivariate studies of CEX risk factors and responses were performed as shown in FIG. 37.

### Example 12. Hydrophobic Interaction Chromatography Development

A hydrophobic interaction chromatography (HIC) unit operation reduces HMW species and HCP levels, including HCPs as well as a specific HCP, PLBD2. In exemplary embodiments, HIC separation is the fourth chromatography step and is performed downstream of cation exchange chromatography. This unit operation is performed in flow through mode where hydrophobic species are adsorbed to the immobilized, phenyl ligand (column), and the product flows through. Hydrophobic interactions are driven by presence of citrate, a kosmotrope.

Repeated use of chromatography resin was studied through univariate studies to demonstrate minimal change to quality attributes or process performance after 100 cycles. Raw material lot-to-lot variability was considered in this risk assessment, especially variability of the chromatography resin. Raw material variation was defined as low risk by the risk assessment due to platform experience with exemplary HIC resins in other monoclonal antibody processes, and resin lot was not directly investigated for Dupilumab in multivariate or univariate studies.

Performance of the intended commercial process was verified in four pilot-scale (500 L) confirmation batches that were produced with the intended commercial upstream process. Compared to the first three confirmation batches, for the fourth confirmation batch the HIC process was adjusted to increase maximum HIC loading. Increase in maximum HIC loading from 120 g/L to 180 g/L resin was intended to enhance product recovery and was supported by multivariate characterization studies showing HMW dimer and HCP remained comparable to alternative processes and met development considerations for HIC pool and FCP. Maximum loading of 180 g/L was predicted to result in typical 10,000 L scale loading of approximately 140 g/L, which was targeted in Confirmation Batch 4.

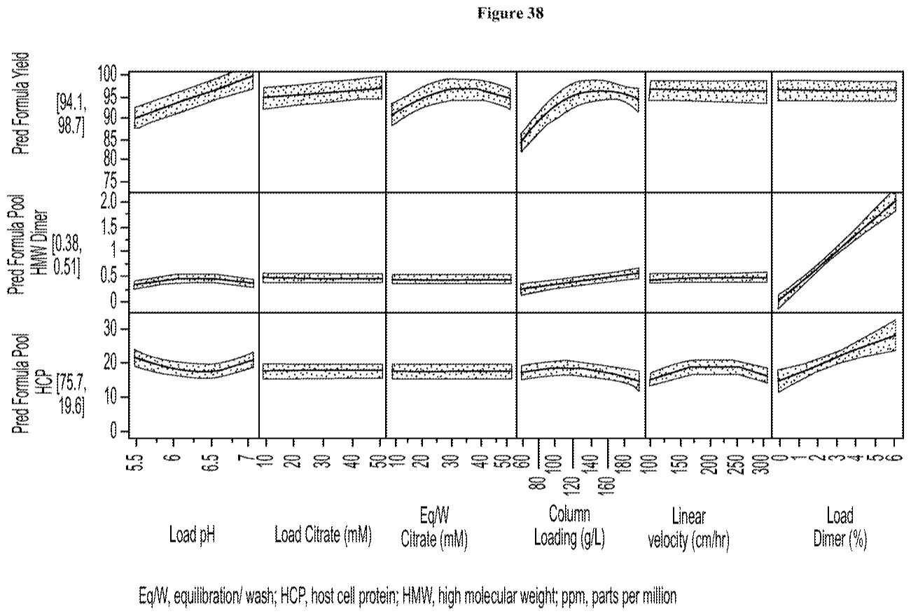

HIC process performance during these confirmation batches was compared to the performance of the process run at set point with estimated variation in input parameters derived from Monte Carlo simulations. Comparison of the confirmation batch responses with Monte Carlo simulations showed that the predicted range generated from the scale-down multivariate model for Dupilumab produced was very similar to the confirmation batches using the optimized process of the present invention, illustrating process robustness to scale-up and appropriateness of the small-scale model to predict pilot scale performance. Confirmation batches resulted in HIC yield (%) of about 90.5%-93.5%, HIC pool SE-UPLC HMW dimer (%) of about 0.3%-0.5% and HIC pool HCP (ppm) of about 9 (ppm)-14 (ppm). The successful scale-up from bench scale to 500 L scale provides support and confidence of successful scale-up to 10,000 L scale. Multivariate studies of HIC risk factors and responses were performed as shown in FIG. 38.

### Example 13. Virus Retentive Filtration Development

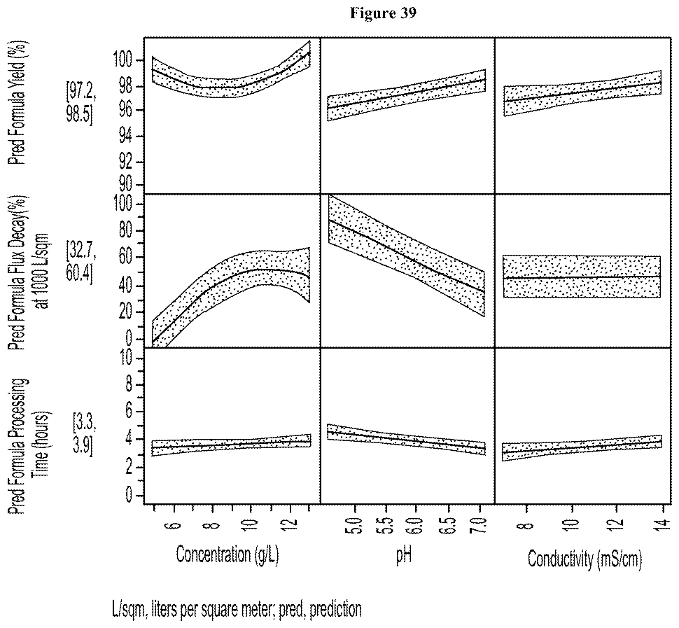

Virus reduction by virus retentive membranes is based on a size exclusion mechanism by which a protein of interest, for example a monoclonal antibody (˜10 nm in hydrodynamic diameter), passes through the filter while the larger virus (>18 nm) is retained by the membrane. In exemplary embodiments, a virus retentive filter (VRF) for the optimized process is after a HIC step. The unit operation is performed under constant pressure, where the product flows through the membrane and a variety of model viruses are retained. Multivariate studies of VRF risk factors and responses were performed as shown in FIG. 39.

In exemplary embodiments, the virus retentive pre-filter of the optimized process is selected based on, for example, not requiring the addition of excipients, and being compatible with the optimized process after HIC with no feed adjustment. Virus retentive filters of the optimized process were selected on the basis of effectively removing small (18-24 nm), non-enveloped viruses such as Minute Virus of Mice (MVM) from the process stream after chromatographic purification. This removal has been verified for a number (N>60) of monoclonal antibodies in a variety of feed stream compositions.

The accumulated knowledge through development and virus spiking trials resulted in an initial maximum transmitted volumetric loading capacity of ≤900 L/m² for GMP manufacturing. This capacity provided a 12% safety factor over the loading of 1,005 L/m² evaluated during virus spiking trials with a predicted loading of 588±92 L/m² during initial production. In subsequent production trials, additional virus spiking tests have been performed to show validated effective clearance up to 2021 L/m².

A univariate study was performed to evaluate the effect of air/liquid interfaces on process attributes. An additional univariate test was performed at a virus spiking study to assess process pause and post-processing buffer chase to verify no impact to virus removal (based on preliminary HIC development conditions and not the transmitted process). No detectable virus was observed and >4 log₁₀ reduction value (LRV) was achieved for the conditions evaluated.

Scale-up of the optimized process was verified in four 500 L confirmation batches. Compared to the first three batches, the VRF process was adjusted for the fourth batch to remove an adsorptive depth filter. Removal was intended to simplify the process and enhance yield and was within the characterized process development. The adsorptive depth filter conditions the load and can remove species that foul the virus filter; however, these species often result from artificial handing such as freezing and thawing in-process intermediate. Bench and pilot scale data show acceptable virus-filter flow properties and comparable host cell protein profile with and without the adsorptive depth filter. Viral clearance was subsequently validated with representative load material sampled from 10,000 L scale, without an adsorptive depth filter.

### Example 14. Ultrafiltration and Diafiltration Development

An ultrafiltration and diafiltration (UF/DF) step uses tangential flow filtration (TFF) to condition a protein of interest, for example Dupilumab, to achieve desired FCP pH, excipient content, and protein concentration to facilitate storage and formulation. In exemplary embodiments, the UF/DF for the optimized process is located immediately after a VRF unit operation. During processing, protein solution is pumped tangentially across the surface of a semi-permeable, parallel, flat sheet membrane. The manufacturing step uses a 50 kDa pore size membrane; thus, the membrane is permeable to water and buffer salts but generally impermeable to monoclonal antibodies such as Dupilumab (147 kDa). The driving force for permeation is applied transmembrane pressure induced by flow restriction at the outlet of the membrane flow channel.

Two raw material considerations were identified: (i) UF/DF membrane type and (ii) UF/DF product pool sterile filter. For UF/DF membrane type, exemplary molecular weight cutoff filters and membranes were chosen based on experience from other monoclonal antibody programs. The UF/DF pool final filter controls bioburden as well as pool quality through reduction of subvisible particle (SVP) level. Final filter types were evaluated based on previous experience (N>18 programs). Exemplary final filters may be selected based on, for example, their ability to provide acceptable volumetric throughput and product quality through reduction of SVP in FCP.

A final consideration of raw material selection was control of arginine during UF/DF and in FDS. Dupilumab FDS includes 25 mM arginine to reduce viscosity. Alternative processes add arginine prior to concentration, and the level in Final Concentrated Pool is measured so that Drug Substance could be controlled to 25 mM. Based on acceptable viscosity in preliminary Final Concentrated Pool in the optimized process in absence of arginine, process development proceeded without arginine adjustment prior to final concentration to simplify the process and reduce hold time during arginine quantitation.

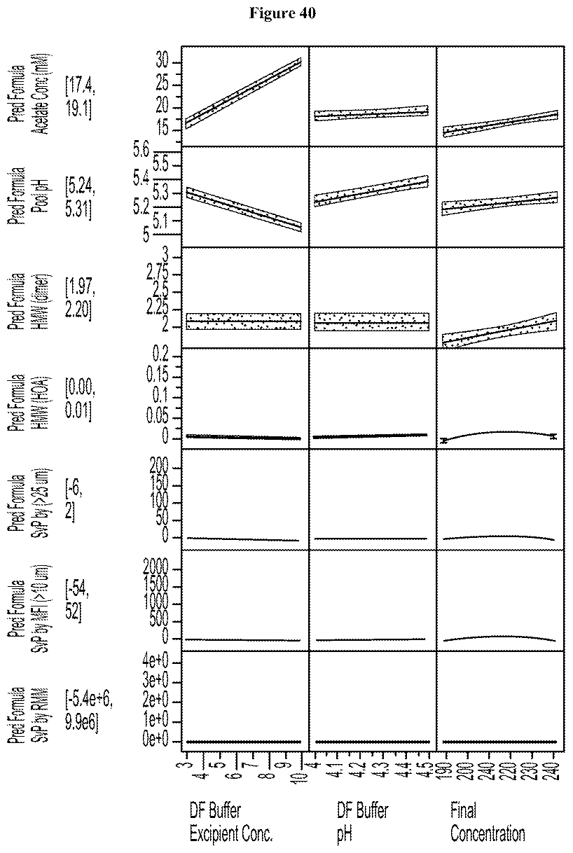

Scale-up of the optimized process was demonstrated in four 500 L confirmation batches using the final process. UF/DF process performance during these batches were compared to small scale model predictions derived from Monte Carlo simulations of the process run at set point with expected variation in input parameters and model RMSE. Confirmation batch for FCP acetate concentration, FCP pH, FCP (%) HMW higher order, SVP by RMM, SVP by MFI (>10 and SVP by MFI (>25 μM) were within the predicted range generated from the scale-down multivariate model. FCP % HMW dimer was lower than predicted, which can be attributed to optimized preceding purification steps. Confirmation batches resulted in FCP concentration (g/L) of about 230-252, FCP acetate concentration (mM) of about 16.5-20, FCP pH of about 5.2-5.3, FCP SE-UPLC HMW dimer (%) of about 0.7-0.9, FCP SE-UPLC HMW higher order (%) of about 0.02, FCP sub-visible particle (SVP) by mean fluorescence intensity (MFI)>10 μM (#/mL) of about 50, FCP SVP by MFI>25 μM (#/mL) of about 5-25, and of FCP SVP by resonant mass measurement (RMM) of about 2e6-2e7. Accounting for expected process variability, pilot-scale confirmation batch data was comparable to predictive model simulations at final process set points. Multivariate studies of UF/DF risk factors and responses were performed as shown in FIG. 40.

### Example 15. 10,000 L GMP Scale Development

During scale-up from 500 L scale to 10,000 L GMP scale, continued process understanding was applied to enhance robustness and promote process efficiency. Modifications to the bioreactor starting volume, air sparge, and agitation setpoints were made to improve process robustness through minimization of shear stress on the cell culture. Furthermore, glucose feeding strategy was modified to prevent high osmolality conditions that could impact the cell culture performance.

The modified setpoints were within the transmitted range and are outlined in Table 19.

TABLE 19
Modifications to the 10,000 L bioreactor starting
volume, air sparge and agitation setpoints

| | 10,000 L, Initially | 10,000 L, Modified |
|---|---|---|
| Process Setpoints | Transmitted | Transmitted |
| Bioreactor Starting Volume (L) | 6500-7200 | 7200-8000 |
| Air Sparge (slpm) | 400-500 | 25 to 300 (Cascade) |
| Agitation (rpm) | Inoculation to Day 2: 28 | Inoculation to Day 1.5^{a}: 22 |
| | Day 2 to Harvest: 40 | Day 1.5 to Day 4.5: 28 |
| | | Day 4.5 to Day 5.5: 34 |
| | | Day 5.5 to Harvest: 40 |
| Glucose Target (g/L) | Inoculation to Day 2: 5-6 | Inoculation to Day 2: 5-6 |
| | Day 3: 7-8 | Day 3: 7-8 |
| | Day 4 to Harvest: 9-10 | Day 4 to Harvest: 5-7^{b} |

^{a}Shift occurs at day 1.5 or when dissolved oxygen reaches setpoint, whichever occurs first.
^{b}Glucose is fed twice per day and targets 6 g/L each time.
^{c} slpm, standard liters per minute

During the transfer to a 10,000 L facility, maximum allowable processed volume was increased from 900 L/m² to 1,500 L/m² to reduce virus-retentive filter consumption. The increase in processed volume was supported by viral clearance data from the optimized process showing effective removal of minute virus of mice at up to 2,021 L/m² loading. The modified setpoints were within the characterized space and are outlined in Table 20.

TABLE 20
Modifications to the 10,000 L virus-filter area

| | 10,000 L, Initially | 10,000 L, Modified |
|---|---|---|
| Process Setpoints | Transmitted | Transmitted |
| Total Virus Pre-filter Area | 6.12 | 3.06 |
| (m²) | | |
| Total Virus Filter Area (m²) | 6.12 | 3.06 |
| Maximum Volumetric Loading | Up to 900 | Up to 1,500 |
| (L/m²) | | |

GMP, good manufacturing practices

During the transfer to a 10,000 L facility, the protein concentration range for FCP was shifted from 228-255 g/L (target 240 g/L) to 224-255 g/L (target 232 g/L) to reduce processing time and increase product recovery by slightly reducing viscosity. The modified setpoints were within the characterized space and are outlined in Table 21.

TABLE 21
Modifications to the 10,000 L final concentrated
pool protein concentration range

| | 10,000 L, Initially | 10,000 L, Modified |
|---|---|---|
| Process Setpoints | Transmitted | Transmitted |
| Final Concentrated Pool | 228-255 | 224-255 |
| Concentration (g/L) | (Target 240) | (Target 232) |

GMP, good manufacturing practices

### Example 16. Process Development Confirmation

Upon completion of confirmation batches at the conclusion of process development, outcomes of development of the optimized process were assessed.

As described in the above Examples, the conclusion of exemplary Dupilumab cell culture and purification process development activities included production of four confirmation batches at 500 L pilot-scale using GMP WCB and transmitted set-point and ranges. The levels of NGHC, LMW, and charge variants observed in 500 L scale FCP produced with the optimized process using QC assays were well within historical Dupilumab clinical experience.

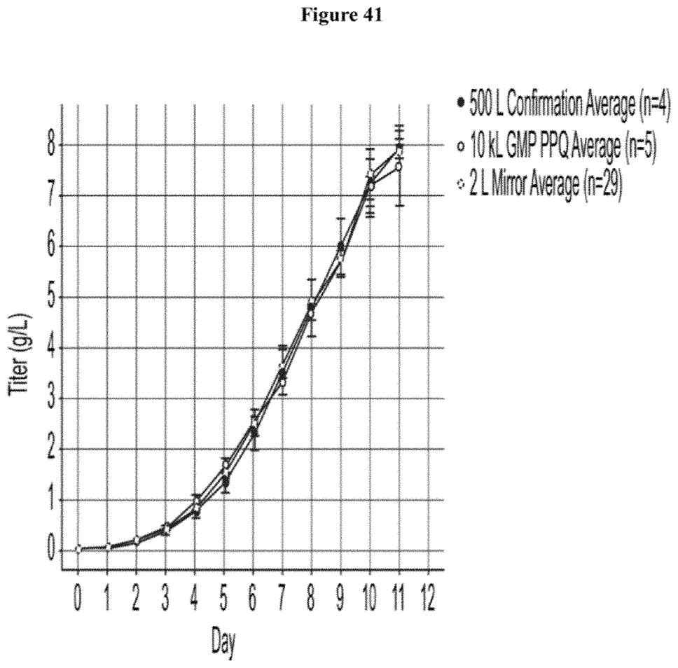

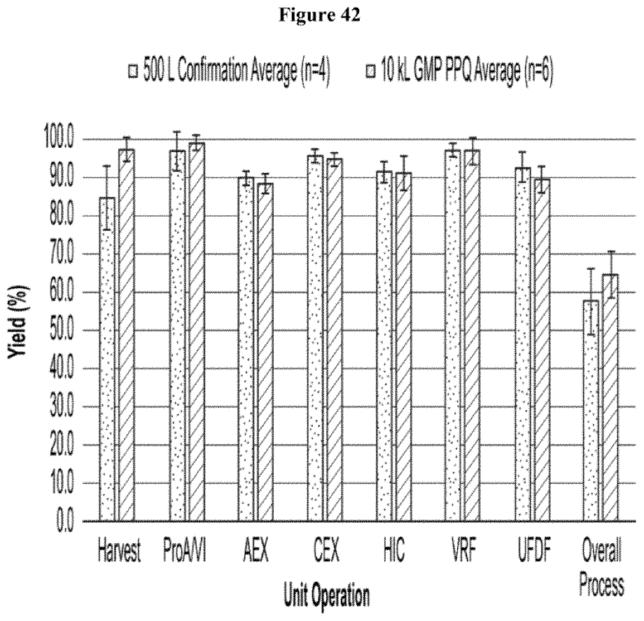

The confirmation batches also illustrated that cell culture and purification process related expectations were met. Consistent titer (shown in FIG. 41), growth, viability, and metabolic profiles were observed for the production bioreactor as well as reproducible growth during seed train operations. Regarding the performance of the purification train, step yields were consistent for all steps, as shown in FIG. 42. Error bars represent 1 standard deviation.

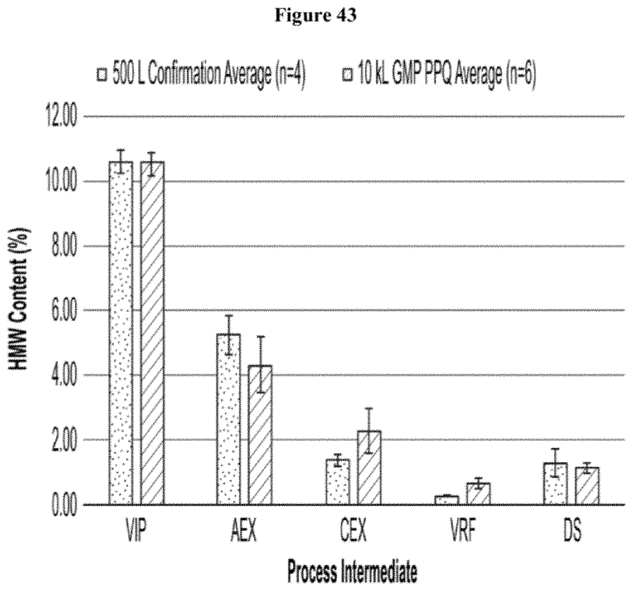

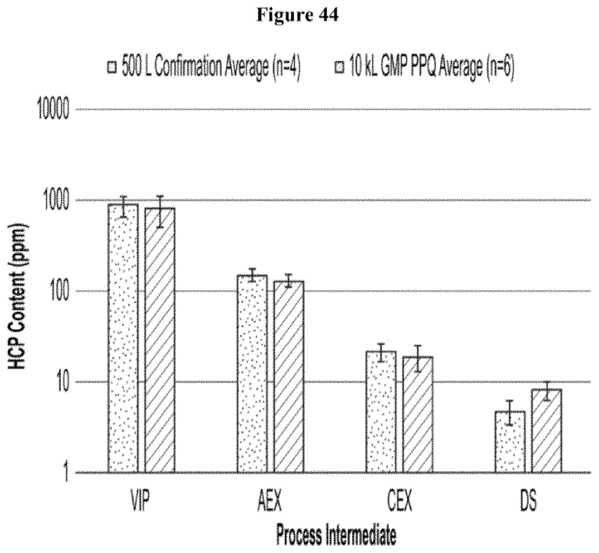

In addition to meeting product quality and performance standards, the performance of pilot-scale confirmation batches has been compared to GMP process performance qualification (PPQ) manufacturing data from the 10,000 L manufacturing area where Dupilumab has been validated with regards to bioreactor titer (FIG. 41), step yield (FIG. 42), and impurity removal (FIG. 43 and FIG. 44). Error bars represent 1 standard deviation. 500 L scale confirmation runs were characterized up to FCP while 10,000 L GMP batches were characterized through DS and FDS. No impurity removal is expected between FCP and FDS impurity content.

Overall, comparable performance between 10,000 L GMP production, 500 L confirmation batches and 2 L bench scale mirrors has been demonstrated for the optimized process of the present invention, which illustrates suitability of scale-down models used to design the process and define pCPP, and further demonstrates effective control of pCPP during scale-up.

### Example 17. Comparability Testing of a Novel Manufacturing Process

Analytical comparability of antibody manufactured using the optimized process versus an existing alternative process was performed using multiple approaches, using Dupilumab as an exemplary antibody product. One approach included evaluating pilot scale material (500 L) of the optimized process to GMP material manufactured at large-scale production (10,000 L) of the alternative process. Another approach included evaluating GMP large-scale production lots from the optimized process compared to the alternative process.

Both assessments demonstrated that the optimized process material is comparable to the alternative process material per ICH Q5E. These studies demonstrate not only that the process improvements to the optimized process have no impact on Dupilumab potency, physicochemical attributes, biochemical attributes, and stability, but that characterization of the 500 L pilot scale material is predictive of protein quality at the 10,000 L scale.

The optimized process DS and 150 mg/mL and 175 mg/mL FDS were compared to the alternative process DS and FDS from approved process areas with the following results.

In-process testing: FDS lots produced via the optimized process in Process Area 1 met the predefined limits for more than 99% of the tests performed. All critical IPCs were met and excursions were found not to be impactful to product quality. This demonstrated that the optimized process to manufacture Dupilumab DS and FDS operated as intended. Evaluation of process and product-related impurities in optimized process material demonstrated that these impurities are cleared to acceptable levels that are consistent with Dupilumab manufacturing experience. Results demonstrate that optimized process and alternative process FDS are comparable with respect to molecular weight and associated molecular features.

Stability: Optimized process FDS long-term stability results met the end of shelf-life specification acceptance criteria throughout a period of long-term storage (−30° C.). Accelerated (25° C.) and stress (45° C.) stability studies indicated that the FDS lots manufactured via the optimized process undergo highly similar changes in fragmentation based on qualitative and quantitative review of the overall degradation profiles.

This disclosure provides a novel method for manufacturing an antibody drug at high titer, with high yield and comparable quality to existing alternative methods. Optimizations to pre-treatment, chromatography, filtration and concentration steps as described herein may be used individually or in combination to improve protein titer and yield. It is to be understood that this invention is not limited to particular methods and experimental conditions described, as such methods and conditions may vary.

### Example 18. Production Using CO2 Sparging of a Human IgG4 Monoclonal Antibody that Binds Interleukin 4 (IL-4) Receptor

The following study was conducted to evaluate the effect of culture pCO₂ on the charge variant profile of a human IgG4 monoclonal antibody that binds to the IL-4R alpha (a) subunit and thereby inhibits Interleukin 4 (IL-4) and Interleukin 13 (IL-13) signalling.

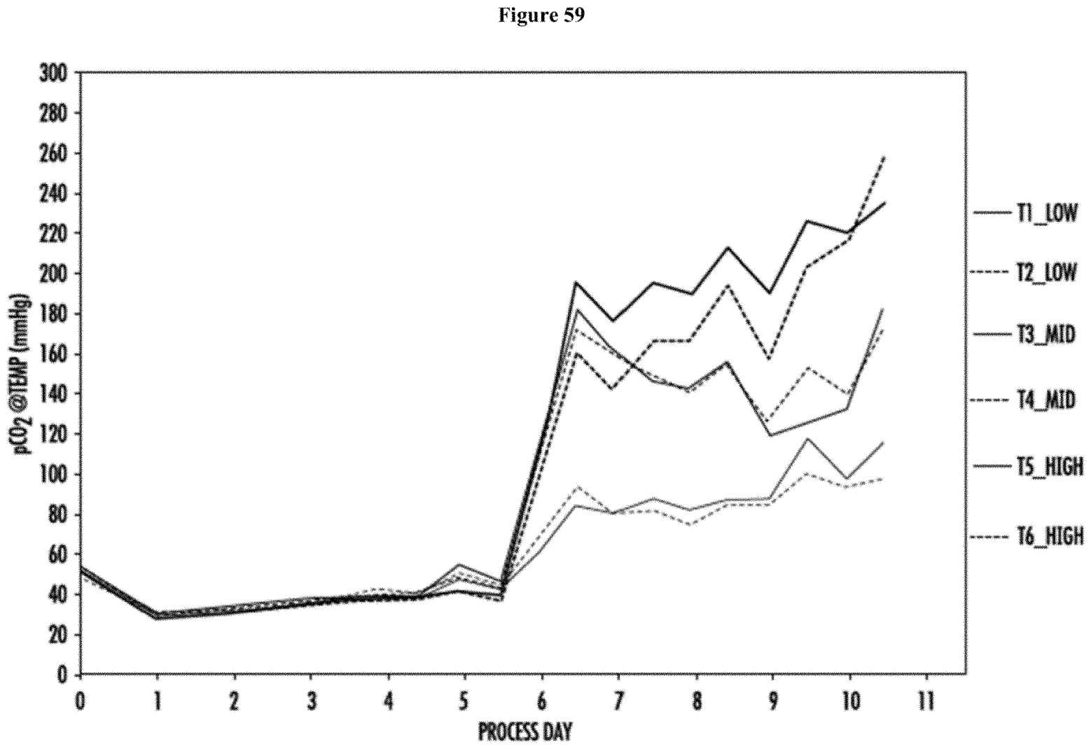

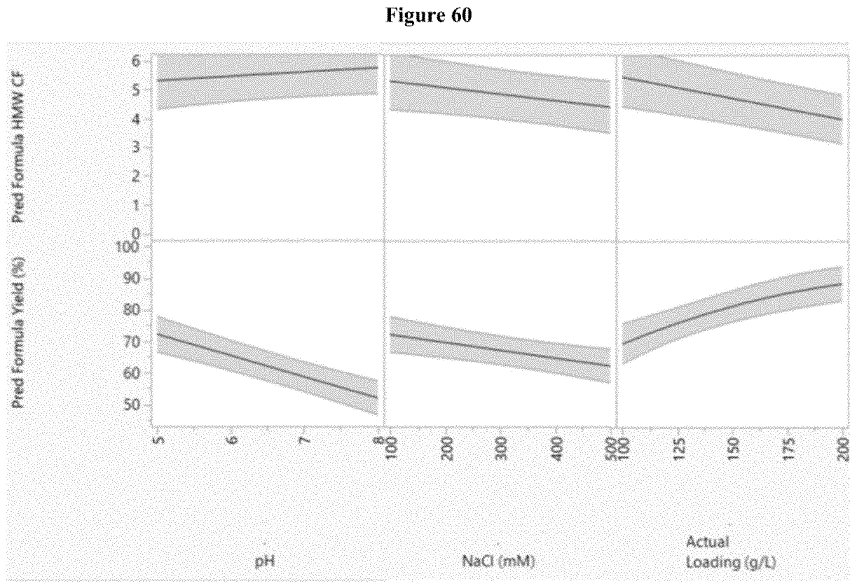

Culture media within a production bioreactor was inoculated with cells at a concentration of about 12×10⁵ cells/mL, and allowed to grow in a fed-batch process. Once peak Viable Cell Density (VCD) of 200×10⁵ cells/mL was reached on Day 5.5, CO₂ sparging was modified as defined in Table 22 to vary pCO₂ levels within the cell culture. The resultant pCO₂ profiles of the three experimental conditions are provided in FIG. 59.

TABLE 22
(Percentage Minimum CO₂ Sparge Flow Rate of Low and High
pCO₂ Conditions Relative to Medium pCO₂ Condition (Control))

| Day | 0-5.5 | 5.5-6.0 | 6.0-10.5 |
|---|---|---|---|
| Low pCO₂ Condition | 100 | 40 | 33 |
| Medium pCO₂ Condition | 100 | 100 | 100 |
| High pCO₂ Condition | 100 | 160 | 167 |

Following 10.5 days of culture, the bioreactors were harvested and the monoclonal antibody was purified. The glycosylation and charge variant profiles were determined. It was noted that as pCO₂ levels within the production bioreactor increased, there was a concomitant decrease in levels of basic variants, as measured by imaged-capillary isoelectric focusing (iCIEF) (Table 23). In addition, an increase in pCO₂ led to a concave acidic variant profile, peaking with mid pCO₂ condition, but dropping to the lowest percentage at the high pCO₂ condition (Table 24). The overall trend was a lower percentage of acidic charge variants, and the medium pCO₂ measure was likely a result of error.

TABLE 23
(Effect of Culture pCO₂ on Basic Charge Variants)

| Condition | Basic Variants (%) |
|---|---|
| Low pCO₂ | 9.0 |
| Medium pCO₂ | 8.3 |
| High pCO₂ | 7.9 |

TABLE 24
(Effect of Culture pCO₂ on Acidic Charge Variants)

| Condition | Acidic Variants (%) |
|---|---|
| Low pCO₂ | 37.0 |
| Medium pCO₂ | 39.0 |
| High pCO₂ | 36.0 |

An analysis of another antibody not shown established that increased pCO₂, and not decreased pH, was the only statistically significant term for lowering the percentage of acidic charge variants (Region 1) with the human IgG4 monoclonal antibody. Thus, for the IgG class, represented by a human IgG4 monoclonal antibody here, increased pCO₂ itself lowers the percentage of acidic charge variants, and the lowering of the percentage of acidic charge variants in IgG4 antibodies is not caused by decreased pH values. Dupilumab is a human monoclonal antibody of the IgG4 subclass that binds to the IL-4R alpha (a) subunit and thereby inhibits Interleukin 4 (IL-4) and Interleukin 13 (IL-13) signalling.

It is to be understood that the description, specific examples and data, while indicating exemplary embodiments, are given by way of illustration and are not intended to limit the present invention. Various changes and modifications within the present inventions, including combining embodiments in whole and in part, will become apparent to the skilled artisan from the discussion, disclosure and data contained herein, and thus are considered part of the inventions.

### Example 19. Comparability Testing of Optical Probe and Electrochemical Probe

Cell culture runs, Runs 1-6 (shown in Table 25), were performed in large scale stainless-steel stirred-tank production bioreactors utilizing mammalian cells. Each bioreactor was cleaned-in-place and steamed-in-place prior to the addition of medium. The medium was sparged with pharmaceutical grade air and subsequently inoculated with cells from the inoculating seed train bioreactors. The bioreactors were operated in fed-batch mode, with typical processing activities such as feed additions, agitation and/or gas sparging adjustments performed as dictated by the applicable process descriptions. Dissolved oxygen was controlled to a setpoint via sparging of air and/or oxygen gas on a cascade control loop during the runs. Cultures were harvested after specified periods of time depending the specific processes and although the process in Run 1 was different from Runs 2-6, those differences were not relevant to the methods and analysis in this example.

Dissolved oxygen probes were calibrated, installed in the bioreactor post-CIP, and standardized to 100% saturation in the air-saturated medium immediately prior to inoculation. It may be assumed that any dissolved oxygen gradient within the bioreactor is negligible. Dissolved oxygen probes were installed into ports located along the probe-belt in the lower third of the bioreactor. It was observed in that there is no discernible difference associated with probe performance between probe ports located within the probe belt, allowing for greater flexibility regarding the location of the probes.

The measurements from one selected dissolved oxygen probe, designated the controlling probe, were used for control of the air/oxygen mass flow controller output via a cascade control loop. All other installed probes were used for monitoring purposes only. The experimental runs shown in Table 25 were executed in two phases. Runs 1-4 were executed as part of Phase 1 “Passive monitoring”, whereby the two different optical DO probes were installed as monitoring probes only, and the electrochemical probe was utilized as the controlling probe (with an additional, secondary electrochemical probe installed as a monitoring probe, available for use in the event of failure of the primary probe). Based on the outcome of these runs, one of the optical probes was selected for use in Phase 2 of the experiment, whereby the optical probe was implemented as a controlling probe for Runs 5 and 6, with an electrochemical probe installed as a monitoring probe. During Phase 2, the protocol allowed for tuning of PID parameters if necessary, including adjustment of the proportional gain, integral time and derivative time as well as the sampling interval.

TABLE 25
(Summary of the methodology for cell culture runs)

| | | Controlling | Monitoring |
|---|---|---|---|
| Run no. | Phase | Probe | probes |
| Run 1 | 1 - Passive monitoring | Electrochemical | Electrochemical |
| Run 2 | | | Optical Probe A |
| Run 3 | | | Optical Probe B |
| Run 4 | | | |
| Run 5 | 2 - Controlling | Optical Probe A | Electrochemical |
| Run 6 | | | |

As illustrated in Table 25 above, optical dissolved oxygen technology was tested in a total of six cell culture runs in a large-scale stainless-steel production bioreactor. Both optical probes under evaluation were implemented in a monitoring capacity for Runs 1-4 and assessed against the electrochemical probe performance in a side-by-side comparison. Optical Probe A was then tested in a controlling capacity for Runs 5 and 6, and performance was consistent with Phase 1. In addition, benchtop assessments were performed.

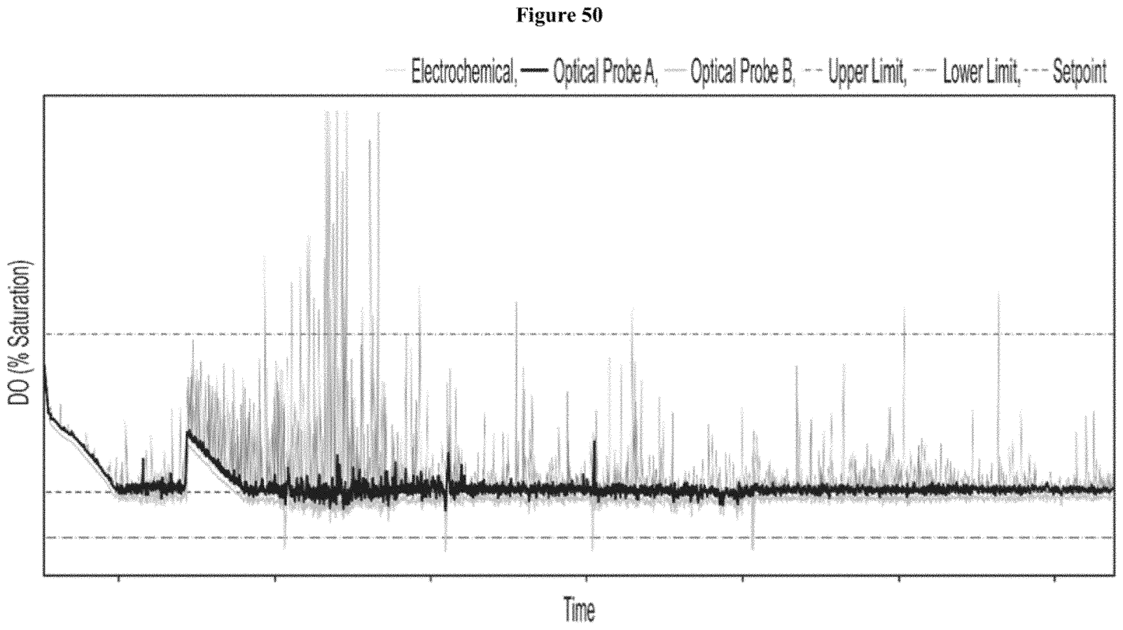

The Phase 1 data results are presented in FIG. 50-FIG. 53. All sensor data was imported into a process data analytics application (henceforth referred to as PDAA). Both Optical Probe A and Optical Probe B show dramatic reduction in signal noise, in all cell culture runs (see, Runs 1-4). The upper and lower DO limits of the normal operating range for the applicable processes are indicated in the graphs by dashed lines. There were no observed “spikes” above the upper limit for either optical probe in each of the four runs, whereas multiple excursions above 100% were observed for the electrochemical probes. This data shows that optical probes are not as susceptible to signal noise as electrochemical probes.

With all three probes, some additional noise was observed approximately 25-50% of the way through each run (slightly earlier for Run 1). It is was found that the combination of processing parameters used at this point in the run may have resulted in sub-optimal dispersion of bubbles. Even during these periods of additional noise, the optical probes exhibited superior performance in terms of noise reduction, in comparison to the electrochemical probe. Thus, optical probes can be used as an alternative to electrochemical probes with reduced maintenance requirements and lower incidence of false positive excursion events.

To quantitatively evaluate the difference in noise between the three different types of probe, data from each cell culture run were exported from PDAA to Microsoft Excel, and the average and standard deviation for each data set was calculated as shown in Table 26. To avoid interference with calculation of the average and standard deviation, the data during the decline to the DO setpoint at the start of each run was not included. Accordingly, the exported time range for each cell culture run encompassed the time from when the DO target setpoint had been reached, to the end of the cell culture run only. Note, that in the case of Run 1, where a processing activity resulted in a significant increase in the DO % saturation early in the run (see FIG. 50), the time range selected for export spanned from the time when the DO concentration had reestablished at the target setpoint.

TABLE 26
(Standard deviation of dissolved oxygen measurements
by each probe type for Phase 1)

| | Standard Deviation | | |
|---|---|---|---|
| | Optical Probe A | Optical Probe B | Electrochemical |
| | (% DO | (% DO | (% DO |
| Run | Saturation) | Saturation) | Saturation) |
| 1 | 2.6 | 2.6 | 4.3 |
| 2 | 3.8 | 3.8 | 8.0 |
| 3 | 3.6 | 3.6 | 7.0 |
| 4 | 4.2 | 4.3 | 8.5 |
| Average | 3.6 | 3.6 | 7.0 |
| Runs 1-4 | | | |

On average, the standard deviations of the electrochemical probe was almost twice the standard deviations of the optical probes, across the four runs and show that the electrochemical signal behavior was much noisier, with standard deviations on average approximately twice as large as the optical probes. Thus, overall, it may be inferred that both Optical Probe A and Optical Probe B were successful in reducing the noise associated with the dissolved oxygen signal, achieving trends similar to the filtered electrochemical signal.

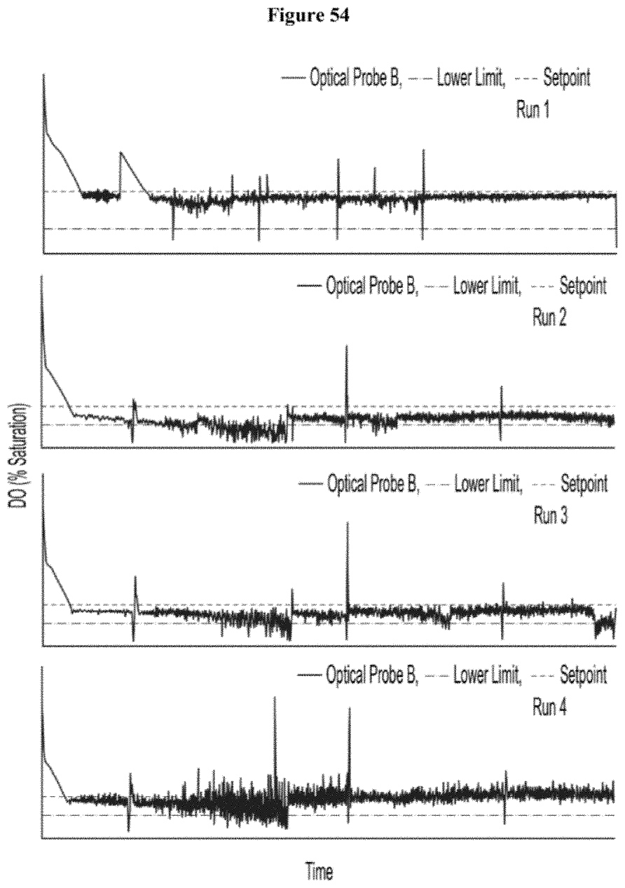

It should be noted that a greater offset was observed with the anti-bubble cap compared to the standard cap. Although the offset was greater, the anti-bubble cap resulted in greater noise-reduction as shown in FIG. 54 (anti-bubble cap for Optical Probe B was utilized for Runs 1-3, and a standard cap was utilized for Run 4).

### Example 20. Electrochemical Probe Signal Processing

An assessment of offset between probes was performed to gain an understanding of comparability of the measurement. This consisted of visual analysis of data overlays presented in PDAA, as well as analysis of segments of exported data in Excel from the start, middle and end of the run, to identify the magnitude of the present offset through comparison of averages. The segments for analysis were selected based on time intervals where the electrochemical noise was considered minimal.

It was found that a method for offset identification is vital. If a probe was operating at a significant offset to the electrochemical probe, the probe could under or overestimate the true level of dissolved oxygen in the bioreactor. Exposure of the cells to conditions of particularly low or high oxygen concentration could have detrimental effects to the cell culture. A preliminary assessment of drift was also performed, by reviewing the data for any increasing or decreasing trend across the examined periods at the start, middle and end of the batch. Based on the offset assessment from Phase 1, an offset correction was proposed for each probe, and a new signal for each probe during each run was created in PDAA. These were also analyzed visually through data overlays as well as qualitatively as described above to evaluate improvement.

The ability of the probe to detect a chance in oxygen concentration in a timely manner was also found to be important in maintaining proper dissolved oxygen levels within the bioreactor. The behavior of the optical probes was contrasted two ways: (1) visually with the electrochemical probe behavior and (2) by analyzing the time for an optical and an electrical probe to stabilize upon exposure to nitrogen (e.g., zero concentration).

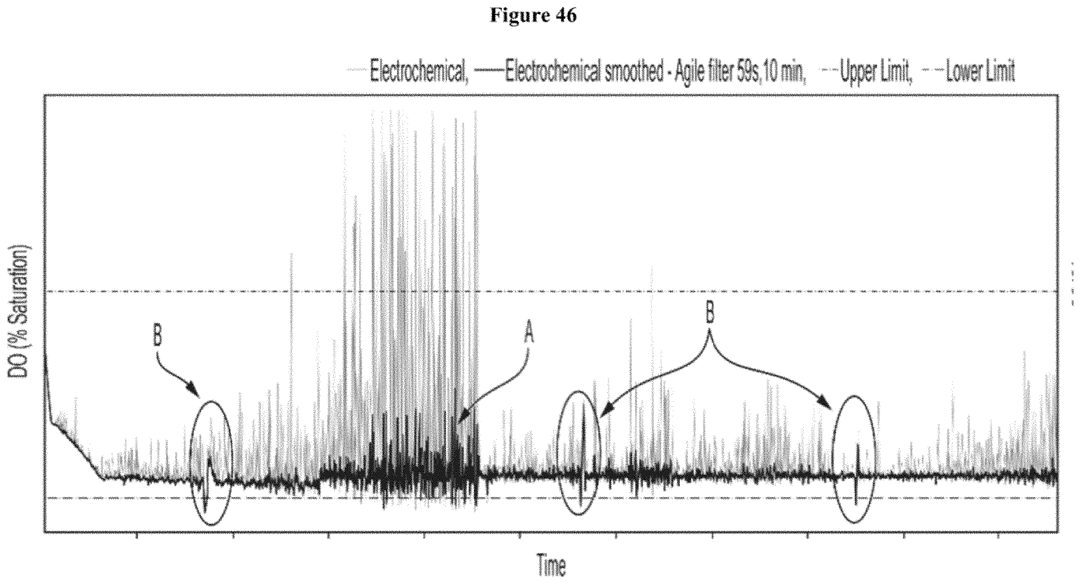

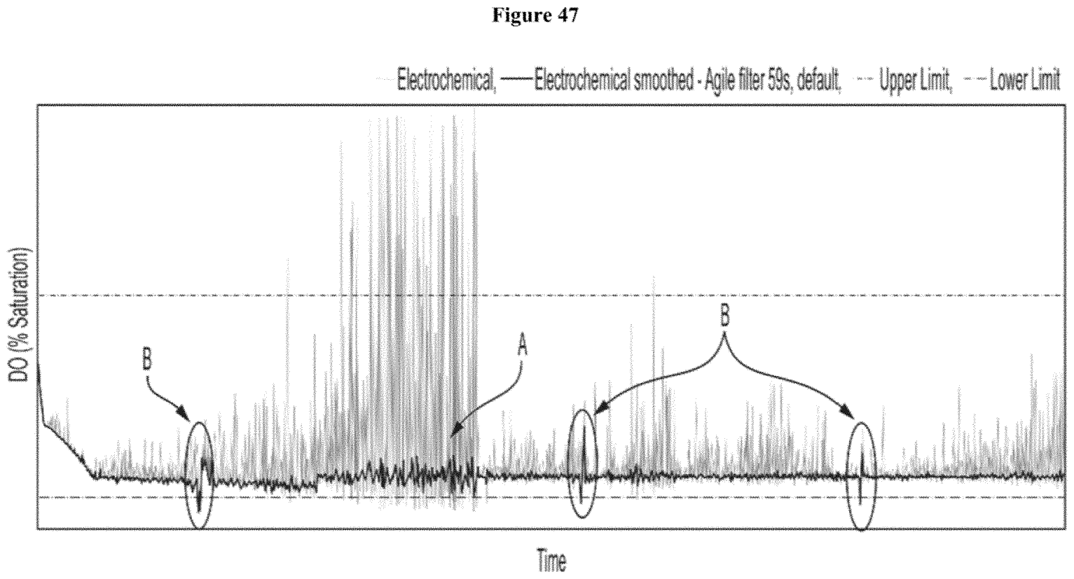

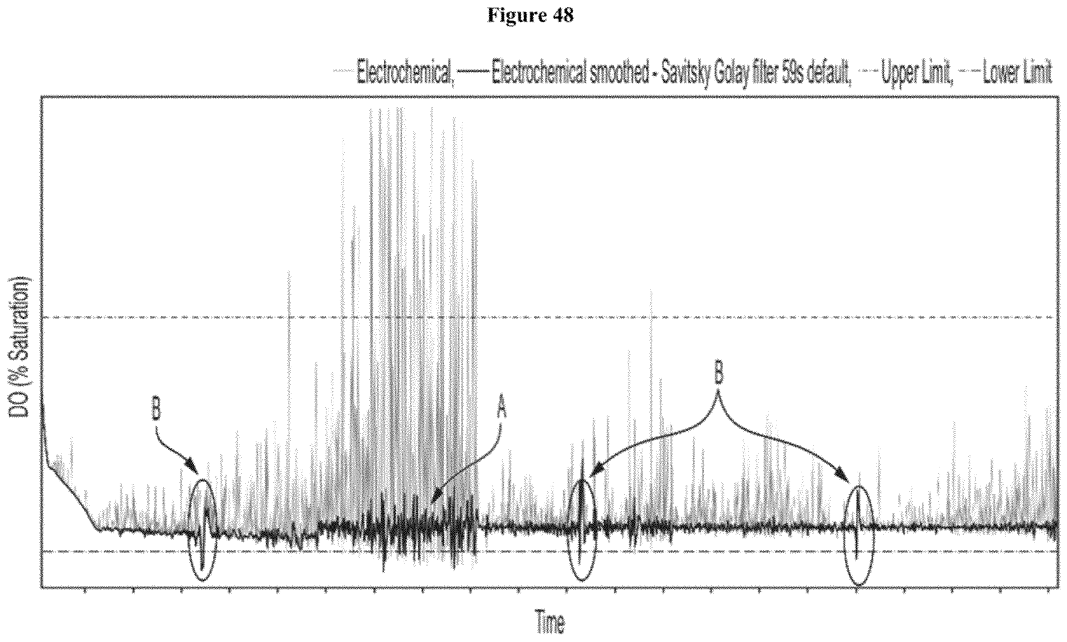

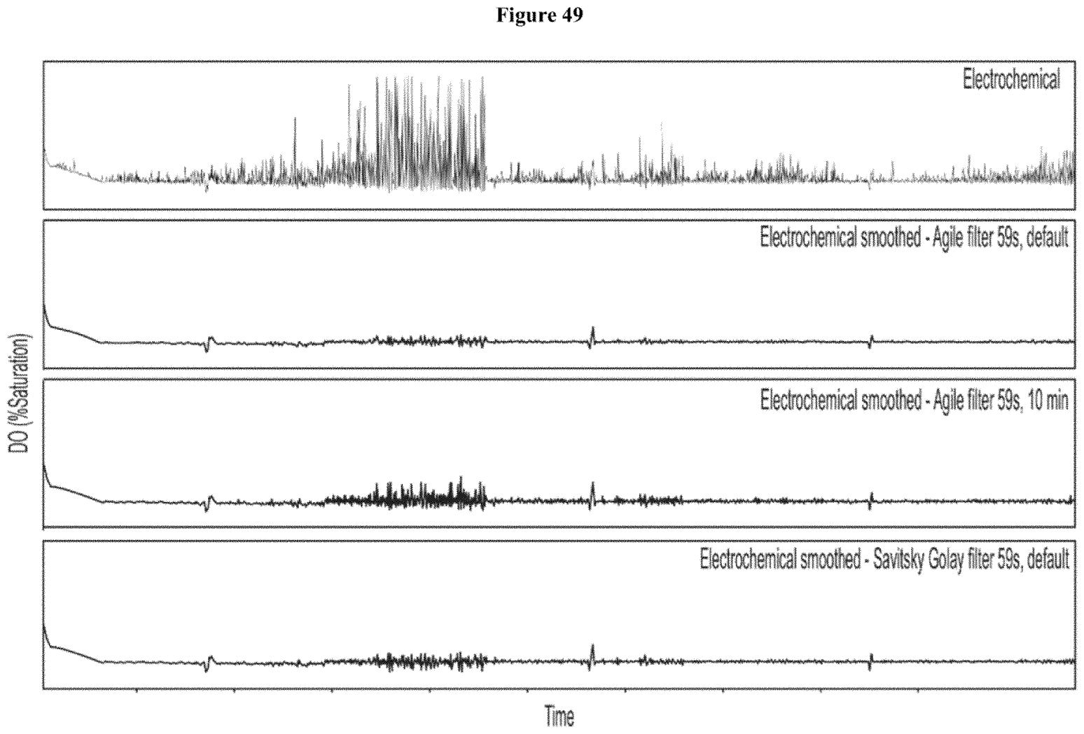

Alternate signal processing methods was also performed to alleviate signal noise for electrochemical probes. In one embodiment smoothing was used to reduce individual data points that are higher than adjacent data points, and increasing of individual datapoints that are lower than adjacent points. It is important however, that the signal smoothing does not remove or obscure true data disturbances. A review of several of the conventional acceptable processing disturbances revealed that many of these last less than 33 minutes, meaning that the smoothing for this timeframe would include substantial data that is not reflective of the disturbed state and could be “oversmoothed”. To solve this problem, a smaller sampling window of 10 minutes was applied initially, and as shown in FIG. 46, for example, a filter was applied to cell culture Run 2. It was found that increasing the sampling window did not significantly oversmooth these processing disturbances, but did have the effect of eliminating excursions on the low side that were not necessarily accurate reflections of the process. This is illustrated in FIG. 47 for cell culture Run 2. A Savitzky-Golay filter was also tested as illustrated in FIG. 48, however, it was not as effective as the Agile filter. A stacked comparison of the filters and sampling windows is shown in FIG. 49. Based on the results observed from application of the filters, it was found that signal processing provides an alternative solution to improving noisy or erratic signals from electrochemical dissolved oxygen sensors. Although the above signal filtering was performed on the received data, it is understood real-time signal filtering may be applied. Additional filters may also be applied.

### Example 21. Optical Probe Offset Assessment

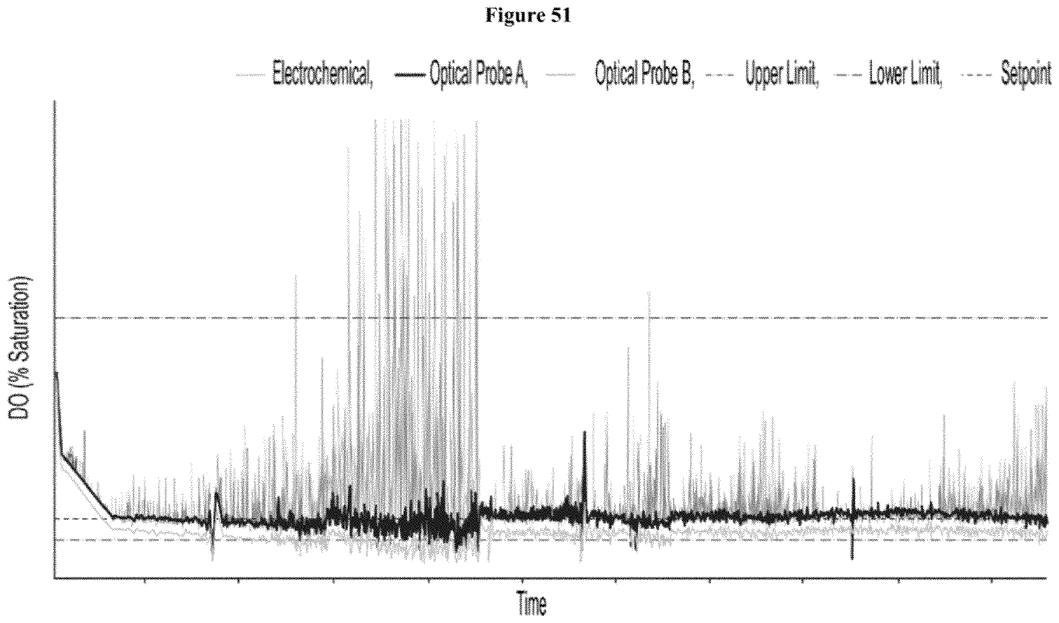

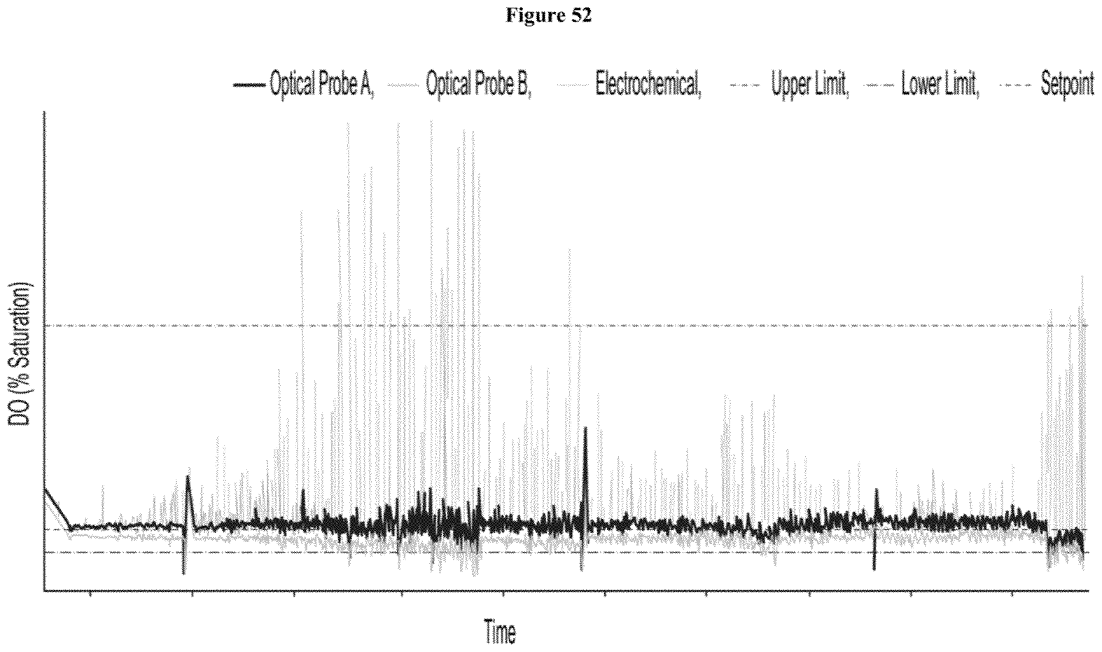

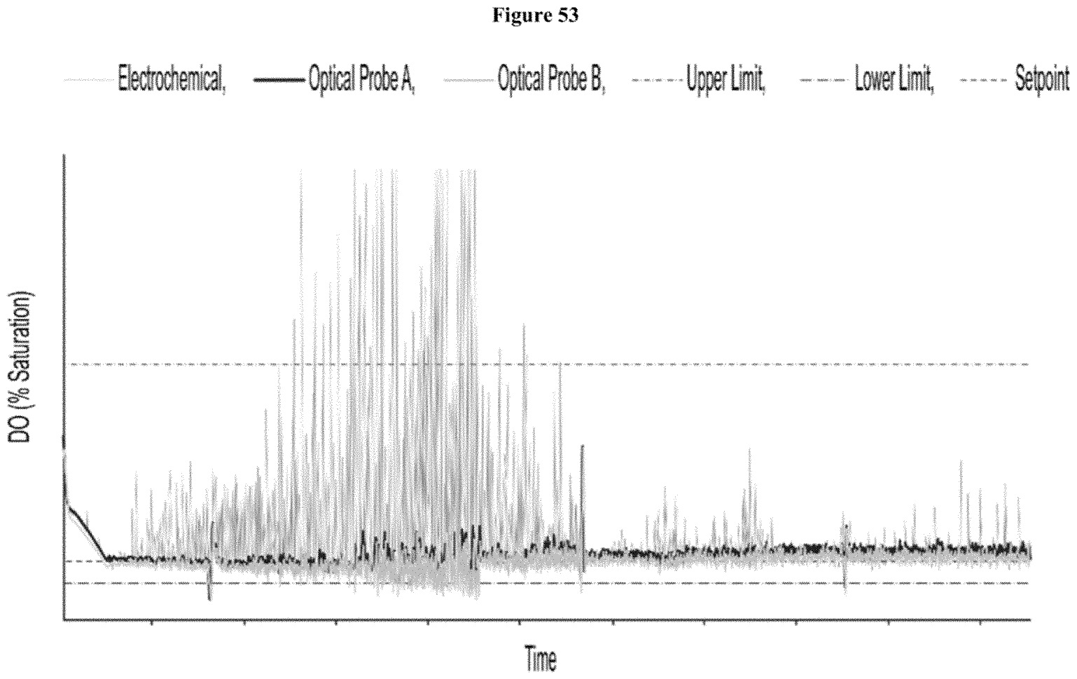

As shown in FIG. 50, FIG. 51, FIG. 52, FIG. 53, an offset exists between the different types of probes. Optical Probe B reads approximately 2-3% lower than the electrochemical probe. Optical Probe A reads much closer to the electrochemical probe, although typically −1% higher. These results were observed for the majority of the data for all cell culture runs executed as part of Phase 1 (see Runs 1-4).

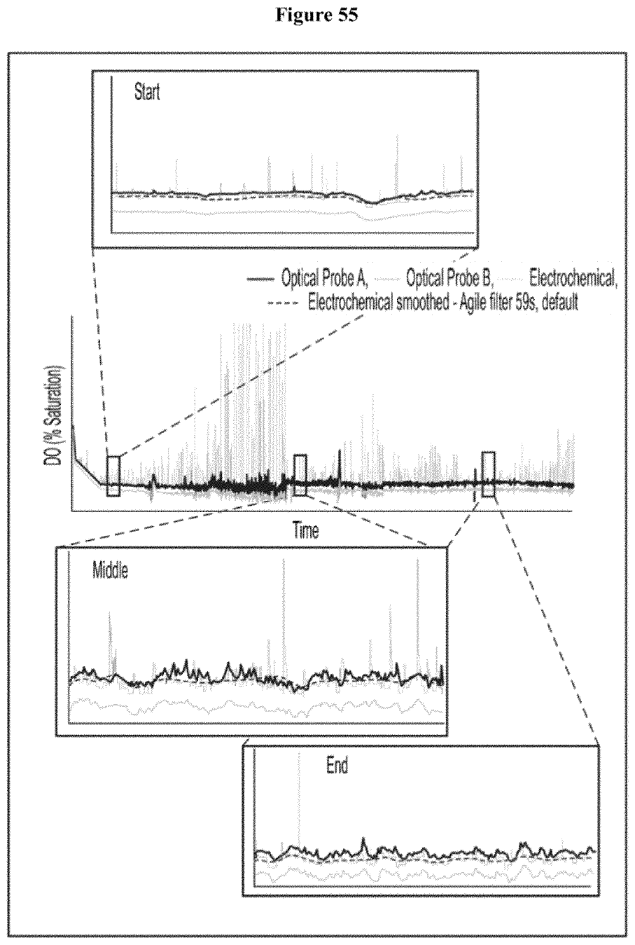

To quantify the offset and understand if the offset increases or decreases with time, each optical probe signal was compared with the electrochemical probe signal. Data at the start (e.g., first third), middle (e.g., middle third) and end (e.g., last third), of each run during a selected period of 5 hours, when the electrochemical signal was not substantially noisy was exported to Microsoft Excel, and the average value was determined for each signal. As shown in FIG. 55, the offset over time was analyzed. The standard deviation is reported based on the average for the entire lot (excluding initial DO concentration to setpoint), by systematically focusing on time periods where the electrochemical signal is relatively stable, potential for significant distortion of the average value, due to the presence of noise is minimized. Although an averaged value was determined, a variable offset could be determined at different time points and it is believed that it could be applied to improve accuracy.

The offset assessment results for Phase 1 are summarized in Table 27. Where the calculated delta between the electrochemical probe average and the optical probe average lies within the standard deviation of the electrochemical probe, the delta has been shaded, indicating good alignment between the probes.

TABLE 27

| | | Delta Optical A | Delta Optical |
|---|---|---|---|
| | Electrochemical | average vs | B vs Electro- |
| | Standard | Electrochemical | chemical |
| | deviation | average | Average |
| | (% saturation) | (% saturation) | (% saturation) |
| Run 1 Start | ±2.6 | 0.9 | −2.4 |
| Run 1 Middle | ±1.6 | 0.7 | −2.9 |
| Run 1 End | ±1.2 | 0.6 | −2.7 |
| Run 2 Start | ±1.4 | 1.1 | −4.7 |
| Run 2 Middle | ±3.0 | 0.8 | −6.6 |
| Run 2 End | ±1.3 | 2.1 | −5.2 |
| Run 3 Start | ±0.5 | 1.5 | −3.2 |
| Run 3 Middle | ±3.6 | 1.6 | −3.8 |
| Run 3 End | ±2.4 | 3.2 | −2.7 |
| Run 4 Start | ±0.6 | 0.9 | −1.9 |
| Run 4 Middle | ±1.5 | 2.1 | −0.4 |
| Run 4 End | ±1.6 | 3.5 | 1.1 |

Comparison of electrochemical probe standard deviation and delta between adjusted optical probe signal average and electrochemical probe average. Data taken from selected 5-hour periods, during which electrochemical signal noise was considered minimal at the start, middle and end of each run. The shaded cells indicate that the delta lies within the standard deviation of the electrochemical probe data and is therefore considered good alignment.

As shown in Table 27, 50% of the calculated delta values for Optical Probe A fell within the standard deviation of the electrochemical probe, compared with only 25% of the calculated delta values for Optical Probe B (refer to delta values shaded in Table 27).

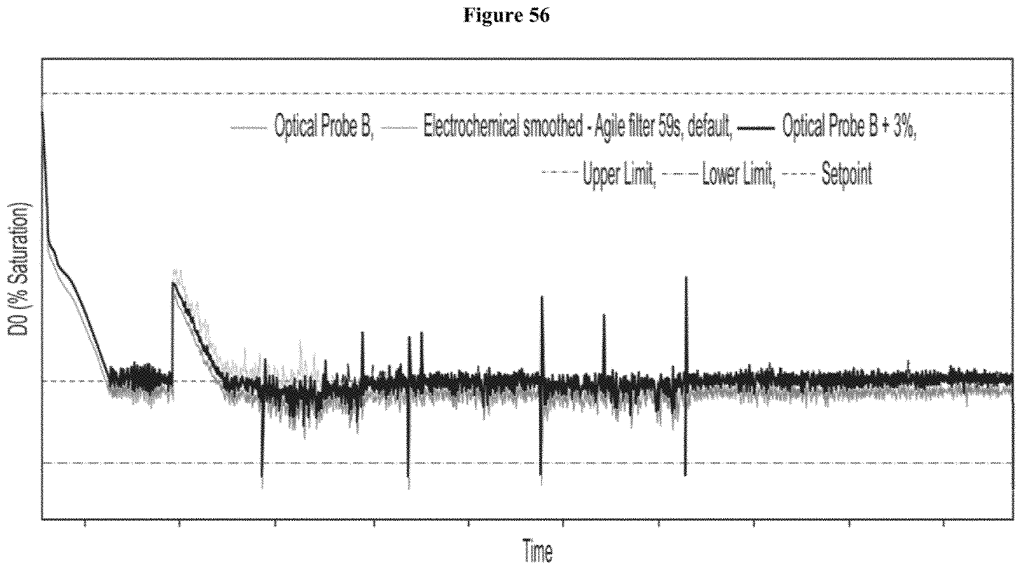

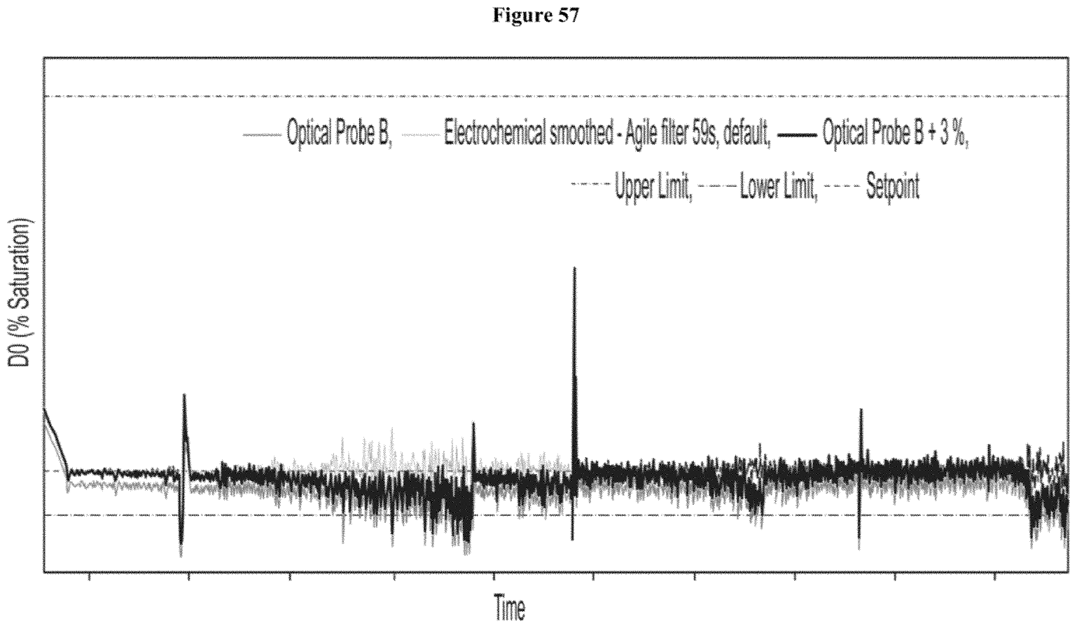

Based on the data for Run 1 and Run 3 in Table 27, the average offset observed for Optical Probe B, using the anti-bubble cap was −3.0% saturation (Run 2 was not counted due to the lower-than-expected readings, as mentioned previously). A new signal was generated in PDAA by adding 3.0% to the Optical Probe B measurement for both Run 1 and Run 3. As can be seen in FIG. 56 and FIG. 57, the adjusted signal aligns quite well with the smoothed electrochemical signal during the initial few days as well as at the latter stages of the run.

Based on the results, both optical probes performed well, were compatible with standard sterilization procedures and could be considered as an appropriate alternative to the electrochemical probe. Both Optical Probe A and Optical Probe B produced less noisy signals than the electrochemical probe, and there was no notable difference in noise between the optical probes. Both optical probes generated signals with slight offsets compared with the electrochemical probe. The offset associated with Optical Probe B was larger in magnitude, with an approximate offset value of −3.0% (saturation), based on assessment of segments at the start, middle and end of the run, during periods where the electrochemical noise was minimal. As noted above, however, a variable offset may further improve accuracy.

### Example 22. Seed Train Optimization

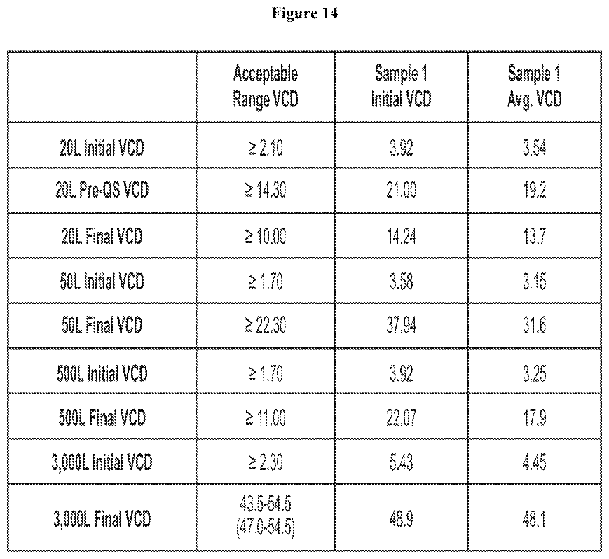

CHO cells were transfected with DNA expressing Dupilumab. The CHO cells were incubated in CDM media, including various supplements described above. As shown in FIG. 14, these CHO cells were cultured in 20 L, 50 L, 500 L, 3,000 L and 10,000 L vessels using an optimized seed train wherein the initial VCD was increased compared to a standard initial VCD (shown as Acceptable Range VCD). The final VCD in the 3000 L vessel for the optimized seed train did not change and the initial 10,000L VCD in the optimized seed train was comparable to the standard seed train (e.g., 10.38×10⁵-14.63×10⁵ cells/mL).

Using the same CHO cells and media, Samples 2-9 were cultured wherein the initial VCD for the seed train fell within the Acceptable Range VCD as shown in FIG. 14. The CHO cells for Samples 1-9 were then harvested and purified using the process described herein (see e.g., FIG. 30) and the average titer (g/L) was calculated for Sample 1 (optimized seed train) and Samples 2-9 (standard seed train falling within the Acceptable Range VCD).

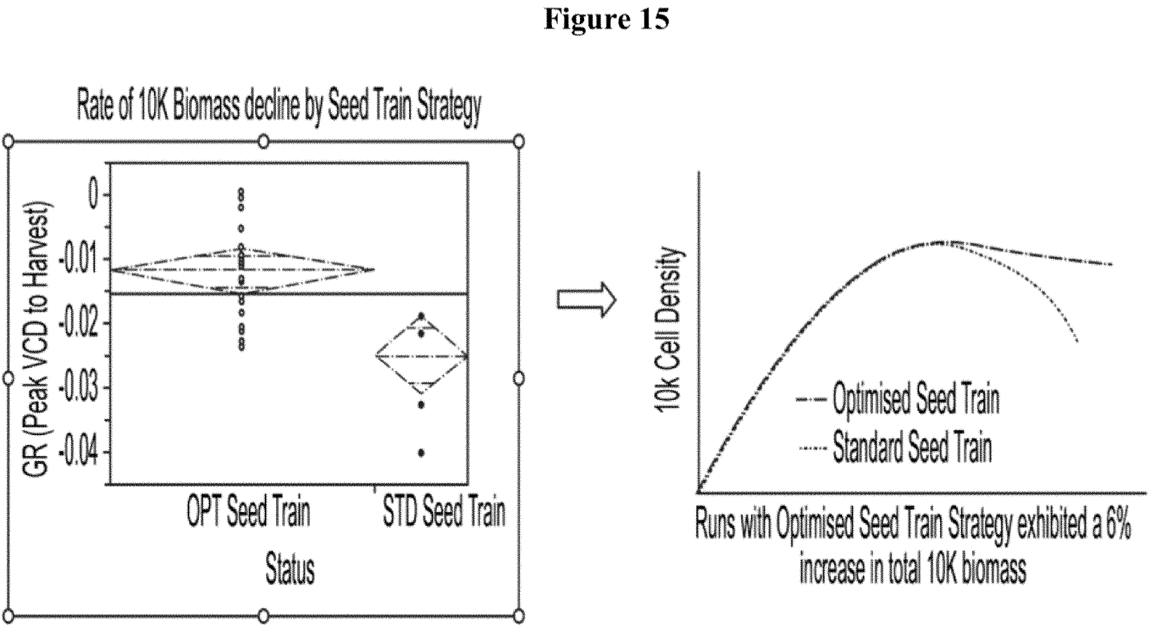

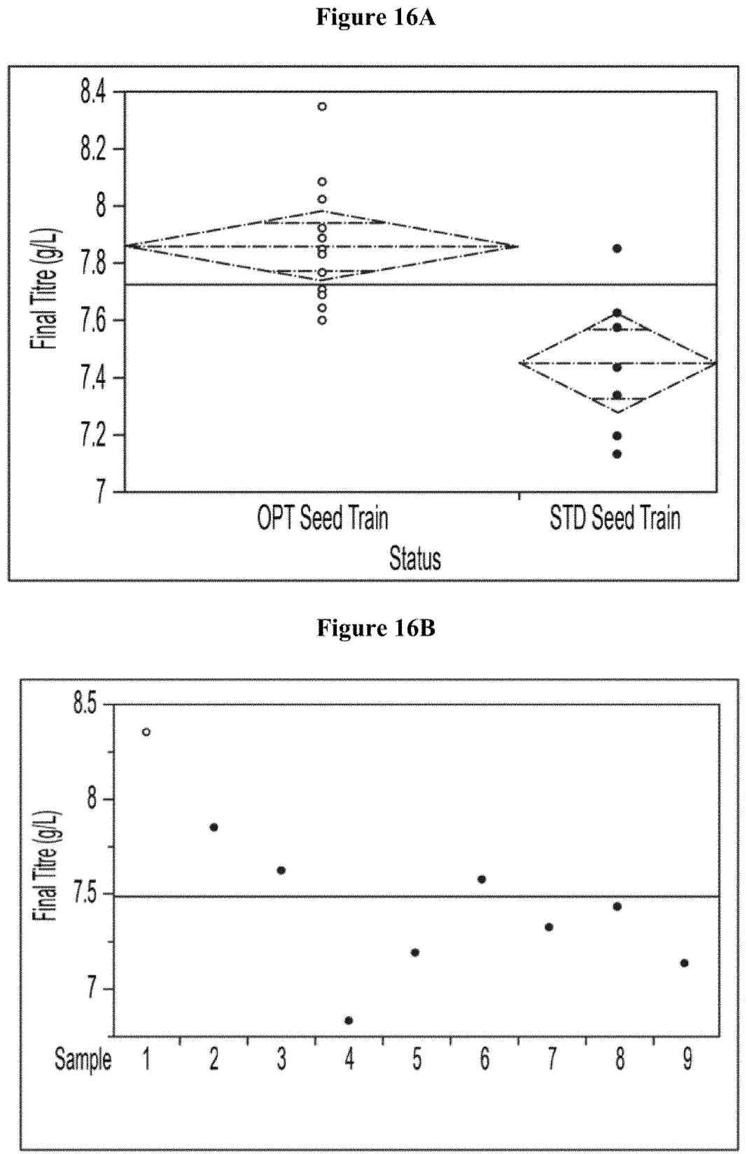

As shown in FIG. 15, the biomass of the optimized seed train declined less than the standard seed train, exhibiting a 6% increase in total biomass. As shown in FIG. 16A and FIG. 16B, the optimized seed train led to an increase in final titer (g/L) of at least 0.4 g/L compared to the standard seed train.

### Example 23. VCD Capacitance Probe

The investigation was split into two phases. Phase 1 assessed two different cell lines, cell line A and cell line B, throughout seed train. Two bioreactors were run for each cell line, one bioreactor with a capacitance probe and one without. Phase 2 investigated cell line B throughout seed train and production. Three bioreactors were run, two with capacitance probes and one without.

Two different mammalian cell lines, cell line A and cell line B, each expressing a different therapeutic modality, were used. For cell line A, chemically defined seed medium (CDSM) was used. For cell line B, soy-based seed medium (SBSM), soy-based production medium (SBPM), feed medium A (FMA) and feed medium B (FMB) were used.

### Media Preparation

### Cell Line A

All raw material component lots were kept identical across all conditions. A calculated volume of chemically defined seed medium was prepared. All media was stored in a refrigerated unit.

### Cell Line B

All raw material component lots were kept identical across all conditions. A calculated volume of soy-based seed medium was prepared. A set volume of feed medium A was prepared for each bioreactor. Feed medium A was prepared the day it was required and added to the reactor within seven hours of initiating preparation of the feed. All media was stored in a refrigerated unit.

### Seed Culture

### Cell Line A

Phase 1 consisted of assessing the viable cell density of cell line A throughout seed train (e.g., the N-3, N-2 and N-1 stages). Cell culture was extracted from a separate study. The cell suspension was taken from a wave bioreactor and used to inoculate the two N-3 bioreactors.

### Cell Line B

Phase 1 consisted of assessing the viable cell density of cell line B throughout seed train (e.g., the N-3, N-2 and N-1 stages). One vial of cell line B was thawed in the biosafety cabinet (BSC). The thawed cells were transferred into a shake-flask with the required volume of pre-warmed soy-based seed medium. Following successful vial thaw, the shake flask was placed on an appropriate shaker platform within an incubator set to the required temperature and CO₂ setpoints for cell line B. After a set time-point after the transfer, a sample was taken from the shake flask and a bioanalyzer analysis was performed on the sample.

### Seed Expansion in Wave Bioreactor 1

The required volume of soy-based seed medium was aliquoted into a media bag and transferred into an incubator to warm prior to use. After the required shake-flask expansion time, a sample was taken from the shake flask and a bioanalyzer analysis was performed. The required volume of soy-based seed medium was added to an inflated wave bioreactor 1 and the cell culture was transferred from the shake flask into the wave bioreactor 1. The wave bioreactor was placed on a wave rocker with suitable set-points for cell line B. Post-inoculation, a sample was taken from the wave bioreactor 1 and analyzed on the bioanalyzer. Once the expansion times were met, a sample was taken from the wave bioreactor 1 and analyzed using the bioanalyzer. The required volume of pre-warmed soy-based seed medium was transferred to the wave bioreactor 1, carrying out the media top-up. Post addition of the media, a sample was taken from the wave bioreactor and analyzed on the bioanalyzer.

### Seed Expansion in Wave Bioreactor 2

The required volume of soy-based seed medium was aliquoted into a media bag and placed in an incubator to warm prior to the transfer. Once the required expansion times were met, a sample was taken from the wave bioreactor 1 and a bioanalyzer analysis was performed. A wave bioreactor 2 was placed on a suitable wave platform and the required set-points for cell line B. The required volume of pre-warmed soy-based seed medium was transferred to an inflated wave bioreactor 2 and the cell culture was transferred from the wave bioreactor 1 into the wave bioreactor 2. Post-inoculation, a sample was taken from the wave bioreactor 2 and a bioanalyzer analysis was performed. A required volume of soy-based seed medium was aliquoted into a media bag and placed in an incubator to warm for a suitable period. Once expansion times were met, a sample was taken from the wave bioreactor 2 and a bioanalyzer analysis was performed. The required volume of pre-warmed soy-based seed medium was added to the wave bioreactor 2, carrying out the media top-up. Post addition of the media, a sample was taken from the wave bioreactor 2 and analyzed on the bioanalyzer.

### Seed Expansion in the Bioreactors—Preparation, Inoculation and Sampling

### Cell Line A

The pH probes and the zero percent value for the dissolved oxygen (DO) probes were calibrated. Two bioreactors were washed, built, bioreactor packs were added, and they were autoclaved. The appropriate volumes of chemically defined seed medium were aliquoted into suitable media bags and warmed for a set period. Once bioreactors finished in the autoclave, calibration chemically defined seed medium was transferred into the bioreactors. The suitable cell line A setpoint parameters were input into the bioreactor's control tower. The required control parameters were turned ON. The dissolved oxygen probes were calibrated at 100% saturation. Appropriate volumes of three solution additions were aliquoted into three transfer bottles, per bioreactor. The transfer bottles were sterile welded onto the bioreactors and the lines were primed. The calibration media was drained from the bioreactors and the pre-warmed chemically defined seed medium was transferred into the bioreactors. Once expansion times were met, a sample was taken from the wave bioreactor 2 and a bioanalyzer analysis was performed. A suitable volume of cells was aliquoted from the wave bioreactor 2 into a suitable media bag and then transferred into the N-3 bioreactors. The required control parameters left were turned ON. Post inoculation, a sample was taken from the bioreactor and analyzed on the bioanalyzer. Once expansion times were met for each seed train step, a sample was taken from the bioreactor and a bioanalyzer analysis was performed. Once viable cell density in-process control ranges were met, transfers occurred. The bioreactors were sampled three times daily, and pH adjustments were performed when necessary.

### Cell Line B

Both the pH probes and the zero percent value for the dissolved oxygen (DO) probes were calibrated. Two bioreactors were washed, built, bioreactor packs were added, and they were autoclaved. The appropriate volumes of soy-based seed medium were aliquoted into media bags and warmed for a suitable period. Once bioreactors finished in the autoclave, calibration soy-based seed medium was transferred into the bioreactors. The suitable cell line A setpoint parameters were input into the bioreactor's control tower. The required control parameters were turned ON. The dissolved oxygen probes were calibrated at 100% saturation. Appropriate volumes of two solution additions were aliquoted into two transfer bottles, per bioreactor. The transfer bottles were sterile welded onto the bioreactors and the lines were primed. The calibration media was drained from the bioreactors and the pre-warmed soy-based seed medium was transferred into the bioreactors. Once expansion times were met, a sample was taken from the wave bioreactor 2 and a bioanalyzer analysis was performed. A suitable volume of cells was aliquoted from the wave bioreactor 2 into a media bag and then transferred into the bioreactors. The required control parameters left were turned ON. Post inoculation, a sample was taken from the N-3 bioreactor and analyzed on the bioanalyzer. Once expansion times were met for each seed train step, a sample was taken from the bioreactor and a bioanalyzer analysis was performed. Once viable cell density in-process control ranges were met, transfers occurred. The bioreactors were sampled three times daily, and pH adjustments were performed when necessary.

### Production Bioreactor—Preparation, Inoculation and Sampling

### Cell Line B

Phase 2 consisted of the bioprocessing of cell line B throughout seed train and production. The production bioreactor settings were input into the bioreactor control tower. The appropriate volume of soy-based seed medium was aliquoted into media bags and warmed for a suitable period. Once the N-1 expansion time was met, a sample was taken from the bioreactor and a bioanalyzer analysis was performed. Prior to the transfer, a pH check was performed on each pending production bioreactor by adjusting the on-line pH to match the bioanalyzer pH. A suitable volume of feed medium A was prepared, and appropriate volumes were aliquoted into transfer bottles. The bioreactors were drained of their culture and set volumes were aliquoted from the culture into media bags. Pre-warmed appropriate aliquots of soy-based production medium were transferred into the bioreactors, followed by the aliquots of cell culture. After inoculation of the production bioreactors, the transfer bottles containing feed medium A aliquots were sterile welded to the bioreactors and added to each bioreactor simultaneously. Post-inoculation of the production bioreactor, a sample was taken from the bioreactor and a bioanalyzer analysis was performed. For each day of production three daily samples were taken. Daily adjustments to the on-line pH were made when necessary to account for the drift in probe measurements. Daily additions of calculated feed medium B were given if necessary. Volumes given are based on bioanalyzer values obtained and resulted calculated volume of feed medium B. Once appropriate expansion time of production was met, a final sample was taken a full analysis was performed utilizing the bioanalyzer. All bioreactor controls were turned OFF. The cell culture was drained and disposed of the bioreactors were deconstructed and washed appropriately.

The viable cell density along with all other nutrients, metabolites, pH and gases of both cell line A and cell line B were analyzed using the automated bioanalyzer. Prior to collecting the sample for analysis, an initial sample was taken through the sample line and discarded. This was completed to clear the sample line prior to sample collection. The sample for analysis was then aspirated through the sample line and analyzed using the bioanalyzer, the bioanalyzer results were recorded when obtained. The correct temperature, cell dilution and nutrient/metabolite dilution dependent on temperature and expected viable cell density were input to the analyzer. Both time of sampling and time of analysis was recorded.

The Capacitance probe software scanned the capacitance measurements at set continuous time-points throughout the entire bioprocess, collecting capacitance measurements throughout seed train and production. During Phase 1, capacitance measurements were taken continuously at a set time interval. Throughout Phase 2, capacitance measurements were taken continuously at a longer interval. The probe was connected through a VP8 connector to an external laptop. This laptop had the associated capacitance probe software, it continuously recorded data at specific interval setpoints over the period of the experiments. The data was stored on the software and was exported directly.

Prior to performing a transfer, a series of stepwise tasks were performed on the external laptop capacitance probe software, simultaneous to performing the transfer operations. Before draining of the culture begins, the ‘Stop Experiment’ button was pressed. Once all the new media is added into the emptied bioreactor, the ‘Start Experiment’ button was pressed. After roughly a few minutes of the probes being in contact with the new media, the ‘Mark Zero’ button was pressed. Once all the cells are added into the new media, the ‘Inoculation’ button was pressed.

The model consisted of capacitance readings and the respective off-line viable cell density measurements. All the off-line time points recorded and available were identified, summarized, and compared with on-line data points available. The data was generated and trended using on-line software. Three linear regression models were developed from the on-line capacitance readings and the off-line viable cell density values for cell line A and cell line B seed train for Phase 1. The data points were compiled which generated a larger data set, this generated a linear regression model for both molecules together (combined). Three linear equations were generated and used to predict viable cell density values. The viable cell density trajectories for both cell line A and cell line B and combined were plotted and graphed using on-line software. The cell line B bivariate fit correlation equation generated in Phase 1 was used to generate molecule specific on-line viable cell density prediction measurements throughout the seed train and production period. Off-line bioanalyzer viable cell density values were obtained, noted in Runsheets, and graphed against the on-line viable cell density and compared visually using on-line software.

### Cell Line A Seed Train Results

Capacitance readings were generated using an industrial scaled-down fed-batch process utilizing two different cell lines expressing different therapeutic modalities. Cell line A used a chemically defined medium and cell line B used a soy-based medium. The capacitance probe sensors were integrated into the bioreactors. Standard seed train cultivations were performed on cell line A and cell line B. An off-line automated bioanalyzer performed the trypan blue exclusion test to determine the viable cell density values, and the viable cell density values were plotted against the corresponding on-line capacitance values to produce linear regression models. Cell-line-specific and combined linear models were developed and their transferability was assessed based on accuracy in predicting viable cell density trajectories. The method of correlating viable cell density and permittivity was entirely a data driven approach.

The work was split into two phases. Phase 1 consisted of a proof-of-concept study. Phase 1 assessed the accuracy of the capacitance probe in determining on-line viable cell density predictions for two cell lines specifically within the seed train. Phase 2 assessed the accuracy of the capacitance probe in determining on-line viable cell density predictions for predicting transfer decisions within the seed train, as well as the applicability and accuracy of determining viable cell density throughout production. In phase 1, four standard fed-batch processes were analyzed. A fed-batch process using a capacitance probe and a fed-batch process without a capacitance probe were used for each of cell line A and cell line B. Seed medium was kept constant for each cell line bioprocess. The capacitance readings were correlated against the off-line viable cell density measurements obtained using the bioanalyzer.

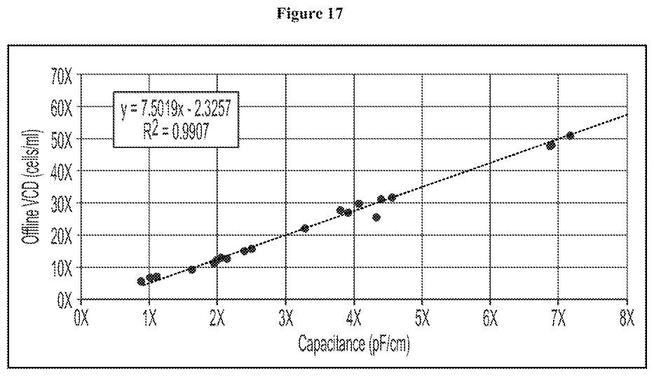

A cell line A linear regression model that correlates the off-line viable cell density values and on-line capacitance measurements for cell line A during seed train (the Cell Line A Linear Regression Model) is presented in FIG. 17, according to an exemplary embodiment of the present invention. On-line capacitance measurements were obtained using a capacitance probe in a bioreactor and off-line viable cell density values were obtained using a bioanalyzer. On-line capacitance measurements were compared to the off-line viable cell density values of cell culture samples taken at the times capacitance was measured. The Cell Line A Linear Regression Model equation is y=7.5019x−2.3257, which has an r² value (e.g., the coefficient of determination) of 0.9907. A coefficient of determination value represents how well differences in one variable explain differences in another variable. The differences between the observations and the predicted value are small and unbiased. The Cell Line A Linear Regression Model fits the data and has an acceptable goodness-of-fit based on visual observations. The Cell Line A Linear Regression Model coefficient of determination value of 0.9907 indicates the on-line capacitance data is effective in predicting real-time viable cell density for cell line A during seed train.

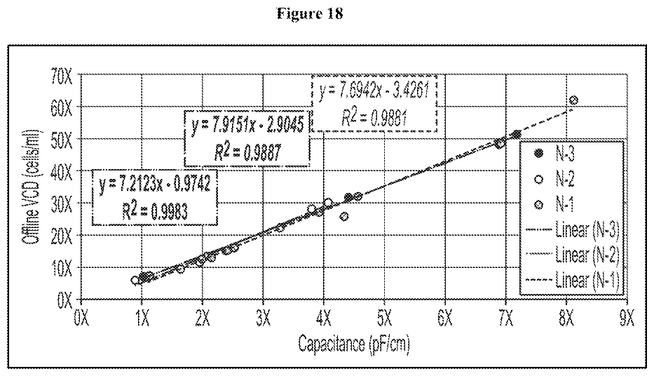

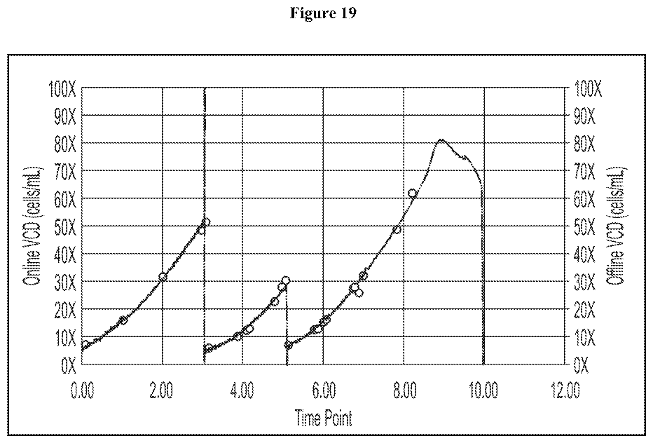

Three separate linear regression models that correlate off-line viable cell density values and on-line capacitance measurements for cell line A during the N-3, N-2 or N-1 phase of seed train are presented in FIG. 18, according to an exemplary embodiment of the present invention. The three different linear regression correlation equations for cell line A during N-3, N-2 and N-1 seed train phases highlights slight differences of the three phases. The linear regression model equation generated for the N-3 phase of seed train is y=7.2123x−0.9742, which has a coefficient of determination value of 0.9983. The linear regression model equation generated for the N-2 phase of seed train is y=7.9151x−2.9045, which has a coefficient of determination value of 0.9887. The linear regression model equation generated for the N-1 phase of seed train is y=7.6942x−3.4261, which has a coefficient of determination value of 0.9881. The coefficient of determination value decreases with each successive phase of seed train, though the differences are minimal and could be due to varied sampling points. The on-line viable cell density values of cell line A during seed train predicted by the on-line capacitance measurements and Cell Line A Linear Regression Model are presented in FIG. 19, according to an exemplary embodiment of the present invention. The off-line viable cell density values that were determined using the bioanalyzer are depicted using the dots. The on-line viable cell density predictions and off-line viable cell density measurements strongly correlate based upon a visual comparison.

### Cell Line B Seed Train Results

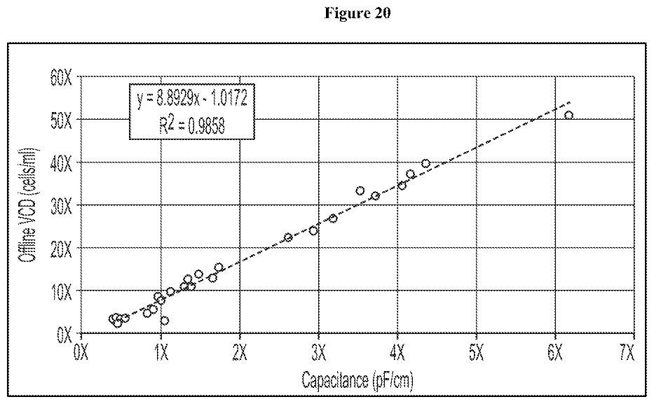

A cell line B linear regression model that correlates the off-line viable cell density values and on-line capacitance measurements for cell line B during seed train (the Cell Line B Linear Regression Model) is presented in FIG. 20, according to an exemplary embodiment of the present invention. On-line capacitance measurements were obtained using a capacitance probe and off-line viable cell density values were obtained using a bioanalyzer. On-line capacitance measurements were compared to the off-line viable cell density values of cell culture samples taken at the times capacitance was measured. The Cell Line B Linear Regression Model equation is y=8.8929x−1.0172, which has a coefficient of determination value of 0.9858. The differences between the observations and the predicted value are small and unbiased. The Cell Line B Linear Regression Model fits the data and has an acceptable goodness-of-fit based on visual observations. The Cell Line B Linear Regression Model coefficient of determination value of 0.9858 indicates the on-line capacitance data is effective in predicating real-time viable cell density for cell line B during seed train.

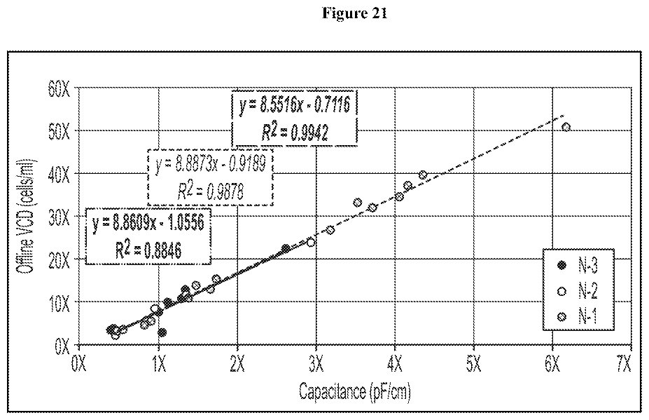

Three separate linear regression models that correlate off-line viable cell density values and on-line capacitance measurements for cell line B during the N-3, N-2 or N-1 phase of seed train are presented in FIG. 21, according to an exemplary embodiment of the present invention. The three different linear regression correlation equations for cell line B during N-3, N-2 and N-1 seed train phases highlights slight differences between the three phases. The linear regression model equation generated for the N-3 phase of seed train is y=8.8609x−1.0556, which has a coefficient of determination value of 0.8846. The linear regression model equation generated for the N-1 phase of seed train is y=8.8873x−0.9189, which has a coefficient of determination value of 0.9878. The linear regression model equation generated for the N-2 phase of seed train is y=8.5516x−0.7116, which has a coefficient of determination value of 0.9942. The differences between each successive phase of seed train were minimal and could be due to varied sampling points

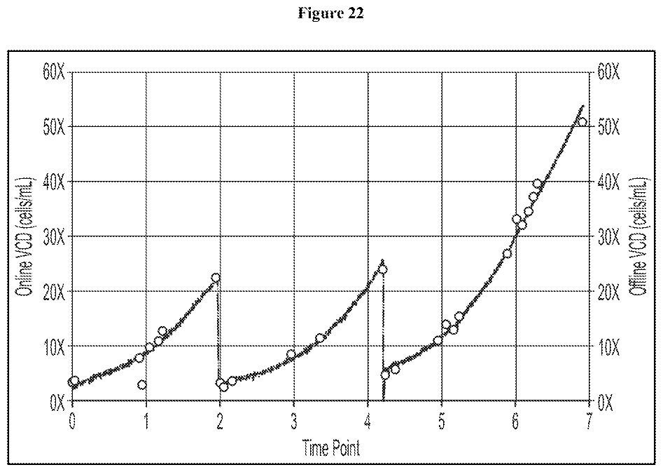

The on-line viable cell density values of cell line B during seed train predicted by the on-line capacitance measurements and Cell Line B Linear Regression Model are presented in FIG. 22, according to an exemplary embodiment of the present invention. The off-line viable cell density values that were determined using the bioanalyzer are depicted using the dots. The on-line viable cell density predictions and off-line viable cell density measurements strongly correlate based upon a visual comparison, indicating that capacitance probes can accurately predict the on-line viable cell density of a mammalian cell line during seed train bioprocessing, which could potentially be useful in assessing cell culture health and in dictating transfer decisions.

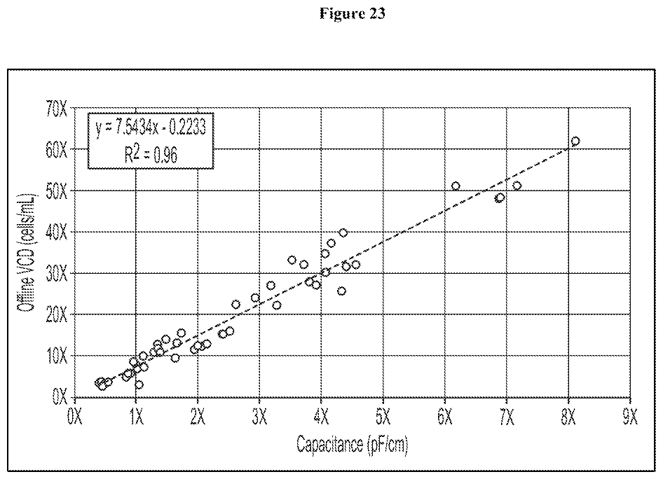

A linear regression model that correlates the off-line viable cell density values and on-line capacitance measurements for cell line A and cell line B during seed train (the Combined Linear Regression Model) is presented in FIG. 23, according to an exemplary embodiment of the present invention. The off-line viable cell density values and on-line capacitance measurements used to produce the Cell Line A Linear Regression Model and Cell Line B Linear Regression Model were combined to produce the Combined Linear Regression Model. The Combined Linear Regression Model equation is y=7.5434x−0.2233, which has a coefficient of determination value of 0.96. The differences between the observations and the predicted value are small and unbiased. The Combined Linear Regression Model fits the data and has an acceptable goodness-of-fit based on visual observations. The Combined Linear Regression Model coefficient of determination value of 0.96 indicates that the Combined Linear Regression Model equation generated using the cell line A and cell line B data during seed train can accurately predict viable cell density values.

Cell line specific linear regression models, the Cell Line A and Cell Line B Linear Regression Models, and a linear regression model produced using the off-line viable cell density values and on-line capacitance measurements for cell line A and cell line B during seed train, the Combined Linear Regression Model, were generated and used to predict on-line viable cell density values. Subsequently, the transferability of the cell line specific linear regression models and the Combined Linear Regression Model for predicting on-line viable cell density for cell line A and cell line was assessed.

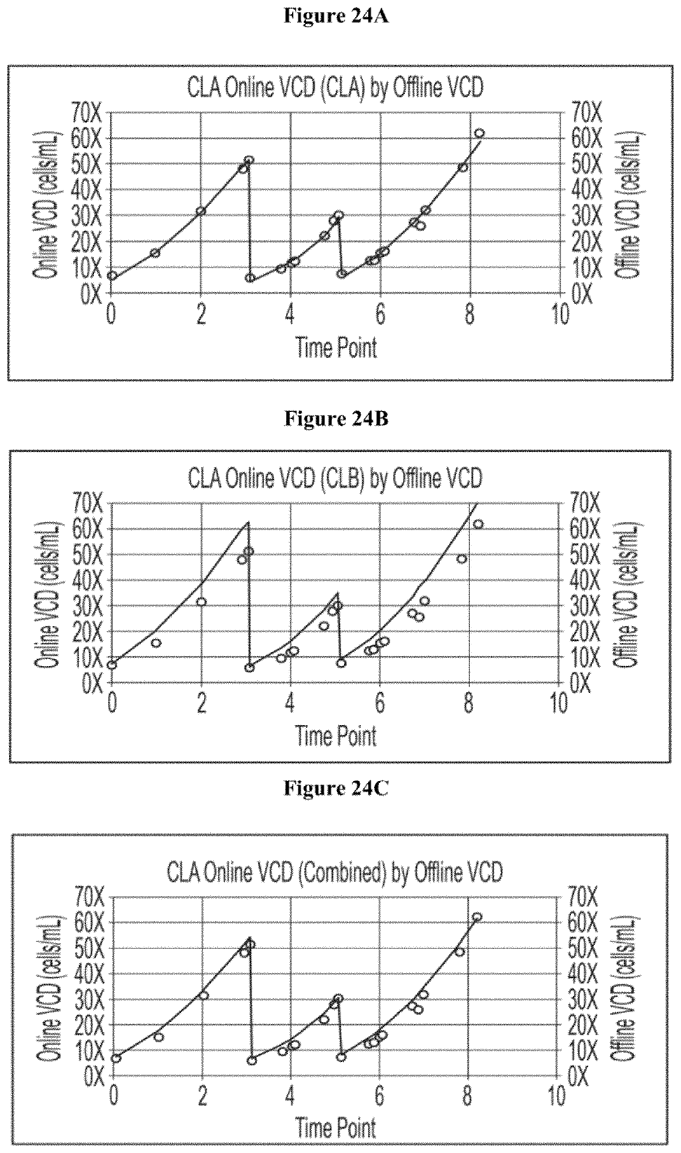

FIG. 24A shows the on-line viable cell density values of cell line A during seed train predicted using the Cell Line A Linear Regression Model illustrated in FIG. 17, which provided the most accurate prediction of on-line viable cell density values, according to an exemplary embodiment of the present invention. FIG. 24B and FIG. 24C show the on-line viable cell density values of cell line A during seed train predicted using the Cell Line B Linear Regression Model illustrated in FIG. 20 and Combined Linear Regression Model illustrated in FIG. 23, respectively, according to exemplary embodiments of the present invention. The Cell Line A Linear Regression Model predicted the on-line viable cell density values of cell line A during seed train most accurately, followed by the Combined Linear Regression Model and the Cell Line B Linear Regression Model, respectively.

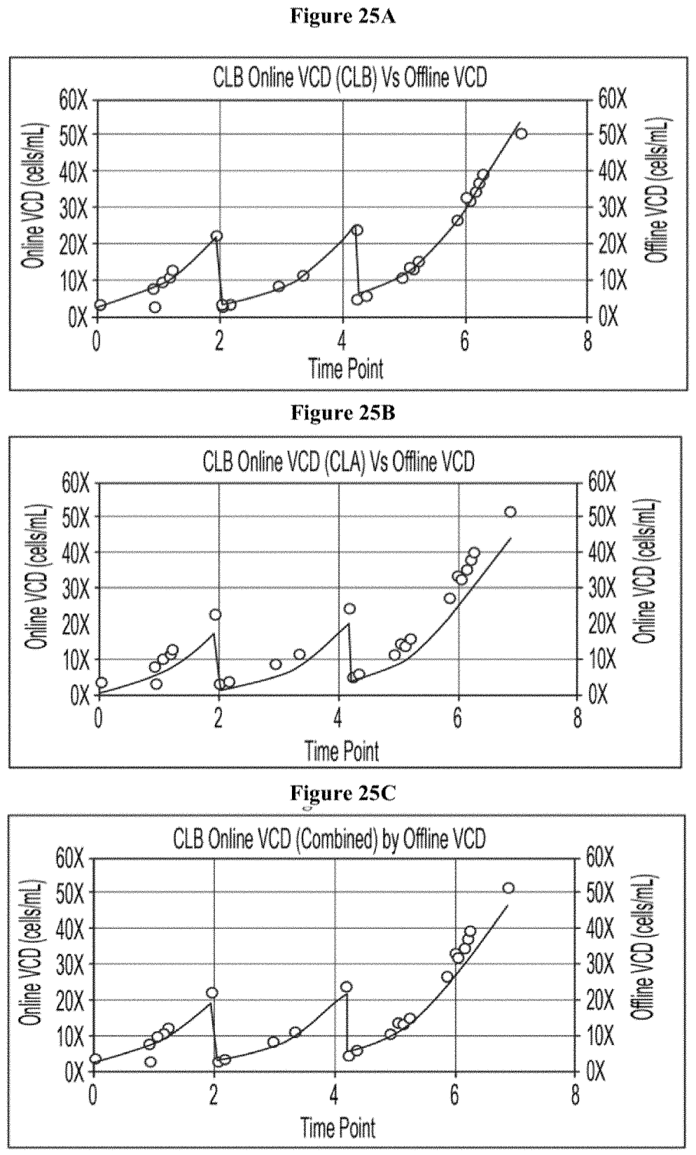

FIG. 25A shows the on-line viable cell density values of cell line B during seed train predicted using the Cell Line B Linear Regression Model illustrated in FIG. 20, which provided the most accurate prediction of on-line viable cell density values, according to an exemplary embodiment of the present invention. FIG. 25B and FIG. 25C show the on-line viable cell density values of cell line B during seed train predicted using the Cell Line A Linear Regression Model illustrated in FIG. 17 and Combined Linear Regression Model illustrated in FIG. 23, respectively, according to exemplary embodiments of the present invention. The Cell Line B Linear Regression Model predicted the on-line viable cell density values of cell line B during seed train most accurately, followed by the Combined Linear Regression Model and the Cell Line A Linear Regression Model, respectively.

Cell line specific and combined linear regression models can accurately predict on-line viable cell density during seed train for both cell line A and cell line B. A comparison of model transferability showed that cell line specific linear regression models are most accurate when used with the corresponding cell line, followed by the combined linear regression model and the alternative cell line specific linear regression model, respectively.

A root-mean-square error (RMSE) analysis verified the conclusions drawn from experimental results observed in FIG. 24A, FIG. 24B, FIG. 24C, FIG. 25A, FIG. 25B, and FIG. 25C, according to exemplary embodiments of the present invention. The predicted data points are the on-line viable cell density values obtained using the Cell Line A, Cell Line B, or Combined Linear Regression Model and the values obtained off-line using the bioanalyzer are the observed values.

Table 28 shows that the on-line viable cell density values of cell line A during seed train predicted using the Cell Line A Linear Regression Model resulted in a root-mean-square error value of 1.5206. Additionally, Table 28 shows that the on-line viable cell density values of cell line A during seed train predicted using the Cell Line B Linear Regression Model and Combined Linear Regression Model were 7.0428 and 2.7471, respectively.

Table 28 shows that the on-line viable cell density values of cell line B during seed train predicted using the Cell Line B Linear Regression Model resulted in a root-mean-square error value of 1.5919. Additionally, Table 28 shows that the on-line viable cell density values of cell line A during seed train predicted using the Cell Line B Linear Regression Model and Combined Linear Regression Model were 4.8601 and 3.1717, respectively.

Thus, using the cell line specific models to predict the on-line viable cell density values of the cell line used to produce the corresponding cell line specific model provided the most accurate predictions, followed by the Combined Linear Regression Model and the cell line specific model produced using the alternative cell line.

TABLE 28
Cell Line A and Cell Line B Root-Mean-Square Error Values

| Cell Line | Linear Regression Model | Root-Mean-Square Error |
|---|---|---|
| A | Cell Line A | 1.5206 |
| | Cell Line B | 7.0428 |
| | Combined | 2.7471 |
| B | Cell Line A | 4.8601 |
| | Cell Line B | 1.5919 |
| | Combined | 3.1717 |

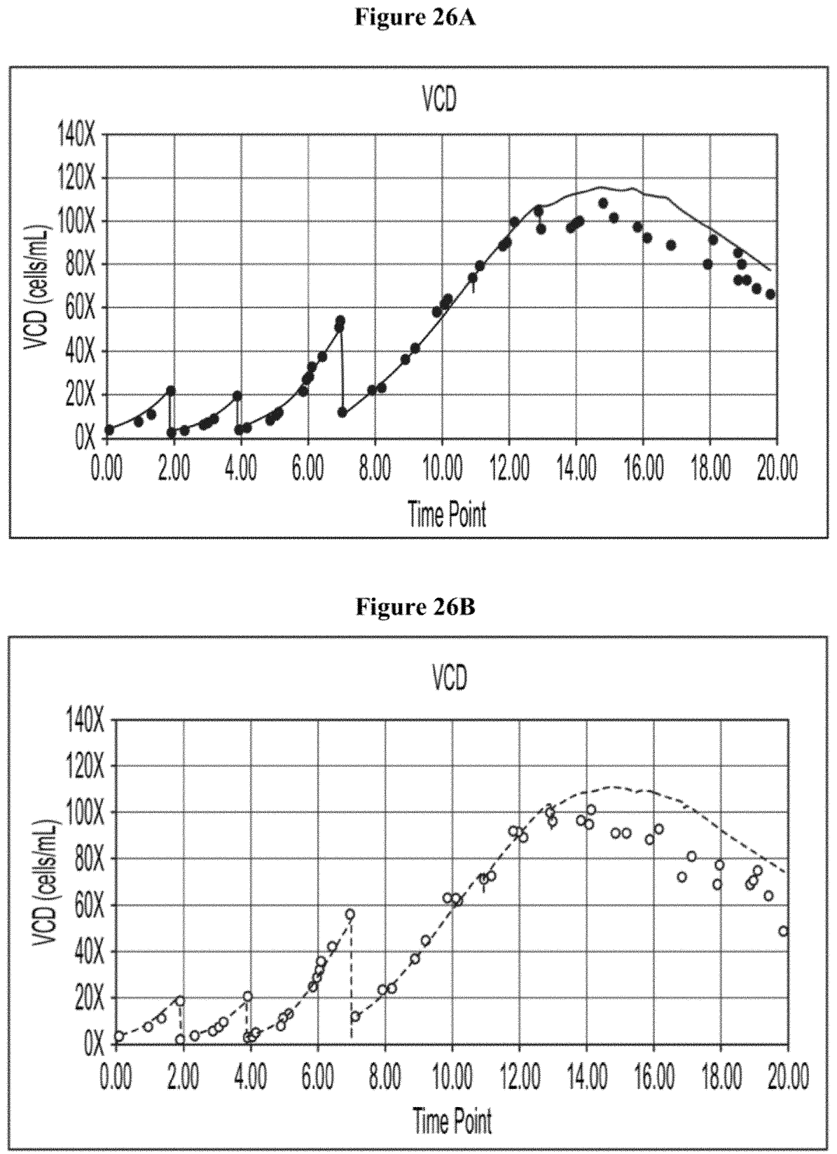

The Cell Line B Linear Regression Model shown in FIG. 20 was used to predict on-line viable cell density values for cell line B during seed train and production phases in Phase 2. The on-line viable cell density predictions for two separate seed train and production phases generated using two different capacitance probes are shown in FIG. 26A and FIG. 26B, according to an exemplary embodiment of the present invention. The viable cell density values measured off-line using the bioanalyzer are depicted as dots in FIG. 26A and FIG. 26B, respectively. The permittivity signal decreased at times 11 and 13 in FIG. 26A and FIG. 26B.

### Example 24. Mixed Mode Chromatography Development

A novel process was developed for producing Dupilumab using mixed-mode chromatography (MMC). A range of buffer pH, salt concentrations, protein loading, and resin types for MMC were evaluated for achieving a suitable Dupilumab yield as well as a suitable reduction in HMW impurities, over the course of two 16-run D-Optimal design of experiments (DoE) run in triplicate. MMC process steps included equilibration, load, wash, strip 1, and strip 2. An incubation time for equilibration was 1 minute. An incubation time for loading was 60 minutes. An incubation time for washing, strip 1, and strip 2 was 1 minute each. Liquid handling was automated. A protein load used for MMC was a pH-adjusted Dupilumab load following Protein A and viral inactivation steps as described above.

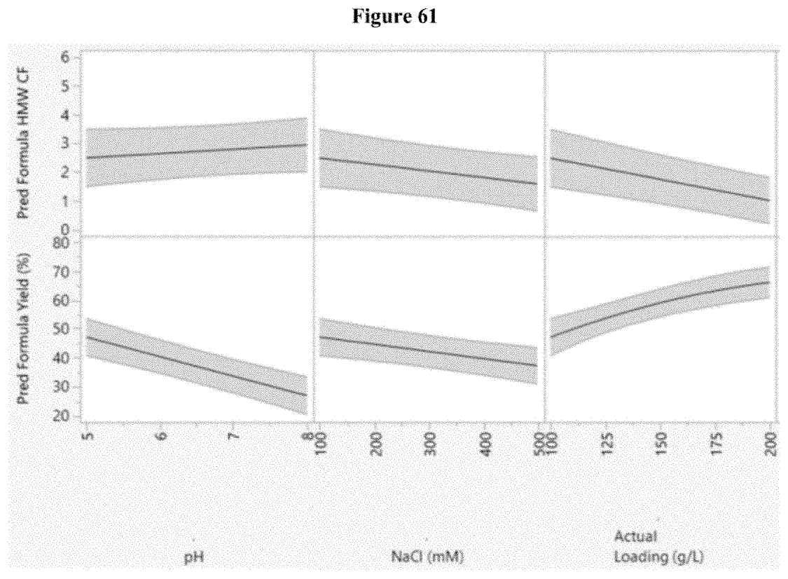

The relationship between tested MMC parameters and outcomes in terms of BMW species and yield are shown for a Capto Adhere resin in FIG. 60 and for a PPA HyperCel resin in FIG. 61. Optimal conditions when using a Capto Adhere resin included an equilibration buffer at pH 5.0 comprising 100 mM NaCl, and a protein load of 110 g/L. Optimal conditions when using a PPA HyperCel resin included an equilibration buffer at pH 5.0 comprising 100 mM NaCl, and a protein load of 100 g/L. The MMC process may be further optimized, for example, by substituting HEA HyperCel, MEP HyperCel, or Capto MMC as a resin. Optimal conditions for producing Dupilumab include an incubation time from about 2 to about 10 minutes for equilibration, washing, strip 1, and strip 2 steps, preferably about 6 minutes.

The results demonstrate that an MMC process resulted in a suitable yield and a suitable BMW species reduction in a Dupilumab load and is a viable additional process for Dupilumab production that may replace the CEX and/or AEX processing steps described above. In particular, the results demonstrate that a Dupilumab production process using a protein A step followed by a mixed-mode chromatography step and an anion exchange chromatography step as described above is a suitable process for production of Dupilumab. The use of a protein A wash selected to reduce HCP and BMW impurities, as described in Example 9, would further optimize a Dupilumab production process with chromatography steps comprising protein A chromatography, mixed-mode chromatography and anion exchange chromatography, and without a need for CEX or HIC.

### ENUMERATED EXAMPLES

The following Enumerated Example set forth below provide additional aspects of the present disclosure.
1. A method of purifying an anti-IL4Rα antibody, comprising the steps of:
   (a) subjecting said antibody to affinity chromatography;
   (b) subjecting said antibody pooled from eluate of step (a) to viral inactivation at a pH from about 3 to about 4.5 and then adjusting the pH to from about 5 to about 8;
   (c) subjecting said antibody pooled from step (b) to anion exchange chromatography (AEX) in flowthrough mode;
   (d) subjecting said antibody pooled from flowthrough fractions of step (c) to hydrophobic interaction chromatography (HIC) in flowthrough mode; and
   (e) subjecting said antibody pooled from flowthrough fractions of step (d) to virus retentive filtration (VRF) to purify said anti-IL4Rα antibody.
2. The method of example 1, further comprising a harvest pre-treatment step prior to step (a).
3. The method of any one of examples 1-2, wherein the harvest pre-treatment step includes adjusting said antibody to a transient pH level from about 4 to 5.5.
4. The method of any one of examples 1-3, wherein said antibody of step (e) is further subjected to concentration and diafiltration using a diafiltration buffer having a pH between 4.0 and 4.5.
5. The method of any one of examples 1-4, wherein the pH of a final concentrated pool (FCP) is between 5.2 and 5.3±0.1.
6. The method of example 4, wherein said diafiltration buffer comprises about 4 mM acetate to about 6 mM acetate.
7. The method of any one of examples 1-6, wherein said affinity chromatography is Protein A chromatography.
8. The method any one of examples 1-7, wherein a Protein A resin is selected from the group consisting of MabSelect SuRe, MabSelect PrismA, MabSelect SuRe LX, Mab Select, Mab Select SuRe pcc, Mab Select Xtra, rProtein A Sepharose, ProSep HC, ProSep Ultra, ProSep Ultra Plus, MabCapture, and Amsphere A3.
9. The method of any one of examples 7 or 8, wherein the protein load on said Protein A chromatography column is at least 55 g/L-resin.
10. The method of any one of examples 1-9, wherein the pH of a FDS is between 5.8 and 6.0±0.1.
11. The method of any one of examples 1-10, wherein about 180 g to 200 g of antibody is loaded per liter of HIC resin.
12. The method of any one of examples 1-11, wherein the amount of PLBD2 in the HIC eluate is reduced by about 60x-310x compared to the amount of PLBD2 in the HIC load.
13. The method of any one of examples 7-12, wherein a Protein A column load pH is between 7 and 8.
14. The method of any one of examples 1-13, wherein said antibody of step (e) is further subjected to ultrafiltration and diafiltration (UF/DF) after virus filtration.
15. The method of any one of examples 1-14, wherein said anti-IL-4Ra antibody comprises three heavy chain complementarity determining region (HCDR) sequences comprising SEQ ID NOs: 3, 4, and 5, and three light chain complementarity determining region (LCDR) sequences comprising SEQ ID NOs: 6, 7, and 8.
16. The method of any one of examples 1-15, wherein said anti-IL-4Rα antibody comprises a heavy chain variable region (HCVR) comprising the amino acid sequence of SEQ ID NO: 1 and a light chain variable region (LCVR) comprising the amino acid sequence of SEQ ID NO: 2.
17. The method of any one of examples 1-16, wherein said anti-IL-4Rα antibody is Dupilumab.
18. A method comprising the steps of:
   (a) culturing cells expressing an anti-IL-4Rα antibody or antigen-binding fragment thereof,
   (b) subjecting said cells to transient pH levels from about 4.5 to 5.0, then raising pH levels to from about 5.5 to 6.5;
   (c) harvesting said cells by centrifugation to separate cell debris from clarified media comprising the anti-IL-4Rα antibody or antigen binding fragment thereof;
   (d) subjecting said clarified media to affinity chromatography;
   (e) subjecting said anti-IL-4Rα antibody or antigen-binding fragment thereof pooled from eluate of step (d) to viral inactivation at a pH from about 3 to about 4.4 and then adjusting the pH to from about 5 to about 8;
   (f) subjecting said anti-IL-4Rα antibody or antigen-binding fragment thereof pooled from step (e) to anion exchange chromatography in flowthrough mode;
   (g) subjecting said anti-IL-4Rα antibody or antigen-binding fragment thereof pooled from flowthrough fractions of step (f) to cation exchange chromatography in bind and elute mode;
   (h) subjecting said anti-IL-4Rα antibody or antigen-binding fragment thereof pooled from eluate of step (g) to hydrophobic interaction chromatography in flowthrough mode; and
   (i) subjecting said anti-IL-4Rα antibody or antigen-binding fragment thereof pooled from flowthrough fractions of step (h) to virus retentive filtration to produce said anti-IL-4Rα antibody or antigen-binding fragment thereof.
19. The method of example 18, further comprising subjecting the anti-IL-4Rα antibody or antigen-binding fragment thereof to ultrafiltration and diafiltration (UF/DF) after step (i).
20. The method of example 18 or 19, wherein said affinity chromatography is Protein A chromatography.
21. The method of example 20, wherein a Protein A resin is selected from the group consisting of MabSelect PrismA, MabSelect SuRe, MabSelect SuRe LX, MabSelect, Mab Select SuRe pcc, Mab Select Xtra, rProtein A Sepharose, ProSep HC, ProSep Ultra, ProSep Ultra Plus, MabCapture, and AmsphereA3.
22. The method of any one of examples 1-21, wherein an anion exchange resin is selected from the group consisting of Poros 50PI, Poros 50HQ, Capto Q Impres, Capto DEAE, Toyopearl QAE-550, Toyopearl DEAE-650, Toyopearl GigaCap Q-650, Fractogel EMD TMAE Hicap, Sartobind STIC PA nano, Sartobind Q nano, CUNO BioCap, and XOHC.
23. The method of any one of examples 1-22, wherein a cation exchange resin is selected from the group consisting of Capto SP ImpRes, Capto S ImpAc, CM Hyper D grade F, Eshmuno S, Nuvia C Prime, Nuvia S, Poros HS, and Poros XS.
24. The method of any one of examples 18-23, further comprising passing said anti-IL-4Rα antibody or antigen-binding fragment thereof through a LifeAssure filter after the viral inactivation of step (e) and prior to the anion exchange chromatography of step (f).
25. The method of any one of examples 14-17 or 19-24, wherein said UF/DF step comprises a membrane filter device selected from the group consisting of Pellicon 2, Pellicon 3 cassettes with 10 kD, 30 kD or 50 kD membranes, Kvick 10 kD, 30 kD or 50 kD membrane cassettes, and Centramate and Centrasette 10 kD, 30 kD or 50 kD cassettes, and does not include addition of arginine.
26. The method of any one of examples 1-25, wherein said HIC step comprises HIC media selected from the group consisting of Capto Phenyl, Capto Phenyl High Sub, Phenyl Sepharose™ 6 Fast Flow, Phenyl Sepharose™ High Performance, Octyl Sepharose High Performance, Fractogel EMD Propyl, Fractogel EMD Phenyl, Macro-Prep Methyl, Macro-Prep t-Butyl columns, WP HI-Propyl (C3), Toyopearl ether, phenyl or butyl, Toyo PPG; Toyo Phenyl; Toyo Butyl, and Toyo Hexyl.
27. The method of any one of examples 1-14 or 18-26, wherein said anti-IL-4Rα antibody or antigen-binding fragment thereof comprises three heavy chain complementarity determining region (HCDR) sequences comprising SEQ ID NOs: 3, 4, and 5, and three light chain complementarity determining region (LCDR) sequences comprising SEQ ID NOs: 6, 7, and 8.
28. The method of any one of examples 1-14 or 18-27, wherein said anti-IL-4Rα antibody or antigen-binding fragment thereof comprises a heavy chain variable region (HCVR) comprising the amino acid sequence of SEQ ID NO: 1 and a light chain variable region (LCVR) comprising the amino acid sequence of SEQ ID NO: 2.
29. The method of any one of examples 1-14 or 18-28, wherein said anti-IL-4Rα antibody is Dupilumab.
30. A method comprising the steps of:
   (a) subjecting a harvested antibody to affinity chromatography;
   (b) subjecting antibody pooled from eluate of step (a) to viral inactivation at a pH from about 3 to about 4.5 and then adjusting the pH to from about 5 to about 8;
   (c) subjecting said antibody pooled from step (b) to anion exchange chromatography in flowthrough mode;
   (d) subjecting said antibody pooled from flowthrough fractions of step (c) to cation exchange chromatography in bind and elute mode;
   (e) subjecting said antibody pooled from eluate of step (d) to hydrophobic interaction chromatography in flowthrough mode; and
   (f) subjecting said antibody pooled from flowthrough fractions of step (e) to virus retentive filtration to produce said anti-IL4Rα antibody.
31. A serum-free cell culture medium comprising ≥0.09 mM±0.014 mM ornithine.
32. The cell culture medium of any one of examples 1-31, comprising ≥0.20±0.03 mM putrescine.
33. The cell culture medium of any one of examples 1-32, comprising between 0.09±0.014 mM and 0.9-±0.14 mM ornithine.
34. The cell culture medium of any one of examples 1-33, comprising 0.09±0.014 mM, 0.3-±0.05 mM, 0.6-±0.09 mM, or 0.9-±0.14 mM ornithine.
35. The cell culture medium of any one of examples 1-34, comprising between 0.20±0.03 mM and 0.714±0.11 mM putrescine.
36. The cell culture of any one of examples 1-34, comprising 0.20±0.03 mM, 0.35-±0.06, or 0.714±0.11 mM putrescine.
37. The cell culture medium of any one of examples 1-36, wherein the medium is hydrolysate-free.
38. The cell culture medium of any one of examples 1-37, wherein the medium is chemically defined.
39. The cell culture medium of any one of examples 1-38, comprising ≥40±6 mM of a mixture of amino acids or salts thereof.
40. The cell culture medium of example 39, wherein said mixture of amino acids comprises two or more amino acids selected from the group of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and combinations thereof
41. The cell culture medium of any one of examples 1-40, comprising one or more fatty acids.
42. The cell culture medium of example 41, wherein said one or more fatty acids are selected from the group of linoleic acid, linolenic acid, thioctic acid, oleic acid, palmitic acid, stearic acid, arachidic acid, arachidonic acid, lauric acid, behenic acid, decanoic acid, dodecanoic acid, hexanoic acid, lignoceric acid, myristic acid, octanoic acid, and combinations thereof
43. The cell culture medium of any one of examples 1-42, comprising a mixture of nucleosides.
44. The cell culture medium of example 43, wherein the mixture of nucleosides comprises of two or more nucleosides selected from the group of adenosine, guanosine, cytidine, uridine, thymidine, hypoxanthine, and combinations thereof
45. The cell culture medium of any one of examples 1-44, comprising adenosine, guanosine, cytidine, uridine, thymidine, and hypoxanthine.
46. The cell culture medium of any one of examples 1-45, comprising one or more divalent cations.
47. The cell culture medium of example 46, wherein said divalent cation is magnesium, calcium, or both.
48. The cell culture medium of any one of examples 1-47, comprising Ca²⁺ and Mg²⁺.
49. A method for cultivating a cell, comprising the steps of: (a) providing a cell culture medium according to any one of examples 31-48, and (b) propagating or maintaining a cell in said cell culture medium to form a cell culture.
50. The method of example 49, wherein said cell is selected from the group consisting of a mammalian cell, primate cell, avian cell, insect cell, bacterial cell, and yeast cell.
51. The method of example 49 or example 50, wherein said cell is a CHO cell.
52. The method of any one of examples 49-51, wherein said cell expresses a protein of interest.
53. The method of example 52, wherein said protein of interest is an antigen-binding protein.
54. The method of examples 52 or 53, wherein said protein of interest comprises an Fc domain.
55. The method of examples 52 or 53, wherein said protein of interest is an IgG4 antibody or an antibody fragment.
56. The method of example 55, wherein said antibody or antibody fragment is a recombinant human antibody or fragment thereof
57. The method of any one of examples 49-56, wherein said cell has an average doubling time of ≤30 hours.
58. The method of any one of examples 49-57, wherein said cell has an average doubling time of ≤24 hours.
59. The method of any one of examples 49-58, wherein said cell has an average doubling time that is no more than one third that of cells grown in a cell culture medium that comprises <0.3±0.045 mM ornithine and <0.2±0.03 mM putrescine.
60. The method of any one of examples 49-59, wherein said cell culture is capable of attaining a viable cell density that is at least 15% greater than a similar cell culture in media that comprises <0.09±0.014 mM ornithine and <0.2±0.03 mM putrescine.
61. The method of any one of examples 49-60, wherein said cell culture is capable of attaining a viable cell density that is at least 3-fold greater than a similar cell culture in a similar cell culture medium that comprises <0.09±0.014 mM ornithine and <0.2±0.03 mM putrescine.
62. The method of any one of examples 49-61, further comprising the step of adding one or more point-of-use additions to the cell culture medium.
63. The method of example 62, wherein said point-of-use additions comprise one or more of NaHCO₃, Na₂HPO₄, taurine, glutamine, poloxamer 188, insulin, glucose, CuSO₄, ZnSO₄, FeCl₃, NiSO₄, Na₄ EDTA, and Na₃ citrate.
64. The method of examples 62 or 63, wherein said point-of-use additions comprise NaHCO₃, glutamine, insulin, glucose, CuSO₄, ZnSO₄, FeCl₃, NiSO₄, Na₄ EDTA, and Na₃ citrate.
65. A method of producing Dupilumab, comprising:
   a) culturing Chinese Hamster Ovary (CHO) cells in serum-free medium, wherein said medium comprises insulin and one or more polynucleotides encoding Dupilumab;
   b) supplementing said medium on at least two different days with additional insulin to a concentration of about 7.5 mg/L;
   c) isolating Dupilumab at about 10 and 14 days after the start of culture.
66. A method of producing Dupilumab, comprising:
   a) culturing Chinese Hamster Ovary (CHO) cells in serum-free medium, wherein said medium comprises insulin and one or more polynucleotides encoding Dupilumab;
   b) supplementing the medium on about day 3 and on about day 7 with additional insulin to a concentration of about 7.5 mg/L;
   c) isolating Dupilumab at about days 10-14 after the start of culture.
67. The method of any one of examples 49-66, wherein Dupilumab is produced at a titer of at least about 1.5 g/L in said cell culture production medium on day 4.
68. The method of any one of examples 49-67, wherein Dupilumab is produced at a titer of at least about 0.5 g/L in said cell culture production medium on day 3.
69. The method of any one of examples 49-68, wherein Dupilumab is produced at a titer of at least about 2.0 g/L in said cell culture production medium on day 5.
70. The method of any one of examples 49-69, wherein Dupilumab is produced at a titer of at least about 4.0 g/L in said cell culture production medium on day 6.
71. The method of any one of examples 49-70, wherein said culture is maintained at a temperature ranging from about 32° C. to about 38° C.
72. The method of any one of examples 65-71, where said polynucleotide encoding Dupilumab comprises a heavy chain variable region (HCVR) comprising the amino acid sequence of SEQ ID NO: 1 and a light chain variable region (LCVR) comprising the amino acid sequence of SEQ ID NO: 2.
73. The method of any one of examples 49-72, wherein the media further comprises tyrosine.
74. The method of any one of examples 49-73, further comprising supplementing the medium on about day 3 with additional tyrosine to a concentration of about 2.0 g/L.
75. The method of any of examples 49-74, further comprising supplementing the media on about days 0, 2 4, 6 and 8 with additional sodium phosphate to a concentration of about 250-200, about 500-550, about 500-550, about 225-275, and about 225-375 mg/L, respectively.
76. A method for producing a protein, comprising: (a) introducing into a cell a nucleic acid comprising a sequence encoding a protein of interest; (b) selecting a cell carrying said nucleic acid; (c) culturing said selected cell in a cell culture medium of any one of examples 31-48 or according to the method of any one of examples 49-75; and (d) expressing said protein of interest in said cell, wherein said protein of interest is secreted into said cell culture medium.
77. The method of any one of examples 49, 50, 52-64, or 67-76, wherein said cell is a CHO cell, HEK293 cell, or BHK cell.
78. The method of example 65 or 77, wherein said protein of interest is an antigen-binding protein.
79. The method of any one of examples 76-78, wherein said protein of interest comprises an Fc domain.
80. The method of any one of examples 76-79, wherein said protein of interest is an antibody or ScFv protein.
81. The method according to any one of examples 76-80, wherein said protein of interest is produced at an average 7-day titer that is at least 7% greater than the average 7-day titer produced by a similar cell in a cell culture medium that comprises less than 0.09±0.014 mM ornithine and less than 0.2±0.03 mM putrescine.
82. The method according to any one of examples 76-81, wherein said protein of interest is produced at an average 7-day titer that is at least 14% greater than the average 7-day titer produced by a similar cell in a cell culture medium that comprises less than 0.09±0.014 mM ornithine and less than 0.2±0.03 mM putrescine.
83. The method according to any one of examples 76-82, wherein said protein of interest is produced at an average 7-day titer that is at least 80% greater than the average 7-day titer produced by a similar cell in a cell culture medium that comprises less than 0.09±0.014 mM ornithine and less than 0.2±0.03 mM putrescine.
84. The method according to any one of examples 76-83, wherein said protein of interest is produced at an average 7-day titer that is at least 2-fold greater than the average 7-day titer produced by a similar cell in a cell culture medium that comprises less than 0.09±0.014 mM ornithine and less than 0.2±0.03 mM putrescine.
85. The method according to any one of examples 76-84, wherein said protein of interest is produced at an average 7-day titer that is at least 3-fold greater than the average 7-day titer produced by a similar cell in a cell culture medium that comprises less than 0.09±0.014 mM ornithine and less than 0.2±0.03 mM putrescine.
86. The method according to any one of examples 76-85, wherein said protein of interest is a recombinant human antibody.
87. A fed-batch production method of making Dupilumab, comprising culturing Chinese Hamster Ovary (CHO) cells comprising a nucleic acid encoding Dupilumab in a cell culture production medium in large-scale, wherein insulin in said medium is supplemented on days 2 and 4 to a concentration of about 7.5 mg/L, such that Dupilumab is produced at a titer of at least about 0.5 g/L in said cell culture production medium on day 4.
88. The method of example 87, wherein said Dupilumab is produced at a titer of at least 1.5 g/L in said cell culture production medium on day 4.
89. The method of example 87 or 88, wherein said Dupilumab is produced at a titer of at least 2 g/L in said cell culture production medium on day 4.
90. The method of any one of examples 87-89, wherein said culturing lasts about 10-18 days.
91. The method of any one of examples 87-90, wherein said culturing lasts about 10 days.
92. The method of any one of examples 87-91, wherein said cell culture production medium is a serum-free medium.
93. The method of example 92, wherein said serum-free medium comprises a recombinant growth factor, an osmolarity regulator, a pH buffer agent, glutamine, and a cell protectant.
94. The method of any one of examples 51-93, wherein said CHO cells are cultured at a temperature ranging from about 32° C. to about 38° C.
95. The method of any one of examples 87 or 99, wherein said large-scale production is >1,000 L.
96. The method of example 87 or 99, wherein said large-scale production is >3,000 L.
97. The method of example 87 or 99, wherein said large-scale production is >10,000 L.
98. The method of example 87, wherein said large-scale production is between 3,000 L and 25,000 L.
99. A fed-batch production method of making Dupilumab, comprising culturing Chinese Hamster Ovary (CHO) cells comprising a nucleic acid encoding Dupilumab in a cell culture production medium in large-scale, wherein insulin in said medium is supplemented on days 2 and 4 to a concentration of about 7.5 mg/L, such that viability of said cells is at least about 95% in said cell culture production medium on day 4.
100. The method of example 99, wherein viability of said cells is about 100% in said cell culture production medium.
101. The method of example 99, wherein said culturing lasts about 10-15 days.
102. The method of any one of examples 87, wherein said culturing lasts about 10 days.
103. The method of any one of examples 99-102, wherein said cell culture production medium is a serum-free medium.
104. The method of example 87, wherein said serum-free medium comprises a recombinant growth factor, an osmolarity regulator, a pH buffer agent, glutamine, and a cell protectant.
105. The method of any one of examples 51-104, wherein said CHO cells are cultured at a temperature ranging from about 32° C. to about 38° C.
106. A fed-batch production method of making Dupilumab, comprising culturing Chinese Hamster Ovary (CHO) cells comprising a nucleic acid encoding Dupilumab in a cell culture production medium in large-scale, wherein insulin in said medium is supplemented on days 2 and 4 to a concentration of about 7.5 mg/L, such that the concentration of ammonia in said cell culture production medium on day 4 is less than about 5 mM.
107. The method of any one of examples 49-106, wherein the concentration of ammonia in said cell culture production medium on day 4 is less than about 2 mM.
108. The method of any one of examples 49-101, 103-107106, wherein said culturing lasts about 10-15 days.
109. The method of any one of examples 49-108, wherein said culturing lasts about 10 days.
110. The method of any one of examples 49-109, wherein said cell culture production medium is a serum-free medium.
111. The method of example 110, wherein said serum-free medium comprises a recombinant growth factor, an osmolarity regulator, a pH buffer agent, glutamine, and a cell protectant.
112. The method of any one of examples 51-111, wherein said CHO cells are cultured at a temperature ranging from about 32° C. to about 38° C.
113. A fed-batch production method of making Dupilumab, comprising culturing Chinese Hamster Ovary (CHO) cells comprising a nucleic acid encoding Dupilumab in a cell culture production medium in large-scale, wherein tyrosine in said medium is supplemented on day 3 to a concentration of about 2 g/L, such that Dupilumab is produced at a titer of at least about 8 g/L in said cell culture production medium on day 14.
114. The method of example 113, wherein said Dupilumab is produced at a titer of at least 9 g/L in said cell culture production medium.
115. The method of example 113 or 114, wherein said Dupilumab is produced at a titer of at least 10 g/L in said cell culture production medium.
116. The method of any one of examples 113-115, wherein said cell culture production medium is a serum-free medium.
117. The method of example 116, wherein said serum-free medium comprises a recombinant growth factor, an osmolarity regulator, a pH buffer agent, glutamine, methotrexate, and a cell protectant.
118. The method of any one of examples 113-117, wherein said CHO cells are cultured at a temperature ranging from about 32° C. to about 38° C.
119. A fed-batch production method of making Dupilumab, comprising culturing Chinese Hamster Ovary (CHO) cells comprising a nucleic acid encoding Dupilumab in a cell culture production medium in large-scale, wherein tyrosine in said medium is supplemented on days 3 and 7 to a concentration of about 1 g/L, such that the concentration of ammonia in said cell culture production medium on day 14 is less than about 10 mM.
120. The method of example 119, wherein the concentration of ammonia in said cell culture production medium on day 14 is less than about 8 mM.
121. The method of example 119 or 120, wherein said cell culture production medium is a serum-free medium.
122. The method of example 121, wherein said serum-free medium comprises a recombinant growth factor, an osmolarity regulator, a pH buffer agent, glutamine, and a cell protectant.
123. The method of any one of examples 119-122, wherein said CHO cells are cultured at a temperature ranging from about 32° C. to about 38° C.
124. A fed-batch production method of making Dupilumab, comprising culturing Chinese Hamster Ovary (CHO) cells comprising a nucleic acid encoding Dupilumab in a cell culture production medium in large-scale, wherein tyrosine in said medium is supplemented on day 3 to a concentration of about 1 g/L, such that Dupilumab is produced at a titer of at least about 8 g/L in said cell culture production medium on day 14.
125. The method of any one of examples 113-124, wherein said Dupilumab is produced at a titer of at least 9 g/L in said cell culture production medium.
126. The method of any one of examples 113-124, wherein said Dupilumab is produced at a titer of at least 10 g/L in said cell culture production medium.
127. The method of any one of examples 113-126, wherein said cell culture production medium is a serum-free medium.
128. The method of example 127, wherein said serum-free medium comprises a recombinant growth factor, an osmolarity regulator, a pH buffer agent, glutamine, and a cell protectant.
129. The method of any one of examples 113-128, wherein said CHO cells are cultured at a temperature ranging from about 32° C. to about 38° C.
130. The method of any one of examples 99-129, wherein said large-scale production is >1,000 L.
131. The method of any one of examples 99-129, wherein said large-scale production is >3,000 L.
132. The method of any one of examples 99-129, wherein said large-scale production is >10,000 L.
133. The method of any one of examples 99-129, wherein said large-scale production is between 3,000 L and 25,000 L.
134. A fed-batch production method of making Dupilumab, comprising culturing Chinese Hamster Ovary (CHO) cells comprising a nucleic acid encoding Dupilumab in a cell culture production medium in large-scale, wherein sodium phosphate in said medium is supplemented on days 0, 2, 4, 6, and 8 to a concentration of about 250-200, about 500-550, about 500-550, about 225-275, and about 225-375 mg/L, respectively, such that a titer of said Dupilumab in said cell culture production medium on days 10-14 is about 5 g/L to 8 g/L.
135. The method of example 134, wherein said cell culture production medium is a serum-free medium.
136. The method of example 135, wherein said serum-free medium comprises a recombinant growth factor, an osmolarity regulator, a pH buffer agent, glutamine, and a cell protectant.
137. The method of any one of examples 134-136, wherein said CHO cells are cultured at a temperature ranging from about 32° C. to about 38° C.
138. The method of any one of examples 134-137, wherein sodium phosphate in said medium is supplemented on days 0, 2, 4, 6, and 8 to a concentration of about 267, about 525, about 525, about 250 and about 250 mg/L, respectively.
139. A fed-batch production method of making Dupilumab, comprising culturing Chinese Hamster Ovary (CHO) cells comprising a nucleic acid encoding Dupilumab in a cell culture production medium in large-scale, wherein sodium phosphate in said medium is supplemented on days 0, 2, 4, 6, and 8 to a concentration of about 250-200, about 500-550, about 500-550, about 225-275, and about 225-375 mg/L, respectively, wherein tyrosine in said medium is supplemented on day 3 to a concentration of about 2 g/L, and wherein insulin in said medium is supplemented on days 2 and 4 to a concentration of about 7.5 mg/L, such that titer of said Dupilumab in said cell culture production medium on day 10 is about 5 g/L.
140. The method of example 139, wherein said cell culture production medium is a serum-free medium.
141. The method of example 140, wherein said serum-free medium comprises a recombinant growth factor, an osmolarity regulator, a pH buffer agent, glutamine, methotrexate, and a cell protectant.
142. The method of any one of examples 139-141, wherein said CHO cells are cultured at a temperature ranging from about 32° C. to about 38° C.
143. The method of any one of examples 139-142, wherein sodium phosphate in said medium is supplemented on days 0, 2, 4, 6, and 8 to a concentration of about 267, about 525, about 525, about 250 and about 250 mg/L, respectively.
144. The method of any one of examples 139-143, wherein said culturing lasts about 10 days.
145. The method of any one of examples 134-144, wherein said large-scale production is >1,000 L.
146. The method of any one of examples 134-144, wherein said large-scale production is >3,000 L.
147. The method of any one of examples 134-144, wherein said large-scale production is >10,000 L.
148. The method of any one of examples 134-144, wherein said large-scale production is between 3,000 L and 25,000 L.
149. A system for producing Dupilumab, comprising:
   (a) a bioreactor for culturing cells capable of expressing Dupilumab;
   (b) one or more agitating elements; and
   (c) one or more gas control assemblies.
150. The system of example 149, wherein said one or more agitating elements comprises one or more impeller assemblies and the uppermost impeller is positioned below the surface of the initial working volume
151. The system of example 149 or 150. Wherein a first agitation rate is configured between 20 rpm and 150 rpm.
152. The system of any one of examples 149-151, wherein a second agitation rate is configured to increase by 25%, 50%, 75%, 100%, 125%, 150%, 175% or 200% on one or more days selected from the group of day 0.5, 1, 1.5, 2, 2.5, 3, 3.5, 4, 4.5, 5, 5.5, 6, 6.5, 7, 7.5, 8, 8.5, 9, 9.5, 10, 10.5, and 11.
153. The system of any one of examples 149-152, wherein said one or more gas control assemblies comprises one or more spargers.
154. The system of example 153, wherein said one or more spargers is configured at a sparging rate of about 25-75 slpm.
155. The system of any one of examples 149-154, wherein said sparging rate is configured to increase by 25%, 50%, 75%, 100%, 125%, 150%, 175%, 200%, 225%, 250%, 300%, 325%, 350%, 375%, 400%, 425%, 450%, 475%, or 500% on one or more days selected from the group of day 0.5, 1, 1.5, 2, 2.5, 3, 3.5, 4, 4.5, 5, 5.5, 6, 6.5, 7, 7.5, 8, 8.5, 9, 9.5, 10, 10.5, and 11.
156. The system of any one of examples 149-155, wherein said sparging rate is automatically configured based on dissolved oxygen levels,
157. The system of any one of examples 153-156, wherein said one or more spargers include between 146 and 292 holes sized between 0.5 mm and 2 mm.
158. A method for enhancing cell growth, cell viability, cell density, and/or production of Dupilumab in a mammalian cell culture process, comprising the steps of:
   (a) varying agitation rates in said cell culture process at different points during the growth and production phases;
   (b) varying sparging rates in said cell culture process at different points during the growth and production phases; and
   (c) varying dextrose target levels in said cell culture process during the growth and production phases.
159. The method of example 158, wherein a first agitation rate is set between 0.017 hp/1000 L, and 0.076 hp/1000 L.
160. The method of example 158 or 159, wherein a second agitation rate is configured to increase by 25%, 50%, 75%, 100%, 125%, 150%, 175% or 200% on one or more days selected from the group of day 0.5, 1, 1.5, 2, 2.5, 3, 3.5, 4.5, 5, 5.5, 6, 6.5, 7, 7.5, 8, 8.5, 9, 9.5, 10, 10.5, and 11.
161. The method of any one of examples 158-160, wherein more than one sparger is used to vary the sparging rate.
162. The method of example 161, wherein said more than one sparger is set at a first sparging rate of about 25-75 slpm.
163. The method of any one of examples 158-162, wherein a second sparging rate is increased by 25%, 50%, 75%, 100%, 175%, 150%, 175%, 700%, 275%. 250%, 300%, 325%, 350%, 375%, 400%, 425%, 450%, 475%, or 500% on one or more days selected from the group of day 0.5, 1, 1.5, 2, 2.5, 3, 3.5, 4, 4.5, 5, 5.5, 6, 6.5, 7, 7.5, 8, 8.5, 9, 9.5, 10, 10.5, and 11.
164. The method of any one of examples 158-1.63, wherein said first and second sparging rate is automatically configured based on dissolved oxygen levels.
165. The method of any one of examples 161-164, wherein said spargers comprise between 146 and 292 holes sized between 0.5 mm and 2 mm.
166. The method of any one of examples 158-165, wherein an initial dextrose target level is configured between 5 g/L and 7 g/L.
167. The method of any one of examples 158-166, wherein a dextrose target level is configured to vary between 5 g/L and 7 g/L on day 0 and then stepped up to vary between 7 g/L and 9 g/L on day 2 and then stepped up to vary between 9 g/L and 11 g/L on day 4.
168. The method of any one of examples 158-166, wherein a dextrose target level is configured to vary between 5 g/L and 7 g/L on day 0 and then stepped up to vary between 7 g/L and 11 g/L on day 2 and then decreased to vary between 5 g/L and 7 g/L on day 4.
169. A method of measuring dissolved oxygen using one or more electrochemical probes, wherein data from said electrochemical probe is processed to reduce signal noise.
170. The method of example 169, further comprising the step of applying an Agile filter to reduce signal noise.
171. The method of example 169 or 170, further comprising the step of applying a. Savitzky-Golay filter to reduce signal noise.
172. The method of any one of examples 169-171, further comprising the step of reducing signal noise by smoothing data.
173. The method of example 172, wherein the sampling window is between 10 minutes and 33 minutes.
174. The method of example 172 or 173, wherein the sampling rate is about 59 seconds.
175. A method of measuring dissolved oxygen using one or more optical probes, wherein data from said optical probe is processed to improve accuracy.
176. A method of measuring dissolved oxygen using one or more optical probes, wherein data from said optical probe is processed by applying an offset or smoothing the data.
177. The method of example 175 or 176, further comprising use of a fixed offset.
178. The method of any one of examples 175-177, further comprising use of a variable offset, wherein said offset changes at some point during the run time.
179. The method of any one of examples 176-178, wherein said offset is calculated based on a correlation to an electrochemical probe.
180. A bioreactor with reduced maintenance requirements, comprising:
   bioreactor;
   one or more optical probes in said bioreactor for measuring dissolved oxygen and generating a data signal; and
   a processor configured to process said data signal and apply an offset to align performance of said optical probe with an electrochemical probe.
181. The bioreactor of example 180, further comprising two or more optical probes configured at different locations within a bioreactor.
182. The bioreactor of example 180 or 181, further comprising at least one optical probe with an anti babble cap.
183. A bioreactor comprising:
   one or more optical probes for generating a data signal with reduced signal noise compared to an electrochemical probe.
184. The bioreactor of example 183, further comprising two or more optical probes.
185. The bioreactor of example 184, further comprising two or more optical probes configured in the lower third of the bioreactor.
186. The bioreactor of example 184, further comprising two or more optical probes configured at two different locations along a probe belt.
187. The bioreactor of any one of examples 183-186, further comprising an agitating element comprising one or more impeller assemblies, wherein an uppermost impeller is positioned below the surface of the initial working volume.
188. A method of culturing a cell, comprising:
   a) using an on-line sensor to measure a first property of a cell culture;
   b) using said measure of said first property of said cell culture and a correlation equation to predict at least one measure of a second property of said cell culture; and
   c) adjusting a culturing condition based on said at least one predicted measure of said second property of said cell culture to culture said cell.
189. A method of culturing a cell, comprising:
   a) using at least one on-line sensor to measure a first property of a first cell culture;
   b) using at least one off-line assay to measure a second property of said first cell culture;
   c) correlating said measure of said first property of said first cell culture with said measure of said second property of said first cell culture to determine a correlation equation;
   d) using an on-line sensor to measure said first property of a second cell culture;
   e) using said measure of said first property of said second cell culture and said correlation equation to predict at least one measure of said second property of said second cell culture; and
   f) adjusting a culturing condition based on said at least one predicted measure of said second property of said second cell culture to culture the cell.
190. A method of culturing a cell, comprising:
   a) using at least one on-line capacitance probe to measure a first capacitance value of a first cell culture;
   b) using at least one off-line assay to measure a first viable cell density value of said first cell culture;
   c) correlating said first capacitance value with said first viable cell density value to determine a correlation equation;
   d) using an on-line capacitance probe to determine a second capacitance value of a second cell culture;
   e) using said second capacitance value and said correlation equation to predict at least one second viable cell density value of said second cell culture; and
   f) adjusting a culturing condition based on said second viable cell density value to culture the cell.
191. The method of any one of examples 188-190, wherein said cell is from a cell line that is the same as a cell line used to derive said correlation equation.
192. The method of any one of examples 188-190, wherein said cell is from a cell line that is different from a cell line used to derive said correlation equation.
193. The method of any one of examples 188-192, wherein said correlation equation is derived using more than one cell line.
194. The method of example 193, wherein said cell is from a cell line that is the same as a cell line used to derive said correlation equation.
195. The method of example 193, wherein said cell is from a cell line that is different from a cell line used to derive said correlation equation.
196. The method of any one of examples 190-195, wherein more than one first capacitance value of said first cell culture is measured.
197. The method of any one of examples 190-196, wherein more than one first viable cell density value of said first cell culture is measured.
198. The method of any one of examples 190-197, wherein at least about 50 percent of variability in said first viable cell density values is due to variance in said first capacitance values.
199. The method of any one of examples 188-198, wherein said correlation equation is produced using multivariate data analysis.
200. A method for measuring viable cell density of cells cultured in a bioreactor, comprising:
   a) applying an electric field to said cells cultured in a bioreactor;
   b) measuring capacitance; and
   c) correlating capacitance to viable cell density.
201. A method of culturing cells wherein the initial viable cell density (VCD) at the seed train N-5 to N-1 is 2.5×10⁵ to 4.0×10⁵ cells/mL.
202. A method of producing Dupilumab, comprising:
   a. culturing Chinese Hamster Ovary (CHO) cells capable of expressing Dupilumab in serum-free medium, wherein said medium comprises insulin, tyrosine, sodium phosphate and one or amino acids;
   b. supplementing the medium on about day 2 and on about day 4 with additional insulin at a concentration of about 7.5 mg/L;
   c. supplementing the medium on about day 3 with additional tyrosine at a concentration of about 2.0 mg/L;
   d. supplementing the medium on about day 0, 2, 4, 6 and on about day 8 with additional sodium phosphate at a concentration of about 250-200, about 500-550, about 500-550, about 225-275, or about 225-375 mg/L;
   e. harvesting Dupilumab at about 10 to 14 days after the start of culture;
   f. subjecting said harvested antibody to affinity chromatography;
   g. subjecting said antibody pooled from eluate in step f. to viral inactivation at a pH from about 3 to about 4 and adjust pH to about 5 to about 6;
   h. subjecting said antibody pooled from step g. to anion exchange chromatography in flowthrough mode;
   i. subjecting said antibody pooled from eluate of step h. to hydrophobic interaction chromatography in flowthrough mode; and
   j. subjecting said antibody pooled from flowthrough fractions of step i. to virus retentive filtration to produce Dupilumab, and wherein the titer of Dupilumab is at least 5 g/L.
203. The method of example 202, further comprising a harvest pre-treatment step wherein harvest pre-treatment includes subjecting said antibody to transient pH levels from about 4.5 to 5.0 to about 5.5 to 6.5.
204. The method of example 202 or 203, wherein said antibody of step j. is further subjected to concentration and diafiltration using a diafiltration buffer having a pH between 4.0 and 4.5.
205. The method of example 204, wherein said diafiltration buffer comprises about 4 mM acetate to about 6 mM acetate.
206. The method of any one of examples 202-205, wherein said affinity chromatography is Protein A.
207. The method of example 206, wherein the Protein A resin is selected from the group consisting of Mab Select PrismA, Mab Select SuRe, Mab Select SuRe LX, Mab Select, Mab Select SuRe pcc, Mab Select Xtra, rProtein A Sepharose, ProSep HC, ProSep Ultra, ProSep Ultra Plus, MabCapture, and Amsphere A3.
208. The method of any one of examples 202-207, wherein said cell culture medium further comprises ornithine between 0.03 mM and about 0.9 mM, amino acids, nucleosides, salts of divalent cations, fatty acids, tocopherol, and vitamins.
209. The method of example 208, wherein said amino acids are selected from a group consisting of alanine, arginine, asparagine, aspartic acid, cysteine, glutamic acid, glycine, histidine, isoleucine, leucine, lysine, methionine, phenylalanine, proline, serine, threonine, tryptophan, tyrosine, and valine.
210. The method of example 209, wherein a concentration of amino acids having a non-polar side group is at least 15 mM, at least 24 mM, at least 25 mM, at least 26 mM, at least 27 mM, at least 28 mM, at least 29 mM, or at least 30 mM.
211. The method of example 209 or 210, wherein a concentration of amino acids having an uncharged polar side group is between about 10 mM and about 34 mM, between about 15 mM and about 30 mM, between about 20 mM and about 25 mM, or about 22 mM.
212. The method of any one of examples 209-211, wherein a concentration of acidic amino acids is between about 4 mM and about 14 mM, about 4 mM, or about 9 mM.
213. The method of any one of examples 209-212, wherein a concentration of basic amino acids is at least 3.5 mM, at least 4 mM, at least 5 mM, at least 6 mM, at least 7 mM, at least 8 mM, at least 9 mM, at least 10 mM, at least 11 mM, or about 11 mM.
214. The method of example 209, wherein a concentration of non-polar amino acids is about 30 mM, a concentration of uncharged polar amino acids is about 22 mM, a concentration of acidic amino acids is about 9 mM, and a concentration of basic amino acids is about 11 mM.
215. The method of any one of examples 208-214, wherein a concentration of said nucleosides is at least 50 at least 100 at least 150 or at least 170 μM.
216. The method of any one of examples 208-215, wherein said nucleosides comprise purine derivatives, wherein a concentration of said purine derivatives is at least 40 at least 60 at least 80 at least 100 at least 105 or about 106 μM.
217. The method of any one of examples 208-216, wherein said nucleosides comprise pyrimidine derivatives, wherein a concentration of said pyrimidine derivatives is at least 30 at least 50 at least 65 or about 68 μM.
218. The method of any one of examples 208-217, wherein said nucleosides comprise one or more of the following: adenosine, guanosine, cytidine, uridine, thymidine, and hypoxanthine.
219. The method of any one of examples 208-218, wherein said fatty acids comprise one or more of the following: linoleic acid, linolenic acid, thioctic acid, oleic acid, palmitic acid, stearic acid, arachidic acid, arachidonic acid, lauric acid, behenic acid, decanoic acid, dodecanoic acid, hexanoic acid, lignoceric acid, myristic acid, and octanoic acid.
220. The method of any one of examples 208-219, wherein said salts comprise one or more of the following: Ca²⁺ and Mg²⁺.
221. The method of any one of examples 208-220, wherein a concentration of said vitamins is at least about 700 μM or at least about 2 mM.
222. The method of any one of examples 208-221, wherein said vitamins comprise one or more of the following: D-biotin, choline chloride, folic acid, myo-inositol, niacinamide, pyridoxine HCl, D-pantothenic acid (hemiCa), riboflavin, thiamine HCl, and vitamin B12.
223. The method of any one of examples 208-222, wherein said cell culture medium further comprises one or more of taurine or hypotaurine.
224. The method of any one of examples 208-223, wherein said cell culture medium further comprises at least one recombinant growth factor.
225. The method of example 224, wherein said at least one recombinant growth factor is insulin.
226. A method of producing an anti-IL-4Rα antibody or antigen-binding fragment thereof, comprising the steps of:
   culturing cells expressing the anti-IL-4Rα antibody or antigen-binding fragment thereof using a cell culture medium comprising ornithine at between about 0.03 and about 0.9 mM and/or putrescine at between about 0.20 mM and about 0.9 mM in a bioreactor comprising:
   one or more agitating elements; and
   one or more gas control assemblies.
227. The method of example 226, wherein said agitating element comprises two or more impeller assemblies and the midpoint of the uppermost impeller is positioned below the surface of the initial working volume.
228. The method of example 226 or 227, wherein an initial agitation rate is configured between 20 rpm and 150 rpm.
229. The method of any one of examples 226-228, wherein the agitation rate is configured to increase by 25%, 50%, 75%, 100%, 125%, 150%, 175% or 200% on one or more days selected from the group of day 0.5, 1, 1.5, 2, 2.5, 3, 3.5, 4, 4.5, 5, 5.5, 6, 6.5, 7, 7.5, 8, 8.5, 9, 9.5, 10, 10.5, and 11.
230. The method of any one of examples 226-229, wherein said gas control assemblies comprises one or more spargers.
231. The method of example 230, wherein said one or more spargers is configured at an initial sparging rate of about 25-75 slpm.
232. The method of example 230, wherein said sparging rate is configured to be increased by 25%, 50%, 75%, 100%, 125%, 150%, 175%, 200%, 225%, 250%, 300%, 325%, 350%, 375%, 400%, 425%, 450%, 475%, or 500% on one or more days selected from the group of day 0.5, 1, 1.5, 2, 2.5, 3, 3.5, 4, 4.5, 5, 5.5, 6, 6.5, 7, 7.5, 8, 8.5, 9, 9.5, 10, 10.5, and 11.
233. The method of any one of examples 230-232, wherein the sparging rate is automatically configured based on dissolved oxygen levels of about 20%.
234. A method comprising the steps of:
   (a) culturing cells expressing the anti-IL-4Rα antibody or antigen-binding fragment thereof using a cell culture medium comprising ornithine at between about 0.09 and about 0.9 mM and/or putrescine at between about 0.20 mM and about 0.9 mM,
   (b) harvesting said cells by centrifugation to separate cell debris from clarified media comprising the anti-IL-4Rα antibody or antigen-binding fragment thereof;
   (c) subjecting said clarified media and anti-IL-4Rα antibody or antigen-binding fragment thereof to affinity chromatography;
   (d) subjecting the anti-IL-4Rα antibody or antigen-binding fragment thereof pooled from eluate in step (c) to viral inactivation;
   (e) subjecting the anti-IL-4Rα antibody or antigen-binding fragment thereof pooled from step (d) to anion exchange chromatography in flowthrough mode;
   (f) subjecting the anti-IL-4Rα antibody or antigen-binding fragment thereof pooled from flowthrough fractions of step (e) to cation exchange chromatography in bind and elute mode;
   (g) subjecting the anti-IL-4Rα antibody or antigen-binding fragment thereof pooled from eluate of step (f) to hydrophobic interaction chromatography in flowthrough mode; and
   (h) subjecting the anti-IL-4Rα antibody or antigen-binding fragment thereof pooled from flowthrough fractions of step (g) to virus retentive filtration, thereby producing an anti-IL-4Rα antibody or antigen-binding fragment thereof.
235. A method for producing an antibody or antigen-binding fragment thereof, comprising:
   (a) subjecting harvested cell culture media to affinity chromatography;
   (b) subjecting eluate from step (a) to a second chromatography step to produce an eluate or flowthrough fraction comprising said antibody or antigen-binding fragment thereof; and
   (c) subjecting said eluate or flowthrough fraction from step (b) to a third chromatography step to produce an antibody or antigen-binding fragment thereof.
236. The method of example 235, wherein said second chromatography step is mixed-mode chromatography, anion exchange chromatography, cation exchange chromatography, or hydrophobic interaction chromatography.
237. The method of example 235, wherein said third chromatography step is mixed-mode chromatography, anion exchange chromatography, cation exchange chromatography, or hydrophobic interaction chromatography.
238. The method of example 235, further comprising subject an eluate or flowthrough fraction comprising said antibody or antigen-binding fragment thereof from step (c) to a fourth chromatography step.
239. The method of example 238, wherein said fourth chromatography step is mixed-mode chromatography, anion exchange chromatography, cation exchange chromatography, or hydrophobic interaction chromatography.
240. The method of example 235, further comprising subjecting said antibody or antigen-binding fragment thereof to virus retentive filtration.
241. A method comprising the steps of:
   (a) subjecting a harvested antibody to affinity chromatography;
   (b) subjecting said antibody pooled from eluate of step (a) to viral inactivation at a pH from about 3 to about 4 and then adjusting the pH to from about 5 to about 8;
   (c) subjecting said antibody pooled from step (b) to anion exchange chromatography in flowthrough mode;
   (d) subjecting said antibody pooled from flowthrough fractions of step (c) to cation exchange chromatography in bind and elute mode;
   (e) subjecting said antibody pooled from eluate of step (d) to hydrophobic interaction chromatography in flowthrough mode; and
   (f) subjecting said antibody pooled from flowthrough fractions of step (e) to virus retentive filtration to produce an anti-IL4Rα antibody.
242. The method of example 241, wherein said viral inactivation includes a buffer comprising from about 0.25 M to about 1 M phosphoric acid, optionally wherein said buffer comprises about 0.25 M phosphoric acid or about 1 M phosphoric acid.
243. The method of any one of examples 241-242, wherein said viral inactivation is at a pH from about 3.5 to about 3.7 or from 3.45 to about 3.65.
244. The method of any one of examples 241-243, wherein said adjusting the pH is at a pH from about 5.4 to about 5.8 or about 5.8 to about 6.2.
245. The method of any one of examples 235-244, further comprising a harvest pre-treatment step prior to step (a).
246. The method of example 245, wherein said harvest pre-treatment step includes adjusting said antibody to a transient pH level from about 4 to 5.5.
247. The method of example 245, wherein said harvest pre-treatment step includes adjusting said antibody to a pH level from about 4 to about 5.5 for about 30 to about 60 minutes, and then adjusting said antibody to a pH level of about 6 for about 30 to about 60 minutes.
248. The method of example 245, wherein the method does not include depth filtration.
249. The method of any one of examples 241-247, further comprising a step of subjecting a harvested antibody to polish filtration prior to step (a).
250. The method of example 249, wherein said polish filter is a multi-mechanism device or a functionalized filter.
251. The method of example 249 or 250, wherein loading of a polish filter is from about 255 L/m² to about 270 L/m².
252. The method of any one of examples 1-30, 202-225, 234-240, 241-251, 284-408, or 509-532, wherein a quantity of protein loaded on an AEX resin is between about between about 50 g/L-resin and about 200 g/L-resin, between about 100 g/L-resin and about 150 g/L-resin, less than about 120 g/L-resin, about 50 g/L-resin, about 55 g/L-resin, about 60 g/L-resin, about 65 g/L-resin, about 70 g/L-resin, about 75 g/L-resin, about 80 g/L-resin, about 85 g/L-resin, about 90 g/L-resin, about 95 g/L-resin, about 100 g/L-resin, about 105 g/L-resin, about 110 g/L-resin, about 115 g/L-resin, about 120 g/L-resin, about 125 g/L-resin, about 130 g/L-resin, about 135 g/L-resin, about 140 g/L-resin, about 145 g/L-resin, about 150 g/L-resin, about 155 g/L-resin, about 160 g/L-resin, about 165 g/L-resin, about 170 g/L-resin, about 175 g/L-resin, about 180 g/L-resin, about 185 g/L-resin, about 190 g/L-resin, about 195 g/L-resin, or about 200 g/L-resin.
253. The method of any one of examples 1-30, 202-225, 234-240, 241-252, 284-408, or 509-532, wherein a pH of an AEX load is between about 7.40 and about 8.30, between about 7.50 and about 7.70, between about 7.55 and about 7.65, about 7.40, about 7.45, about 7.50, about 7.51, about 7.52, about 7.53, about 7.54, about 7.55, about 7.56, about 7.57, about 7.58, about 7.59, about 7.60, about 7.61, about 7.62, about 7.63, about 7.64, about 7.65, about 7.66, about 7.67, about 7.68, about 7.69, about 7.70, about 7.75, about 7.80, about 7.85, about 7.90, about 7.95, about 8.00, about 8.05, about 8.10, about 8.15, about 8.20, about 8.25, or about 8.30.
254. The method of any one of examples 1-30, 202-225, 234-240, 241-253, 284-408, or 509-532, wherein a concentration of protein loaded onto an AEX column is between about 10.0 g/L-resin and about 30.0 g/L-resin, between about 12 g/L-resin and about 25 g/L-resin, about 10.0 g/L-resin, about 11.0 g/L-resin, about 12.0 g/L-resin, about 13.0 g/L-resin, about 14.0 g/L-resin, about 15.0 g/L-resin, about 16.0 g/L-resin, about 17.0 g/L-resin, about 18.0 g/L-resin, about 19.0 g/L-resin, about 20.0 g/L-resin, about 21.0 g/L-resin, about 22.0 g/L-resin, about 23.0 g/L-resin, about 24.0 g/L-resin, about 25.0 g/L-resin, about 26.0 g/L-resin, about 27.0 g/L-resin, about 28.0 g/L-resin, about 29.0 g/L-resin, or about 30.0 g/L-resin.
255. The method of any one of examples 1-30, 202-225, 234-240, 241-254, 284-408, or 509-532, wherein an AEX wash buffer comprises about 50 mM Tris and about 60 mM acetate, at a pH between about 7.50 and about 7.70, and a conductivity between about 3.00 mS/cm and about 4.00 mS/cm.
256. The method of any one of examples 1-30, 202-225, 234-240, 241-255, 284-408, or 509-532, wherein said AEX step includes a pre-equilibration step, optionally wherein a pre-equilibration buffer comprises about 2 M sodium chloride, water for injection, or a combination thereof
257. The method of any one of examples 1-30, 202-225, 234-240, 241-256, 284-408, or 509-532, wherein said AEX step includes an equilibration step, optionally wherein an equilibration buffer comprises about 50 mM Tris and about 60 mM acetate, a pH between about 7.50 and about 7.70, and/or a conductivity between about 3.00 mS/cm and about 4.00 mS/cm.
258. The method of any one of examples 1-30, 202-225, 234-240, 241-257, 284-344, or 509-532, wherein a pH of a CEX load is between about 4.00 and about 6.50, between about 5.00 and about 6.00, between about 5.90 and about 6.10, about 4.00, about 4.10, about 4.20, about 4.30, about 4.40, about 4.50, about 4.60, about 4.70, about 4.80, about 4.90, about 5.00, about 5.10, about 5.20, about 5.30, about 5.40, about 5.50, about 5.60, about 5.70, about 5.80, about 5.90, about 6.00, about 6.10, about 6.20, about 6.30, about 6.40, or about 6.50.
259. The method of any one of examples 1-30, 202-225, 234-240, 241-258, 284-344, or 509-532, wherein a CEX wash buffer comprises about 40 mM sodium acetate at a pH between about 5.90 and about 6.10, and a conductivity between about 2.00 mS/cm and about 4.00 mS/cm.
260. The method of any one of examples 1-30, 202-225, 234-240, 241-259, 284-344, or 509-532, wherein a CEX elution buffer comprises about 20 mM Tris and about 120 mM sodium acetate at a pH between about 5.90 and about 6.20, and a conductivity between about 9.00 mS/cm and about 11.00 mS/cm.
261. The method of any one of examples 1-30, 202-225, 234-240, 241-260, 284-309, or 509-518, wherein a concentration of protein loaded onto a HIC column is between about 80-100 g/L-resin or about 180-200 g/L-resin.
262. The method of any one of examples 241-261, wherein a HIC equilibration buffer and/or a HIC wash buffer comprises about 30 mM sodium citrate, about 40 mM sodium citrate, or no more than about 40 mM sodium citrate.
263. The method of any one of examples 241-262, wherein the concentration of said antibody pooled from flowthrough fractions in step (f) is between about 4 g/L to 12 g/L.
264. The method of any one of examples 241-263, further comprising subjecting said antibody to ultrafiltration and diafiltration (UF/DF) after step (f).
265. The method of example 264, wherein said UF/DF includes a diafiltration buffer having a pH between 4.0 and 4.5.
266. The method of example 264 or 265, wherein a concentrated antibody pool following UF/DF has a pH of about 5.3.
267. The method of any one of examples 264-266, wherein said UF/DF includes a diafiltration buffer comprising between about 4 mM acetate and about 6 mM acetate.
268. The method of any one of examples 241-267, further comprising adjusting a sample with a load adjustment solution, optionally wherein said load adjustment solution comprises about 10% (w/v) super refined polysorbate 80 or polysorbate 20 and/or is added as about 50 μL/L of load.
269. The method of any one of examples 235-268, wherein said affinity chromatography is Protein A chromatography.
270. The method of example 269, wherein a Protein A resin is selected from the group consisting of Mab Select PrismA, Mab Select SuRe, Mab Select SuRe LX, MabSelect, Mab Select SuRe pcc, Mab Select Xtra, rProtein A Sepharose, ProSep HC, ProSep Ultra, ProSep Ultra Plus, MabCapture, and Amsphere A3.
271. The method of example 269 or 270, wherein a Protein A resin is selected that is capable of receiving a protein load at a concentration above 55 g/L-resin.
272. The method of any one of examples 269-271, wherein a pH of a Protein A load is about 6.
273. The method of any one of examples 269-271, wherein a pH of a Protein A load is from about 6 to about 8.
274. The method of any one of examples 241-273, wherein about 180 g to 200 g of antibody is loaded per liter of HIC resin.
275. The method of any one of examples 241-274, wherein the amount of PLBD2 in the HIC eluate is reduced compared to the amount of PLBD2 in the HIC load, optionally wherein the amount of PLBD2 in the HIC eluate is reduced to below 100 ppm, reduced to below 30 ppm, reduced to below 4 ppm, reduced to below 1 ppm, or reduced by about 40x-310x compared to the amount of PLBD2 in the HIC load.
276. The method of any one of examples 241-274, wherein the amount of PLBD2 in the HIC eluate is reduced compared to the amount of PLBD2 in the HIC load.
277. The method of any one of examples 241-274, wherein the amount of PLBD2 in the HIC eluate is reduced to below 100 ppm.
278. The method of any one of examples 241-274, wherein the amount of PLBD2 in the HIC eluate is reduced to below 30 ppm.
279. The method of any one of examples 241-274, wherein the amount of PLBD2 in the HIC eluate is reduced to below 4 ppm.
280. The method of any one of examples 241-274, wherein the amount of PLBD2 in the HIC eluate is reduced to below 1 ppm.
281. The method of any one of examples 241-280, wherein said anti-IL-4Rα antibody comprises three heavy chain complementarity determining region (HCDR) sequences comprising SEQ ID NOs: 3, 4, and 5, and three light chain complementarity determining region (LCDR) sequences comprising SEQ ID NOs: 6, 7, and 8.
282. The method of any one of examples 241-281, wherein said anti-IL-4Rα antibody comprises a heavy chain variable region (HCVR) comprising the amino acid sequence of SEQ ID NO: 1 and a light chain variable region (LCVR) comprising the amino acid sequence of SEQ ID NO: 2.
283. The method of any one of examples 241-282, wherein said anti-IL-4Rα antibody is Dupilumab.
284. A method comprising the steps of:
   (a) culturing cells expressing an anti-IL-4Rα antibody or antigen-binding fragment thereof;
   (b) subjecting said cells to transient pH levels from about 4.0 to 5.5, then raising pH levels to from about 5.5 to 6.5;
   (c) harvesting said cells by centrifugation to separate cell debris from clarified media comprising said anti-IL-4Rα antibody or antigen-binding fragment thereof;
   (d) subjecting said clarified media to affinity chromatography;
   (e) subjecting said anti-IL-4Rα antibody or antigen-binding fragment thereof pooled from eluate of step (d) to viral inactivation at a pH from about 3 to about 4 and then adjusting the pH to from about 5 to about 8;
   (f) subjecting said anti-IL-4Rα antibody or antigen-binding fragment thereof pooled from step (e) to anion exchange chromatography in flowthrough mode;
   (g) subjecting said anti-IL-4Rα antibody or antigen-binding fragment thereof pooled from flowthrough fractions of step (f) to cation exchange chromatography in bind and elute mode;
   (h) subjecting said anti-IL-4Rα antibody or antigen-binding fragment thereof pooled from eluate of step (g) to hydrophobic interaction chromatography in flowthrough mode; and
   (i) subjecting said anti-IL-4Rα antibody or antigen-binding fragment thereof pooled from flowthrough fractions of step (h) to virus retentive filtration to produce an anti-IL-4Rα antibody or antigen-binding fragment thereof.
285. The method of example 284, further comprising subjecting said anti-IL-4Rα antibody or antigen-binding fragment thereof to ultrafiltration and diafiltration (UF/DF) after step (i).
286. The method of example 284 or 285, wherein said affinity chromatography is Protein A chromatography.
287. The method of example 286, wherein a Protein A resin is selected from the group consisting of Mab Select PrismA, Mab Select SuRe, Mab Select SuRe LX, MabSelect, Mab Select SuRe pcc, Mab Select Xtra, rProtein A Sepharose, ProSep HC, ProSep Ultra, ProSep Ultra Plus, MabCapture, and AmsphereA3.
288. The method of any one of examples 284-287, wherein an anion exchange resin is selected from the group consisting of Q Sepharose Fast Flow, Poros 50PI, Poros 50HQ, Capto Q Impres, Capto DEAE, Toyopearl QAE-550, Toyopearl DEAE-650, Toyopearl GigaCap Q-650, Fractogel EMD TMAE Hicap, Sartobind STIC PA nano, Sartobind Q nano, CUNO BioCap, and XOHC.
289. The method of any one of examples 284-288, wherein an anion exchange resin is Poros 50HQ.
290. The method of any one of examples 284-288, wherein an anion exchange resin is Q Sepharose Fast Flow.
291. The method of any one of examples 284-290, wherein a cation exchange resin is selected from the group consisting of Fractogel Hicap, Capto SP ImpRes, Capto S ImpAc, CM Hyper D grade F, Eshmuno S, Nuvia C Prime, Nuvia S, Poros HS, and Poros XS.
292. The method of any one of examples 284-291, wherein a cation exchange resin is Capto ImpRes.
293. The method of any one of examples 284-291, wherein a cation exchange resin is Fractogel Hicap.
294. The method of any one of examples 284-293, further comprising passing said anti-IL-4Rα antibody or antigen-binding fragment thereof through a LifeAssure filter after the viral inactivation of step (e) and prior to the anion exchange chromatography of step (f).
295. The method of any one of examples 285-294, wherein said UF/DF step comprises a membrane filter device selected from the group consisting of Pellicon 2, Pellicon 3 cassettes with 10 kD, 30 kD or 50 kD membranes, Kvick 10 kD, 30 kD or 50 kD membrane cassettes, and Centramate and Centrasette 10 kD, 30 kD or 50 kD cassettes.
296. The method of any one of examples 285-295, wherein said UF/DF step does not include addition of arginine.
297. The method of any one of examples 284-296, wherein said HIC step comprises HIC media selected from the group consisting of Capto Phenyl, Capto Phenyl High Sub, Phenyl Sepharose™ 6 Fast Flow, Phenyl Sepharose™ High Performance, Octyl Sepharose High Performance, Fractogel EMD Propyl, Fractogel EMD Phenyl, Macro-Prep Methyl, Macro-Prep t-Butyl columns, WP HI-Propyl (C3), Toyopearl ether, phenyl or butyl, Toyo PPG; Toyo Phenyl; Toyo Butyl, and Toyo Hexyl.
298. The method of any one of examples 284-297, wherein said anti-IL-4Rα antibody or antigen-binding fragment thereof comprises three heavy chain complementarity determining region (HCDR) sequences comprising SEQ ID NOs: 3, 4, and 5, and three light chain complementarity determining region (LCDR) sequences comprising SEQ ID NOs: 6, 7, and 8.
299. The method of any one of examples 284-298, wherein said anti-IL-4Rα antibody or antigen-binding fragment thereof comprises a heavy chain variable region (HCVR) comprising the amino acid sequence of SEQ ID NO: 1 and a light chain variable region (LCVR) comprising the amino acid sequence of SEQ ID NO: 2.
300. The method of any one of examples 284-299, wherein said anti-IL-4Rα antibody is Dupilumab.
301. A method for producing an anti-IL4Rα antibody or antigen-binding fragment thereof, comprising:
   (a) subjecting a harvested antibody to affinity chromatography;
   (b) subjecting said antibody pooled from eluate of step (a) to viral inactivation;
   (c) subjecting said antibody pooled from step (b) to anion exchange chromatography in flowthrough mode;
   (d) subjecting said antibody pooled from flowthrough fractions of step (c) to cation exchange chromatography in bind and elute mode;
   (e) subjecting said antibody pooled from eluate of step (d) to hydrophobic interaction chromatography in flowthrough mode;
   (f) subjecting said antibody pooled from flowthrough fractions of step (e) to virus retentive filtration; and
   (g) subjecting said antibody pooled from step (f) to ultrafiltration and diafiltration (UF/DF) to produce an anti-IL4Rα antibody or antigen-binding fragment thereof, wherein said UF/DF step does not include addition of arginine.
302. The method of example 301, wherein a diafiltration buffer of step (g) has a pH between 4.0 and 4.5.
303. The method of example 301 or 302, wherein said diafiltration buffer comprises between about 4 mM acetate and about 6 mM acetate.
304. The method of any one of examples 301-303, wherein a concentrated antibody pool following UF/DF has a pH of about 5.3.
305. The method of any one of examples 301-304, wherein said UF/DF step comprises a membrane filter device selected from the group consisting of Pellicon 2, Pellicon 3 cassettes with 10 kD, 30 kD or 50 kD membranes, Kvick 10 kD, 30 kD or 50 kD membrane cassettes, and Centramate and Centrasette 10 kD, 30 kD or 50 kD cassettes.
306. A method for treating a patient having a type 2 inflammatory disease, comprising administering to a patient an anti-IL4Rα antibody produced according to the method of any one of examples 235-305.
307. The method of example 306, wherein said type 2 inflammatory disease is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.
308. A method for treating a disease or disorder associated with IL-4R activity, comprising administering to a patient an anti-IL4Rα antibody produced according to the method of any one of examples 235-307.
309. The method of example 308, wherein said disease or disorder associated with IL-4R activity is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.
310. A method comprising the steps of:
   (a) subjecting a harvested antibody to affinity chromatography;
   (b) subjecting said antibody pooled from eluate of step (a) to viral inactivation at a pH from about 3 to about 4 and then adjusting the pH to from about 5 to about 8;
   (c) subjecting said antibody pooled from step (b) to cation exchange chromatography in bind and elute mode;
   (d) subjecting said antibody pooled from eluate of step (c) to anion exchange chromatography in flowthrough mode; and
   (e) subjecting said antibody pooled from flowthrough fractions of step (d) to virus retentive filtration to produce an anti-IL4Rα antibody.
311. The method of example 310, further comprising a harvest pre-treatment step prior to step (a).
312. The method of example 311, wherein said harvest pre-treatment step includes adjusting said antibody to a transient pH level from about 4 to 5.5.
313. The method of any one of examples 310-312, further comprising subjecting said antibody to ultrafiltration and diafiltration (UF/DF) after step (e).
314. The method of example 313, wherein said UF/DF includes a diafiltration buffer having a pH between 4.0 and 4.5.
315. The method of example 313 or 314, wherein a concentrated antibody pool following UF/DF has a pH of about 5.3.
316. The method of any one of examples 313-315, wherein said UF/DF includes a diafiltration buffer comprising between about 4 mM acetate and about 6 mM acetate.
317. The method of any one of examples 310-316, wherein said affinity chromatography is Protein A chromatography.
318. The method of example 317, wherein a Protein A resin is selected from the group consisting of MabSelect PrismA, MabSelect SuRe, MabSelect SuRe LX, MabSelect, MabSelect SuRe pcc, MabSelect Xtra, rProtein A Sepharose, ProSep HC, ProSep Ultra, ProSep Ultra Plus, MabCapture, and Amsphere A3.
319. The method of example 317 or 318, wherein a Protein A resin is selected that is capable of receiving a protein load at a concentration above 55 g/L-resin.
320. The method of any one of examples 317-319, wherein a Protein A column load pH is between 6 and 8.
321. The method of any one of examples 310-320, wherein said anti-IL-4Rα antibody comprises three heavy chain complementarity determining region (HCDR) sequences comprising SEQ ID NOs: 3, 4, and 5, and three light chain complementarity determining region (LCDR) sequences comprising SEQ ID NOs: 6, 7, and 8.
322. The method of any one of examples 310-321, wherein said anti-IL-4Rα antibody comprises a heavy chain variable region (HCVR) comprising the amino acid sequence of SEQ ID NO: 1 and a light chain variable region (LCVR) comprising the amino acid sequence of SEQ ID NO: 2.
323. The method of any one of examples 310-322, wherein said anti-IL-4Rα antibody is Dupilumab.
324. A method comprising the steps of:
   (a) culturing cells expressing an anti-IL-4Rα antibody or antigen-binding fragment thereof;
   (b) subjecting said cells to transient pH levels from about 4 to 5.5, then raising pH levels to from about 5.5 to 6.5;
   (c) harvesting said cells by centrifugation to separate cell debris from clarified media comprising said anti-IL-4Rα antibody or antigen-binding fragment thereof;
   (d) subjecting said clarified media to affinity chromatography;
   (e) subjecting said anti-IL-4Rα antibody or antigen-binding fragment thereof pooled from eluate of step (d) to viral inactivation at a pH from about 3 to about 4 and then adjusting the pH to from about 5 to about 8;
   (f) subjecting said anti-IL-4Rα antibody or antigen-binding fragment thereof pooled from step (e) to cation exchange chromatography in bind and elute mode;
   (g) subjecting said anti-IL-4Rα antibody or antigen-binding fragment thereof pooled from eluate of step (f) to anion exchange chromatography in flowthrough mode; and
   (h) subjecting said anti-IL-4Rα antibody or antigen-binding fragment thereof pooled from flowthrough fractions of step (g) to virus retentive filtration to produce an anti-IL-4Rα antibody or antigen-binding fragment thereof.
325. The method of example 324, further comprising subjecting said anti-IL-4Rα antibody or antigen-binding fragment thereof to ultrafiltration and diafiltration (UF/DF) after step (h).
326. The method of example 324 or 325, wherein said affinity chromatography is Protein A chromatography.
327. The method of example 326, wherein a Protein A resin is selected from the group consisting of Mab Select PrismA, Mab Select SuRe, Mab Select SuRe LX, MabSelect, Mab Select SuRe pcc, Mab Select Xtra, rProtein A Sepharose, ProSep HC, ProSep Ultra, ProSep Ultra Plus, MabCapture, and AmsphereA3.
328. The method of any one of examples 324-327, wherein an anion exchange resin is selected from the group consisting of Q Sepharose Fast Flow, Poros 50PI, Poros 50HQ, Capto Q Impres, Capto DEAE, Toyopearl QAE-550, Toyopearl DEAE-650, Toyopearl GigaCap Q-650, Fractogel EMD TMAE Hicap, Sartobind STIC PA nano, Sartobind Q nano, CUNO BioCap, and XOHC.
329. The method of any one of examples 324-328, wherein a cation exchange resin is selected from the group consisting of Fractogel Hicap, Capto SP ImpRes, Capto S ImpAc, CM Hyper D grade F, Eshmuno S, Nuvia C Prime, Nuvia S, Poros HS, and Poros XS.
330. The method of example 324, further comprising passing said anti-IL-4Rα antibody or antigen-binding fragment thereof through a LifeAssure filter after the viral inactivation of step (e) and prior to the cation exchange chromatography of step (f).
331. The method of any one of examples 325-330, wherein said UF/DF step comprises a membrane filter device selected from the group consisting of Pellicon 2, Pellicon 3 cassettes with 10 kD, 30 kD or 50 kD membranes, Kvick 10 kD, 30 kD or 50 kD membrane cassettes, and Centramate and Centrasette 10 kD, 30 kD or 50 kD cassettes.
332. The method of any one of examples 325-331, wherein said UF/DF step does not include addition of arginine.
333. The method of any one of examples 324-332, wherein said anti-IL-4Rα antibody or antigen-binding fragment thereof comprises three heavy chain complementarity determining region (HCDR) sequences comprising SEQ ID NOs: 3, 4, and 5, and three light chain complementarity determining region (LCDR) sequences comprising SEQ ID NOs: 6, 7, and 8.
334. The method of any one of examples 324-333, wherein said anti-IL-4Rα antibody or antigen-binding fragment thereof comprises a heavy chain variable region (HCVR) comprising the amino acid sequence of SEQ ID NO: 1 and a light chain variable region (LCVR) comprising the amino acid sequence of SEQ ID NO: 2.
335. The method of any one of examples 324-334, wherein said anti-IL-4Rα antibody is Dupilumab.
336. A method for producing an anti-IL4Rα antibody or antigen-binding fragment thereof, comprising:
   (a) subjecting a harvested antibody to affinity chromatography;
   (b) subjecting said antibody pooled from eluate of step (a) to viral inactivation;
   (c) subjecting said antibody pooled from step (b) to cation exchange chromatography in bind and elute mode;
   (d) subjecting said antibody pooled from eluate of step (c) to anion exchange chromatography in flowthrough mode;
   (e) subjecting said antibody pooled from flowthrough fractions of step (d) to virus retentive filtration; and
   (f) subjecting said antibody pooled from step (e) to ultrafiltration and diafiltration (UF/DF) to produce an anti-IL4Rα antibody or antigen-binding fragment thereof, wherein said UF/DF step does not include addition of arginine.
   337. The method of example 336, wherein said UF/DF step includes a diafiltration buffer having a pH between 4.0 and 4.5.
   338. The method of example 336 or 337, wherein said UF/DF step includes a diafiltration buffer comprising between about 4 mM acetate and about 6 mM acetate.
   339. The method of any one of examples 14-17, 19-29, 264-283, 285-309, 313-323, 325-335, 336-338, 348-391, or 393-408, wherein a concentrated antibody pool following UF/DF has a pH of about 5.3.
   340. The method of any one of examples 14-17, 19-29, 264-283, 285-309, 313-323, 325-335, 336-339, 348-391, or 393-408, wherein said UF/DF step comprises a membrane filter device selected from the group consisting of Pellicon 2, Pellicon 3 cassettes with 10 kD, 30 kD or 50 kD membranes, Kvick 10 kD, 30 kD or 50 kD membrane cassettes, and Centramate and Centrasette 10 kD, 30 kD or 50 kD cassettes.
   341. A method for treating a patient having a type 2 inflammatory disease, comprising administering to a patient an anti-IL4Rα antibody produced according to the method of any one of examples 310-340.
   342. The method of example 341, wherein said type 2 inflammatory disease is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.
   343. A method for treating a disease or disorder associated with IL-4R activity, comprising administering to a patient an anti-IL4Rα antibody produced according to the method of any one of examples 310-342.
344. The method of example 343, wherein said disease or disorder associated with IL-4R activity is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.
345. A method comprising the steps of:
   a. subjecting a harvested antibody to affinity chromatography;
   b. subjecting said antibody pooled from eluate of step (a) to viral inactivation at a pH from about 3 to about 4 and then adjusting the pH to from about 5 to about 8;
   c. subjecting said antibody pooled from step (b) to mixed-mode chromatography;
   d. subjecting said antibody pooled from step (c) to anion exchange chromatography in flowthrough mode; and
   e. subjecting said antibody pooled from flowthrough fractions of step (d) to virus retentive filtration to produce an anti-IL4Rα antibody.
346. The method of example 345, further comprising a harvest pre-treatment step prior to step (a).
347. The method of example 346, wherein said harvest pre-treatment step includes adjusting said antibody to a transient pH level from about 4 to 5.5.
348. The method of any one of examples 345-347, further comprising subjecting said antibody to ultrafiltration and diafiltration (UF/DF) after step (e).
349. The method of example 348, wherein said UF/DF includes a diafiltration buffer having a pH between 4.0 and 4.5.
350. The method of example 348 or 349, wherein a concentrated antibody pool following UF/DF has a pH of about 5.3.
351. The method of any one of examples 348-350, wherein said UF/DF includes a diafiltration buffer comprising between about 4 mM acetate and about 6 mM acetate.
352. The method of any one of examples 1-30, 202-225, 234-344, 345-351, 392-408, or 509-532, wherein said affinity chromatography is Protein A chromatography.
353. The method of example 352, wherein a Protein A resin is selected from the group consisting of MabSelect PrismA, MabSelect SuRe, MabSelect SuRe LX, MabSelect, MabSelect SuRe pcc, MabSelect Xtra, rProtein A Sepharose, ProSep HC, ProSep Ultra, ProSep Ultra Plus, MabCapture, and Amsphere A3.
354. The method of example 352 or 353, wherein a Protein A resin is selected that is capable of receiving a protein load at a concentration above 55 g/L-resin.
355. The method of any one of examples 352-354, wherein a Protein A column load pH is between 6 and 8.
356. The method of any one of examples 352-355, wherein a Protein A wash buffer is selected for removing host cell proteins bound to said anti-IL-4Rα antibody.
357. The method of any one of examples 352-356, wherein a Protein A wash buffer is selected for removing host cell proteins interacting with said anti-IL-4Rα antibody.
358. The method of any one of examples 352-357, wherein a Protein A wash buffer is selected for removing high molecular weight species.
359. The method of any one of examples 352-358, wherein said Protein A wash buffer has a pH between about 5 and about 9.
360. The method of any one of examples 352-359, wherein said Protein A wash buffer has a pH corresponding to the pI of an HCP of concern.
361. The method of any one of examples 352-360, wherein said Protein A wash buffer comprises arginine, potassium sorbate, sodium benzoate, guanidine, Tris, isopropanol, urea, sodium carbonate, or a combination thereof.
362. The method of any one of examples 352-361, wherein said Protein A wash buffer comprises from about 100 mM to about 450 mM arginine, optionally wherein said Protein A wash buffer comprises about 450 mM arginine.
363. The method of example 362, wherein said Protein A wash buffer further comprises about 20 mM Tris and/or has a pH of about 6.0.
364. The method of example 362, wherein said Protein A wash buffer further comprises about 30 mM Tris and/or has a pH of about 8.0.
365. The method of any one of examples 352-361, wherein said Protein A wash buffer comprises from about 100 mM to about 1.2 M potassium sorbate, optionally wherein said Protein A wash buffer comprises about 1 M potassium sorbate.
366. The method of any one of examples 352-365, wherein said Protein A wash buffer has a pH of about 7.2.
367. The method of any one of examples 352-361, wherein said Protein A wash buffer comprises from about 0.5 M to about 1.0 M sodium benzoate, optionally wherein said Protein A wash buffer comprises about 0.5 M sodium benzoate.
368. The method of any one of examples 352-367, wherein said Protein A wash buffer has a pH of about 6.0.
369. The method of any one of examples 352-361, wherein said Protein A wash buffer comprises from about 0.5 M to about 1.0 M guanidine, optionally wherein said Protein A wash buffer comprises about 0.5 M guanidine.
370. The method of example 369, wherein said Protein A wash buffer comprises from about 0.05 M to about 0.5 M NaCl, optionally wherein said Protein A wash buffer comprises about 0.5 M NaCl.
371. The method of any one of examples 352-370, wherein said Protein A wash buffer has a pH of about 8.0.
372. The method of any one of examples 352-361, wherein said Protein A wash buffer comprises from about 1% to about 20% isopropanol, optionally wherein said Protein A wash buffer comprises about 10% isopropanol.
373. The method of example 372, wherein said Protein A wash buffer comprises from about 0.5 M to about 0.6 M urea, optionally wherein said Protein A wash buffer comprises about 0.5 M urea.
374. The method of any one of examples 352-373, wherein said Protein A wash buffer comprises Tris, optionally wherein said Protein A wash buffer comprises about 25 mM Tris.
375. The method of any one of examples 352-374, wherein said Protein A wash buffer has a pH of about 9.0.
376. The method of any one of examples 352-361, wherein said Protein A wash buffer comprises from about 10 mM to about 500 mM sodium carbonate, optionally wherein said Protein A wash buffer comprises about 100 mM sodium carbonate.
377. The method of any one of examples 352-376, wherein said Protein A wash buffer has a pH of about 10.0.
378. The method of any one of examples 236-240, 345-377, or 392-408, wherein a mixed-mode chromatography resin is selected from the group consisting of Capto Adhere, Capto Adhere ImpRes, Capto MMC, PPA HyperCel, HEA HyperCel, MEP HyperCel, MBI HyperCel, CMM HyperCel, Capto Core 700, Nuvia C Prime, Toyo Pearl MX Trp 650M, and Eshmuno HCX.
379. The method of any one of examples 236-240, 345-378, or 392-408, wherein said mixed-mode chromatography is operated in flowthrough mode or bind and elute mode.
380. The method of any one of examples 236-240, 345-379, or 392-408, wherein an equilibration step, washing step, strip 1 step, and/or strip 2 step for mixed-mode chromatography has an incubation time from about 2 to about 10 minutes, optionally about 6 minutes.
381. The method of any one of examples 236-240, 345-380, or 392-408, wherein an equilibration buffer for mixed-mode chromatography comprises about 100 mM NaCl at a pH of about 5.
382. The method of any one of examples 236-240, 345-381, or 392-408, wherein quantity of protein loaded on a mixed-mode chromatography resin is between about 10 g/L-resin and about 80 g/L-resin, between about 50 g/L-resin and about 200 g/L-resin, between about 100 g/L-resin and about 150 g/L-resin, between about 100 g/L-resin and about 110 g/L-resin, less than about 120 g/L-resin, about 10 g/L-resin, about 15 g/L-resin, about 20 g/L-resin, about 25 g/L-resin, about 30 g/L-resin, about 35 g/L-resin, about 40 g/L-resin, about 45 g/L-resin, about 50 g/L-resin, about 55 g/L-resin, about 60 g/L-resin, about 65 g/L-resin, about 70 g/L-resin, about 75 g/L-resin, about 80 g/L-resin, about 85 g/L-resin, about 90 g/L-resin, about 95 g/L-resin, about 100 g/L-resin, about 105 g/L-resin, about 110 g/L-resin, about 115 g/L-resin, about 120 g/L-resin, about 125 g/L-resin, about 130 g/L-resin, about 135 g/L-resin, about 140 g/L-resin, about 145 g/L-resin, about 150 g/L-resin, about 155 g/L-resin, about 160 g/L-resin, about 165 g/L-resin, about 170 g/L-resin, about 175 g/L-resin, about 180 g/L-resin, about 185 g/L-resin, about 190 g/L-resin, about 195 g/L-resin, or about 200 g/L-resin.
383. The method of any one of examples 236-240, 345-382, or 392-408, wherein an equilibration buffer and/or a wash buffer for mixed-mode chromatography has a pH between about 4.50 and about 9.00, between about 4.50 and about 8.00, between about 4.50 and about 5.50, between about 5.00 and about 6.00, about 4.50, about 4.60, about 4.70, about 4.80, about 4.90, about 5.00, about 5.10, about 5.20, about 5.30, about 5.40, about 5.50, about 5.60, about 5.70, about 5.80, about 5.90, about 6.00, about 6.10, about 6.20, about 6.30, about 6.40, about 6.50, about 6.60, about 6.70, about 6.80, about 6.90, about 7.00, about 7.10, about 7.20, about 7.30, about 7.40, about 7.50, about 7.60, about 7.70, about 7.80, about 7.90, about 8.00, about 8.10, about 8.20, about 8.30, about 8.40, about 8.50, about 8.60, about 8.70, about 8.80, about 8.90, or about 9.00.
384. The method of any one of examples 236-240, 345-383, or 392-408, wherein an equilibration buffer and/or a wash buffer for mixed-mode chromatography comprises NaCl at between about 0 mM and about 100 mM, between about 100 mM and about 500 mM, between about 100 mM and about 250 mM, between about 100 mM and about 150 mM, between about 80 mM and about 120 mM, between about 95 mM and about 105 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 175 mM, about 200 mM, about 225 mM, about 250 mM, about 275 mM, about 300 mM, about 325 mM, about 350 mM, about 375 mM, about 400 mM, about 425 mM, about 450 mM, about 475 mM, or about 500 mM.
385. The method of any one of examples 236-240, 345-384, or 392-408, wherein an equilibration buffer, a wash buffer, and/or an elution buffer for mixed-mode chromatography comprises arginine or citrate.
386. The method of any one of examples 236-240, 345-385, or 392-408, wherein an elution buffer for mixed-mode chromatography comprises a pH between about 4.50 and about 9.00, between about 4.50 and about 8.00, between about 4.50 and about 5.50, between about 5.00 and about 6.00, about 4.50, about 4.60, about 4.70, about 4.80, about 4.90, about 5.00, about 5.10, about 5.20, about 5.30, about 5.40, about 5.50, about 5.60, about 5.70, about 5.80, about 5.90, about 6.00, about 6.10, about 6.20, about 6.30, about 6.40, about 6.50, about 6.60, about 6.70, about 6.80, about 6.90, about 7.00, about 7.10, about 7.20, about 7.30, about 7.40, about 7.50, about 7.60, about 7.70, about 7.80, about 7.90, about 8.00, about 8.10, about 8.20, about 8.30, about 8.40, about 8.50, about 8.60, about 8.70, about 8.80, about 8.90, or about 9.00.
387. The method of any one of examples 236-240, 345-386, or 392-408, wherein an elution buffer for mixed-mode chromatography comprises NaCl at between about 0 mM and about 500 mM, between about 100 mM and about 250 mM, between about 100 mM and about 150 mM, about 0 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 175 mM, about 200 mM, about 225 mM, about 250 mM, about 275 mM, about 300 mM, about 325 mM, about 350 mM, about 375 mM, about 400 mM, about 425 mM, about 450 mM, about 475 mM, or about 500 mM.
388. The method of any one of examples 236-240, 345-387, or 392-408, wherein said mixed-mode chromatography is operated using a plate-based format or a robocolumn-based format.
389. The method of any one of examples 345-388, wherein said anti-IL-4Rα antibody comprises three heavy chain complementarity determining region (HCDR) sequences comprising SEQ ID NOs: 3, 4, and 5, and three light chain complementarity determining region (LCDR) sequences comprising SEQ ID NOs: 6, 7, and 8.
390. The method of any one of examples 345-389, wherein said anti-IL-4Rα antibody comprises a heavy chain variable region (HCVR) comprising the amino acid sequence of SEQ ID NO: 1 and a light chain variable region (LCVR) comprising the amino acid sequence of SEQ ID NO: 2.
391. The method of any one of examples 345-390, wherein said anti-IL-4Rα antibody is Dupilumab.
392. A method comprising the steps of:
   (a) culturing cells expressing an anti-IL-4Rα antibody or antigen-binding fragment thereof;
   (b) subjecting said cells to transient pH levels from about 4 to 5.5, then raising pH levels to from about 5.5 to 6.5;
   (c) harvesting said cells by centrifugation to separate cell debris from clarified media comprising said anti-IL-4Rα antibody or antigen-binding fragment thereof;
   (d) subjecting said clarified media to affinity chromatography;
   (e) subjecting said anti-IL-4Rα antibody or antigen-binding fragment thereof pooled from eluate of step (d) to viral inactivation at a pH from about 3 to about 4 and then adjusting the pH to from about 5 to about 8;
   (f) subjecting said anti-IL-4Rα antibody or antigen-binding fragment thereof pooled from step (e) to mixed-mode chromatography in flowthrough mode;
   (g) subjecting said anti-IL-4Rα antibody or antigen-binding fragment thereof pooled from flowthrough fractions of step (f) to anion exchange chromatography in flowthrough mode; and
   (h) subjecting said anti-IL-4Rα antibody or antigen-binding fragment thereof pooled from flowthrough fractions of step (g) to virus retentive filtration to produce an anti-IL-4Rα antibody or antigen-binding fragment thereof.
393. The method of example 392, further comprising subjecting said anti-IL-4Rα antibody or antigen-binding fragment thereof to ultrafiltration and diafiltration (UF/DF) after step (h).
394. The method of example 392 or 393, wherein said affinity chromatography is Protein A chromatography.
395. The method of example 394, wherein a Protein A resin is selected from the group consisting of Mab Select PrismA, Mab Select SuRe, Mab Select SuRe LX, MabSelect, Mab Select SuRe pcc, Mab Select Xtra, rProtein A Sepharose, ProSep HC, ProSep Ultra, ProSep Ultra Plus, MabCapture, and AmsphereA3.
396. The method of example 394 or 395, wherein a Protein A wash buffer is selected for removing host cell proteins bound to said anti-IL-4Rα antibody or antigen-binding fragment thereof.
397. The method of any one of examples 236-240 or 392-396, wherein a mixed-mode chromatography resin is selected from the group consisting of Capto Adhere, Capto Adhere ImpRes, Capto MMC, PPA HyperCel, HEA HyperCel, MEP HyperCel, MBI HyperCel, CMM HyperCel, Capto Core 700, Nuvia C Prime, Toyo Pearl MX Trp 650M, and Eshmuno HCX.
398. The method of any one of examples 1-30, 202-225, 234-391, 392-397, or 509-532, wherein an anion exchange resin is selected from the group consisting of Q Sepharose Fast Flow, Poros 50PI, Poros 50HQ, Capto Q Impres, Capto DEAE, Toyopearl QAE-550, Toyopearl DEAE-650, Toyopearl GigaCap Q-650, Fractogel EMD TMAE Hicap, Sartobind STIC PA nano, Sartobind Q nano, CUNO BioCap, and XOHC.
399. The method of any one of examples 392-398, further comprising passing said anti-IL-4Rα antibody or antigen-binding fragment thereof through a LifeAssure filter after the viral inactivation of step (e) and prior to the mixed-mode chromatography of step (f).
400. The method of any one of examples 393-399, wherein said UF/DF step comprises a membrane filter device selected from the group consisting of Pellicon 2, Pellicon 3 cassettes with 10 kD, 30 kD or 50 kD membranes, Kvick 10 kD, 30 kD or 50 kD membrane cassettes, and Centramate and Centrasette 10 kD, 30 kD or 50 kD cassettes.
401. The method of any one of examples 393-400, wherein said UF/DF step does not include addition of arginine.
402. The method of any one of examples 392-401, wherein said anti-IL-4Rα antibody or antigen-binding fragment thereof comprises three heavy chain complementarity determining region (HCDR) sequences comprising SEQ ID NOs: 3, 4, and 5, and three light chain complementarity determining region (LCDR) sequences comprising SEQ ID NOs: 6, 7, and 8.
403. The method of any one of examples 392-402, wherein said anti-IL-4Rα antibody or antigen-binding fragment thereof comprises a heavy chain variable region (HCVR) comprising the amino acid sequence of SEQ ID NO: 1 and a light chain variable region (LCVR) comprising the amino acid sequence of SEQ ID NO: 2.
404. The method of any one of examples 392-403, wherein said anti-IL-4Rα antibody is Dupilumab.
405. A method for treating a patient having a type 2 inflammatory disease, comprising administering to a patient an anti-IL4Rα antibody produced according to the method of any one of examples 392-404.
406. The method of example 405, wherein said type 2 inflammatory disease is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.
407. A method for treating a disease or disorder associated with IL-4R activity, comprising administering to a patient an anti-IL4Rα antibody produced according to the method of any one of examples 392-406.
408. The method of example 407, wherein said disease or disorder associated with IL-4R activity is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.
409. A method of producing Dupilumab, comprising culturing cells with an initial viable cell density (VCD) in a seed train adjusted to be at least 2.5×10⁵ cells/mL.
410. A method of producing Dupilumab, comprising culturing cells with an initial VCD in a seed train adjusted to be at least 3.0×10⁵ cells/mL.

411. A method of producing Dupilumab, comprising culturing cells with an initial VCD in a seed train adjusted to be at least 3.5×10⁵ cells/mL.
412. The method of any of examples 409-411, wherein said seed train includes cell cultures in N-5 to N-1 vessels or bioreactors, wherein the initial VCD is adjusted between 3.5×10⁵ to 5.43×10⁵ cells/mL in each vessel or bioreactor.
413. The method of any one of examples 409-412, wherein a final Dupilumab titer is about 2%, 3%, 4%, 5%, 6%, 7%, 8%, 9%, 10%, 11%, 12%, 13%, 14%, 15%, 1%, 17%, 18%, 19%, or 20% greater than a titer produced from a cell culture where the initial VCD in said N-5 to N-1 vessel or bioreactor is below 2.5×10⁵ cells/mL.
414. The method of any one of examples 409-413, wherein the initial VCD is about 1.3×, 1.4×, 1.5×, 1.6, 1.7×, 1.8×, 1.9×, 2.0×, 2.1×, 2.2×, 2.3×, 2.4×, 2.5×, 2.6×, 2.7×, 2.8×, 2.9×, or 3.0× greater than an alternative initial VCD in a standard seed train.
415. The method of example 413 or 414, wherein an increased final Dupilumab titer is not dependent on the final VCD in the N-1 seed train vessels or bioreactors.
416. The method of any one of examples 413-415, wherein an increased final Dupilumab titer is not dependent on the initial VCD in a production vessel or bioreactor.
417. The method of any one of examples 409-416, wherein there is no substantial difference in peak lactate observed in a final production vessel compared to a final production vessel where the initial VCD vessels or bioreactors of the seed train is below 2.5×10⁵ cells/mL.
418. The method of example 417, wherein the seed train resulted in a 2%, 3%, 4%, 5%, 6%, 7%, 8%, 9%, 10%, 11%, 12%, 13%, 14%, 15%, 1%, 17%, 18%, 19%, 20% increase in final titer (g/L).
419. The method of example 418, wherein the initial VCD in a seed train is adjusted between 3.5×10⁵ to 5.43×10⁵ cells/mL.
420. A method of culturing a cell, the method comprising:
   a) using at least one on-line capacitance probe to measure a first capacitance value of a first cell culture;
   b) using at least one off-line assay to measure a first viable cell density value of the said first cell culture;
   c) correlating said first capacitance value with the said first viable cell density value to determine a correlation equation;
   d) using an on-line capacitance probe to determine a second capacitance value of a second cell culture;
   e) using said second capacitance value and said correlation equation to predict at least one second viable cell density value of said second cell culture; and
   f) adjusting a working volume or viable cell density (VCD) based on said second viable cell density value to culture the cell.
421. The method of example 420, wherein said cell is from a cell line that is the same as a cell line used to derive said correlation equation.
422. The method of example 420 or 421, wherein said correlation equation is generated using multivariate data analysis or a linear regression.
423. A method of producing Dupilumab comprising the steps of:
   (a) culturing cells wherein an initial viable cell density (VCD) in a seed train in vessels or bioreactors is adjusted to at least 2.5×10⁵ cells/mL;
   (b) measuring viable cell density by
   (i) applying an electric field to said cells cultured in a vessel or bioreactor; and (ii) measuring capacitance; and
   (iii) correlating capacitance to viable cell density;
   (c) adjusting initial VCD in each seed train vessel or bioreactor; and
   (d) producing Dupilumab.
424. The method of example 423, wherein the initial VCD in each vessel or bioreactor of an N-5 to N-1 seed train is adjusted between 3.5×10⁵ to 5.43×10⁵ cells/mL.
425. The method of example 423 or 424, wherein a final Dupilumab titer is about 2%, 3%, 4%, 5%, 6%, 7%, 8%, 9%, 10%, 11%, 12%, 13%, 14%, 15%, 1%, 17%, 18%, 19%, or 20% greater than a titer produced from a cell culture where the initial VCD in said seed train is below 2.5×10⁵ cells/mL.
426. The method of any one of examples 423-425, wherein the initial VCD is about 1.3×, 1.4×, 1.5×, 1.6, 1.7×, 1.8×, 1.9×, 2.0×, 2.1×, 2.2×, 2.3×, 2.4×, 2.5×, 2.6×, 2.7×, 2.8×, 2.9×, or 3.0× greater than an alternative initial VCD in a standard seed train.
427. The method of example 425 or 426, wherein an increased final Dupilumab titer is not dependent on a final VCD in N-5 to N-1 vessels or bioreactors.
428. The method of any one of examples 424-427, wherein there is no substantial difference in peak lactate observed in a final production vessel compared to a final production vessel or bioreactor where the initial VCD in each N-5 to N-1 vessel or bioreactor is below 2.5×10⁵ cells/mL.
429. The method of example 428, wherein the seed train resulted in a 2%, 3%, 4%, 5%, 6%, 7%, 8%, 9%, 10%, 11%, 12%, 13%, 14%, 15%, 1%, 17%, 18%, 19%, 20% increase in final titer (g/L).
430. A method of producing an anti-IL-4Rα antibody or antigen-binding fragment thereof, the method comprising:
   a) culturing cells capable of expressing an anti-IL-4Rα antibody or antigen-binding fragment thereof in a vessel or bioreactor;
   b) adjusting an initial viable cell density (VCD) to be 2.5×10⁵ cells/mL or greater;
   c) using at least one on-line sensor to measure a first property of a first cell culture;
   d) using at least one off-line assay to measure a second property of the first cell culture;
   e) correlating the measure of the first property of the first cell culture with the measure of the second property of the first cell culture to determine a correlation equation;
   f) using an on-line sensor to measure the first property of a second cell culture;
   g) using the measure of the first property of the second cell culture and the correlation equation to predict at least one measure of the second property of the second cell culture;
   h) transferring said cells to another vessel or bioreactor based on the at least one predicted measure of the second property of the second cell culture; and
   i) repeating steps b)-h) along a seed train.
431. The method of example 430, wherein said cells are from a cell culture that is the same as the first cell culture.
432. The method of example 430 wherein said cells are from a cell culture that is different from the first cell culture.
433. The method of any one of examples 430-432, wherein said correlation equation is derived using more than one cell line.
434. The method of any one of examples 430-433, wherein more than one measure of said first property of said first cell culture is taken.
435. The method of any one of examples 430-434, wherein more than one measure of said second property of said first cell culture is taken.
436. The method of any one of examples 430-435, wherein at least about 50 percent of variability in said measure of said second property of said first cell culture is due to variance in said measure of said first property of said first cell culture.
437. The method of any one of examples 430-436, wherein said correlation equation is generated using multivariate data analysis or a linear regression.
438. The method of any one of examples 188-199, 420, or 430-436, wherein said correlation equation is generated for a production vessel or bioreactor using multivariate data analysis.
439. The method of any one of examples 188-199, 420, or 430-436, wherein said correlation equation is generated for a seed train vessel or bioreactor using a linear regression.
440. The method of any one of examples 430-439, wherein said first property is a capacitance.
441. The method of any one of examples 430-440, wherein said second property is a VCD.
442. The method of any one of examples 430-441, wherein the initial VCD is about 1.3×, 1.4×, 1.5×, 1.6, 1.7×, 1.8×, 1.9×, 2.0×, 2.1×, 2.2×, 2.3×, 2.4×, 2.5×, 2.6×, 2.7×, 2.8×, 2.9×, or 3.0× greater than an alternative initial VCD in a standard seed train.
443. The method of any one of examples 430-442, wherein there is no substantial difference in peak lactate observed in the final production vessel or bioreactor compared to a final production vessel where the initial VCD in N-5 to N-1 vessels is below 2.5×10⁵ cells/mL.
444. The method of example 443, wherein lactate consumption is increased in a 3000 L vessel or bioreactor.
445. The method of any one of examples 430-444, wherein the seed train resulted in a 2%, 3%, 4%, 5%, 6%, 7%, 8%, 9%, 10%, 11%, 12%, 13%, 14%, 15%, 16%, 17%, 18%, 19%, 20% increase in final titer (g/L).
446. The method of any one of examples 430-445, wherein the initial VCD is about 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, or 3.0× greater than an alternative initial VCD in a standard seed train.
447. The method of any one of examples 430-446, wherein said cells are CHO cells.
448. The method of any one of examples 430-447, wherein said anti-IL-4Rα antibody is Dupilumab.
449. A method for treating a patient having a type 2 inflammatory disease, comprising administering to a patient an anti-IL4Rα antibody, an antigen-binding fragment thereof, or Dupilumab produced according to the method of any of examples 409-419 or 423-448.
450. The method of example 449, wherein said type 2 inflammatory disease is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.
451. A method for treating a disease or disorder associated with IL-4R activity, comprising administering to a patient an anti-IL4Rα antibody, an antigen-binding fragment thereof, or Dupilumab produced according to the method of any of examples 409-419 or 423-448.
452. The method of example 451, wherein said disease or disorder associated with IL-4R activity is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.
453. A system for producing Dupilumab, comprising:
   (d) a bioreactor for culturing cells capable of expressing Dupilumab;
   (e) one or more agitating elements, wherein said one or more agitating elements are configured below a working volume of said bioreactor; and
   (f) one or more gas control assemblies coupled to said bioreactor for controlling dissolved gases.
454. The system of example 453, wherein said bioreactor volume is greater than or equal to 500 L.
455. The system of example 453 and 454, wherein said bioreactor volume is greater than or equal to 3,000 L.
456. The system of any one of examples 453-455, wherein said bioreactor volume is greater than or equal to 10,000 L.
457. The system of any one of examples 453-456, wherein said one or more agitating elements comprise one or more impeller assemblies.
458. The system of any one of examples 453-457, wherein said one or more agitating elements are configured to have an first agitation rate between 20 rpm and 150 rpm.
459. The system of any one of examples 453-458, wherein said one or more agitating elements are operated at a power per unit volume from about 0.017 to about 0.076 hp/1000 L.
460. The system of any one of examples 453-459, wherein said one or more agitating elements are configured to have a second agitation rate that may increase by 25%, 50%, 75%, 100%, 125%, 150%, 175% or 200% on one or more days selected from the group of day 0.5, 1, 1.5, 2, 2.5, 3, 3.5, 4, 4.5, 5, 5.5, 6, 6.5, 7, 7.5, 8, 8.5, 9, 9.5, 10, 10.5, and 11.
461. The system of any one of examples 453-460, wherein said one or more gas control assemblies comprise one or more spargers.
462. The system of example 461, wherein said one or more spargers are configured with an initial sparging rate of about 25-75 slpm.
463. The system of example 461, wherein said one or more spargers are configured with a sparging rate of about 25 to about 150 slpm.
464. The system of any one of examples 461-463, wherein said one or more spargers are configured with a sparging rate of about 0.0025 to 0.0075 vessel volumes per minutes (vvm).
465. The system of any one of examples 461-464, wherein said one or more spargers are configured with a sparging rate that may increase by 25%, 50%, 75%, 100%, 125%, 150%, 175%, 200%, 225%, 250%, 275%, 300%, 325%, 350%, 375%, 400%, 425%, 450%, 475%, or 500% on one or more days selected from the group of day 0.5, 1, 1.5, 2, 2.5, 3, 3.5, 4, 4.5, 5, 5.5, 6, 6.5, 7, 7.5, 8, 8.5, 9, 9.5, 10, 10.5, and 11.
466. The system of any one of examples 461-465, wherein said one or more spargers are configured to automatically adjust the sparging rate based on dissolved oxygen levels.
467. The system of any one of examples 461-466, wherein said one or more spargers include between 146 and 292 holes sized between 0.5 mm and 2 mm.
468. A method for enhancing cell growth, cell viability, cell density, or production of Dupilumab in a mammalian cell culture process, comprising the steps of:
   (a) varying agitation rates at different points during the growth and production phases;
   (b) varying sparging rates at different points during the growth and production phases; and
   (c) varying dextrose target levels at different points during the growth and production phases.
469. The method of example 468, wherein an initial agitation rate is set between 20 rpm and 150 rpm.
470. The method of example 468 or 469, wherein said agitation rate is configured to increase by 25%, 50%, 75%, 100%, 125%, 150%, 175% or 200% on one or more days selected from the group of day 0.5, 1, 1.5, 2, 2.5, 3, 3.5, 4, 4.5, 5, 5.5, 6, 6.5, 7, 7.5, 8, 8.5, 9, 9.5, 10, 10.5, and 11.
471. The method of any one of examples 468-470, wherein more than one sparger is used to vary the sparging rate.
472. The method of example 471, wherein said spargers are set to an initial sparging rate of about 25-75 slpm.
473. The method of any one of examples 468-472, wherein said sparging rate is increased by 25%, 50%, 75%, 100%, 125%, 150%, 175%, 200%, 225%, 250%, 275%, 300%, 325%, 350%, 375%, 400%, 425%, 450%, 475%, or 500% on one or more days selected from the group of day 0.5, 1, 1.5, 2, 2.5, 3, 3.5, 4, 4.5, 5, 5.5, 6, 6.5, 7, 7.5, 8, 8.5, 9, 9.5, 10, 10.5, and 11.
474. The method of any one of examples 468-473, wherein said sparging rate is automatically adjusted to maintain desired dissolved oxygen and pCO2 levels.
475. The method of any one of examples 471-474, wherein said spargers comprise between 146 and 292 holes sized between 0.5 mm and 2 mm.
476. The method of any one of examples 468-475, wherein an initial dextrose target level is set between 5 g/L and 7 g/L.
477. The method of any one of examples 49-148, 158-168, 188-234, 409-452, 468-476, 480-495, or 509-536, wherein a dextrose target level is set to vary between 5 g/L and 7 g/L on day 0 and then stepped-up to vary between 7 g/L and 9 g/L on day 2 and then stepped-up to vary between 9 g/L and 11 g/L on day 4.
478. The method of any one of examples 49-148, 158-168, 188-234, 409-452, 468-476, 480-495, or 509-536, wherein a dextrose target level is set to vary between 5 g/L and 7 g/L on day 0 and then stepped-up to vary between 7 g/L and 11 g/L on day 2 and then decreased to vary between 5 g/L and 7 g/L on day 4.
479, The method of any one of examples 49-148, 158-168, 188-234, 409-452, 468-476, 480-495, or 509-536, wherein a glucose target level is from about 5 g/L to about 6 from inoculation to about day 2, from about 7 to about 8 g/L, for about day 3, and from about 5 g/L to about 7 g/L from about day 4 to harvest.
480. A method of producing an anti-IL-4Rα antibody or antigen-binding fragment thereof, comprising the steps of:
   (a) culturing cells expressing an anti-IL-4Rα antibody or antigen-binding fragment thereof in a cell culture medium wherein a cumulative concentration of one or more polyamines in said cell culture medium is between about 0.03 and about 0.9 mM;
   (b) agitating said cell culture; and
   (c) controlling dissolved gas concentrations in said cell culture.
481. The method of any one of examples 49-148, 158-168, 188-234, 409-452, 468-479, 480, 486-495, or 509-536, wherein said cell culture medium is subjected to High Temperature Short Time (HTST) treatment at about 101° C. to 106° C. for 8 to 15 seconds.
482. The method of example 480 or 481, wherein two or more impeller assemblies are positioned below a surface of an initial working volume.
433. The method of any one of examples 49-148, 158-168, 188-234, 409-452, 468-479, 480-482, 486-495, or 509-536, wherein said cell culture medium is subjected to High Temperature Short Time (HTST) treatment at about 101° C. to 103° C. for 8 to 12 seconds.
484. The method of any one of examples 49-148, 158-168, 188-234, 409-452, 468-479, 480-482, 486-495, or 509-536, wherein said cell culture medium is subjected to High Temperature Short Time (HTST) treatment at about 102° C. for about 10 seconds.

485. The method of any one of examples 480-484, wherein agitation of said cell culture is performed using one or more impeller assemblies and an uppermost impeller is positioned below a surface of an initial working volume.
486 The method of any one of examples 480-485, wherein an initial agitation rate is configured between 0.017 hp/1000 L and 0.076 hp/1000 L in a bioreactor that is 10,000 L or greater.
487. The method of any one of examples 480-486, wherein said agitation rate is configured to increase by 25%, 50%, 75%, 100%, 125%, 150%, 175% or 200% on one or more days selected from the group of day 0.5, 1, 1.5, 2, 2.5, 3, 3.5, 4, 4.5, 5, 5.5, 6, 6.5, 7, 7.5, 8, 8.5, 9, 9.5, 10, 10.5, and 11.
488. The method of any one of examples 480-487, wherein an agitation rate is from about 28 to about 40 rpm or from about 22 to about 40 rpm.
489. The method of any one of examples 480-488, wherein an agitation rate in a bioreactor is about 22 rpm from inoculation to about day 1.5 or when dissolved oxygen reaches a setpoint, about 28 rpm from about day 1.5 or when dissolved oxygen reaches a setpoint to about day 4.5, about 34 rpm from about day 4.5 to about day 5.5, and about 40 rpm from about day 5.5 to harvest.
490. The method of any one of examples 480-489, wherein said dissolved gas concentrations are controlled by one or more spargers.
491. The method of example 490, wherein said one or more spargers are configured at an initial sparging rate of about 25-75 slpm.
492. The method of any one of examples 490-491, wherein said sparging rate is configured to increase by 25%, 50%, 75%, 100%, 125%, 150%, 175%, 200%, 225%, 250%, 275%, 300%, 325%, 350%, 375%, 400%, 425%, 450%, 475%, or 500% on one or more days selected from the group of day 0.5, 1, 1.5, 2, 2.5, 3, 3.5, 4, 4.5, 5, 5.5, 6, 6.5, 7, 7.5, 8, 8.5, 9, 9.5, 10, 10.5, and 11.
493. The method of any one of examples 490-492, wherein a sparging rate is automatically configured based on dissolved oxygen levels.
494. The method of any one of examples 490-493, wherein a sparging rate is from about 400 to about 500 slpm.
495. The method of any one of examples 490-493, wherein a sparging rate increases from about 25 to about 300 slpm.
496. A bioreactor, comprising:
   one or more optical probes in a reservoir of said bioreactor for generating a data signal with reduced signal noise compared to an electrochemical probe.
497. A bioreactor of example 496, comprising two or more optical probes.
498. A bioreactor of example 496, with two or more optical probes configured in the lower one third of the reservoir of the bioreactor.
499. A bioreactor of example 496 or 498, with two or more optical probes configured at two different locations along a probe belt.
500. A bioreactor of any one of examples 496-499, further comprising an agitating element comprising one or more impeller assemblies, wherein an uppermost impeller is positioned below a surface of the initial working volume.
501. A method for treating a patient having a type 2 inflammatory disease, comprising administering to a patient Dupilumab produced according to the system of any one of examples 149-157 or 453-467.
502. The method of example 501, wherein said type 2 inflammatory disease is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.
503. A method for treating a disease or disorder associated with IL-4R activity, comprising administering to a patient Dupilumab produced according to the system of any one of examples 149-157 or 453-467.
504. The method of example 503, wherein said disease or disorder associated with IL-4R activity is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.
505. A method for treating a patient having a type 2 inflammatory disease, comprising administering to a patient Dupilumab produced according to the method of any one of examples 1-30, 65-148, 158-168, 202-419, 423-452, 468-495, or 509-555.
506. The method of example 505, wherein said type 2 inflammatory disease is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.
507. A method for treating a disease or disorder associated with IL-4R activity, comprising administering to a patient Dupilumab produced according to the method of any one of examples 1-30, 65-148, 158-168, 202-419, 423-452, 468-495, or 509-555.
508. The method of example 507, wherein said disease or disorder associated with IL-4R activity is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.
509. A method for producing an anti-IL-4Rα antibody or antigen-binding fragment thereof, comprising:
   (a) culturing cells expressing anti-IL-4Rα antibody or antigen-binding fragment thereof using a cell culture medium comprising ornithine at between about 0.09 and about 0.9 mM and/or putrescine at between about 0.20 mM and about 0.9 mM,
   (b) harvesting said cells by centrifugation to separate cell debris from clarified media comprising the anti-IL-4Rα antibody or antigen binding fragment thereof;
   (c) subjecting said clarified media to affinity chromatography;
   (d) subjecting said antibody pooled from eluate of step (c) to viral inactivation at a pH from about 3 to about 4 and then adjusting the pH to from about 5 to about 8;
   (e) subjecting said anti-IL-4Rα antibody or antigen-binding fragment thereof pooled from step (d) to anion exchange chromatography in flowthrough mode;
   (f) subjecting said anti-IL-4Rα antibody or antigen-binding fragment thereof pooled from flowthrough fractions of step (e) to cation exchange chromatography in bind and elute mode;
   (g) subjecting said anti-IL-4Rα antibody or antigen-binding fragment thereof pooled from eluate of step (f) to hydrophobic interaction chromatography in flowthrough mode;
   (h) subjecting said anti-IL-4Rα antibody or antigen-binding fragment thereof pooled from flowthrough fractions of step (g) to virus retentive filtration to produce an anti-IL4Rα antibody or antigen-binding fragment thereof; and
   (i) collecting said anti-IL-4Rα antibody or antigen-binding fragment thereof.
510. The method of example 509, wherein said cell culture medium comprises one or more fatty acids.
511. The method of example 510, wherein said one or more fatty acids are selected from the group consisting of linoleic acid, linolenic acid, thioctic acid, oleic acid, palmitic acid, stearic acid, arachidic acid, arachidonic acid, lauric acid, behenic acid, decanoic acid, dodecanoic acid, hexanoic acid, lignoceric acid, myristic acid, octanoic acid, and combinations thereof
512. The method of any one of examples 509-511, wherein said culture medium comprises nucleosides selected from the group consisting of adenosine, guanosine, cytidine, uridine, thymidine, hypoxanthine, and combinations thereof
513. The method of any one of examples 509-512, wherein said culture medium comprises amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and combinations thereof
514. The method of any one of examples 49-148, 158-168, 188-234, 409-452, 468-495, or 509-513, further comprising the step of adding one or more point-of-use additions to the cell culture medium.
515. The method of example 514, wherein said point-of-use additions comprise one or more of NaHCO₃, Na₂HPO₄, taurine, glutamine, poloxamer 188, insulin, glucose, CuSO₄, ZnSO₄, FeCl₃, NiSO₄, Na₄ EDTA, and Na₃ citrate EDTA.
516. The method of any one of examples 509-515, wherein the culture medium is hydrolysate-free.
517. The method of any one of examples 49-148, 158-168, 188-234, 409-452, 468-495, or 509-516, wherein said cell culture medium comprises about 7.14 mM putrescine.
518. The method of any one of examples 514-517, wherein said point-of-use additions comprise one or more of angiopoietins, bone morphogenetic proteins (BMPs), brain-derived neurotrophic factor (BDNF), epidermal growth factor (EGF), erythropoietin (EPO), fibroblast growth factor (FGF), glial cell line-derived neurotrophic factor (GDNF), granulocyte colony-stimulating factor (G-CSF), granulocyte macrophage colony-stimulating factor (GM-CSF), growth differentiation factor-9 (GDF9), hepatocyte growth factor (HGF), hepatoma-derived growth factor (HDGF), insulin, insulin-like growth factor (IGF), migration-stimulating factor, myostatin (GDF-8), nerve growth factor (NGF), platelet-derived growth factor (PDGF), thrombopoietin (TPO), transforming growth factor α (TGF-α), transforming growth factor beta (TGF-β), tumor necrosis factor-α (TNF-α), vascular endothelial growth factor (VEGF), Wnt signaling pathway agonists, placental growth factor (PIGF), fetal Bovine somatotrophin (FBS), interleukin-1 (IL-1), IL-2, IL-3, IL-4, IL-5, IL-6, and IL-7.
519. A method of producing an anti-IL-4Rα antibody or antigen-binding fragment thereof, comprising the steps of:
   (a) culturing cells expressing the anti-IL-4Rα antibody or antigen-binding fragment thereof using a cell culture medium comprising ornithine at between about 0.09 and about 0.9 mM and/or putrescine at between about 0.20 mM and about 0.9 mM,
   (b) harvesting said cells by centrifugation to separate cell debris from clarified media comprising the anti-IL-4Rα antibody or antigen-binding fragment thereof;
   (c) subjecting said clarified media to affinity chromatography;
   (d) subjecting said anti-IL-4Rα antibody or antigen-binding fragment thereof pooled from eluate of step (c) to viral inactivation at a pH from about 3 to about 4 and then adjusting the pH to from about 5 to about 8;
   (e) subjecting said anti-IL-4Rα antibody or antigen-binding fragment thereof pooled from step (d) to cation exchange chromatography in bind and elute mode;
   (f) subjecting said anti-IL-4Rα antibody or antigen-binding fragment thereof pooled from eluate of step (e) to anion exchange chromatography in flowthrough mode; and
   (g) subjecting said anti-IL-4Rα antibody or antigen-binding fragment thereof pooled from flowthrough fractions of step (f) to virus retentive filtration to produce an anti-IL4Rα antibody or antigen-binding fragment thereof.
520. The method of any one of examples 49-148, 158-168, 188-234, 409-452, 468-495, 509-518, or 519, wherein said cell culture medium comprises one or more fatty acids selected from the group consisting of linoleic acid, linolenic acid, thioctic acid, oleic acid, palmitic acid, stearic acid, arachidic acid, arachidonic acid, lauric acid, behenic acid, decanoic acid, dodecanoic acid, hexanoic acid, lignoceric acid, myristic acid, octanoic acid, and combinations thereof.
521. The method of any one of examples 49-148, 158-168, 188-234, 409-452, 468-495, 509-518, 519 or 520, wherein said culture medium comprises nucleosides selected from the group consisting of adenosine, guanosine, cytidine, uridine, thymidine, hypoxanthine, and combinations thereof
522. The method of any one of examples 49-148, 158-168, 188-234, 409-452, 468-495, 509-518, or 519-521, wherein said culture medium comprises insulin.
523. The method of any one of examples 49-148, 158-168, 188-234, 409-452, 468-495, 509-518, or 519-522, wherein said culture medium comprises amino acids selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and combinations thereof.
524. The method of any one of examples 49-148, 158-168, 188-234, 409-452, 468-495, or 509-523, wherein said culture medium is supplemented with tyrosine, optionally wherein said supplementing is on day 3 of production.
525. The method of example 524, wherein a concentration of said tyrosine is between about 1.8 g/L and about 2.2 g/L.
526. The method of any one of examples 49-148, 158-168, 188-234, 409-452, 468-495, 509-518, or 519-525, further comprising the step of adding one or more point-of-use additions to the cell culture medium.
527. The method of example 526, wherein said point-of-use additions comprise one or more of NaHCO₃, Na₂HPO₄, taurine, glutamine, poloxamer 188, insulin, glucose, CuSO₄, ZnSO₄, FeCl₃, NiSO₄, Na₄ EDTA, and Na₃ citrate EDTA.
528. The method of any one of examples 49-148, 158-168, 188-234, 409-452, 468-495, or 509-527, wherein a concentration of sodium phosphate in said culture medium is about 267 mg/L.
529. The method of any one of examples 49-148, 158-168, 188-234, 409-452, 468-495, or 509-527, wherein said cell culture medium is supplemented with sodium phosphate, optionally wherein said supplementing is done on one or more days selected from the group consisting of day 0, 1, 2, 3, 4, 5, 6, 7, 8, 9, 10, 11, and 12.
530. The method of example 529, wherein said supplementing is done on day 2, day 4, day 6, and day 8.
531. The method of example 529 or 530, wherein a concentration of sodium phosphate in a feed is between about 0 and about 525 mg/L, about 0 mg/L, about 250 mg/L, about 350 mg/L, or about 525 mg/L.
532. The method of any one of examples 509-531, wherein the culture medium is hydrolysate-free.
533. A method of producing an anti-IL-4Rα antibody or antigen-binding fragment thereof, comprising the steps of:
   (a) culturing cells expressing the anti-IL-4Rα antibody or antigen-binding fragment thereof in a large-scale bioreactor, wherein said bioreactor includes one or more optical probes for measuring dissolved gases;
   (b) culturing said cells in a culture medium comprising ornithine at between about 0.09 and about 0.9 mM and/or putrescine at between about 0.20 mM and about 0.9 mM; and
   (c) producing an anti-IL-4Rα antibody or antigen-binding fragment thereof.
534. The method of example 533, wherein said optical probe is used to measure dissolved oxygen.
535. The method of example 533 or 534, further comprising the step of agitating said culture medium with one or more impeller assemblies, wherein an uppermost impeller is positioned below the surface of an initial working volume of said bioreactor.
536. The method of any one of examples 49-148, 158-168, 188-234, 409-452, 468-495, 509-532, or 533-535, further comprising the step of adjusting dissolved oxygen levels by sparging said culture medium.
537. The method of any one of examples 49-148, 158-168, 188-234, 409-452, 468-495, 509-532, or 533-536, further comprising adjusting pCO2 levels by sparging said culture medium.
538. The method of any one of examples 49-148, 158-168, 188-234, 409-452, 468-495, or 509-537, further comprising the step of adding taurine or hypotaurine to culture medium.
539. The method of any one of examples 49-148, 158-168, 188-234, 409-452, 468-495, or 509-538, further comprising the step of adding at least one recombinant growth factor.
540. The method of any one of examples 49-148, 158-168, 188-234, 409-452, 468-495, or 509-539, further comprising the step of adding one or more of the following: adenosine, guanosine, cytidine, uridine, thymidine, and hypoxanthine.
541. The method of any one of examples 533-540, further comprising the step of adding fatty acids comprising one or more of the following: linoleic acid, linolenic acid, thioctic acid, oleic acid, palmitic acid, stearic acid, arachidic acid, arachidonic acid, lauric acid, behenic acid, decanoic acid, dodecanoic acid, hexanoic acid, lignoceric acid, myristic acid, and octanoic acid.
542. The method of any one of examples 533-541, further comprising the step of adding one or more salts selected from the group of divalent cations, such as calcium, magnesium, and a combination thereof
543. The method of any one of examples 509-542, further comprising the step of adding amino acids having a non-polar side chain.
544. The method of any one of examples 509-543, further comprising the step of adding basic amino acids.
545. The method of any one of examples 509-544, further comprising the step of adding nucleosides, salts of divalent cations, tocopherol, and vitamins.
546. A method of producing an anti-IL-4Rα antibody or antigen-binding fragment thereof in an improved bioreactor, comprising the steps of:
   (a) culturing cells expressing the anti-IL-4Rα antibody or antigen-binding fragment thereof in a vessel or bioreactor, wherein said bioreactor includes at least one on-line capacitance probe;
   (b) culturing said cells in a culture medium comprising one or more polyamines; and
   (c) producing an anti-IL-4Rα antibody or antigen-binding fragment thereof
547. The method of example 546, further comprising the steps of:
   i) applying an electric field to said cells cultured in a bioreactor;
   ii) measuring capacitance; and
   iii) correlating capacitance to viable cell density.
548. The method of example 547, further comprising the step of transferring said cells when a final VCD reaches a target cell density.
549. The method of any one of examples 509-548, further comprising the step of adjusting an initial VCD of a seed train to be at least 2.5×10⁵ cells/mL.
550. A method for treating a patient having a type 2 inflammatory disease, comprising administering to a patient an anti-IL4Rα antibody or antigen-binding fragment thereof produced according to the method of any one of examples 509-549.
551. The method of example 550, wherein said type 2 inflammatory disease is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.
552. A method for treating a disease or disorder associated with IL-4R activity, comprising administering to a patient an anti-IL4Rα antibody or antigen-binding fragment thereof produced according to the method of any one of examples 509-549.
553. The method of example 552, wherein said disease or disorder associated with IL-4R activity is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.
554. The method of any one of examples 1-553, wherein an energy dissipation rate is calculated using Equation 1 or Equation 2.
555. The method of any one of examples 1-554, wherein a starting volume of a 10,000 L bioreactor is from about 6500 L to about 7200 L or from about 7200 L to about 8000 L.
556. A method for treating a patient having a type 2 inflammatory disease, comprising administering to a patient Dupilumab produced using the bioreactor of any one of examples 180-187 or 496-500.
557. The method of example 556, wherein said type 2 inflammatory disease is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.
558. A method for treating a disease or disorder associated with IL-4R activity, comprising administering to a patient Dupilumab produced using the bioreactor of any one of examples 180-187 or 496-500.
559. The method of example 558, wherein said disease or disorder associated with IL-4R activity is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.
560. A method for treating a patient having a type 2 inflammatory disease, comprising administering to a patient Dupilumab produced using the cell culture medium of any one of examples 31-48.
561. The method of example 560, wherein said type 2 inflammatory disease is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.
562. A method for treating a disease or disorder associated with IL-4R activity, comprising administering to a patient Dupilumab produced using the cell culture medium of any one of examples 31-48.
563. The method of example 562, wherein said disease or disorder associated with IL-4R activity is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.
564. A method for treating a disease or disorder associated with IL-13 activity, comprising administering to a patient Dupilumab produced using the bioreactor of any one of examples 180-187 or 496-500.

What is claimed is:

1. A method of producing Dupilumab, comprising culturing cells with an initial viable cell density (VCD) in a seed train adjusted to be at least 2.5×10⁵ cells/mL.

2. The method of claim 1, wherein said seed train includes cell cultures in N-5 to N-1 vessels or bioreactors, wherein the initial VCD is adjusted between 3.5×10⁵ to 5.43×10⁵ cells/mL in each vessel or bioreactor.

3. The method of claim 2, wherein a final Dupilumab titer is about 2%, 3%, 4%, 5%, 6%, 7%, 8%, 9%, 10%, 11%, 12%, 13%, 14%, 15%, 1%, 17%, 18%, 19%, or 20% greater than a titer produced from a cell culture where the initial VCD in said N-5 to N-1 vessel or bioreactor is below 2.5×10⁵ cells/mL.

4. The method of claim 1, wherein the initial VCD is about 1.3×, 1.4×, 1.5×, 1.6, 1.7×, 1.8×, 1.9×, 2.0×, 2.1×, 2.2×, 2.3×, 2.4×, 2.5×, 2.6×, 2.7×, 2.8×, 2.9×, or 3.0× greater than an alternative initial VCD in a standard seed train.

5. The method of claim 3, wherein an increased final Dupilumab titer is not dependent on the final VCD in the N-1 seed train vessels or bioreactors.

6. The method of claim 2, wherein there is no substantial difference in peak lactate observed in a final production vessel compared to a final production vessel where the initial VCD vessels or bioreactors of the seed train is below 2.5×10⁵ cells/mL.

7. The method of claim 6, wherein the seed train resulted in a 2%, 3%, 4%, 5%, 6%, 7%, 8%, 9%, 10%, 11%, 12%, 13%, 14%, 15%, 1%, 17%, 18%, 19%, 20% increase in final titer (g/L).

8. The method of claim 7, wherein the initial VCD in a seed train is adjusted between 3.5×10⁵ to 5.43×10⁵ cells/mL.

9. A method of culturing a cell, the method comprising:
a) using at least one on-line capacitance probe to measure a first capacitance value of a first cell culture;
b) using at least one off-line assay to measure a first viable cell density value of the said first cell culture;
c) correlating said first capacitance value with the said first viable cell density value to determine a correlation equation;
d) using an on-line capacitance probe to determine a second capacitance value of a second cell culture;
e) using said second capacitance value and said correlation equation to predict at least one second viable cell density value of said second cell culture; and
f) adjusting a working volume or viable cell density (VCD) based on said second viable cell density value to culture the cell.

10. The method of claim 9, wherein said cell is from a cell line that is the same as a cell line used to derive said correlation equation.

11. The method of claim 9, wherein said correlation equation is generated using multivariate data analysis or a linear regression.

12. A method of producing Dupilumab comprising the steps of:
(a) culturing cells wherein an initial viable cell density (VCD) in a seed train in vessels or bioreactors is adjusted to at least 2.5×10⁵ cells/mL;
(b) measuring viable cell density by
(i) applying an electric field to said cells cultured in a vessel or bioreactor; and
(ii) measuring capacitance; and
(iii) correlating capacitance to viable cell density;
(c) adjusting initial VCD in each seed train vessel or bioreactor; and
(d) producing Dupilumab.

13. The method of claim 12, wherein the initial VCD in each vessel or bioreactor of an N-5 to N-1 seed train is adjusted between 3.5×10⁵ to 5.43×10⁵ cells/mL.

14. The method of claim 12, wherein a final Dupilumab titer is about 2%, 3%, 4%, 5%, 6%, 7%, 8%, 9%, 10%, 11%, 12%, 13%, 14%, 15%, 1%, 17%, 18%, 19%, or 20% greater than a titer produced from a cell culture where the initial VCD in said seed train is below 2.5×10⁵ cells/mL.

15. The method of claim 13, wherein the initial VCD is about 1.3×, 1.4×, 1.5×, 1.6, 1.7×, 1.8×, 1.9×, 2.0×, 2.1×, 2.2×, 2.3×, 2.4×, 2.5×, 2.6×, 2.7×, 2.8×, 2.9×, or 3.0× greater than an alternative initial VCD in a standard seed train.

16. The method of claim 15, wherein an increased final Dupilumab titer is not dependent on a final VCD in N-5 to N-1 vessels or bioreactors.

17. The method of claim 12, wherein there is no substantial difference in peak lactate observed in a final production vessel compared to a final production vessel or bioreactor where the initial VCD in each N-5 to N-1 vessel or bioreactor is below 2.5×10⁵ cells/mL.

18. The method of claim 17, wherein the seed train resulted in a 2%, 3%, 4%, 5%, 6%, 7%, 8%, 9%, 10%, 11%, 12%, 13%, 14%, 15%, 1%, 17%, 18%, 19%, 20% increase in final titer (g/L).

19. A method of producing an anti-IL-4Rα antibody or antigen-binding fragment thereof, the method comprising:
a) culturing cells capable of expressing an anti-IL-4Rα antibody or antigen-binding fragment thereof in a vessel or bioreactor;
b) adjusting an initial viable cell density (VCD) to be 2.5×10⁵ cells/mL or greater;
c) using at least one on-line sensor to measure a first property of a first cell culture;
d) using at least one off-line assay to measure a second property of the first cell culture;
e) correlating the measure of the first property of the first cell culture with the measure of the second property of the first cell culture to determine a correlation equation;
f) using an on-line sensor to measure the first property of a second cell culture;
g) using the measure of the first property of the second cell culture and the correlation equation to predict at least one measure of the second property of the second cell culture;
h) transferring said cells to another vessel or bioreactor based on the at least one predicted measure of the second property of the second cell culture; and
i) repeating steps b)-h) along a seed train.

20. The method of claim 19, wherein said cells are from a cell culture that is the same as the first cell culture.

21. The method of claim 19, wherein said cells are from a cell culture that is different from the first cell culture.

22. The method of claim 19, wherein said correlation equation is derived using more than one cell line.

23. The method of claim 19, wherein more than one measure of said first property of said first cell culture is taken.

24. The method of claim 19, wherein more than one measure of said second property of said first cell culture is taken.

25. The method of claim 19, wherein at least about 50 percent of variability in said measure of said second property of said first cell culture is due to variance in said measure of said first property of said first cell culture.

26. The method of claim 19, wherein said correlation equation is generated using multivariate data analysis or a linear regression.

27. The method of claim 19, wherein the initial VCD is about 1.3×, 1.4×, 1.5×, 1.6, 1.7×, 1.8×, 1.9×, 2.0×, 2.1×, 2.2×, 2.3×, 2.4×, 2.5×, 2.6×, 2.7×, 2.8×, 2.9×, or 3.0× greater than an alternative initial VCD in a standard seed train.

28. The method of claim 19, wherein there is no substantial difference in peak lactate observed in the final production vessel or bioreactor compared to a final production vessel where the initial VCD in N-5 to N-1 vessels is below 2.5×10⁵ cells/mL.

29. The method of claim 28, wherein the seed train resulted in a 2%, 3%, 4%, 5%, 6%, 7%, 8%, 9%, 10%, 11%, 12%, 13%, 14%, 15%, 16%, 17%, 18%, 19%, 20% increase in final titer (g/L).

30. The method of claim 28, wherein the initial VCD is about 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, or 3.0× greater than an alternative initial VCD in a standard seed train.

31. The method of claim 19, wherein said cells are CHO cells.

32. The method of claim 19, wherein said anti-IL-4Rα antibody is Dupilumab.

33. A method for treating a patient having a type 2 inflammatory disease, comprising administering to a patient an anti-IL4Rα antibody, an antigen-binding fragment thereof, or Dupilumab produced according to the method of any of claim 1-8 or 12-32.

34. The method of claim 33, wherein said type 2 inflammatory disease is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.

35. A method for treating a disease or disorder associated with IL-4R activity, comprising administering to a patient an anti-IL4Rα antibody, an antigen-binding fragment thereof, or Dupilumab produced according to the method of any of claim 1-8 or 12-32.

36. The method of claim 35, wherein said disease or disorder associated with IL-4R activity is atopic dermatitis, moderate-to-severe atopic dermatitis, asthma, moderate-to-severe asthma, allergic rhinitis, chronic rhinosinusitis with nasal polyposis, eosinophilic esophagitis, chronic obstructive pulmonary disease, chronic spontaneous urticaria, prurigo nodularis, allergic fungal rhino-sinusitis, chronic rhinosinusitis without nasal polyps, allergy, grass allergy, peanut allergy, dairy allergy, bullous pemphigoid, hand and foot atopic dermatitis, cold-induced urticaria, chronic inducible urticaria, ulcerative colitis, chronic pruritis of unknown origin, eosinophilic gastroenteritis, allergic bronchopulmonary aspergillosis, bronchiectasis, or alopecia areata.
